(19) United States
(12) Patent Application Publication
(10) Pub. No.: US 2023/0242496 A1
(43) Pub. Date: Aug. 3, 2023

(54) SOX11 INHIBITORS FOR TREATING MANTLE CELL LYMPHOMA

(71) Applicant: Icahn School of Medicine At Mount Sinai, New York, NY (US)

(72) Inventors: Jian JIN, New York, NY (US); Marta FILIZOLA, New York, NY (US); Aneel AGGARWAL, New York, NY (US); Samir PAREKH, New York, NY (US); Abhijeet KAPOOR, Rockville, MD (US); Shashidhar S. JATIANI, New York, NY (US); H. Umit KANISKAN, New York, NY (US); Jianping HU, New York, NY (US); Yudao SHEN, New York, NY (US); Fanye MENG, New York, NY (US); Lihuai QIN, New York, NY (US); Yulin HAN, New York, NY (US); Xufen YU, New York, NY (US); Chengwei ZHANG, Shanghai (CN); Prashasti KUMAR, New York, NY (US); Rinku JAIN, Vernon Hills, IL (US); Clement LEE, Hong Kong (CN)

(73) Assignee: Icahn School of Medicine At Mount Sinai, New York, NY (US)

(21) Appl. No.: 18/001,516

(22) PCT Filed: Jun. 15, 2021
(86) PCT No.: PCT/US21/37405

§ 371 Date: Dec. 12, 2022

Related U.S. Application Data
(60) Provisional application No. 63/039,704, filed on Jun. 16, 2020.

(51) Int. Cl. C07D 279/16; C07D 265/36; C07C 321/28; C07D 249/18; C07D 215/48; C07D 239/74; C07D 231/56; C07D 277/64; C07D 235/06; C07D 335/06; C07D 209/34; C07D 263/58; C07D 285/14; C07D 217/02; C07D 417/12; C07D 239/42; C07D 233/64; C07D 403/04; C07D 215/38; C07D 239/72; C07C 311/46; C07D 295/135; C07D 239/48; C07D 235/16; C07D 215/227; C07D 295/26; C07D 239/54; C07D 405/12

(52) U.S. Cl. CPC C07D 279/16; C07D 265/36; C07C 321/28; C07D 249/18; C07D 215/48; C07D 239/74; C07D 231/56; C07D 277/64; C07D 235/06; C07D 335/06; C07D 209/34; C07D 263/58; C07D 285/14; C07D 217/02; C07D 417/12; C07D 239/42; C07D 233/64; C07D 403/04; C07D 215/38; C07D 239/72; C07C 311/46; C07D 295/135; C07D 239/48; C07D 235/16; C07D 215/227; C07D 295/26; C07D 239/54; C07D 405/12

(57) ABSTRACT

Disclosed are compounds that are chemical inhibitors of SOX11. The compounds disclosed are useful in treatment of various cancers.

This application claims priority of U.S. provisional application 63/039,704, filed Jun. 16, 2020, the entire disclosure of which is hereby incorporated herein by reference.

### FIELD OF THE INVENTION

This invention relates to compounds that are chemical inhibitors of SOX11. The compounds disclosed are useful in treatment of various cancers.

### BACKGROUND OF THE INVENTION

Mantle cell lymphoma (MCL) is a type of non-Hodgkin's lymphoma (NHL), comprising about 6% of NHL cases. MCL is a subtype of B-cell lymphoma, due to CD5 positive antigen-naive pre-germinal center B-cells within the mantle zone that surrounds normal germinal center follicles.

MCL results from the acquisition of a combination of non-inherited genetic mutations in somatic cells, leading to a clonal expansion of malignant B lymphocytes. The factors that initiate the genetic alterations are typically not identifiable, and usually occur in people with no particular risk factors for lymphoma development. A defining characteristic of MCL is mutation and overexpression of cyclin D1, a cell cycle gene, that contributes to the abnormal proliferation of the malignant cells. Cells affected by MCL proliferate in a nodular or diffuse pattern with two main cytologic variants, typical or blastic. Typical cases are small to intermediate-sized cells with irregular nuclei. Blastic (aka blastoid) variants have intermediate- to large-sized cells with finely dispersed chromatin and are more aggressive in nature. The tumor cells accumulate in the lymphoid system, including lymph nodes and the spleen, with non-useful cells eventually rendering the system dysfunctional. MCL may also replace normal cells in the bone marrow, which impairs normal blood cell production.

SOX11 is a transcription factor involved in the regulation of embryonic development and in the determination of the cell fate. SOX11 immuno-histochemical expression is present in 78-93% of lymph node biopsies from MCL patients and is specific for MCL as compared to other NHL. SOX11 expression is present in pre-malignant lymph nodes, suggesting that this is an early event in the malignant transformation of lymphocytes in MCL. SOX11 depletion by RNAi in human MCL cell lines has demonstrated reduced growth in xenograft models. Multiple lines of evidence support SOX11 as an MCL oncogene.

The overall 5-year survival rate for MCL is generally 50% (advanced stage MCL) to 70% (for limited-stage MCL). Prognosis for individuals with MCL is problematic and indexes do not work as well due to patients presenting with advanced stage disease. Staging is used but is not very informative, since the malignant B-cells can travel freely though the lymphatic system and therefore most patients are at stage III or IV at diagnosis.

Regimens to treat MCL are available and often get good response rates, but patients almost always get disease progression after chemotherapy. Each relapse is typically more difficult to treat, and relapse is generally faster. Ibrutinib, a BCR signaling (BTK) inhibitor, has recently demonstrated significant therapeutic activity in MCL, with responses seen in 60% of relapsed patients. However, the majority of patients relapse after Ibrutinib treatment. MCL cells may also be resistant to drug-induced apoptosis, making them harder to cure with chemotherapy or radiation. Thus, there is an urgent need for improved MCL therapies.

### SUMMARY OF THE INVENTION

Described herein are small molecule inhibitors of SOX11 that show potent and specific cytotoxicity in SOX11 expressing cells, thereby indicating significant potential for the treatment of patients suffering from MCL.

### DETAILED DESCRIPTION OF THE INVENTION

In one aspect, the invention relates to compounds of Formula I:

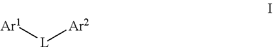

wherein:
i) Ar¹ is

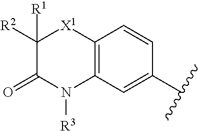

wherein:
   X¹ is S or O;
   R¹ and R² are independently selected from optionally substituted (C₁-C₆)alkyl and H; and
   R³ is H or (C₁-C₁₀)hydrocarbyl;
L is —CONH—, —NHCO—, or —NHCH₂—; and
Ar² is a mono- or di-substituted monocyclic aryl or heteroaryl, wherein the substituents are selected from —SO₂—R¹⁰, —OSO₂—R¹⁰, perfluoro(C₁-C₃)alkyl, halo, (C₁-C₃)alkyl, —C(═O)R¹⁰, —OCH₂R¹¹, —OR¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰,
   wherein R¹⁰ is selected from arylamino, perfluoro(C₁-C₃)alkyl-substituted arylamino, halo-substituted arylamino, (C₁-C₃)alkyl-substituted arylamino, amino, (C₁-C₃)alkyl, heterocyclyl, (C₁-C₆)dialkylamino, pyridylamino, (C₁-C₆)alkylamino, (C₁-C₆)cycloalkylamino, arylamino, oxo-substituted heteroarylamino, heterocyclylamino, hydroxy-substituted arylamino, amino-substituted arylamino, pyridin-2(1H)-one-amino, (C₁-C₆)dihydrocarbylamino, fluoro, (C₁-C₃)alkylarylamino, acetyl-substituted heterocyclyl, and (C₁-C₃)alkylhaloarylamino; and
   wherein R¹¹ is selected from optionally substituted aryl, unsubstituted benzyl, perfluoro(C₁-C₃)alkyl-substituted benzyl, halo-substituted benzyl, (C₁-C₃)alkyl-substituted benzyl, (C₁-C₃)alkyl, heterocyclyl, (C₁-C₆)dialkyl, pyridyl, (C₁-C₆)alkyl, (C₁-C₆)cycloalkyl, benzyl, oxo-substituted heteroarylbenzyl, heterocyclyl, hydroxy-substituted benzyl, amino-substituted benzyl, pyridin-2(1H)-one, (C₁-C₆)dihydrocarbyl, (C₁-C₃)alkylbenzyl, acetyl-substituted heterocyclyl, and (C₁-C₃)alkylhalobenzyl; or
ii) Ar¹ is

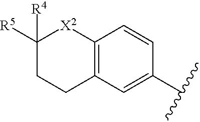

wherein:
   X² is S or O; and
   R⁴ and R⁵ are independently selected from H and (C₁-C₆)alkyl;
L is —CONH— or —NHCO—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰;
   wherein R¹⁰ is selected from arylamino, perfluoro(C₁-C₃)alkyl-substituted arylamino, halo-substituted arylamino, (C₁-C₃)alkyl-substituted arylamino, amino, (C₁-C₃)alkyl, heterocyclyl, (C₁-C₆)dialkylamino, pyridylamino, (C₁-C₆)alkylamino, (C₁-C₆)cycloalkylamino, arylamino, oxo-substituted heteroarylamino, heterocyclylamino, hydroxy-substituted arylamino, amino-substituted arylamino, pyridin-2(1H)-one-amino, (C₁-C₆)dihydrocarbylamino, fluoro, (C₁-C₃)alkylarylamino, acetyl-substituted heterocyclyl, and (C₁-C₃)alkylhaloarylamino; and
   wherein R¹¹ is selected from benzyl, perfluoro(C₁-C₃)alkyl-substituted benzyl, halo-substituted benzyl, (C₁-C₃)alkyl-substituted benzyl, (C₁-C₃)alkyl, heterocyclyl, (C₁-C₆)dialkyl, pyridyl, (C₁-C₆)alkyl, (C₁-C₆)cycloalkyl, benzyl, oxo-substituted heteroarylbenzyl, heterocyclyl, hydroxy-substituted benzyl, amino-substituted benzyl, pyridin-2(1H)-one, (C₁-C₆)dihydrocarbyl, (C₁-C₃)alkylbenzyl, acetyl-substituted heterocyclyl, and (C₁-C₃)alkylhalobenzyl; or
iii) Ar¹ is

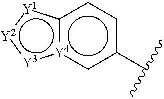

wherein:
   all backbone atoms of the 6,5-bicyclic structure are sp²-hybridized;
   Y¹ is selected from S, CH, N, NH, and O;
   Y² is selected from N, NH, C—R⁶, and C═O; wherein R⁶ is H, (C₁-C₃)alkyl, or amino(C₁-C₃)alkyl;
   Y³ is selected from N, NH, CH, and C—CH₃; and
   Y⁴ is C or N;
L is —CONH— or —NHCO—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰; or
iv) Ar¹ is

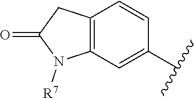

wherein R⁷ is H or optionally substituted (C₁-C₃)alkyl;
L is —CONH— or —NHCO—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰; or
v) Ar¹ is

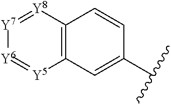

wherein Y⁵, Y⁶, Y⁷, and Y⁸ are independently chosen from C and N;
L is —CONH—, —NHCO—, or —NHCH₂—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰; or
vi) Ar¹ is

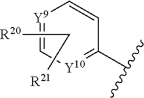

wherein Y⁹ and Y¹⁰ are independently chosen from C and N;
R²⁰ and R²¹ are independently chosen from hydrogen, (C₁-C₃)alkyl, aryl-substituted heterocyclic amino, heteroaryl-substituted heterocyclic amino, unsubstituted heterocyclic amino, amino, —CH═CHCOOH, 4-aminocyclohexylamino, acetylethylenediamino, amino- and/or (C₁-C₃)alkyl-substituted heterocyclic amino, —NHC(═O)(CH₂)ₙ-heterocyclyl wherein n is either 1 or 2, ethylenediamino, (C₁-C₃)alkoxy, and acetylmethylamino;
L is —CONH—,

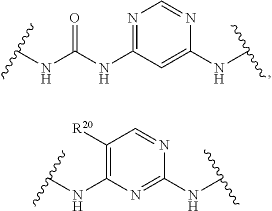

wherein R²⁰ is H or methyl; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰.

In a second aspect, the invention relates to compounds of Formula II:

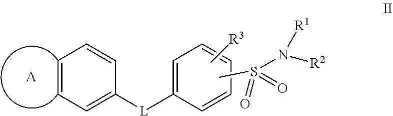

wherein:

R¹ is selected from hydrogen and optionally substituted C₁-C₄ alkyl;

R² is selected from C₁-C₄ alkyl; C₃-C₆ cycloalkyl; tert-butyl piperidine-1-carboxylate; pyridin-2(1H)-one or phenyl optionally substituted with C₁-C₄ alkyl, C₁-C₄ haloalkyl, —OH, or halogen; or

taken together with the nitrogen to which they are attached, R¹ and R² form a five- to seven-membered, non-aromatic heterocyclic ring optionally substituted with tert-butyl carboxylate, wherein said heterocyclic ring contains no additional —NH— group.

R³ is selected from hydrogen, halogen, C₁-C₄ alkyl, or C₁-C₄ haloalkyl;

L is selected from

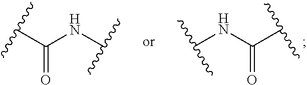 [SMILES: *NC(*)=O ; *NC(*)=O]

Ring A is selected from

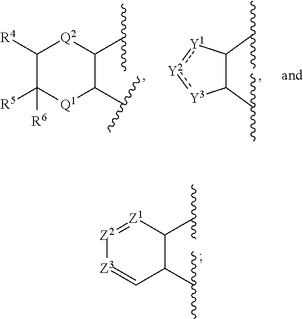

wherein:

Q¹ is selected from NH, NCH₃, or CH₂;

Q² is selected from S or O;

R⁴ is selected from hydrogen and C₁-C₄ alkyl;

R⁵ and R⁶ are each independently hydrogen; or

R⁵ and R⁶ taken together form ═O;

[symbol] represents a single bond or a double bond;

Y¹ is selected from S, CH, NR^{Y1}, or O;

Y² is selected from NR^{Y1}, CR^{Y2}, or C═O;

Y³ is selected from NR^{Y1} or CR^{Y2};

wherein at least one of Y¹, Y², and Y³ is NR^{Y1};

R^{Y1} is either hydrogen or a lone pair on the nitrogen atom to which it is attached;

R^{Y2} is selected from hydrogen or CH₃;

Z¹, Z², and Z³ are each independently selected from CH and N; wherein one of Z¹, Z², and Z³ is N and the remaining two of Z¹, Z², and Z³ are CH;

In a third aspect, the invention relates to a method or medicament for treating cancer in a patient, wherein said cancer is selected from mantle cell lymphoma, basal-cell like breast cancer, and neuroblastoma.

In a fourth aspect, the invention relates to a method or medicament for treating a disease or disorder in a patient where the disease or disorder involves the inhibition of SOX-11.

In a fifth aspect, the invention relates to a method or medicament for inhibiting SOX-11 expression in a patient.

Throughout this specification the terms and substituents retain their definitions. The description provided herein uses certain terms known in the chemical arts. Unless otherwise specified throughout the description herein, terms retain their meaning as understood by one having ordinary skill in the art.

As used herein, the terms “comprising” and “including” or grammatical variants thereof are to be taken as specifying the stated features, integers, steps or components, but do not preclude the addition of one or more additional features, integers, steps, components or groups thereof. This term encompasses the terms “consisting of” and “consisting essentially of”. The phrase “consisting essentially of” or grammatical variants thereof, when used herein, is to be taken as specifying the stated features, integers, steps or components, but does not preclude the addition of one or more additional features, integers, steps, components or groups thereof, but only if the additional features, integers, steps, components or groups thereof do not materially alter the basic and novel characteristics of the claimed composition or method.

As used herein, the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method that “comprises”, “has”, “includes” or “contains” one or more steps or elements possesses those one or more steps or elements but is not limited to possessing only those one or more steps or elements.

A “patient,” as used herein, includes both humans and other animals, particularly mammals. Thus the methods are applicable to both human therapy and veterinary applications. In some embodiments, the patient is a mammal, for example, a primate. In some embodiments, the patient is a human.

Treatment can involve administering a compound described herein to a patient diagnosed with a disease and may involve administering the compound to a patient who does not have active symptoms. Conversely, treatment may involve administering the compositions to a patient at risk of developing a particular disease, or to a patient reporting one or more of the physiological symptoms of a disease, even though a diagnosis of this disease may not have been made.

The terms “administer”, “administering” or “administration” in reference to a dosage form of the invention refers to the act of introducing the dosage form into the system of subject in need of treatment. When a dosage form of the invention is given in combination with one or more other active agents (in their respective dosage forms), “administration” and its variants are each understood to include concurrent and/or sequential introduction of the dosage form and the other active agents. Administration of any of the described dosage forms includes parallel administration, co-administration or sequential administration. In some situations, the therapies are administered at approximately the same time, e.g., within about a few seconds to a few hours of one another.

A “therapeutically effective” amount of a compound described herein is typically one which is sufficient to achieve the desired effect and may vary according to the nature and severity of the disease condition, and the potency of the compound. It will be appreciated that different concentrations may be employed for prophylaxis than for treatment of an active disease. A therapeutic benefit is achieved with the amelioration of one or more of the physiological symptoms associated with the underlying disorder such that an improvement is observed in the patient, notwithstanding that the patient may still be afflicted with the underlying disorder.

Throughout this specification the terms and substituents retain their definitions.

“Hydrocarbon” (or “hydrocarbyl” when it is a residue) includes alkyl, cycloalkyl, polycycloalkyl, alkenyl, alkynyl, aryl and combinations thereof. Examples include benzyl, phenethyl, cyclohexylmethyl, adamantyl, camphoryl and naphthylethyl. Hydrocarbyl refers to any substituent comprised of hydrogen and carbon as the only elemental constituents. A prefix such as “Cₓ-C_{y}” or “(Cₓ-C_{y})” indicates that the group following the prefix has from x to y carbon atoms. For example, a “C₁ to C₂₀ hydrocarbon” indicates a hydrocarbon having 1 to 20 carbon atoms. Aliphatic hydrocarbons are hydrocarbons that are not aromatic; they may be saturated or unsaturated, cyclic, linear or branched. Examples of aliphatic hydrocarbons include isopropyl, 2-butenyl, 2-butynyl, cyclopentyl, norbornyl, etc. Aromatic hydrocarbons include benzene (phenyl), naphthalene (naphthyl), anthracene, etc.

Unless otherwise specified, alkyl (or alkylene when divalent) is intended to include linear or branched saturated hydrocarbon structures and combinations thereof. Unless otherwise defined, “alkyl” refers to alkyl groups from 1 to 20 carbon atoms, preferably 1 to 10 carbon atoms, more preferably 1 to 6 carbon atoms. Examples of alkyl groups include methyl, ethyl, propyl, isopropyl, n-butyl, s-butyl, t-butyl and the like.

Cycloalkyl is a subset of hydrocarbon and includes cyclic hydrocarbon groups of from 3 to 8 carbon atoms. Examples of cycloalkyl groups include cyclopropyl, cyclobutyl, cyclopentyl, norbornyl and the like.

Unless otherwise specified, the term “carbocycle” is intended to include ring systems in which the ring atoms are all carbon but of any oxidation state. Thus (C₃-C₁₀) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

Heterocycle means an aliphatic or aromatic carbocycle residue in which from one to four carbons has been replaced by a heteroatom selected from the group consisting of N, O, and S. Unless otherwise specified, a heterocycle may be non-aromatic (heteroaliphatic) or aromatic (heteroaryl). Examples of heterocycles include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. Examples of heterocyclyl residues include piperazinyl, piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, benzoxazolyl, tetrahydrofuryl, tetrahydropyranyl, thienyl (also historically called thiophenyl), benzothienyl, thiamorpholinyl, oxadiazolyl, triazolyl and tetrahydroquinolinyl.

Monocyclic heterocyclyl or monocyclic heterocycle means an aromatic or non-aromatic heterocycle composed of a single ring. Examples of monocyclic heterocycles include furan, thiophene, pyrrole, pyrazole, oxazole, oxadiazole, thiazole, isoxazole, isothiazole, imidazole, triazole, pyridine, pyrimidine, pyrazine, and pyridazine. Bicyclic heterocyclyl means an aromatic or non-aromatic heterocycle composed of two fused rings wherein one or both of the rings contain a heteroatom. Thus, bicyclic heterocyclyl includes fused bicyclic structures that have no heteroatom in one ring but contain one or more heteroatoms in the other ring. Neither ring need be aromatic but one or both rings may be aromatic. However, if at least one ring is aromatic, then the bicyclic heterocyclyl is considered aromatic. Examples of bicyclic heterocycles include indole, isoindole, benzimidazole, benzofuran, benzothiophene, benzooxadiazole, benzothiazole, pyrazolopyridine, quinoline, isoquinoline, quinazoline, quinoxaline, benzodioxole, dihydrobenzooxazine, and purine.

Hydrocarbyloxy refers to groups of from 1 to 20 carbon atoms, preferably 1 to 10 carbon atoms, more preferably 1 to 6 carbon atoms attached to the parent structure through an oxygen. Alkoxy is a subset of hydrocarbyloxy and includes groups of a straight or branched configuration. Examples include methoxy, ethoxy, propoxy, isopropoxy and the like. Lower-alkoxy refers to groups containing one to four carbons. The term “halogen” means fluorine, chlorine, bromine or iodine atoms.

Unless otherwise specified, acyl refers to formyl and to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. Examples include acetyl, benzoyl, propionyl, isobutyryl and the like. Lower-acyl refers to groups containing one to four carbons.

As used herein, the term “optionally substituted” may be used interchangeably with “unsubstituted or substituted”. The term “substituted” refers to the replacement of one or more hydrogen atoms in a specified group with a specified radical. For example, substituted alkyl, aryl, cycloalkyl, heterocyclyl etc. refer to alkyl, aryl, cycloalkyl, or heterocyclyl wherein one or more H atoms in each residue are replaced with halogen, haloalkyl, alkyl, acyl, alkoxyalkyl, hydroxy lower alkyl, carbonyl, phenyl, heteroaryl, benzenesulfonyl, hydroxy, lower alkoxy, haloalkoxy, oxaalkyl, carboxy, alkoxycarbonyl [—C(═O)O-alkyl], alkoxycarbonylamino [HNC(═O)O-alkyl], aminocarbonyl (also known as carboxamido) [—C(═O)NH₂], oxo [═O] alkylaminocarbonyl [—C(═O)NH-alkyl], cyano, acetoxy, nitro, amino, alkylamino, dialkylamino, (alkyl)(aryl)aminoalkyl, alkylaminoalkyl (including cycloalkylaminoalkyl), dialkylaminoalkyl, dialkylaminoalkoxy, heterocyclylalkoxy, mercapto, alkylthio, sulfoxide, sulfone, sulfonylamino, alkylsulfinyl, alkyl sulfonyl, acylaminoalkyl, acylaminoalkoxy, acylamino, amidino, aryl, benzyl, heterocyclyl, heterocyclylalkyl, phenoxy, benzyloxy, heteroaryloxy, hydroxyimino, alkoxyimino, oxaalkyl, aminosulfonyl, trityl, amidino, guanidino, ureido, benzyloxyphenyl, and benzyloxy. In one embodiment, 1, 2, or 3 hydrogen atoms are replaced with a specified radical. In the case of alkyl and cycloalkyl, more than three hydrogen atoms can be replaced by fluorine; indeed, all available hydrogen atoms could be replaced by fluorine.

Substituents Rn are generally defined when introduced and retain that definition throughout the specification and in all independent claims.

One or more compounds described herein may contain up to two asymmetric centers and may thus give rise to enantiomers, diastereomers, and other stereoisomeric forms which may be defined in terms of absolute stereochemistry as (R)- or (S)-. The present invention is meant to include all such possible isomers as racemates, optically pure forms and intermediate mixtures. Optically active isomers may be prepared using homo-chiral synthons or homo-chiral reagents, or optically resolved using conventional techniques such as chiral chromatography. All tautomeric forms are intended to be included. The graphic representations of racemic, ambiscalemic and scalemic or enantiomerically pure compounds used herein are taken from Maehr J. Chem. Ed. 62, 114-120 (1985): simple, single bond lines convey connectivity only and no stereochemical implication; solid and broken wedges are used to denote the absolute configuration of a chiral element; wavy lines indicate explicit disavowal of any stereochemical implication which the bond it represents could generate; solid and broken bold lines are geometric descriptors indicating the relative configuration shown but do not denote absolute configurations; and wedge outlines and dotted or broken lines denote enantiomerically pure compounds of indeterminate absolute configuration. Enantiomerically pure means greater than 80 e.e., and preferably greater than 90 e.e.

For example, the graphic representation

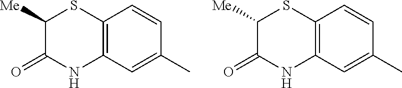

indicates the shown absolute configuration. The graphic representation:

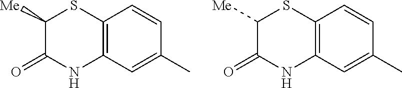

indicates a single enantiomer of unknown absolute stereochemistry, i.e. it could be either of the two preceding structures, as a substantially pure single enantiomer. For the purpose of the present disclosure, a “pure” or “substantially pure” enantiomer is intended to mean that the enantiomer is at least 95% of the configuration shown and 5% or less of other enantiomers.

It may be found upon examination that certain species and genera are not patentable to the inventors in this application. In this case, the exclusion of species and genera in applicants' claims are to be considered artifacts of patent prosecution and not reflective of the inventors' concept or description of their invention, which encompasses all members of the genus that are not in the public's possession.

Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder. In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life). A therapeutic benefit is achieved with the eradication or amelioration of one or more of the physiological systems associated with the underlying disorder such that an improvement is observed in the patient, notwithstanding that the patient may still be afflicted with the underlying disorder.

As used herein, and as would be understood by the person of skill in the art, the recitation of “a compound”—unless expressly further limited—is intended to include salts of that compound. In a particular embodiment, the term “compound of formula” refers to the compound or a pharmaceutically acceptable salt thereof.

The term “pharmaceutically acceptable salt” refers to salts prepared from pharmaceutically acceptable non-toxic acids or bases including inorganic acids and bases and organic acids and bases. When the compounds of the present invention are basic—as they are in most cases—salts may be prepared from pharmaceutically acceptable non-toxic acids including inorganic and organic acids. Preferable examples of salts with inorganic bases include alkali metal salts such as sodium salts, potassium salts and the like; alkali earth metal salts such as calcium salts, magnesium salts and the like; aluminum salts; and ammonium salts. Preferable examples of salts with organic bases include salts with trimethylamine, triethylamine, pyridine, picoline, ethanolamine, diethanolamine, triethanolamine, dicyclohexylamine, N,N-dibenzylethylenediamine and the like. Preferable examples of salts with inorganic acids include salts with hydrochloric acid, hydrobromic acid, nitric acid, sulfuric acid, phosphoric acid and the like. Preferable examples of salts with organic acids include salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid and the like. Preferable examples of salts with basic amino acids include salts with arginine, lysine, ornithine and the like. Preferable examples of salts with acidic amino acids include salts with aspartic acid, glutamic acid and the like.

Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic functionality (e.g. —SO₃H), suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

A compound of the present invention may be also used as a prodrug thereof, which is converted to the compound by a reaction such as oxidation, reduction, hydrolysis, and the like due to an enzyme, gastric acid etc. under the physiological condition in the living body. A prodrug of the compound of the present invention may be obtained by; subjecting an amino group in the compound to an acylation, alkylation or phosphorylation eicosanoylation, alanylation, pentylaminocarbonylation, (e.g. 5-methyl-2-oxo-1,3-dioxolen-4-yl)methoxycarbonylation, tetrahydrofuranylation, pyrrolidylmethylation, pivaloyloxymethylation and tert-butylation, etc.); subjecting a hydroxyl group in the compound to an acylation, alkylation, phosphorylation or boration (e.g. acetylation, palmitoylation, propanoylation, pivaloylation, succinylation, fumarylation, alanylation and dimethylaminomethylcarbonylation)); subjecting a carboxyl group in the compound to an esterification or amidation (e.g., an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification and methylamidation) and the like. Any of these prodrugs of the compound of the present invention can be produced by a method known per se.

A compound of the present invention may be labeled with an isotope (e.g., ²H, ³H, ¹⁴C, ³⁵S, ¹²⁵I, ¹¹C, ¹⁸F) and the like. The compound labeled with or substituted by an isotope can be used, for example, as a tracer used for Positron Emission Tomography (PET) (PET tracer), and is useful in the field of medical diagnosis and the like.

A compound of the present invention may be an anhydrate or a hydrate. The compound may be a solvate or a non-solvate. Furthermore, the compound may be a deuterated compound.

A compound of the present invention may be a crystal, and both a single crystal and crystal mixtures are encompassed in the compound. Crystals can be produced by crystallization according to crystallization methods known per se.

In addition, the compound may be a pharmaceutically acceptable cocrystal or cocrystal salt. Here, the cocrystal or cocrystal salt means a crystalline substance consisting of two or more particular substances which are solids at room temperature, each having different physical properties (e.g., structure, melting point, heat of melting, hygroscopicity, and stability). The cocrystal and cocrystal salt can be produced by cocrystallization method known per se.

The compound of the present invention, salt thereof, or a prodrug thereof (hereinafter sometimes to be simply abbreviated as the compound of the present invention) has low toxicity (e.g., acute toxicity, chronic toxicity, genetic toxicity, reproductive toxicity, cardiotoxicity, carcinogenicity), and can be used as it is or in the form of a pharmaceutical composition to mammals (e.g., human, mouse, rat, rabbit, dog, cat, bovine, horse, swine, monkey) as an agent for the prophylaxis or treatment of diseases as separately mentioned. A pharmaceutical composition comprising a compound of the present invention as disclosed above, together with one or more pharmaceutically carriers thereof and optionally one or more other therapeutic ingredients. The carrier(s) must be “acceptable” in the sense of being compatible with the other ingredients of the formulation and not deleterious to the recipient thereof.

As a pharmaceutically acceptable carrier here, common organic or inorganic carrier substances are used as formulation raw materials. Carriers are added as vehicles (e.g., lactose, sucrose, D-mannitol, D-sorbitol, starch, α-starch, dextrin, crystalline cellulose, low-substituted hydroxypropyl cellulose, sodium carboxymethylcellulose, gum Arabic, pullulan, light anhydrous silicic acid, synthetic aluminum silicate, and magnesium metasilicic aluminate), lubricants (e.g., magnesium stearate, calcium stearate, talc, colloidal silica, and the like), binders (e.g., α-starch, sucrose, gelatin, gum Arabic, methylcellulose, carboxymethylcellulose, sodium carboxymethylcellulose, crystalline cellulose, sucrose, D-mannitol, trehalose, dextrin, pullulan, hydroxypropylcellulose, hydroxypropyl methylcellulose, and polyvinylpyrrolidone) and disintegrants (e.g., lactose, sucrose, starch, carboxymethylcellulose, calcium carboxymethylcellulose, croscarmellose sodium, sodium carboxymethyl starch, light anhydrous silicic acid, and low-substituted hydroxypropylcellulose) in the solid formulations; and as solvents, solubilizing agents, suspending agents, isotonization agents, buffering agents, soothing agents etc. in the liquid formulations. If desired, formulation additives such as preservatives, antioxidants, colorants, sweeteners, etc. can be used.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of formula I or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

The content of a compound of the present invention in the formulation of the present invention varies based on the dosage forms, dosages of the compound of the present invention, and the like. For example, it is approximately about 0.1 to 100 wt %.

The dosage of a compound of the present invention varies depending on the administration subjects, administration routes, target diseases, symptoms, and the like. For example, for oral administration to adult patients with cancer, generally a single dose is about 0.01 to 100 mg/kg body weight, preferably 0.1 to 50 mg/kg body weight, further preferably 0.5 to 20 mg/kg body weight, and this dosage is preferably administered 1 to 3 times daily.

### Chemistry

In general, the production methods for the compounds of the present invention are explained with the following:

The starting materials and reagents used for each step in the following production methods, and the obtained compounds may each form a salt. Examples of the salts include those similar to the aforementioned salts of the compound of the present invention and the like.

When the compound obtained in each step is a free compound, it may be converted to a desired salt by a method known per se. Conversely, when the compound obtained in each step is a salt, it may be converted to a free form or a desired other kind of salt by a method known per se.

The compound obtained in each step may also be used for the next reaction as a reaction mixture thereof or after obtaining a crude product thereof. Alternatively, the compound obtained in each step may be isolated and/or purified from the reaction mixture by a separation means such as concentration, crystallization, recrystallization, distillation, solvent extraction, fractionation, chromatography and the like according to a conventional method.

When the starting materials and reagent compounds of each step are commercially available, the commercially available products are often used as is.

In the reaction of each step, unless otherwise specified, it is performed without solvent or by dissolving or suspending in a suitable solvent. Specific examples of the solvent include those described in Examples and the following:
alcohols: methanol, ethanol, tert-butyl alcohol, 2-methoxyethanol and the like;
ethers: diethyl ether, diphenyl ether, tetrahydrofuran, 1,2-dimethoxyethane and the like;
aromatic hydrocarbons: chlorobenzene, toluene, xylene and the like;
saturated hydrocarbons: cyclohexane, hexane and the like;
amides: N,N-dimethylformamide, N-methylpyrrolidone and the like;
halogenated hydrocarbons: dichloromethane, carbon tetrachloride and the like;
nitriles: acetonitrile and the like;
sulfoxides: dimethyl sulfoxide and the like;
aromatic organic bases: pyridine and the like;
acid anhydrides: acetic anhydride and the like;
organic acids: formic acid, acetic acid, trifluoroacetic acid and the like;
inorganic acids: hydrochloric acid, sulfuric acid and the like;
esters: ethyl acetate and the like;
ketones: acetone, methyl ethyl ketone and the like; and
water.

Two or more kinds of the above-mentioned solvents may be used by mixing at an appropriate ratio.

When a base is used in the reaction of each step, for example, bases shown below or those described in Examples are used:
inorganic bases: sodium hydroxide, magnesium hydroxide, sodium carbonate, calcium carbonate, sodium hydrogen carbonate and the like;
organic bases: triethylamine, diethylamine, pyridine, 4-dimethylaminopyridine, N,N-dimethylaniline, 1,4-diazabicyclo[2.2.2]octane, 1,8-diazabicyclo[5.4.0]-7-undecene, imidazole, piperidine and the like;
metal alkoxides: sodium ethoxide, potassium tert-butoxide and the like;
alkali metal hydrides: sodium hydride and the like;
metal amides: sodium amide, lithium diisopropyl amide, lithium hexamethyl disilazide and the like; and
organic lithiums: n-butyllithium and the like.

When an acid or acidic catalyst is used in the reaction of each step, for example, acids and acidic catalysts shown below or those described in Examples are used:
inorganic acids: hydrochloric acid, sulfuric acid, nitric acid, hydrobromic acid, phosphoric acid and the like;
organic acids: acetic acid, trifluoroacetic acid, citric acid, p-toluenesulfonic acid, 10-camphorsulfonic acid and the like; and
Lewis acids: boron trifluoride diethyl ether complex, zinc iodide, anhydrous aluminum chloride, anhydrous zinc chloride, anhydrous iron chloride and the like.

Unless defined otherwise, all technical and scientific terms used herein have the same meaning as is commonly understood by one of ordinary skill in the art to which this disclosure belongs. A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference. In the event that there is a plurality of definitions for terms cited herein, those in this section prevail unless otherwise stated.

The synthesis of exemplary compounds of the invention are shown below. The examples do not limit the present invention and the present invention can be modified within the scope of the present invention.

TABLE 1
Exemplary Compounds of the Present Invention (Synthesis described below).

| Ex. # | Structure |
|---|---|
| 1 | 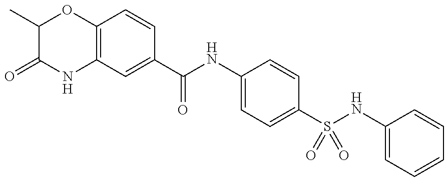 [SMILES: CC1Oc2ccc(C(=O)Nc3ccc(S(=O)(=O)Nc4ccccc4)cc3)cc2NC1=O] |
| 2 | 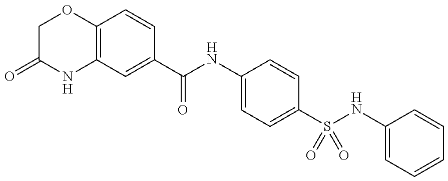 [SMILES: O=C1COc2ccc(C(=O)Nc3ccc(S(=O)(=O)Nc4ccccc4)cc3)cc2N1] |
| 3 | 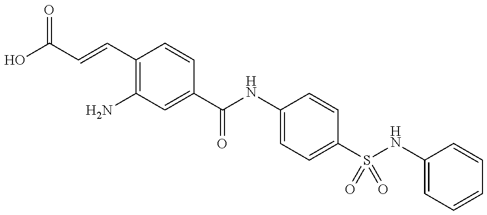 [SMILES: Nc1cc(C(=O)Nc2ccc(S(=O)(=O)Nc3ccccc3)cc2)ccc1/C=C/C(=O)O] |
| 4 | 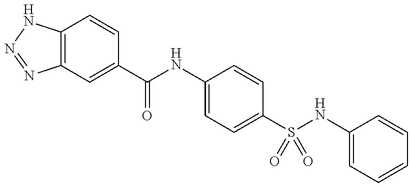 [SMILES: O=C(Nc1ccc(S(=O)(=O)Nc2ccccc2)cc1)c1ccc2[nH]nnc2c1] |
| 5 | 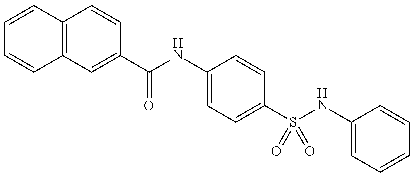 [SMILES: O=C(Nc1ccc(S(=O)(=O)Nc2ccccc2)cc1)c1ccc2ccccc2c1] |
| 6 | 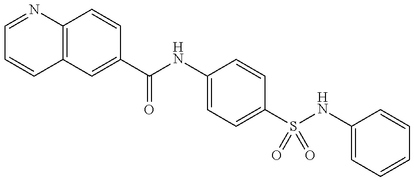 [SMILES: O=C(Nc1ccc(S(=O)(=O)Nc2ccccc2)cc1)c1ccc2ncccc2c1] |
| 7 | 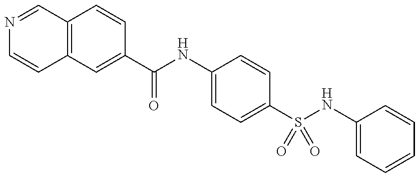 [SMILES: O=C(Nc1ccc(S(=O)(=O)Nc2ccccc2)cc1)c1ccc2cnccc2c1] |
| 8 | 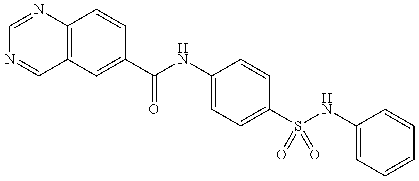 [SMILES: O=C(Nc1ccc(S(=O)(=O)Nc2ccccc2)cc1)c1ccc2ncncc2c1] |
| 9 | 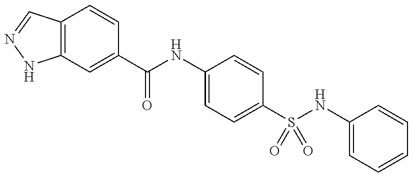 [SMILES: O=C(Nc1ccc(S(=O)(=O)Nc2ccccc2)cc1)c1ccc2cn[nH]c2c1] |
| 10 | 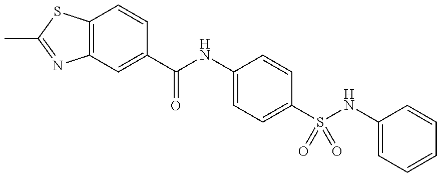 [SMILES: Cc1nc2cc(C(=O)Nc3ccc(S(=O)(=O)Nc4ccccc4)cc3)ccc2s1] |
| 11 | 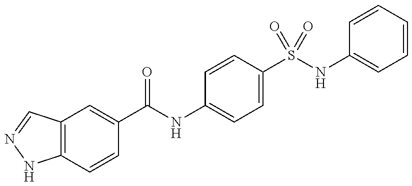 [SMILES: O=C(Nc1ccc(S(=O)(=O)Nc2ccccc2)cc1)c1ccc2[nH]ncc2c1] |
| 12 | 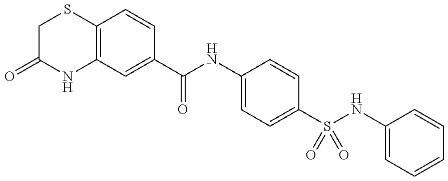 [SMILES: O=C1CSc2ccc(C(=O)Nc3ccc(S(=O)(=O)Nc4ccccc4)cc3)cc2N1] |
| 13 | 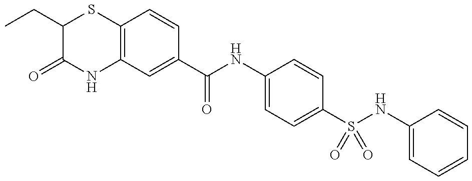 [SMILES: CCC1Sc2ccc(C(=O)Nc3ccc(S(=O)(=O)Nc4ccccc4)cc3)cc2NC1=O] |
| 14 | 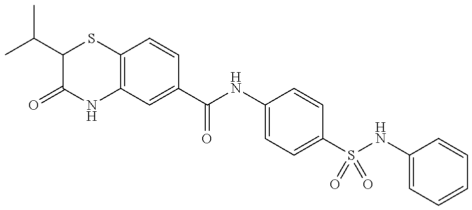 [SMILES: CC(C)C1Sc2ccc(C(=O)Nc3ccc(S(=O)(=O)Nc4ccccc4)cc3)cc2NC1=O] |
| 15 | 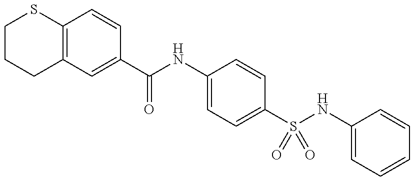 [SMILES: O=C(Nc1ccc(S(=O)(=O)Nc2ccccc2)cc1)c1ccc2c(c1)CCCS2] |
| 16 | 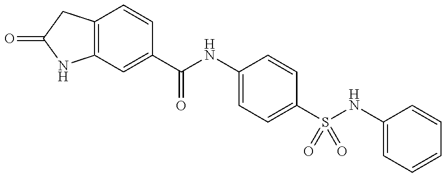 [SMILES: O=C1Cc2ccc(C(=O)Nc3ccc(S(=O)(=O)Nc4ccccc4)cc3)cc2N1] |
| 17 | 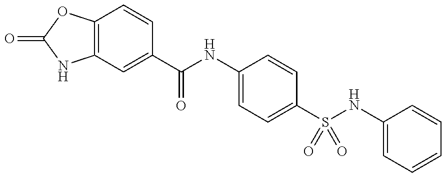 [SMILES: O=C(Nc1ccc(S(=O)(=O)Nc2ccccc2)cc1)c1ccc2oc(=O)[nH]c2c1] |
| 18 | 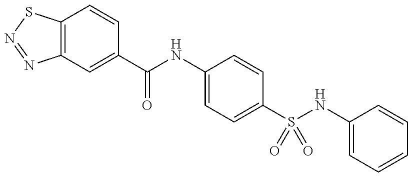 [SMILES: O=C(Nc1ccc(S(=O)(=O)Nc2ccccc2)cc1)c1ccc2snnc2c1] |
| 19 | 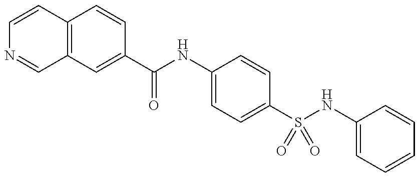 [SMILES: O=C(Nc1ccc(S(=O)(=O)Nc2ccccc2)cc1)c1ccc2ccncc2c1] |
| 20 | 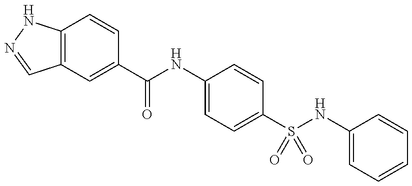 [SMILES: O=C(Nc1ccc(S(=O)(=O)Nc2ccccc2)cc1)c1ccc2[nH]ncc2c1] |
| 21 | 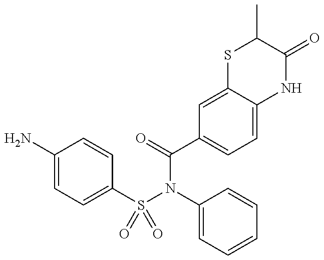 [SMILES: CC1Sc2cc(C(=O)N(c3ccccc3)S(=O)(=O)c3ccc(N)cc3)ccc2NC1=O] |
| 22 | 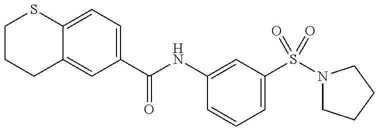 [SMILES: O=C(Nc1cccc(S(=O)(=O)N2CCCC2)c1)c1ccc2c(c1)CCCS2] |
| 23 | 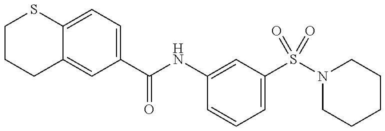 [SMILES: O=C(Nc1cccc(S(=O)(=O)N2CCCCC2)c1)c1ccc2c(c1)CCCS2] |
| 24 | 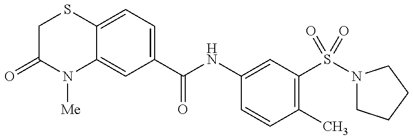 [SMILES: Cc1ccc(NC(=O)c2ccc3c(c2)N(C)C(=O)CS3)cc1S(=O)(=O)N1CCCC1] |
| 25 | 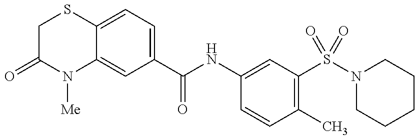 [SMILES: Cc1ccc(NC(=O)c2ccc3c(c2)N(C)C(=O)CS3)cc1S(=O)(=O)N1CCCCC1] |
| 26 | 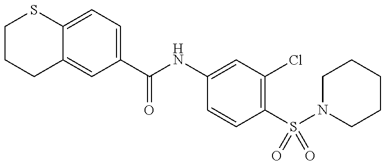 [SMILES: O=C(Nc1ccc(S(=O)(=O)N2CCCCC2)c(Cl)c1)c1ccc2c(c1)CCCS2] |
| 27 | 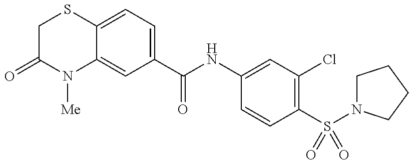 [SMILES: CN1C(=O)CSc2ccc(C(=O)Nc3ccc(S(=O)(=O)N4CCCC4)c(Cl)c3)cc21] |
| 28 | 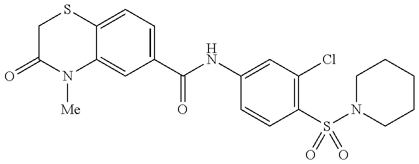 [SMILES: CN1C(=O)CSc2ccc(C(=O)Nc3ccc(S(=O)(=O)N4CCCCC4)c(Cl)c3)cc21] |
| 29 | 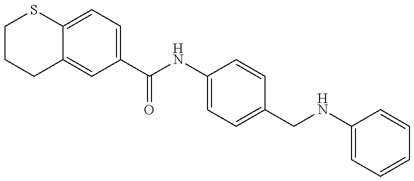 [SMILES: O=C(Nc1ccc(CNc2ccccc2)cc1)c1ccc2c(c1)CCCS2] |
| 30 | 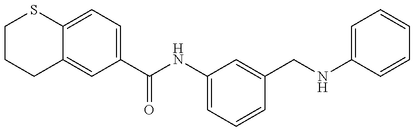 [SMILES: O=C(Nc1cccc(CNc2ccccc2)c1)c1ccc2c(c1)CCCS2] |
| 31 | 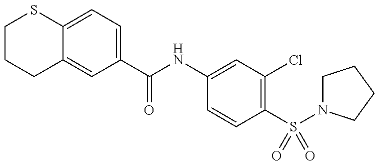 [SMILES: O=C(Nc1ccc(S(=O)(=O)N2CCCC2)c(Cl)c1)c1ccc2c(c1)CCCS2] |
| 32 | 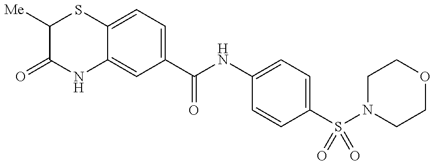 [SMILES: CC1Sc2ccc(C(=O)Nc3ccc(S(=O)(=O)N4CCOCC4)cc3)cc2NC1=O] |
| 33 | 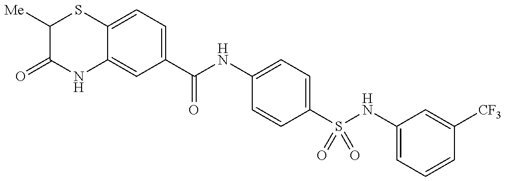 [SMILES: CC1Sc2ccc(C(=O)Nc3ccc(S(=O)(=O)Nc4cccc(C(F)(F)F)c4)cc3)cc2NC1=O] |
| 34 | 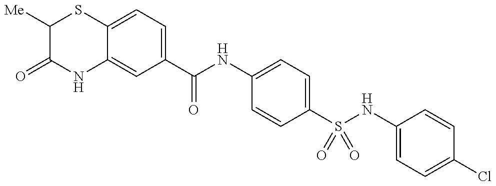 [SMILES: CC1Sc2ccc(C(=O)Nc3ccc(S(=O)(=O)Nc4ccc(Cl)cc4)cc3)cc2NC1=O] |
| 35 | 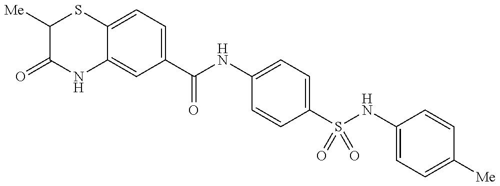 [SMILES: Cc1ccc(NS(=O)(=O)c2ccc(NC(=O)c3ccc4c(c3)NC(=O)C(C)S4)cc2)cc1] |
| 36 | 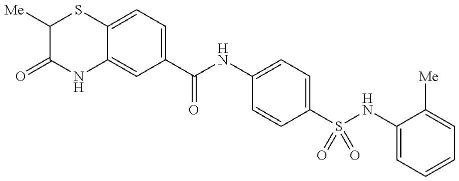 [SMILES: Cc1ccccc1NS(=O)(=O)c1ccc(NC(=O)c2ccc3c(c2)NC(=O)C(C)S3)cc1] |
| 37 | 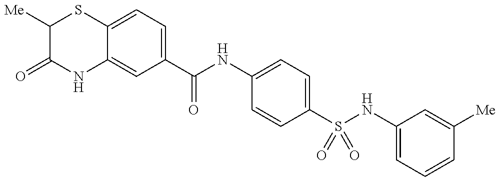 [SMILES: Cc1cccc(NS(=O)(=O)c2ccc(NC(=O)c3ccc4c(c3)NC(=O)C(C)S4)cc2)c1] |
| 38 | 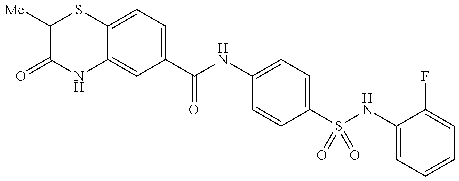 [SMILES: CC1Sc2ccc(C(=O)Nc3ccc(S(=O)(=O)Nc4ccccc4F)cc3)cc2NC1=O] |
| 39 | 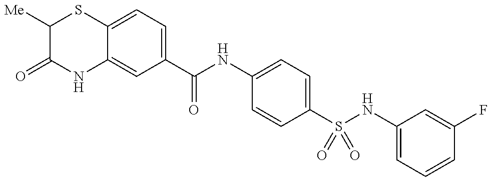 [SMILES: CC1Sc2ccc(C(=O)Nc3ccc(S(=O)(=O)Nc4cccc(F)c4)cc3)cc2NC1=O] |
| 40 | 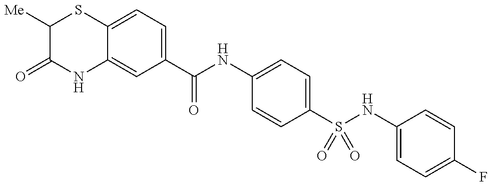 [SMILES: CC1Sc2ccc(C(=O)Nc3ccc(S(=O)(=O)Nc4ccc(F)cc4)cc3)cc2NC1=O] |
| 41 | 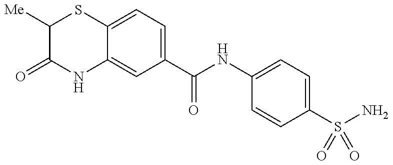 [SMILES: CC1Sc2ccc(C(=O)Nc3ccc(S(N)(=O)=O)cc3)cc2NC1=O] |
| 42 | 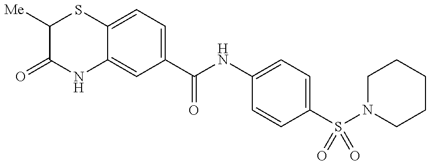 [SMILES: CC1Sc2ccc(C(=O)Nc3ccc(S(=O)(=O)N4CCCCC4)cc3)cc2NC1=O] |
| 43 | 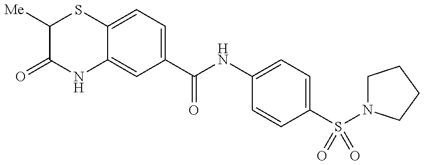 [SMILES: CC1Sc2ccc(C(=O)Nc3ccc(S(=O)(=O)N4CCCC4)cc3)cc2NC1=O] |
| 44 | 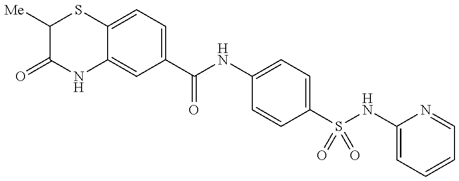 [SMILES: CC1Sc2ccc(C(=O)Nc3ccc(S(=O)(=O)Nc4ccccn4)cc3)cc2NC1=O] |
| 45 | 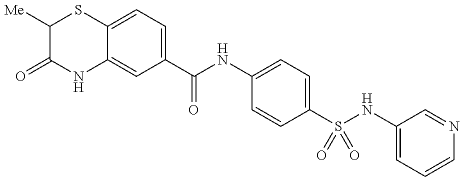 [SMILES: CC1Sc2ccc(C(=O)Nc3ccc(S(=O)(=O)Nc4cccnc4)cc3)cc2NC1=O] |
| 46 | 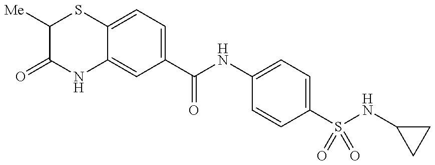 [SMILES: CC1Sc2ccc(C(=O)Nc3ccc(S(=O)(=O)NC4CC4)cc3)cc2NC1=O] |
| 47 | 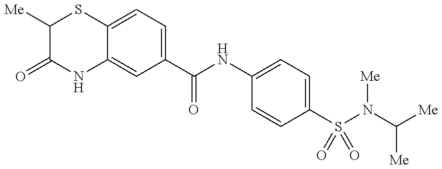 [SMILES: CC1Sc2ccc(C(=O)Nc3ccc(S(=O)(=O)N(C)C(C)C)cc3)cc2NC1=O] |
| 48 | 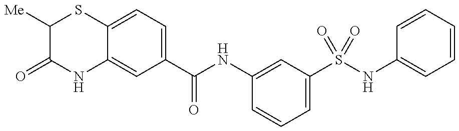 [SMILES: CC1Sc2ccc(C(=O)Nc3cccc(S(=O)(=O)Nc4ccccc4)c3)cc2NC1=O] |
| 49 | 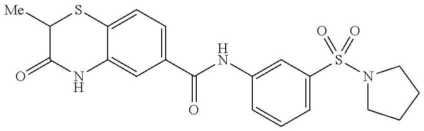 [SMILES: CC1Sc2ccc(C(=O)Nc3cccc(S(=O)(=O)N4CCCC4)c3)cc2NC1=O] |
| 50 | 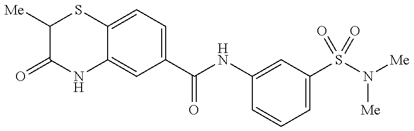 [SMILES: CC1Sc2ccc(C(=O)Nc3cccc(S(=O)(=O)N(C)C)c3)cc2NC1=O] |
| 51 | 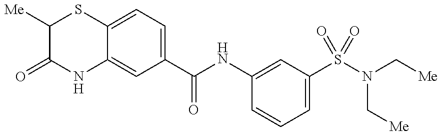 [SMILES: CCN(CC)S(=O)(=O)c1cccc(NC(=O)c2ccc3c(c2)NC(=O)C(C)S3)c1] |
| 52 | 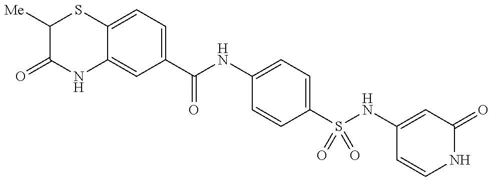 [SMILES: CC1Sc2ccc(C(=O)Nc3ccc(S(=O)(=O)Nc4cc[nH]c(=O)c4)cc3)cc2NC1=O] |
| 53 | 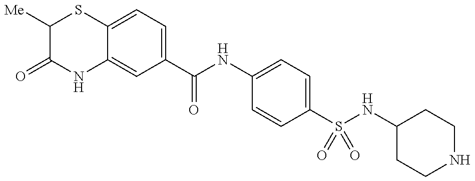 [SMILES: CC1Sc2ccc(C(=O)Nc3ccc(S(=O)(=O)NC4CCNCC4)cc3)cc2NC1=O] |
| 54 | 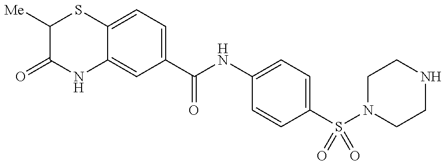 [SMILES: CC1Sc2ccc(C(=O)Nc3ccc(S(=O)(=O)N4CCNCC4)cc3)cc2NC1=O] |
| 55 | 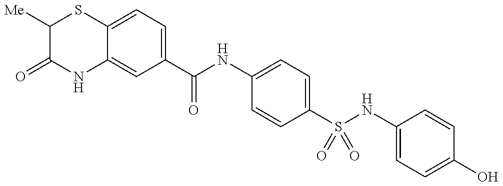 [SMILES: CC1Sc2ccc(C(=O)Nc3ccc(S(=O)(=O)Nc4ccc(O)cc4)cc3)cc2NC1=O] |
| 56 | 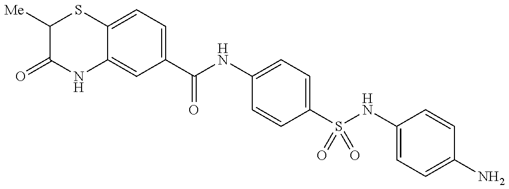 [SMILES: CC1Sc2ccc(C(=O)Nc3ccc(S(=O)(=O)Nc4ccc(N)cc4)cc3)cc2NC1=O] |
| 57 | 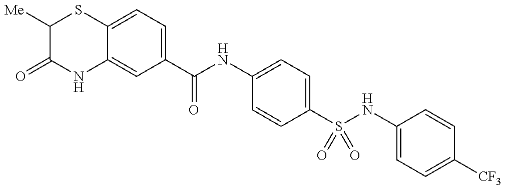 [SMILES: CC1Sc2ccc(C(=O)Nc3ccc(S(=O)(=O)Nc4ccc(C(F)(F)F)cc4)cc3)cc2NC1=O] |
| 58 | 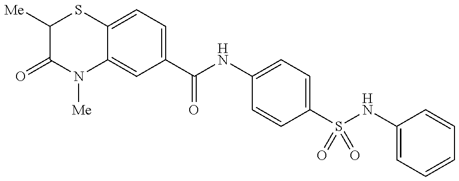 [SMILES: CC1Sc2ccc(C(=O)Nc3ccc(S(=O)(=O)Nc4ccccc4)cc3)cc2N(C)C1=O] |
| 59 | 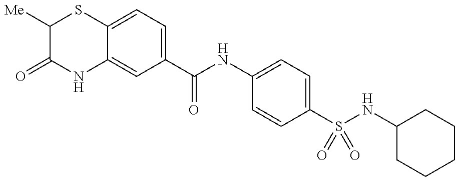 [SMILES: CC1Sc2ccc(C(=O)Nc3ccc(S(=O)(=O)NC4CCCCC4)cc3)cc2NC1=O] |
| 60 | 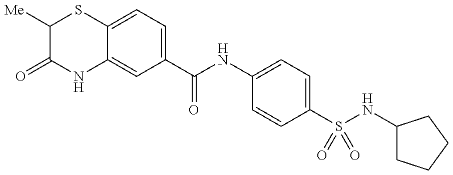 [SMILES: CC1Sc2ccc(C(=O)Nc3ccc(S(=O)(=O)NC4CCCC4)cc3)cc2NC1=O] |
| 61 | 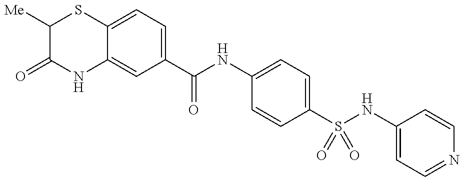 [SMILES: CC1Sc2ccc(C(=O)Nc3ccc(S(=O)(=O)Nc4ccncc4)cc3)cc2NC1=O] |
| 62 | 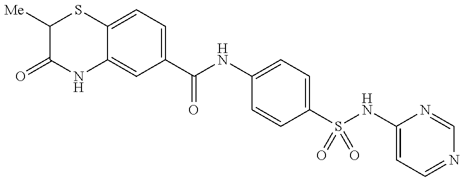 [SMILES: CC1Sc2ccc(C(=O)Nc3ccc(S(=O)(=O)Nc4ccncn4)cc3)cc2NC1=O] |
| 63 | 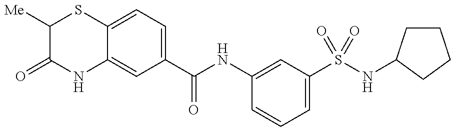 [SMILES: CC1Sc2ccc(C(=O)Nc3cccc(S(=O)(=O)NC4CCCC4)c3)cc2NC1=O] |
| 64 | 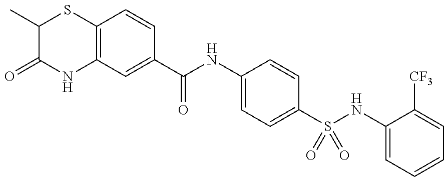 [SMILES: CC1Sc2ccc(C(=O)Nc3ccc(S(=O)(=O)Nc4ccccc4C(F)(F)F)cc3)cc2NC1=O] |
| 65 | 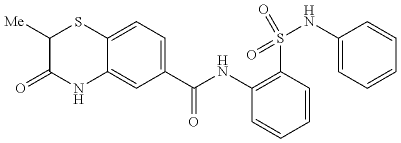 [SMILES: CC1Sc2ccc(C(=O)Nc3ccccc3S(=O)(=O)Nc3ccccc3)cc2NC1=O] |
| 66 | 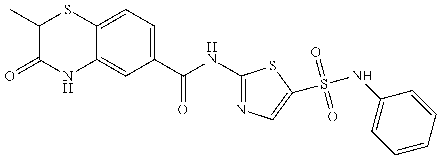 [SMILES: CC1Sc2ccc(C(=O)Nc3ncc(S(=O)(=O)Nc4ccccc4)s3)cc2NC1=O] |
| 67 | 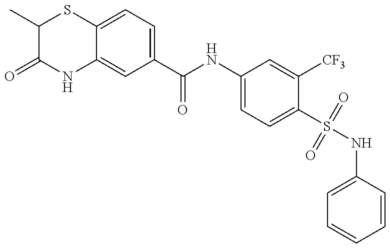 [SMILES: CC1Sc2ccc(C(=O)Nc3ccc(S(=O)(=O)Nc4ccccc4)c(C(F)(F)F)c3)cc2NC1=O] |
| 68 | 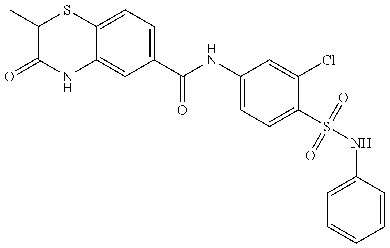 [SMILES: CC1Sc2ccc(C(=O)Nc3ccc(S(=O)(=O)Nc4ccccc4)c(Cl)c3)cc2NC1=O] |
| 69 | 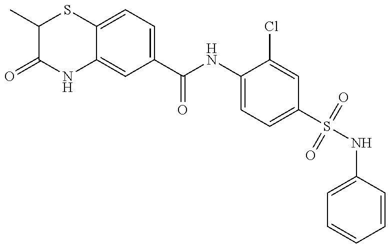 [SMILES: CC1Sc2ccc(C(=O)Nc3ccc(S(=O)(=O)Nc4ccccc4)cc3Cl)cc2NC1=O] |
| 70 | 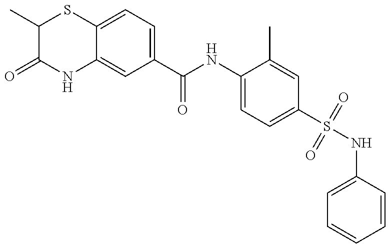 [SMILES: Cc1cc(S(=O)(=O)Nc2ccccc2)ccc1NC(=O)c1ccc2c(c1)NC(=O)C(C)S2] |
| 71 | 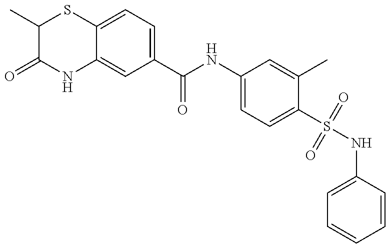 [SMILES: Cc1cc(NC(=O)c2ccc3c(c2)NC(=O)C(C)S3)ccc1S(=O)(=O)Nc1ccccc1] |
| 72 | 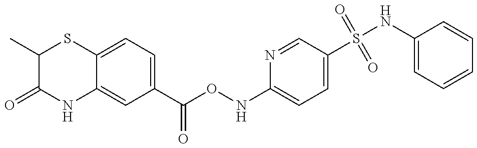 [SMILES: CC1Sc2ccc(C(=O)ONc3ccc(S(=O)(=O)Nc4ccccc4)cn3)cc2NC1=O] |
| 73 | 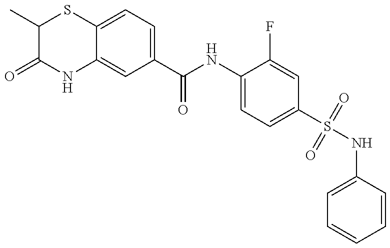 [SMILES: CC1Sc2ccc(C(=O)Nc3ccc(S(=O)(=O)Nc4ccccc4)cc3F)cc2NC1=O] |
| 74 | 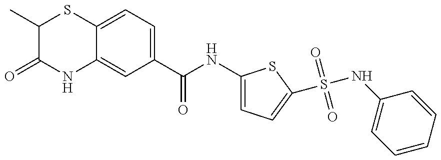 [SMILES: CC1Sc2ccc(C(=O)Nc3ccc(S(=O)(=O)Nc4ccccc4)s3)cc2NC1=O] |
| 75 | 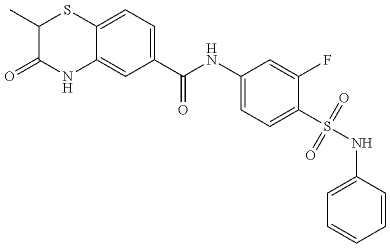 [SMILES: CC1Sc2ccc(C(=O)Nc3ccc(S(=O)(=O)Nc4ccccc4)c(F)c3)cc2NC1=O] |
| 76 | 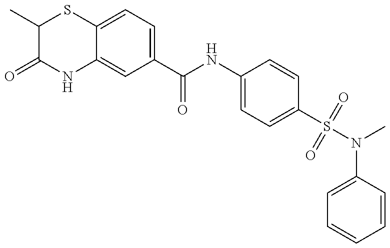 |
| 77 | 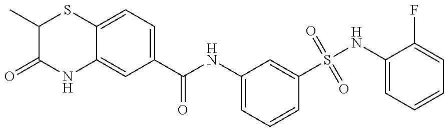 [SMILES: CC1Sc2ccc(C(=O)Nc3cccc(S(=O)(=O)Nc4ccccc4F)c3)cc2NC1=O] |
| 78 | 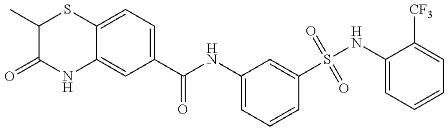 [SMILES: CC1Sc2ccc(C(=O)Nc3cccc(S(=O)(=O)Nc4ccccc4C(F)(F)F)c3)cc2NC1=O] |
| 79 | 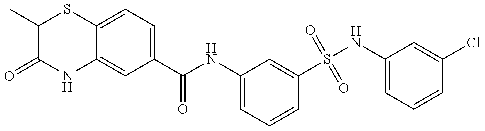 [SMILES: CC1Sc2ccc(C(=O)Nc3cccc(S(=O)(=O)Nc4cccc(Cl)c4)c3)cc2NC1=O] |
| 80 | 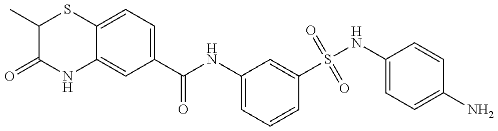 [SMILES: CC1Sc2ccc(C(=O)Nc3cccc(S(=O)(=O)Nc4ccc(N)cc4)c3)cc2NC1=O] |
| 81 | 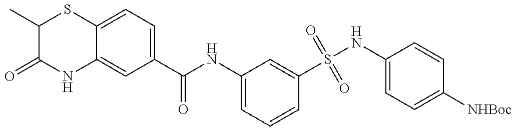 [SMILES: CC1Sc2ccc(C(=O)Nc3cccc(S(=O)(=O)Nc4ccc(NC(=O)OC(C)(C)C)cc4)c3)cc2NC1=O] |
| 82 | 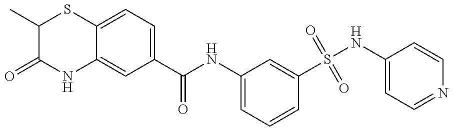 [SMILES: CC1Sc2ccc(C(=O)Nc3cccc(S(=O)(=O)Nc4ccncc4)c3)cc2NC1=O] |
| 83 | 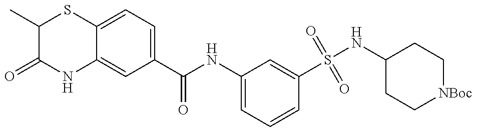 [SMILES: CC1Sc2ccc(C(=O)Nc3cccc(S(=O)(=O)NC4CCN(C(=O)OC(C)(C)C)CC4)c3)cc2NC1=O] |
| 84 | 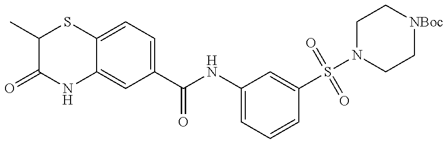 [SMILES: CC1Sc2ccc(C(=O)Nc3cccc(S(=O)(=O)N4CCN(C(=O)OC(C)(C)C)CC4)c3)cc2NC1=O] |
| 85 | 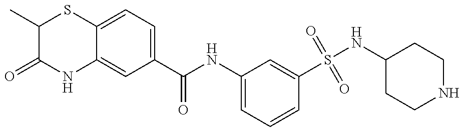 [SMILES: CC1Sc2ccc(C(=O)Nc3cccc(S(=O)(=O)NC4CCNCC4)c3)cc2NC1=O] |
| 86 | 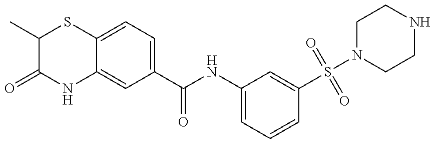 [SMILES: CC1Sc2ccc(C(=O)Nc3cccc(S(=O)(=O)N4CCNCC4)c3)cc2NC1=O] |
| 87 | 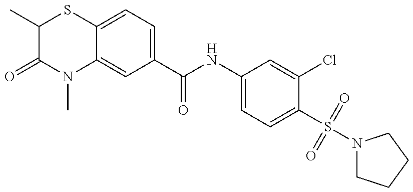 [SMILES: CC1Sc2ccc(C(=O)Nc3ccc(S(=O)(=O)N4CCCC4)c(Cl)c3)cc2N(C)C1=O] |
| 88 | 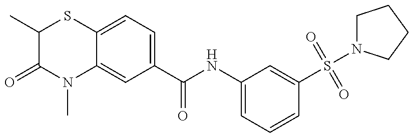 [SMILES: CC1Sc2ccc(C(=O)Nc3cccc(S(=O)(=O)N4CCCC4)c3)cc2N(C)C1=O] |
| 89 | 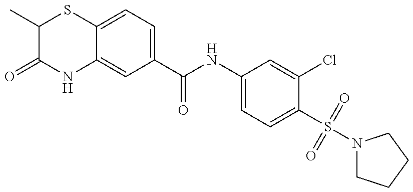 [SMILES: CC1Sc2ccc(C(=O)Nc3ccc(S(=O)(=O)N4CCCC4)c(Cl)c3)cc2NC1=O] |
| 90 | 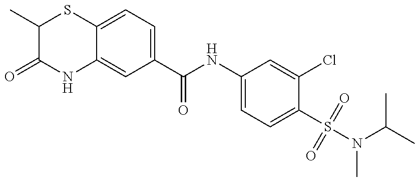 [SMILES: CC1Sc2ccc(C(=O)Nc3ccc(S(=O)(=O)N(C)C(C)C)c(Cl)c3)cc2NC1=O] |
| 91 | 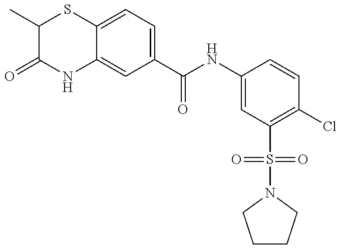 [SMILES: CC1Sc2ccc(C(=O)Nc3ccc(Cl)c(S(=O)(=O)N4CCCC4)c3)cc2NC1=O] |
| 92 | 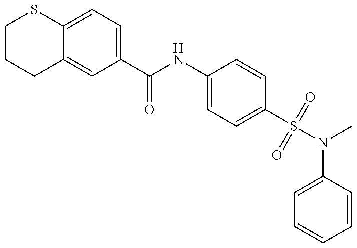 [SMILES: CN(c1ccccc1)S(=O)(=O)c1ccc(NC(=O)c2ccc3c(c2)CCCS3)cc1] |
| 93 | 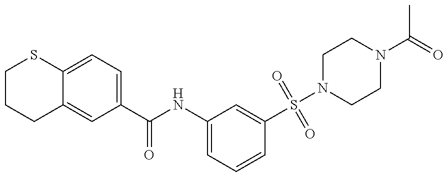 [SMILES: CC(=O)N1CCN(S(=O)(=O)c2cccc(NC(=O)c3ccc4c(c3)CCCS4)c2)CC1] |
| 94 | 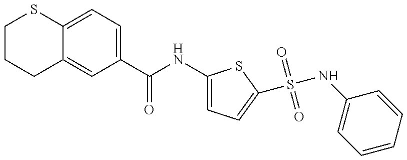 [SMILES: O=C(Nc1ccc(S(=O)(=O)Nc2ccccc2)s1)c1ccc2c(c1)CCCS2] |
| 95 | 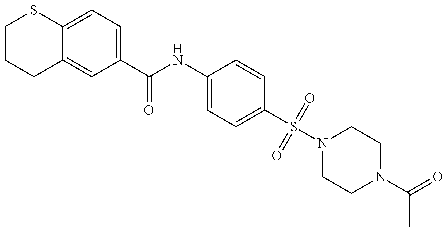 [SMILES: CC(=O)N1CCN(S(=O)(=O)c2ccc(NC(=O)c3ccc4c(c3)CCCS4)cc2)CC1] |
| 96 | 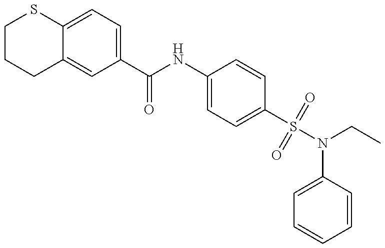 [SMILES: CCN(c1ccccc1)S(=O)(=O)c1ccc(NC(=O)c2ccc3c(c2)CCCS3)cc1] |
| 97 | 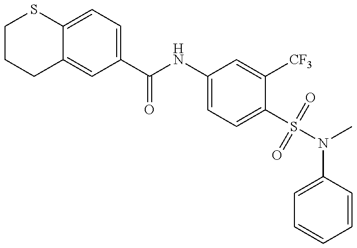 [SMILES: CN(c1ccccc1)S(=O)(=O)c1ccc(NC(=O)c2ccc3c(c2)CCCS3)cc1C(F)(F)F] |
| 98 | 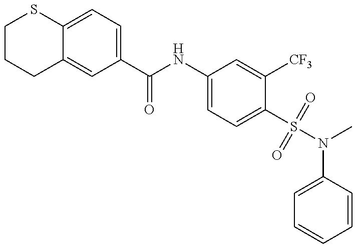 [SMILES: CN(c1ccccc1)S(=O)(=O)c1ccc(NC(=O)c2ccc3c(c2)CCCS3)cc1C(F)(F)F] |
| 99 | 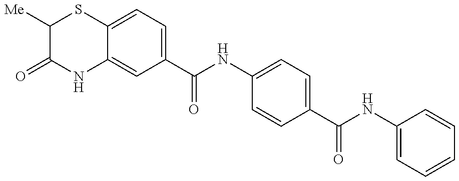 [SMILES: CC1Sc2ccc(C(=O)Nc3ccc(C(=O)Nc4ccccc4)cc3)cc2NC1=O] |
| 100 | 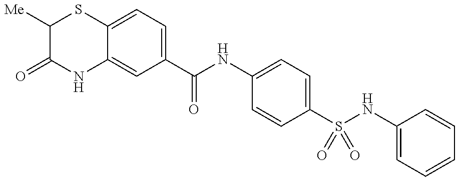 [SMILES: CC1Sc2ccc(C(=O)Nc3ccc(S(=O)(=O)Nc4ccccc4)cc3)cc2NC1=O] |
| 101 | 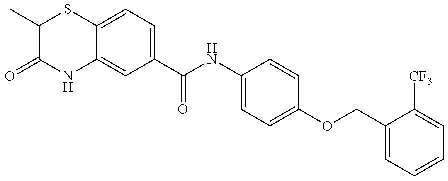 [SMILES: CC1Sc2ccc(C(=O)Nc3ccc(OCc4ccccc4C(F)(F)F)cc3)cc2NC1=O] |
| 102 | 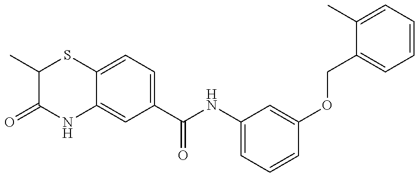 [SMILES: Cc1ccccc1COc1cccc(NC(=O)c2ccc3c(c2)NC(=O)C(C)S3)c1] |
| 103 | 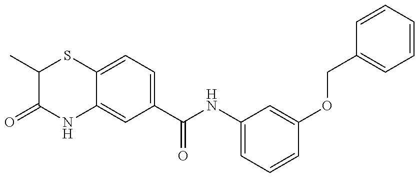 [SMILES: CC1Sc2ccc(C(=O)Nc3cccc(OCc4ccccc4)c3)cc2NC1=O] |
| 104 | 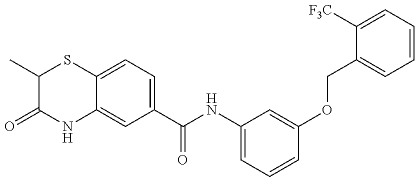 [SMILES: CC1Sc2ccc(C(=O)Nc3cccc(OCc4ccccc4C(F)(F)F)c3)cc2NC1=O] |
| 105 | 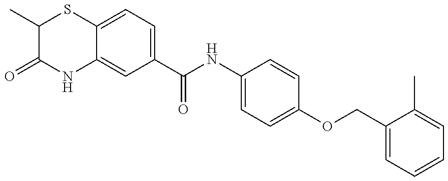 [SMILES: Cc1ccccc1COc1ccc(NC(=O)c2ccc3c(c2)NC(=O)C(C)S3)cc1] |
| 106 | 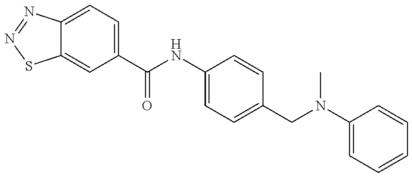 [SMILES: CN(Cc1ccc(NC(=O)c2ccc3nnsc3c2)cc1)c1ccccc1] |
| 107 | 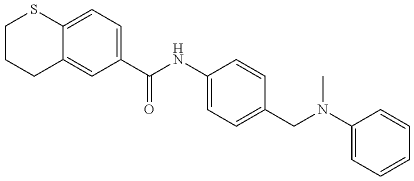 [SMILES: CN(Cc1ccc(NC(=O)c2ccc3c(c2)CCCS3)cc1)c1ccccc1] |
| 108 | 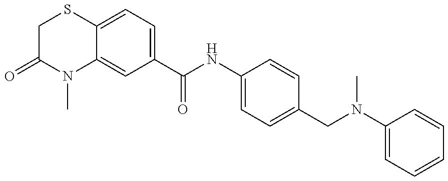 [SMILES: CN(Cc1ccc(NC(=O)c2ccc3c(c2)N(C)C(=O)CS3)cc1)c1ccccc1] |
| 109 | 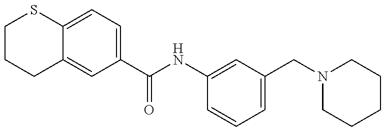 [SMILES: O=C(Nc1cccc(CN2CCCCC2)c1)c1ccc2c(c1)CCCS2] |
| 110 | 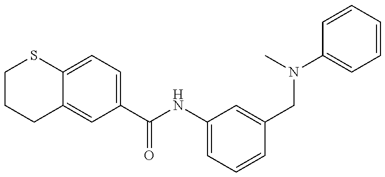 [SMILES: CN(Cc1cccc(NC(=O)c2ccc3c(c2)CCCS3)c1)c1ccccc1] |
| 111 | 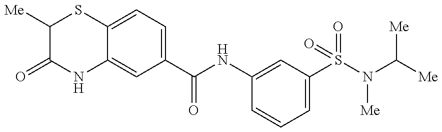 [SMILES: CC1Sc2ccc(C(=O)Nc3cccc(S(=O)(=O)N(C)C(C)C)c3)cc2NC1=O] |
| 112 | 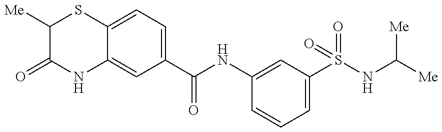 [SMILES: CC(C)NS(=O)(=O)c1cccc(NC(=O)c2ccc3c(c2)NC(=O)C(C)S3)c1] |
| 113 | 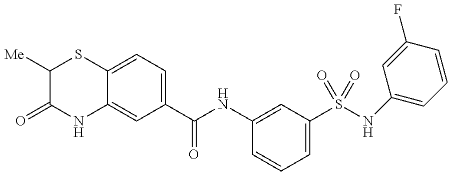 [SMILES: CC1Sc2ccc(C(=O)Nc3cccc(S(=O)(=O)Nc4cccc(F)c4)c3)cc2NC1=O] |
| 114 | 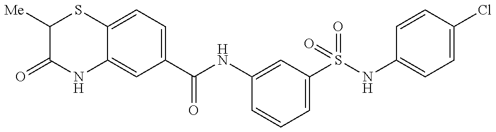 [SMILES: CC1Sc2ccc(C(=O)Nc3cccc(S(=O)(=O)Nc4ccc(Cl)cc4)c3)cc2NC1=O] |
| 115 | 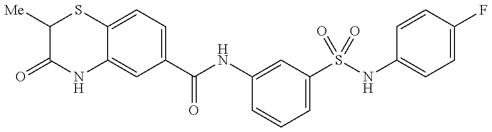 [SMILES: CC1Sc2ccc(C(=O)Nc3cccc(S(=O)(=O)Nc4ccc(F)cc4)c3)cc2NC1=O] |
| 116 | 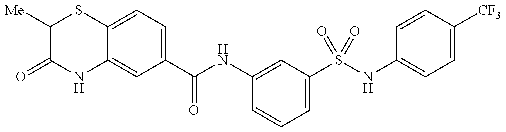 [SMILES: CC1Sc2ccc(C(=O)Nc3cccc(S(=O)(=O)Nc4ccc(C(F)(F)F)cc4)c3)cc2NC1=O] |
| 117 | 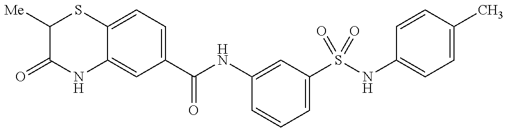 [SMILES: Cc1ccc(NS(=O)(=O)c2cccc(NC(=O)c3ccc4c(c3)NC(=O)C(C)S4)c2)cc1] |
| 118 | 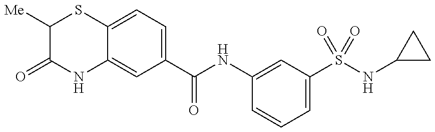 [SMILES: CC1Sc2ccc(C(=O)Nc3cccc(S(=O)(=O)NC4CC4)c3)cc2NC1=O] |
| 119 | 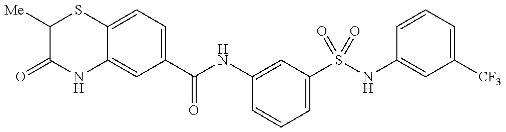 [SMILES: CC1Sc2ccc(C(=O)Nc3cccc(S(=O)(=O)Nc4cccc(C(F)(F)F)c4)c3)cc2NC1=O] |
| 120 | 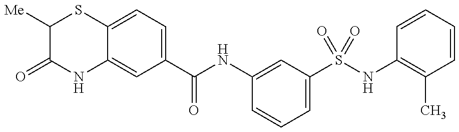 [SMILES: Cc1ccccc1NS(=O)(=O)c1cccc(NC(=O)c2ccc3c(c2)NC(=O)C(C)S3)c1] |
| 121 | 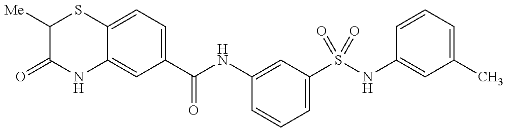 [SMILES: Cc1cccc(NS(=O)(=O)c2cccc(NC(=O)c3ccc4c(c3)NC(=O)C(C)S4)c2)c1] |
| 122 | 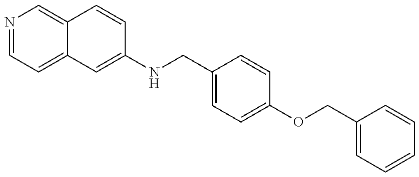 [SMILES: c1ccc(COc2ccc(CNc3ccc4cnccc4c3)cc2)cc1] |
| 123 | 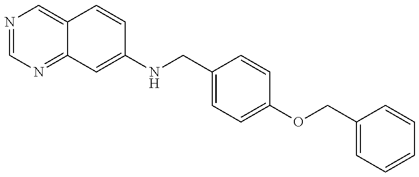 [SMILES: c1ccc(COc2ccc(CNc3ccc4cncnc4c3)cc2)cc1] |
| 124 | 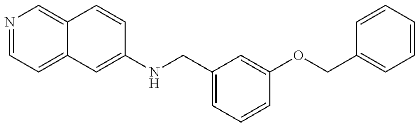 [SMILES: c1ccc(COc2cccc(CNc3ccc4cnccc4c3)c2)cc1] |
| 125 | 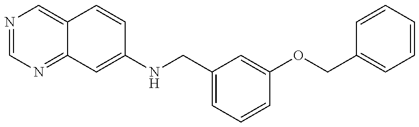 [SMILES: c1ccc(COc2cccc(CNc3ccc4cncnc4c3)c2)cc1] |
| 126 | 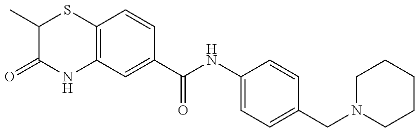 [SMILES: CC1Sc2ccc(C(=O)Nc3ccc(CN4CCCCC4)cc3)cc2NC1=O] |
| 127 | 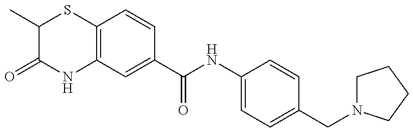 [SMILES: CC1Sc2ccc(C(=O)Nc3ccc(CN4CCCC4)cc3)cc2NC1=O] |
| 128 | 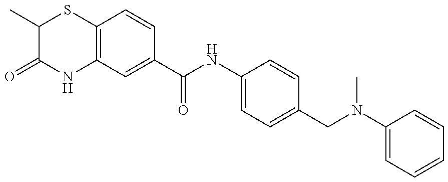 [SMILES: CC1Sc2ccc(C(=O)Nc3ccc(CN(C)c4ccccc4)cc3)cc2NC1=O] |
| 129 | 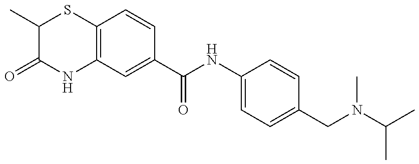 [SMILES: CC1Sc2ccc(C(=O)Nc3ccc(CN(C)C(C)C)cc3)cc2NC1=O] |
| 130 | 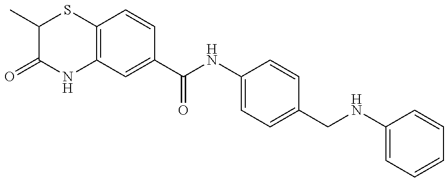 [SMILES: CC1Sc2ccc(C(=O)Nc3ccc(CNc4ccccc4)cc3)cc2NC1=O] |
| 131 | 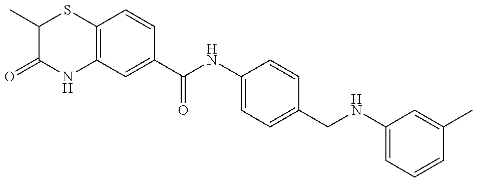 |
| 132 | 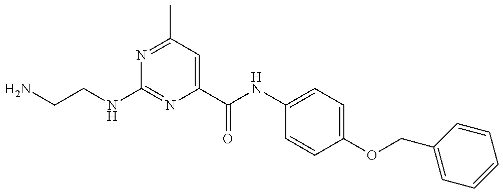 [SMILES: Cc1cc(C(=O)Nc2ccc(OCc3ccccc3)cc2)nc(NCCN)n1] |
| 133 | 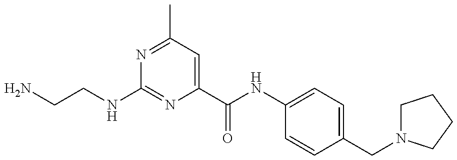 [SMILES: Cc1cc(C(=O)Nc2ccc(CN3CCCC3)cc2)nc(NCCN)n1] |
| 134 | 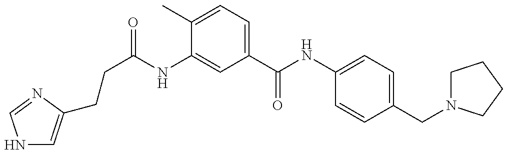 [SMILES: Cc1ccc(C(=O)Nc2ccc(CN3CCCC3)cc2)cc1NC(=O)CCc1c[nH]cn1] |
| 135 | 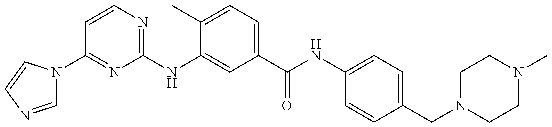 [SMILES: Cc1ccc(C(=O)Nc2ccc(CN3CCN(C)CC3)cc2)cc1Nc1nccc(-n2ccnc2)n1] |
| 136 | 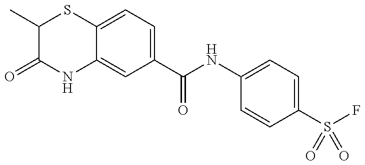 [SMILES: CC1Sc2ccc(C(=O)Nc3ccc(S(=O)(=O)F)cc3)cc2NC1=O] |
| 137 | 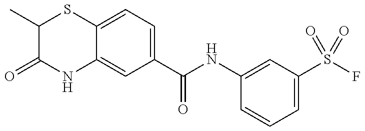 [SMILES: CC1Sc2ccc(C(=O)Nc3cccc(S(=O)(=O)F)c3)cc2NC1=O] |
| 138 | 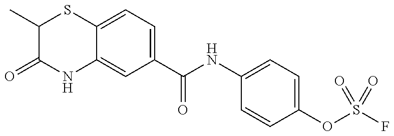 [SMILES: CC1Sc2ccc(C(=O)Nc3ccc(OS(=O)(=O)F)cc3)cc2NC1=O] |
| 139 | 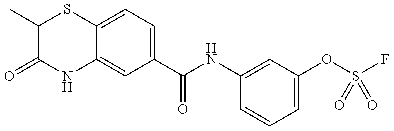 [SMILES: CC1Sc2ccc(C(=O)Nc3cccc(OS(=O)(=O)F)c3)cc2NC1=O] |
| 140 | 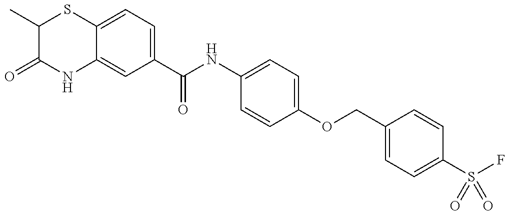 [SMILES: CC1Sc2ccc(C(=O)Nc3ccc(OCc4ccc(S(=O)(=O)F)cc4)cc3)cc2NC1=O] |
| 141 | 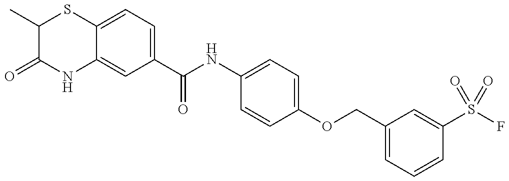 [SMILES: CC1Sc2ccc(C(=O)Nc3ccc(OCc4cccc(S(=O)(=O)F)c4)cc3)cc2NC1=O] |
| 142 | 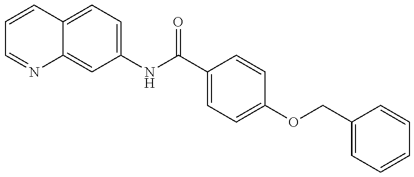 [SMILES: O=C(Nc1ccc2cccnc2c1)c1ccc(OCc2ccccc2)cc1] |
| 143 | 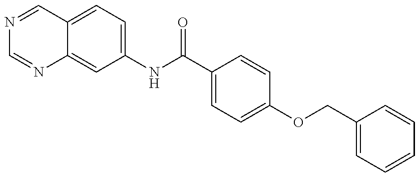 [SMILES: O=C(Nc1ccc2cncnc2c1)c1ccc(OCc2ccccc2)cc1] |
| 144 | 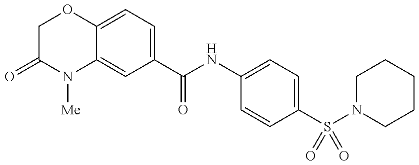 [SMILES: CN1C(=O)COc2ccc(C(=O)Nc3ccc(S(=O)(=O)N4CCCCC4)cc3)cc21] |
| 145 | 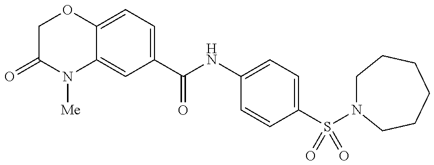 [SMILES: CN1C(=O)COc2ccc(C(=O)Nc3ccc(S(=O)(=O)N4CCCCCC4)cc3)cc21] |
| 146 | 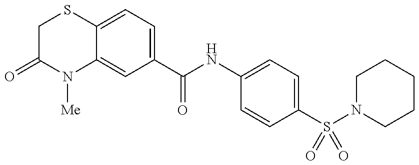 [SMILES: CN1C(=O)CSc2ccc(C(=O)Nc3ccc(S(=O)(=O)N4CCCCC4)cc3)cc21] |
| 147 | 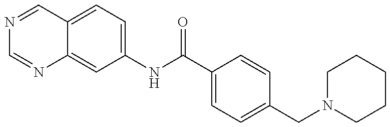 [SMILES: O=C(Nc1ccc2cncnc2c1)c1ccc(CN2CCCCC2)cc1] |
| 148 | 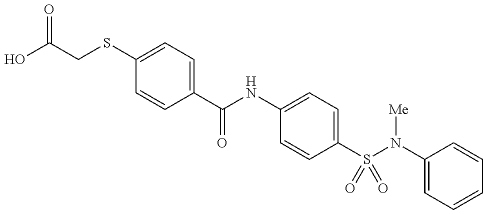 [SMILES: CN(c1ccccc1)S(=O)(=O)c1ccc(NC(=O)c2ccc(SCC(=O)O)cc2)cc1] |
| 149 | 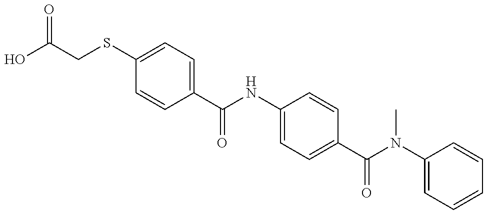 [SMILES: CN(C(=O)c1ccc(NC(=O)c2ccc(SCC(=O)O)cc2)cc1)c1ccccc1] |
| 150 | 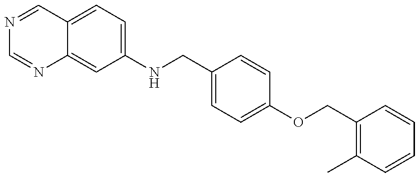 [SMILES: Cc1ccccc1COc1ccc(CNc2ccc3cncnc3c2)cc1] |
| 151 | 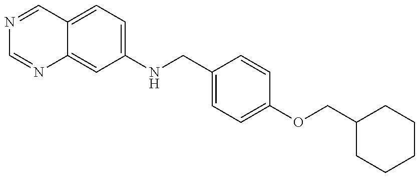 [SMILES: c1ncc2ccc(NCc3ccc(OCC4CCCCC4)cc3)cc2n1] |
| 152 | 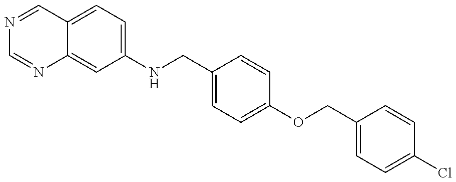 [SMILES: Clc1ccc(COc2ccc(CNc3ccc4cncnc4c3)cc2)cc1] |
| 153 | 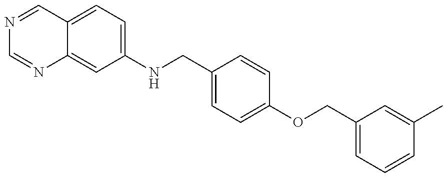 [SMILES: Cc1cccc(COc2ccc(CNc3ccc4cncnc4c3)cc2)c1] |
| 154 | 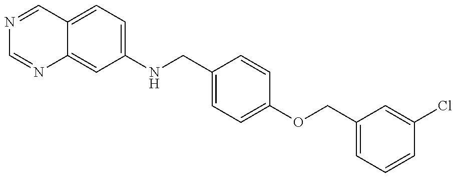 [SMILES: Clc1cccc(COc2ccc(CNc3ccc4cncnc4c3)cc2)c1] |
| 155 | 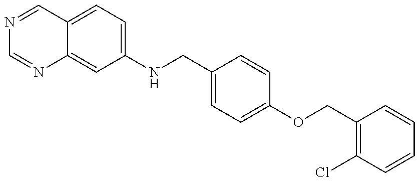 [SMILES: Clc1ccccc1COc1ccc(CNc2ccc3cncnc3c2)cc1] |
| 156 | 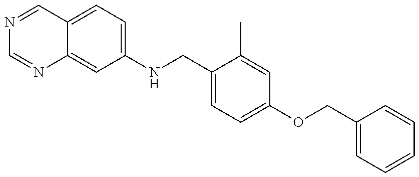 [SMILES: Cc1cc(OCc2ccccc2)ccc1CNc1ccc2cncnc2c1] |
| 157 | 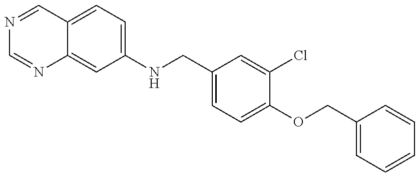 [SMILES: Clc1cc(CNc2ccc3cncnc3c2)ccc1OCc1ccccc1] |
| 158 | 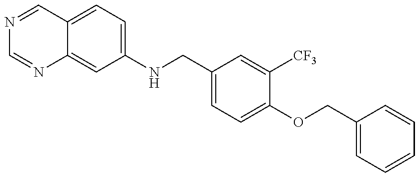 [SMILES: FC(F)(F)c1cc(CNc2ccc3cncnc3c2)ccc1OCc1ccccc1] |
| 159 | 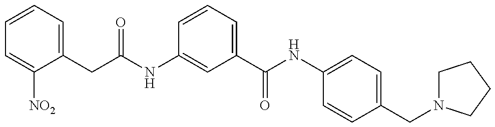 [SMILES: O=C(Cc1ccccc1[N+](=O)[O-])Nc1cccc(C(=O)Nc2ccc(CN3CCCC3)cc2)c1] |
| 160 | 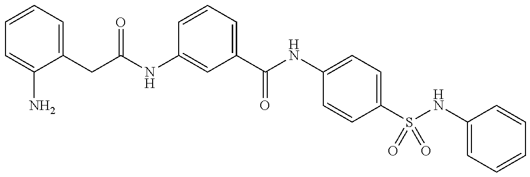 [SMILES: Nc1ccccc1CC(=O)Nc1cccc(C(=O)Nc2ccc(S(=O)(=O)Nc3ccccc3)cc2)c1] |
| 161 | 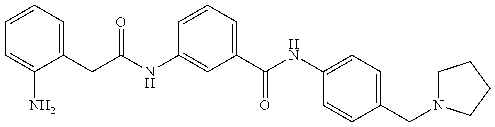 [SMILES: Nc1ccccc1CC(=O)Nc1cccc(C(=O)Nc2ccc(CN3CCCC3)cc2)c1] |
| 162 | 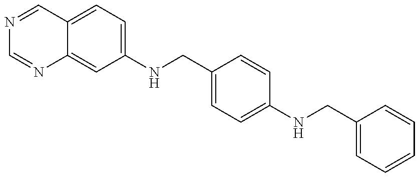 [SMILES: c1ccc(CNc2ccc(CNc3ccc4cncnc4c3)cc2)cc1] |
| 163 | 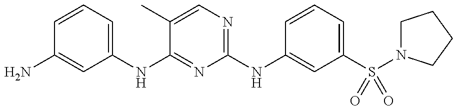 [SMILES: Cc1cnc(Nc2cccc(S(=O)(=O)N3CCCC3)c2)nc1Nc1cccc(N)c1] |
| 164 | 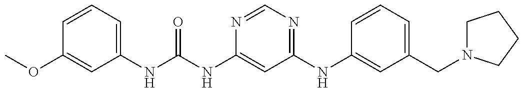 [SMILES: COc1cccc(NC(=O)Nc2cc(Nc3cccc(CN4CCCC4)c3)ncn2)c1] |
| 165 | 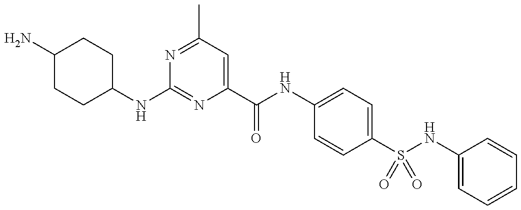 [SMILES: Cc1cc(C(=O)Nc2ccc(S(=O)(=O)Nc3ccccc3)cc2)nc(NC2CCC(N)CC2)n1] |
| 166 | 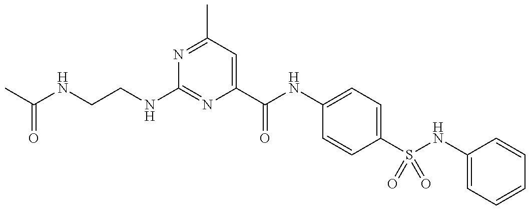 [SMILES: CC(=O)NCCNc1nc(C)cc(C(=O)Nc2ccc(S(=O)(=O)Nc3ccccc3)cc2)n1] |
| 167 | 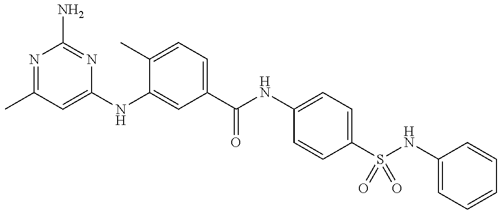 [SMILES: Cc1cc(Nc2cc(C(=O)Nc3ccc(S(=O)(=O)Nc4ccccc4)cc3)ccc2C)nc(N)n1] |
| 168 | 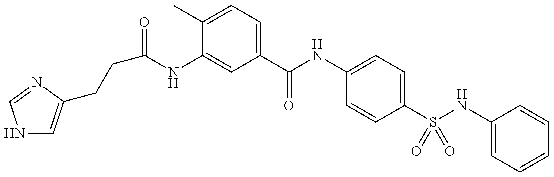 [SMILES: Cc1ccc(C(=O)Nc2ccc(S(=O)(=O)Nc3ccccc3)cc2)cc1NC(=O)CCc1c[nH]cn1] |
| 169 | 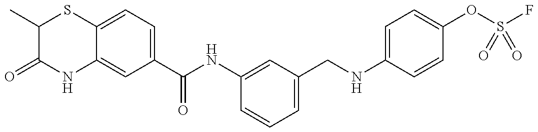 [SMILES: CC1Sc2ccc(C(=O)Nc3cccc(CNc4ccc(OS(=O)(=O)F)cc4)c3)cc2NC1=O] |
| 170 | 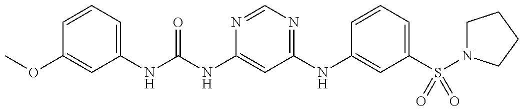 [SMILES: COc1cccc(NC(=O)Nc2cc(Nc3cccc(S(=O)(=O)N4CCCC4)c3)ncn2)c1] |
| 171 | 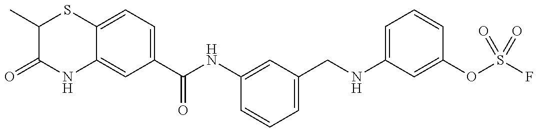 [SMILES: CC1Sc2ccc(C(=O)Nc3cccc(CNc4cccc(OS(=O)(=O)F)c4)c3)cc2NC1=O] |
| 172 | 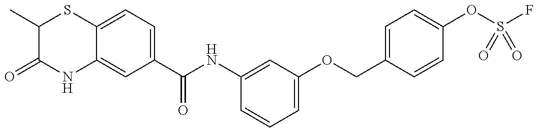 [SMILES: CC1Sc2ccc(C(=O)Nc3cccc(OCc4ccc(OS(=O)(=O)F)cc4)c3)cc2NC1=O] |
| 173 | 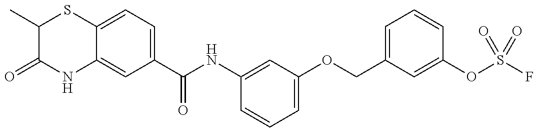 [SMILES: CC1Sc2ccc(C(=O)Nc3cccc(OCc4cccc(OS(=O)(=O)F)c4)c3)cc2NC1=O] |
| 174 | 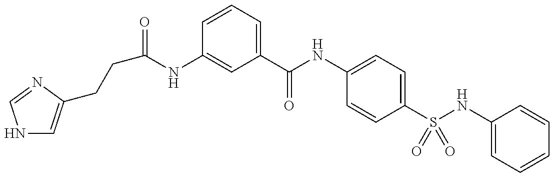 [SMILES: O=C(CCc1c[nH]cn1)Nc1cccc(C(=O)Nc2ccc(S(=O)(=O)Nc3ccccc3)cc2)c1] |
| 175 | 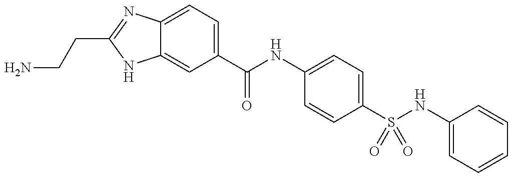 [SMILES: NCCc1nc2ccc(C(=O)Nc3ccc(S(=O)(=O)Nc4ccccc4)cc3)cc2[nH]1] |
| 176 | 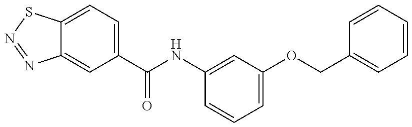 [SMILES: O=C(Nc1cccc(OCc2ccccc2)c1)c1ccc2snnc2c1] |
| 177 | 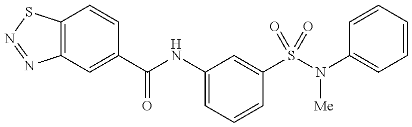 [SMILES: CN(c1ccccc1)S(=O)(=O)c1cccc(NC(=O)c2ccc3snnc3c2)c1] |
| 178 | 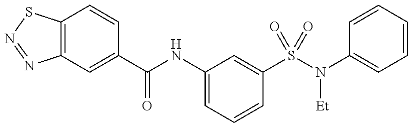 [SMILES: CCN(c1ccccc1)S(=O)(=O)c1cccc(NC(=O)c2ccc3snnc3c2)c1] |
| 179 | 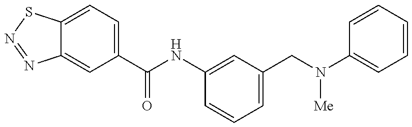 [SMILES: CN(Cc1cccc(NC(=O)c2ccc3snnc3c2)c1)c1ccccc1] |
| 180 | 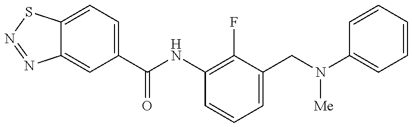 [SMILES: CN(Cc1cccc(NC(=O)c2ccc3snnc3c2)c1F)c1ccccc1] |
| 181 | 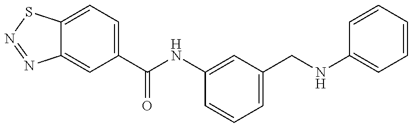 [SMILES: O=C(Nc1cccc(CNc2ccccc2)c1)c1ccc2snnc2c1] |
| 182 | 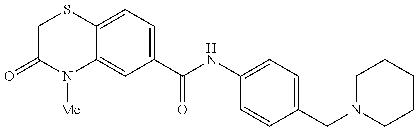 [SMILES: CN1C(=O)CSc2ccc(C(=O)Nc3ccc(CN4CCCCC4)cc3)cc21] |
| 183 | 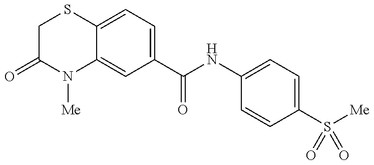 [SMILES: CN1C(=O)CSc2ccc(C(=O)Nc3ccc(S(C)(=O)=O)cc3)cc21] |
| 184 | 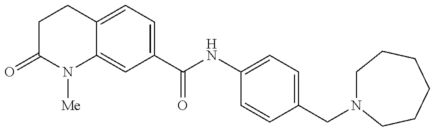 [SMILES: CN1C(=O)CCc2ccc(C(=O)Nc3ccc(CN4CCCCCC4)cc3)cc21] |
| 185 | 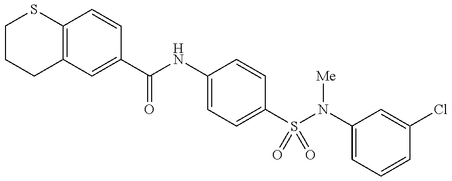 [SMILES: CN(c1cccc(Cl)c1)S(=O)(=O)c1ccc(NC(=O)c2ccc3c(c2)CCCS3)cc1] |
| 186 | 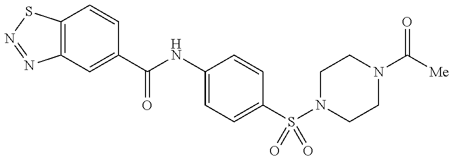 [SMILES: CC(=O)N1CCN(S(=O)(=O)c2ccc(NC(=O)c3ccc4snnc4c3)cc2)CC1] |
| 187 | 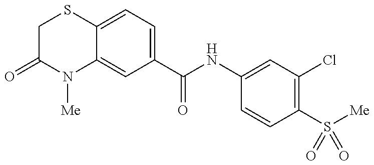 [SMILES: CN1C(=O)CSc2ccc(C(=O)Nc3ccc(S(C)(=O)=O)c(Cl)c3)cc21] |
| 188 | 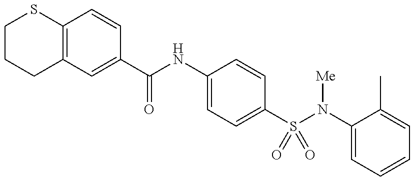 [SMILES: Cc1ccccc1N(C)S(=O)(=O)c1ccc(NC(=O)c2ccc3c(c2)CCCS3)cc1] |
| 189 | 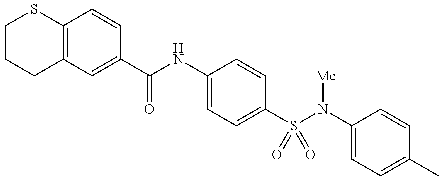 [SMILES: Cc1ccc(N(C)S(=O)(=O)c2ccc(NC(=O)c3ccc4c(c3)CCCS4)cc2)cc1] |
| 190 | 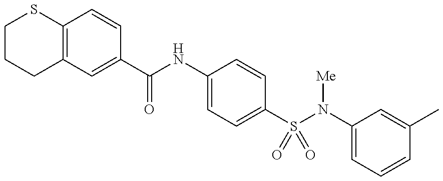 [SMILES: Cc1cccc(N(C)S(=O)(=O)c2ccc(NC(=O)c3ccc4c(c3)CCCS4)cc2)c1] |
| 191 | 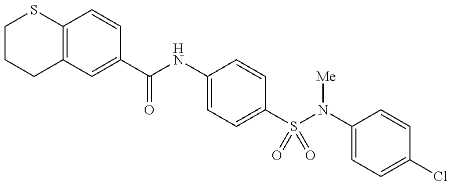 [SMILES: CN(c1ccc(Cl)cc1)S(=O)(=O)c1ccc(NC(=O)c2ccc3c(c2)CCCS3)cc1] |
| 192 | 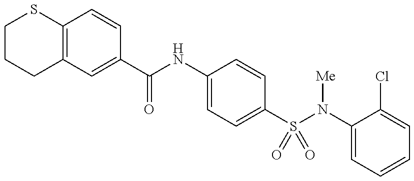 [SMILES: CN(c1ccccc1Cl)S(=O)(=O)c1ccc(NC(=O)c2ccc3c(c2)CCCS3)cc1] |
| 193 | 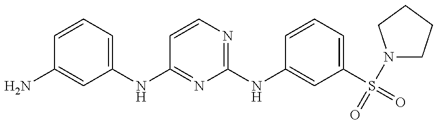 [SMILES: Nc1cccc(Nc2ccnc(Nc3cccc(S(=O)(=O)N4CCCC4)c3)n2)c1] |
| 194 | 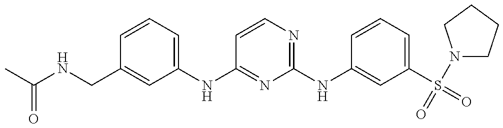 [SMILES: CC(=O)NCc1cccc(Nc2ccnc(Nc3cccc(S(=O)(=O)N4CCCC4)c3)n2)c1] |
| 195 | 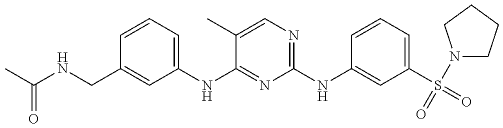 [SMILES: CC(=O)NCc1cccc(Nc2nc(Nc3cccc(S(=O)(=O)N4CCCC4)c3)ncc2C)c1] |
| 196 | 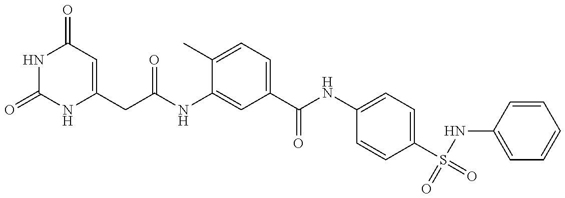 [SMILES: Cc1ccc(C(=O)Nc2ccc(S(=O)(=O)Nc3ccccc3)cc2)cc1NC(=O)Cc1cc(=O)[nH]c(=O)[nH]1] |
| 197 | 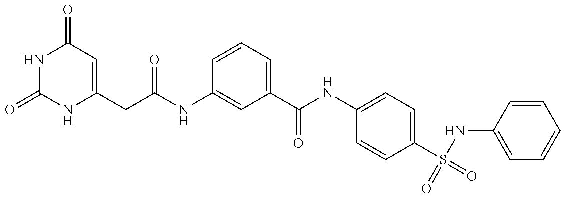 [SMILES: O=C(Cc1cc(=O)[nH]c(=O)[nH]1)Nc1cccc(C(=O)Nc2ccc(S(=O)(=O)Nc3ccccc3)cc2)c1] |
| 198 | 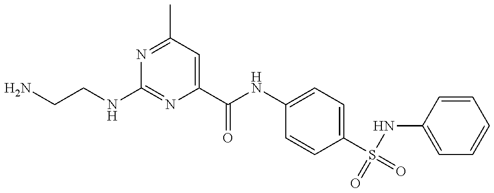 [SMILES: Cc1cc(C(=O)Nc2ccc(S(=O)(=O)Nc3ccccc3)cc2)nc(NCCN)n1] |
| 199 | 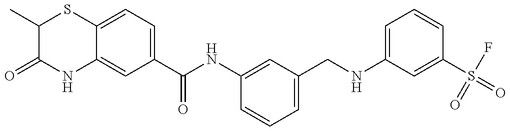 [SMILES: CC1Sc2ccc(C(=O)Nc3cccc(CNc4cccc(S(=O)(=O)F)c4)c3)cc2NC1=O] |
| 200 | 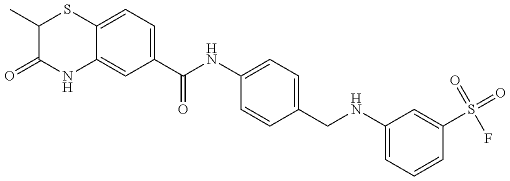 [SMILES: CC1Sc2ccc(C(=O)Nc3ccc(CNc4cccc(S(=O)(=O)F)c4)cc3)cc2NC1=O] |
| 201 | 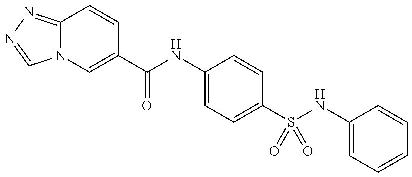 [SMILES: O=C(Nc1ccc(S(=O)(=O)Nc2ccccc2)cc1)c1ccc2nncn2c1] |
| 202 | 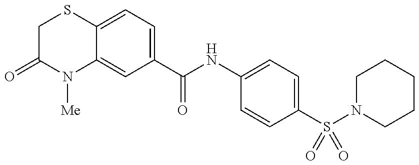 [SMILES: CN1C(=O)CSc2ccc(C(=O)Nc3ccc(S(=O)(=O)N4CCCCC4)cc3)cc21] |
| 203 | 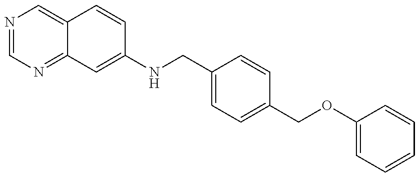 [SMILES: c1ccc(OCc2ccc(CNc3ccc4cncnc4c3)cc2)cc1] |
| 204 | 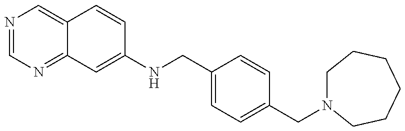 [SMILES: c1ncc2ccc(NCc3ccc(CN4CCCCCC4)cc3)cc2n1] |
| 205 | 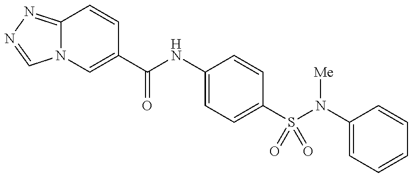 [SMILES: CN(c1ccccc1)S(=O)(=O)c1ccc(NC(=O)c2ccc3nncn3c2)cc1] |
| 206 | 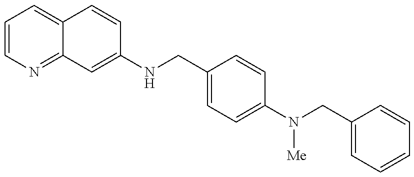 [SMILES: CN(Cc1ccccc1)c1ccc(CNc2ccc3cccnc3c2)cc1] |
| 207 | 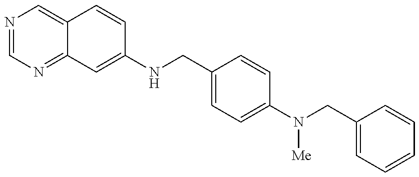 [SMILES: CN(Cc1ccccc1)c1ccc(CNc2ccc3cncnc3c2)cc1] |
| 208 | 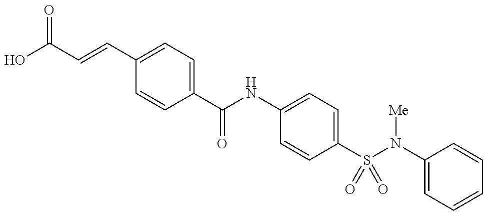 [SMILES: CN(c1ccccc1)S(=O)(=O)c1ccc(NC(=O)c2ccc(/C=C/C(=O)O)cc2)cc1] |
| 209 | 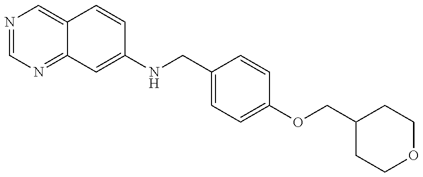 [SMILES: c1ncc2ccc(NCc3ccc(OCC4CCOCC4)cc3)cc2n1] |
| 210 | 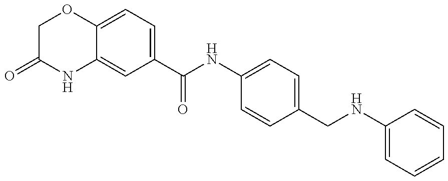 [SMILES: O=C1COc2ccc(C(=O)Nc3ccc(CNc4ccccc4)cc3)cc2N1] |
| 211 | 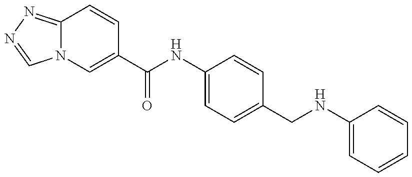 [SMILES: O=C(Nc1ccc(CNc2ccccc2)cc1)c1ccc2nncn2c1] |
| 212 | 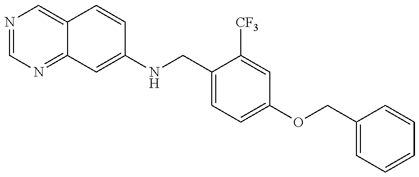 [SMILES: FC(F)(F)c1cc(OCc2ccccc2)ccc1CNc1ccc2cncnc2c1] |
| 213 | 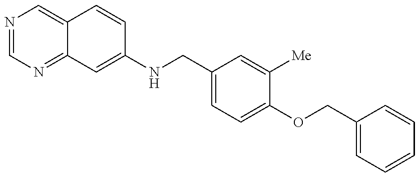 [SMILES: Cc1cc(CNc2ccc3cncnc3c2)ccc1OCc1ccccc1] |
| 214 | 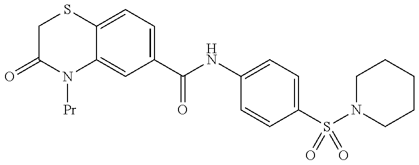 |
| 215 | 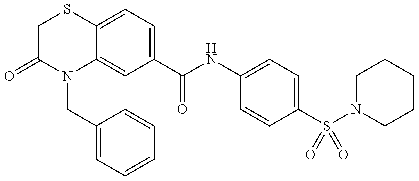 [SMILES: O=C(Nc1ccc(S(=O)(=O)N2CCCCC2)cc1)c1ccc2c(c1)N(Cc1ccccc1)C(=O)CS2] |
| 216 | 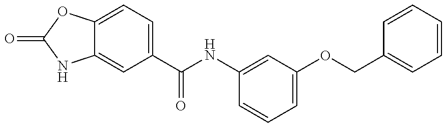 [SMILES: O=C(Nc1cccc(OCc2ccccc2)c1)c1ccc2oc(=O)[nH]c2c1] |
| 217 | 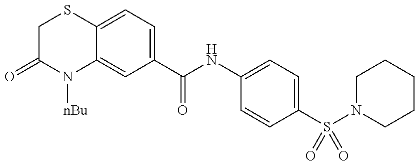 |
| 218 | 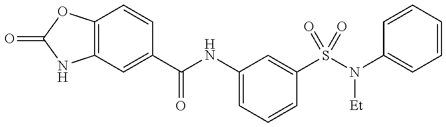 [SMILES: CCN(c1ccccc1)S(=O)(=O)c1cccc(NC(=O)c2ccc3oc(=O)[nH]c3c2)c1] |
| 219 | 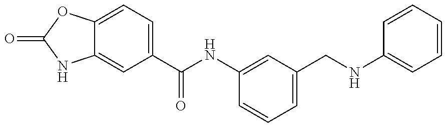 [SMILES: O=C(Nc1cccc(CNc2ccccc2)c1)c1ccc2oc(=O)[nH]c2c1] |
| 220 | 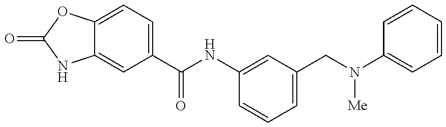 [SMILES: CN(Cc1cccc(NC(=O)c2ccc3oc(=O)[nH]c3c2)c1)c1ccccc1] |
| 221 | 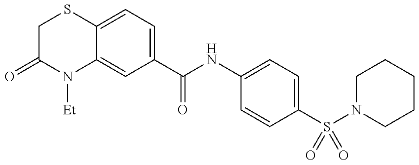 [SMILES: CCN1C(=O)CSc2ccc(C(=O)Nc3ccc(S(=O)(=O)N4CCCCC4)cc3)cc21] |
| 222 | 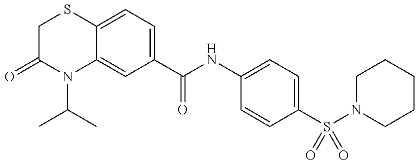 [SMILES: CC(C)N1C(=O)CSc2ccc(C(=O)Nc3ccc(S(=O)(=O)N4CCCCC4)cc3)cc21] |
| 223 | 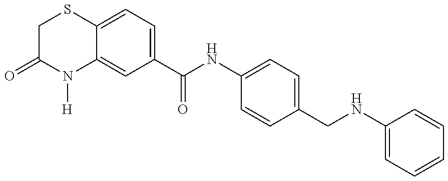 [SMILES: O=C1CSc2ccc(C(=O)Nc3ccc(CNc4ccccc4)cc3)cc2N1] |
| 224 | 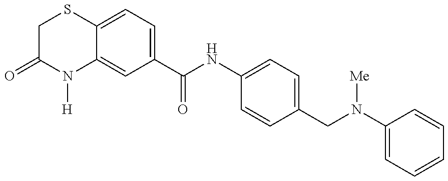 [SMILES: CN(Cc1ccc(NC(=O)c2ccc3c(c2)NC(=O)CS3)cc1)c1ccccc1] |
| 225 | 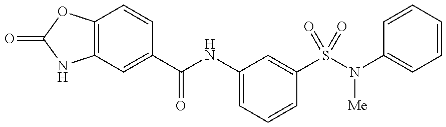 [SMILES: CN(c1ccccc1)S(=O)(=O)c1cccc(NC(=O)c2ccc3oc(=O)[nH]c3c2)c1] |
| 226 | 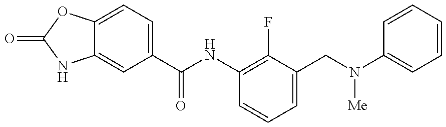 [SMILES: CN(Cc1cccc(NC(=O)c2ccc3oc(=O)[nH]c3c2)c1F)c1ccccc1] |
| 227 | 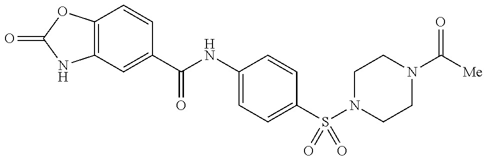 [SMILES: CC(=O)N1CCN(S(=O)(=O)c2ccc(NC(=O)c3ccc4oc(=O)[nH]c4c3)cc2)CC1] |
| 228 | 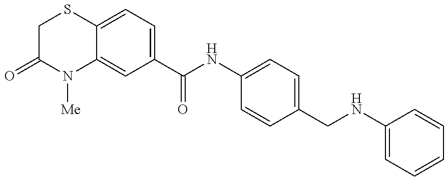 [SMILES: CN1C(=O)CSc2ccc(C(=O)Nc3ccc(CNc4ccccc4)cc3)cc21] |
| 229 | 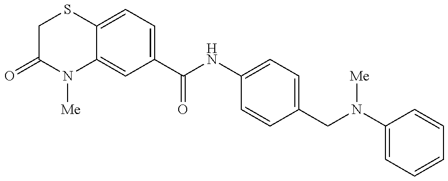 [SMILES: CN(Cc1ccc(NC(=O)c2ccc3c(c2)N(C)C(=O)CS3)cc1)c1ccccc1] |
| 230 | 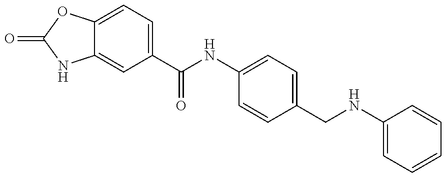 [SMILES: O=C(Nc1ccc(CNc2ccccc2)cc1)c1ccc2oc(=O)[nH]c2c1] |
| 231 | 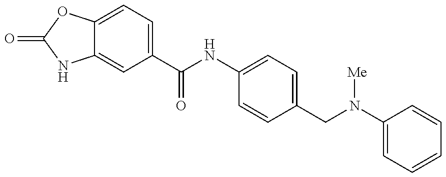 [SMILES: CN(Cc1ccc(NC(=O)c2ccc3oc(=O)[nH]c3c2)cc1)c1ccccc1] |
| 232 | 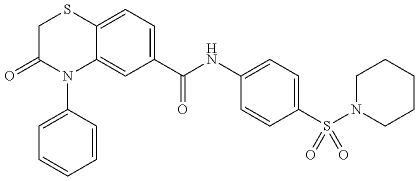 [SMILES: O=C(Nc1ccc(S(=O)(=O)N2CCCCC2)cc1)c1ccc2c(c1)N(c1ccccc1)C(=O)CS2] |

Compounds corresponding to Examples 1-232 have been synthesized and are provided with an Example number in Table 1.

### GENERAL EXPERIMENTAL METHODS

All chemicals and reagents were purchased from commercial suppliers and used without further purification. A Teledyne ISCO CombiF/ash Rf⁺ instrument equipped with a variable wavelength UV detector and a fraction collector was used to conduct flash column chromatography. HP C18 RediSep Rf reverse phase silica columns were also used for purification of certain polar products. Some compounds received final purification with preparative high-performance liquid chromatography (HPLC) on an Agilent Prep 1200 series with the UV detector set to 254 nm. Separation was performed at room temperature with a flow rate of 40 mL/min. Samples were injected into a Phenomenex Luna 750×30 mm, 5 μm C18 column, with the gradient program set to 10% of methanol (A) in H₂O containing 0.1% TFA (B) progressing to 100% of methanol or acetonitrile (A). HPLC spectra for all compounds were acquired using an Agilent 1200 Series system with DAD detector. Chromatography was performed on a 2.1×150 mm Zorbax 300SB-C18 5 μm column with water containing 0.1% formic acid as solvent A and acetonitrile containing 0.1% formic acid as solvent B at a flow rate of 0.4 mL/min. The linear gradient was as follows: 1% B (0-1 min), 1-99% B (1-4 min), and 99% B (4-8 min). HPLC was used to establish the purity of target compounds. All compounds showed >95% purity using the HPLC methods described above. High-resolution mass spectra (HRMS) data were acquired in positive ion mode using an Agilent G1969A API-TOF with an electrospray ionization (ESI) source. Proton Nuclear Magnetic Resonance (¹H-NMR) spectra were recorded on a Bruker DRX-600 spectrometer. Chemical shifts are expressed in parts per million (ppm) and reported as δ value (chemical shift δ). Coupling constants are reported in units of hertz (J value, Hz; Integration and splitting patterns: where s=singlet, d=double, t=triplet, q=quartet, brs=broad singlet).

### Example 1

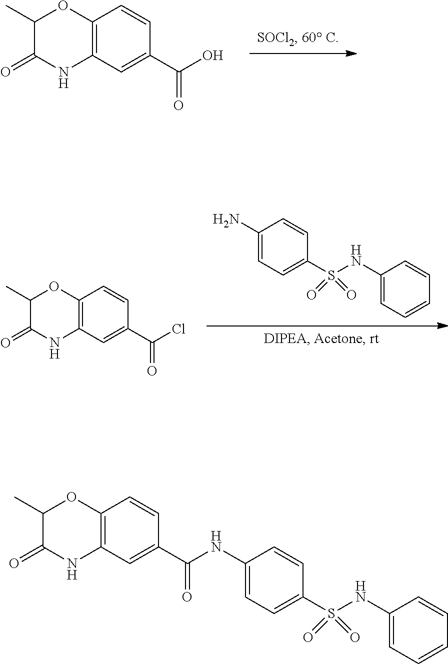

2-methyl-3-oxo-3,4-dihydro-2H-benzo[b][1,4]oxazine-6-carboxylic acid (41.4 mg, 0.2 mmol) was suspended in thionyl chloride (3 mL). The resulting suspension was stirring at 60° C. for 2 h, and then the solvent was removed. The white solid was dried in vacuum and dissolved in acetone (6 mL). 4-amino-N-phenylbenzenesulfonamide (49.6 mg, 0.2 mmol, 1.0 equiv) and DIPEA (N,N-diisopropylethylamine) (116 μL, 0.6 mmol, 3.0 equiv) were added. The reaction was stirring at room temperature overnight. The solvent was evaporated and the mixture was purified with the reverse phase ISCO to a white solid (24.3 mg, yield 28%). ¹H NMR (600 MHz, DMSO-d₆) δ 10.85 (d, J=5.3 Hz, 1H), 10.47 (d, J=5.1 Hz, 1H), 10.18 (d, J=5.1 Hz, 1H), 7.86 (dq, J=8.2, 2.5 Hz, 2H), 7.71 (td, J=5.5, 2.3 Hz, 2H), 7.56 (dt, J=8.3, 2.6 Hz, 1H), 7.50-7.40 (m, 1H), 7.21 (ddt, J=7.3, 5.2, 3.6 Hz, 2H), 7.08 (dd, J=8.1, 5.0 Hz, 3H), 7.00 (td, J=7.3, 3.2 Hz, 1H), 4.84-4.66 (m, 1H), 1.43 (dd, J=8.1, 3.8 Hz, 3H). t_{R}=4.40 min; HRMS m/z [M+H]⁺ calculated for C₂₂H₂₀N₃O₅S⁺ 438.1118, found 438.1132.

### Example 2

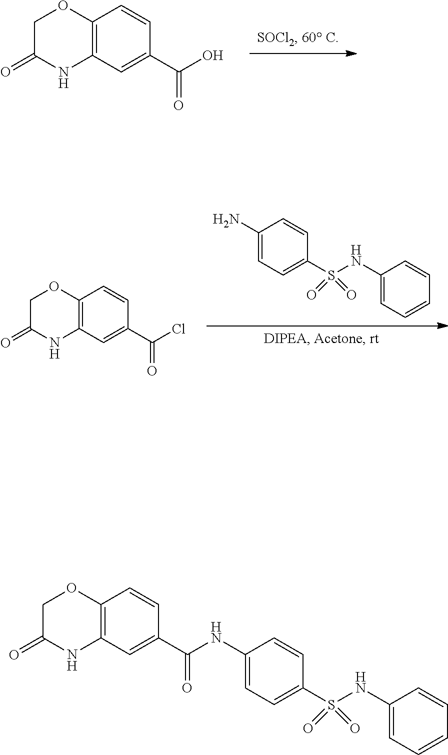

Compound was prepared following the general procedure described above from 3-oxo-3,4-dihydro-2H-benzo[b][1,4]oxazine-6-carboxylic acid (57.9 mg, 0.3 mmol). White solid (35.9 mg, yield 28%). ¹H NMR (600 MHz, CD₃OD) δ 7.80 (d, J=8.5 Hz, 2H), 7.71 (d, J=8.5 Hz, 2H), 7.66 (d, J=8.4 Hz, 1H), 7.56 (dd, J=10.6, 3.3 Hz, 1H), 7.46 (d, J=2.2 Hz, 1H), 7.21 (t, J=7.7 Hz, 1H), 7.13-6.95 (m, 4H), 4.66 (dd, J=4.6, 1.9 Hz, 2H). t_{R}=4.28 min; HRMS m/z [M+H]⁺ calculated for C₂₁H₁₈N₃O₅S⁺ 424.0962, found 424.0910.

### Example 3

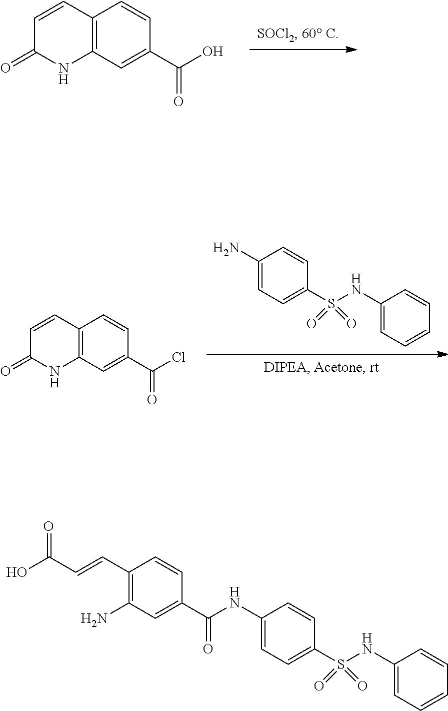 [SMILES: O=C(O)c1ccc2ccc(=O)[nH]c2c1 ; "SOCl2, 60° C." ; O=C(Cl)c1ccc2ccc(=O)[nH]c2c1 ; Nc1ccc(S(=O)(=O)Nc2ccccc2)cc1 ; "DIPEA, Acetone, rt" ; Nc1cc(C(=O)Nc2ccc(S(=O)(=O)Nc3ccccc3)cc2)ccc1/C=C/C(=O)O]

Compound was prepared following the general procedure described above from 2-oxo-1,2-dihydroquinoline-7-carboxylic acid (37.8 mg, 0.2 mmol). White solid (13.8 mg, yield 16%). ¹H NMR (600 MHz, DMSO-d₆) δ 10.84 (s, 1H), 10.21 (s, 1H), 8.62-8.41 (m, 2H), 8.18 (d, J=8.5 Hz, 1H), 8.10 (dd, J=8.5, 1.8 Hz, 1H), 8.02-7.88 (m, 2H), 7.84-7.61 (m, 3H), 7.28-7.13 (m, 2H), 7.15-7.04 (m, 2H), 7.04-6.91 (m, 1H). t_{R}=4.79 min; HRMS m/z [M+H]⁺ calculated for C₂₂H₂ON₃O₅S⁺ 438.1118, found 438.0635.

### Example 4

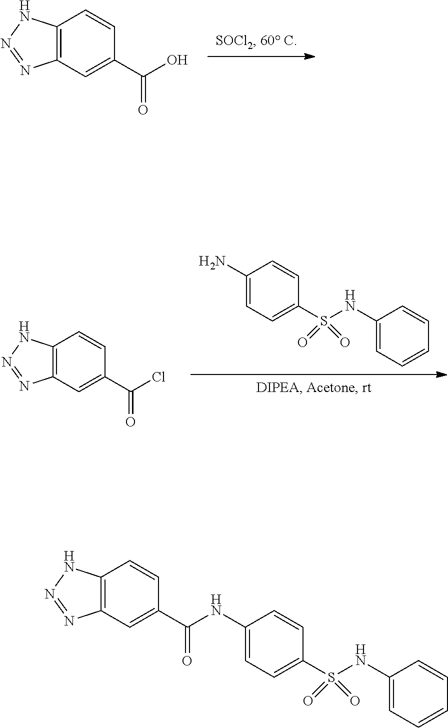

Compound was prepared following the general procedure described above from 1H-benzo[d][1,2,3]triazole-5-carboxylic acid (48.9 mg, 0.3 mmol). White solid (23.2 mg, yield 20%). ¹H NMR (600 MHz, Methanol-d₄) δ 10.50 (s, 1H), 8.53 (s, 1H), 8.04 (d, J=8.7 Hz, 1H), 7.93 (d, J=8.7 Hz, 1H), 7.87 (dd, J=8.9, 2.9 Hz, 2H), 7.74 (d, J=8.5 Hz, 2H), 7.21 (t, J=7.8 Hz, 2H), 7.14-7.01 (m, 3H). t_{R}=4.56 min; HRMS m/z [M+H]⁺ calculated for C₁₉H₁₆N₅O₃S⁺ 394.0968, found 394.0980.

### Example 5

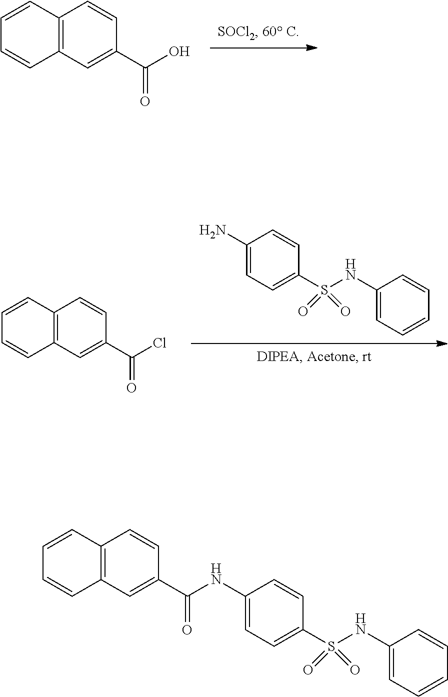

Compound was prepared following the general procedure described above from 2-naphthoic acid (34.4 mg, 0.2 mmol). White solid (22.8 mg, yield 28%). ¹H NMR (600 MHz, DMSO-d₆) δ 10.73 (s, 1H), 10.19 (s, 1H), 8.55 (s, 1H), 8.12-7.84 (m, 6H), 7.74 (d, J=8.7 Hz, 2H), 7.70-7.55 (m, 2H), 7.22 (t, J=7.9 Hz, 2H), 7.09 (d, J=8.0 Hz, 2H), 7.01 (t, J=7.4 Hz, 1H). t_{R}=4.93 min; HRMS m/z [M+H]⁺ calculated for C₂₃H₁₉N₂O₃S⁺ 403.1111, found 403.1118.

### Example 6

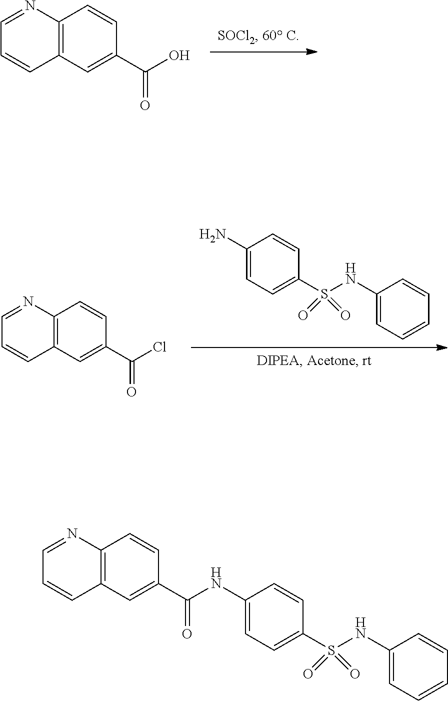

Compound was prepared following the general procedure described above from quinoline-6-carboxylic acid (86.5 mg, 0.5 mmol). White solid (85.3 mg, yield 42%). ¹H NMR (600 MHz, DMSO-d₆) δ 10.93 (d, J=5.2 Hz, 1H), 10.22 (d, J=3.8 Hz, 1H), 9.13 (t, J=5.9 Hz, 1H), 8.75 (d, J=20.7 Hz, 2H), 8.42-8.09 (m, 2H), 7.96 (d, J=9.0 Hz, 2H), 7.85-7.61 (m, 3H), 7.22 (t, J=7.9 Hz, 2H), 7.13-6.86 (m, 3H). t_{R}=4.29 min; HRMS m/z [M+H]⁺ calculated for C₂₂H₁₈N₃O₃S⁺ 404.1063, found 404.1053.

### Example 7

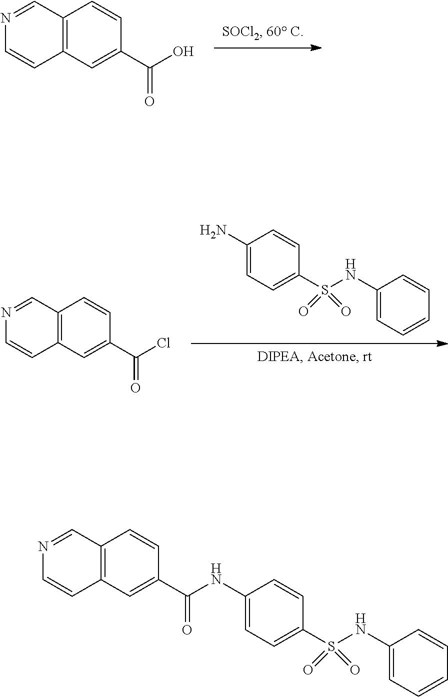

Compound was prepared following the general procedure described above from isoquinoline-6-carboxylic acid (17.3 mg, 0.1 mmol). White solid (15.3 mg, yield 38%). ¹H NMR (600 MHz, DMSO-d₆) δ 10.87 (s, 1H), 10.21 (s, 1H), 9.44 (s, 1H), 8.58 (d, J=34.7 Hz, 2H), 8.27 (d, J=8.4 Hz, 1H), 8.10 (d, J=8.5 Hz, 1H), 7.97 (dd, J=29.4, 7.0 Hz, 2H), 7.76 (d, J=8.3 Hz, 2H), 7.22 (t, J=7.7 Hz, 2H), 7.09 (d, J=7.8 Hz, 2H), 7.01 (t, J=7.3 Hz, 1H). t_{R}=4.37 min; HRMS m/z [M+H]⁺ calculated for C₂₂H₁₈N₃O₃S⁺ 404.1063, found 404.1078.

### Example 8

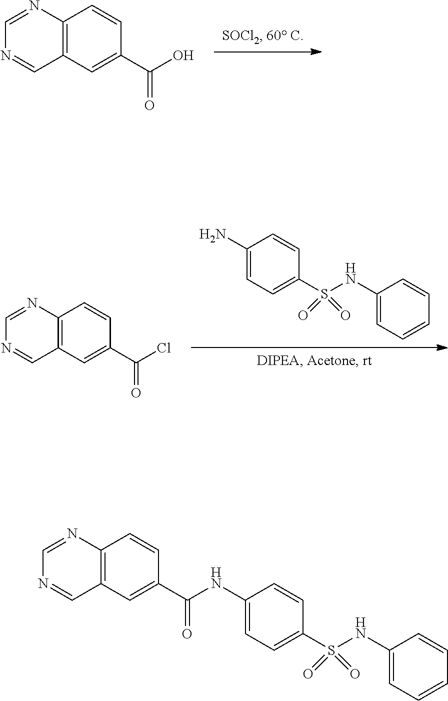

Compound was prepared following the general procedure described above from quinazoline-6-carboxylic acid (34.8 mg, 0.2 mmol). White solid (5.7 mg, yield 7%). ¹H NMR (600 MHz, CD₃OD) δ 9.64 (d, J=4.3 Hz, 1H), 9.35 (d, J=4.7 Hz, 1H), 8.56 (d, J=4.6 Hz, 1H), 8.34-8.14 (m, 2H), 7.90 (dd, J=8.6, 4.3 Hz, 2H), 7.80-7.65 (m, 2H), 7.28-7.16 (m, 2H), 7.16-6.98 (m, 3H). t_{R}=4.53 min; HRMS m/z [M+H]⁺ calculated for C₂₁H₁₇N₄O₃S⁺ 405.1016, found 405.1036.

### Example 9

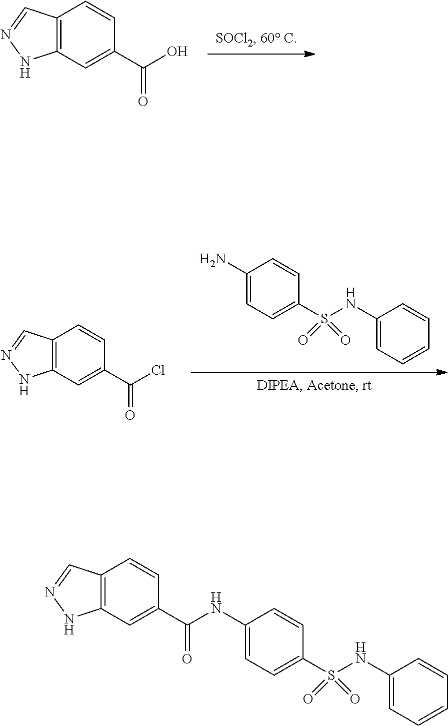

Compound was prepared following the general procedure described above from 1H-indazole-6-carboxylic acid (81 mg, 0.5 mmol). White solid (12.8 mg, yield 7%). ¹H NMR (600 MHz, CD₃OD) δ 8.14 (s, 2H), 7.91-7.83 (m, 3H), 7.76-7.71 (m, 2H), 7.67 (dd, J=8.5, 1.3 Hz, 1H), 7.24-7.17 (m, 2H), 7.14-7.09 (m, 2H), 7.06 (tt, J=7.4, 1.3 Hz, 1H). t_{R}=4.29 min; HRMS m/z [M+H]⁺ calculated for C₂₀H₁₇N₄O₃S⁺ 393.1016, found 393.1024.

### Example 10

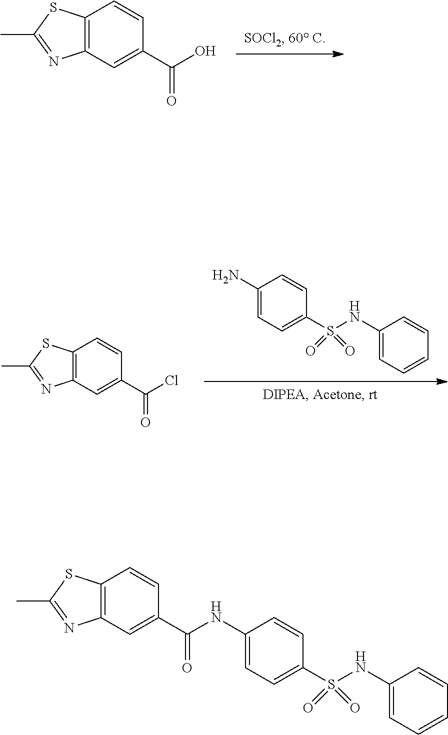

Compound was prepared following the general procedure described above from 2-methylbenzo[d]thiazole-5-carboxylic acid (38.6 mg, 0.2 mmol). White solid (18.2 mg, yield 22%). ¹H NMR (600 MHz, DMSO-d₆) δ 10.67 (s, 1H), 10.19 (s, 1H), 8.50 (d, J=1.7 Hz, 1H), 8.18 (d, J=8.4 Hz, 1H), 8.00-7.84 (m, 3H), 7.82-7.65 (m, 2H), 7.28-7.16 (m, 2H), 7.16-7.04 (m, 2H), 7.04-6.90 (m, 1H), 2.83 (s, 3H). t_{R}=4.62 min; HRMS m/z [M+H]⁺ calculated for C₂₁H₁₈N₃O₃S₂⁺ 424.0784, found 424.0774.

### Example 11

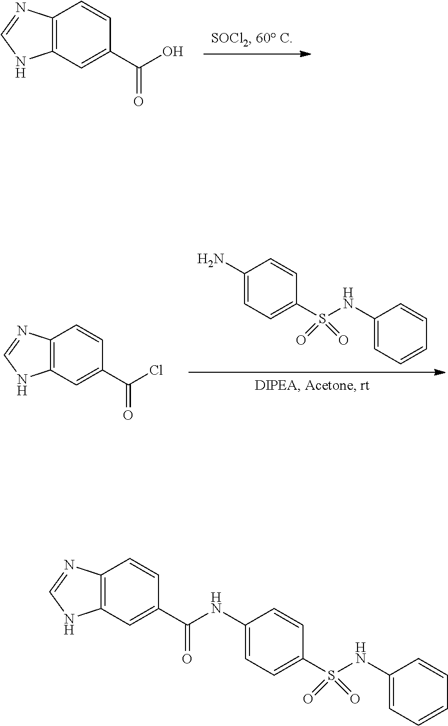

Compound was prepared following the general procedure described above from 1H-benzo[d]imidazole-6-carboxylic acid (48.6 mg, 0.3 mmol). White solid (9.1 mg, yield 8%). ¹H NMR (600 MHz, CD₃OD) δ 9.44 (s, 1H), 8.41 (s, 1H), 8.16 (d, J=8.5 Hz, 1H), 7.94 (d, J=8.7 Hz, 1H), 7.90-7.82 (m, 2H), 7.78-7.70 (m, 2H), 7.25-6.94 (m, 5H). t_{R}=4.05 min; HRMS m/z [M+H]⁺ calculated for C₂₀H₁₇N₄O₃S⁺ 393.1016, found 393.1036.

### Example 12

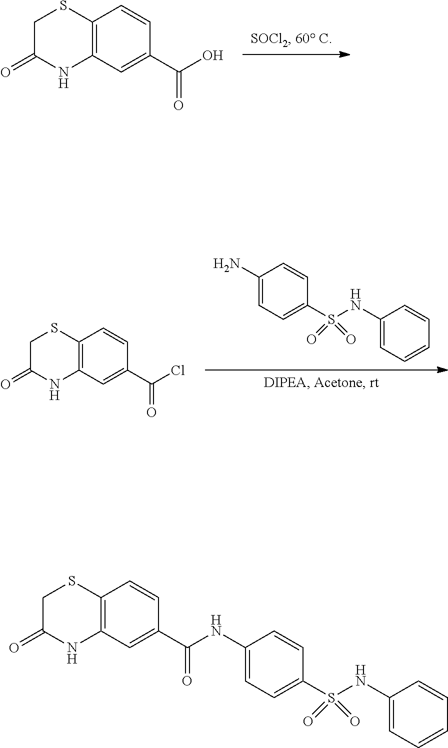

Compound was prepared following the general procedure described above from 3-oxo-3,4-dihydro-2H-benzo[b][1,4]thiazine-6-carboxylic acid (62.7 mg, 0.3 mmol). White solid (38.6 mg, yield 29%). ¹H NMR (600 MHz, DMSO-d₆) δ 10.74 (s, 1H), 10.53 (s, 1H), 10.18 (s, 1H), 7.87 (d, J=8.9 Hz, 2H), 7.71 (d, J=8.8 Hz, 2H), 7.55 (dd, J=8.1, 1.9 Hz, 1H), 7.51-7.43 (m, 2H), 7.21 (dd, J=8.6, 7.3 Hz, 2H), 7.11-7.03 (m, 2H), 7.04-6.93 (m, 1H), 3.52 (s, 2H). t_{R}=4.54 min; HRMS m/z [M+H]⁺ calculated for C₂₁H₁₈N₃O₄S₂⁺ 440.0733, found 440.0724.

### Example 13

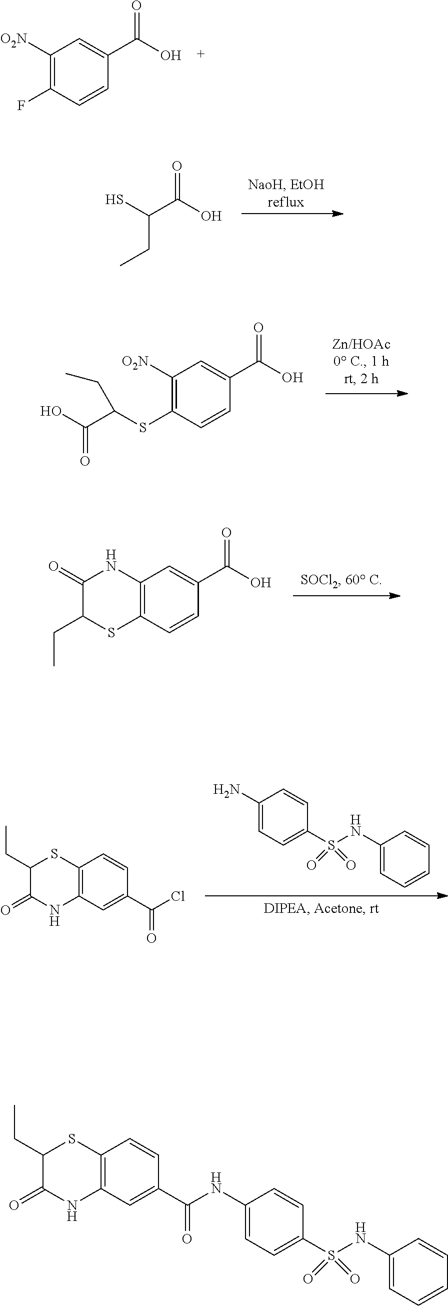

To the solution of 4-fluoro-3-nitrobenzoic acid (185 mg, 1 mmol) and 2-mercaptobutanoic acid (144 mg, 1.2 mmol, 1.2 equiv) in ethanol (3 mL) was added aq. NaOH (6 N, 1.7 mL, 10 mmol). The reaction was reflux for 1 h and cooled to room temperature. The mixture was neutralized with HCl (5 N), extracted with EtOAc (3×10 mL), dried with Na₂SO₄, and evaporated. The resulting crude product was dissolved in acetic acid (3 mL), zinc powder was added at 0° C. The reaction was warmed to room temperature slowly and stirring overnight. Sat. NaHCO₃ was added and extracted with EtOAc (3×10 mL), dried over Na₂SO₄, evaporated and SOCl₂ (3 mL) was added. The reaction was heated to 60° C. for 3 h, and the solvent was removed. The residue was dissolved in acetone, 4-amino-N-phenylbenzenesulfonamide (24.8 mg, 0.1 mmol, 1.0 equiv) and DIPEA (56 μL, 0.3 mmol, 3.0 equiv) were added. The reaction was stirring at room temperature overnight. The solvent was evaporated and the mixture was purified with reverse phase ISCO to obtain Example 13 as white solid (10.9 mg, yield 39%). ¹H NMR (600 MHz, DMSO-d₆) δ 10.75 (s, 1H), 10.55 (s, 1H), 10.19 (s, 1H), 7.95-7.80 (m, 2H), 7.78-7.65 (m, 2H), 7.58-7.51 (m, 1H), 7.51-7.42 (m, 2H), 7.25-7.15 (m, 2H), 7.11-7.06 (m, 2H), 7.04-6.96 (m, 1H), 3.16-3.04 (m, 1H), 1.80-1.68 (m, 1H), 1.54-1.36 (m, 1H), 0.95 (t, J=7.4 Hz, 3H). t_{R}=4.63 min; HRMS m/z [M+H]⁺ calculated for C₂₃H₂₂N₃O₄S₂⁺ 468.1046, found 468.1043.

### Example 14

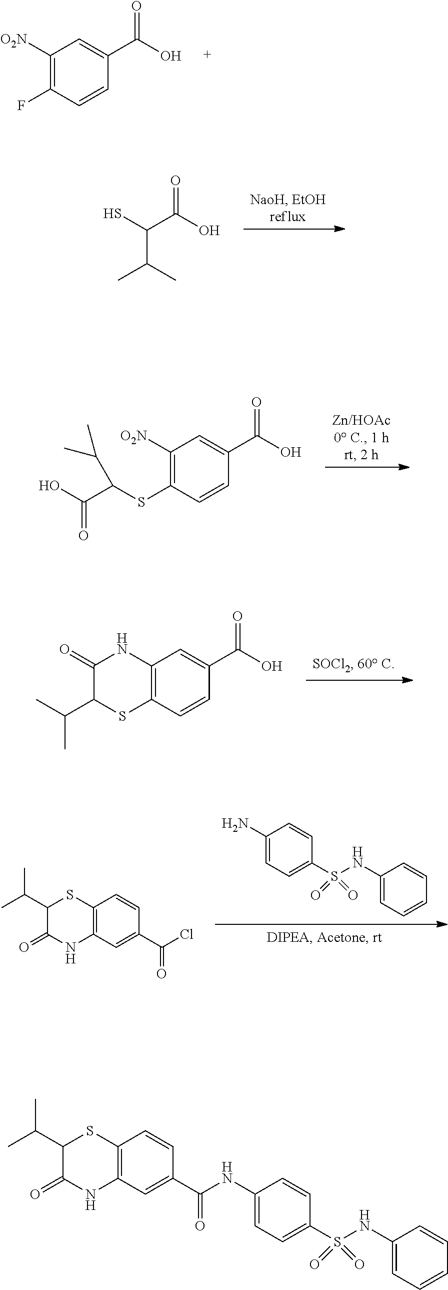

Compound was prepared following the general procedure described above from 4-fluoro-3-nitrobenzoic acid (370 mg, 2 mmol) and 2-mercapto-3-methylbutanoic acid (346 mg, 2.4 mmol). White solid (22.3 mg, yield 15%). ¹H NMR (600 MHz, DMSO-d₆) δ 10.78 (s, 1H), 10.53 (s, 1H), 10.19 (s, 1H), 7.93-7.77 (m, 2H), 7.71 (d, J=8.8 Hz, 2H), 7.58-7.40 (m, 3H), 7.26-7.16 (m, 2H), 7.13-7.04 (m, 2H), 7.00 (t, J=7.4 Hz, 1H), 3.25-3.10 (m, 1H), 1.83-1.68 (m, 1H), 0.95 (dd, J=6.7, 4.5 Hz, 6H). t_{R}=4.66 min; HRMS m/z [M+H]⁺ calculated for C₂₄H₂₄N₃O₄S₂⁺ 482.1203, found 482.1211.

### Example 15

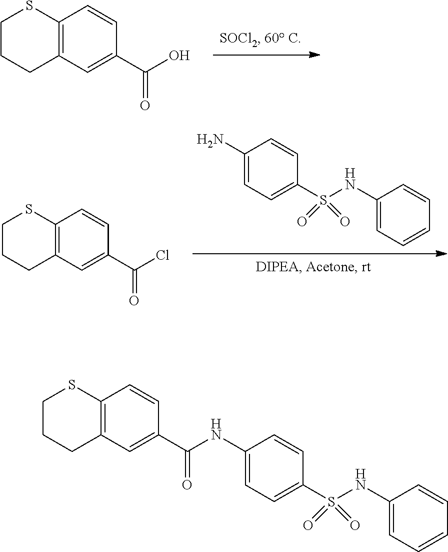 [SMILES: O=C(O)c1ccc2c(c1)CCCS2 ; "SOCl2, 60° C." ; O=C(Cl)c1ccc2c(c1)CCCS2 ; Nc1ccc(S(=O)(=O)Nc2ccccc2)cc1 ; "DIPEA, Acetone, rt" ; O=C(Nc1ccc(S(=O)(=O)Nc2ccccc2)cc1)c1ccc2c(c1)CCCS2]

Compound was prepared following the general procedure described above from thiochromane-6-carboxylic acid (40.4 mg, 0.21 mmol). White solid (48.3 mg, yield 54%). ¹H NMR (600 MHz, DMSO-d₆) δ 10.39 (d, J=3.8 Hz, 1H), 10.17 (d, J=3.7 Hz, 1H), 7.88 (dd, J=8.9, 3.2 Hz, 2H), 7.78-7.53 (m, 3H), 7.20 (tt, J=15.2, 6.5 Hz, 3H), 7.13-6.94 (m, 2H), 3.16-2.96 (m, 2H), 2.84 (dq, J=11.6, 6.4 Hz, 2H), 2.10-1.83 (m, 2H). t_{R}=4.76 min; HRMS m/z [M+H]⁺ calculated for C₂₂H₂₁N₂O₃S₂⁺ 425.0988, found 425.0965.

### Example 16

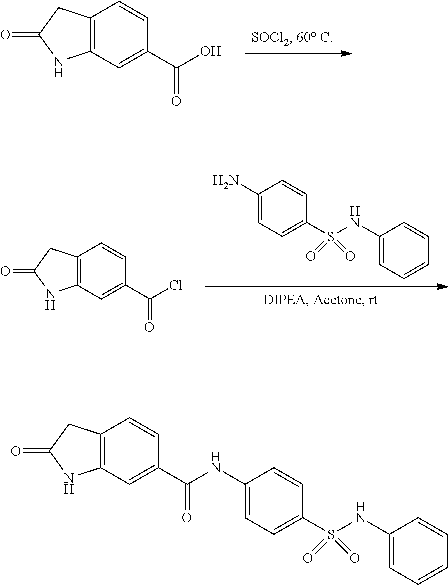 [SMILES: O=C1Cc2ccc(C(=O)O)cc2N1 ; "SOCl2, 60° C." ; O=C1Cc2ccc(C(=O)Cl)cc2N1 ; Nc1ccc(S(=O)(=O)Nc2ccccc2)cc1 ; "DIPEA, Acetone, rt" ; O=C1Cc2ccc(C(=O)Nc3ccc(S(=O)(=O)Nc4ccccc4)cc3)cc2N1]

Compound was prepared following the general procedure described above from 2-oxoindoline-6-carboxylic acid (53.1 mg, 0.3 mmol). White solid (5.6 mg, yield 5%). ¹H NMR (600 MHz, CD₃OD) δ 7.82 (dd, J=8.9, 2.4 Hz, 2H), 7.72 (dd, J=8.9, 2.3 Hz, 2H), 7.57 (dt, J=7.8, 2.1 Hz, 1H), 7.41-7.35 (m, 1H), 7.24-7.18 (m, 2H), 7.13-7.02 (m, 4H), 3.34 (d, J=2.3 Hz, 2H). t_{R}=4.45 min; HRMS m/z [M+H]⁺ calculated for C₂₁H₁₈N₃O₄S⁺ 408.1013, found 408.1024.

### Example 17

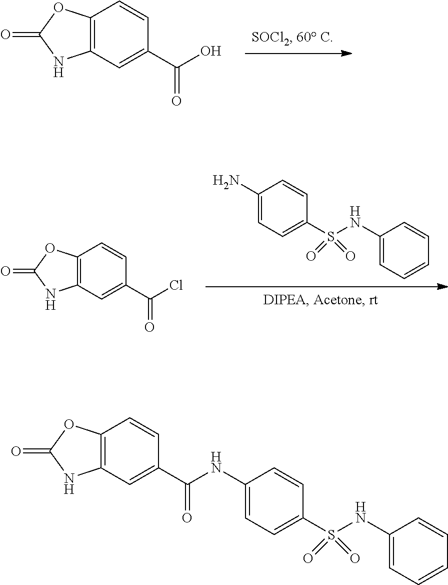 [SMILES: O=C(O)c1ccc2oc(=O)[nH]c2c1 ; "SOCl2, 60° C." ; O=C(Cl)c1ccc2oc(=O)[nH]c2c1 ; Nc1ccc(S(=O)(=O)Nc2ccccc2)cc1 ; "DIPEA, Acetone, rt" ; O=C(Nc1ccc(S(=O)(=O)Nc2ccccc2)cc1)c1ccc2oc(=O)[nH]c2c1]

Compound was prepared following the general procedure described above from 2-oxo-2,3-dihydrobenzo[d]oxazole-5-carboxylic acid (53.7 mg, 0.3 mmol). White solid (8.5 mg, yield 7%). ¹H NMR (600 MHz, DMSO-d₆) δ 11.94 (s, 1H), 10.55 (s, 1H), 10.18 (s, 1H), 7.93-7.78 (m, 2H), 7.78-7.65 (m, 3H), 7.62 (d, J=1.8 Hz, 1H), 7.42 (d, J=8.4 Hz, 1H), 7.21 (dd, J=8.5, 7.3 Hz, 2H), 7.13-7.04 (m, 2H), 7.00 (t, J=7.4 Hz, 1H). t_{R}=4.36 min; HRMS m/z [M+H]⁺ calculated for C₂₀H₁₆N₂O₅S⁺ 410.0805, found 410.0812.

### Example 18

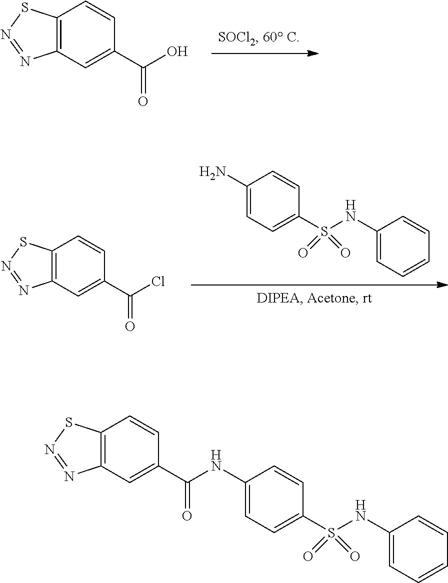 [SMILES: O=C(O)c1ccc2snnc2c1 ; "SOCl2, 60° C." ; O=C(Cl)c1ccc2snnc2c1 ; Nc1ccc(S(=O)(=O)Nc2ccccc2)cc1 ; "DIPEA, Acetone, rt" ; O=C(Nc1ccc(S(=O)(=O)Nc2ccccc2)cc1)c1ccc2snnc2c1]

Compound was prepared following the general procedure described above from benzo[d][1,2,3]thiadiazole-5-carboxylic acid (54 mg, 0.3 mmol). White solid (108 mg, yield 88%). ¹H NMR (600 MHz, DMSO-d₆) δ 10.89 (s, 1H), 10.21 (s, 1H), 9.31 (d, J=1.6 Hz, 1H), 8.55 (d, J=8.5 Hz, 1H), 8.28 (dd, J=8.5, 1.6 Hz, 1H), 8.03-7.90 (m, 2H), 7.85-7.71 (m, 2H), 7.28-7.16 (m, 2H), 7.15-7.04 (m, 2H), 7.01 (t, J=7.4 Hz, 1H). t_{R}=4.43 min; HRMS m/z [M+H]⁺ calculated for C₁₉H₁₅N₄O₃S₂⁺ 411.0580, found 411.0576.

### Example 19

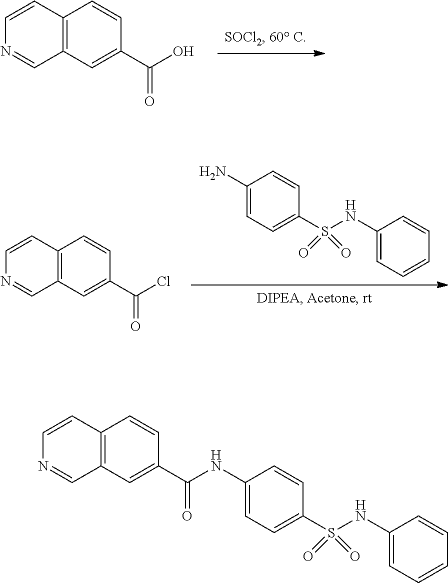 [SMILES: O=C(O)c1ccc2ccncc2c1 ; "SOCl2, 60° C." ; O=C(Cl)c1ccc2ccncc2c1 ; Nc1ccc(S(=O)(=O)Nc2ccccc2)cc1 ; "DIPEA, Acetone, rt" ; O=C(Nc1ccc(S(=O)(=O)Nc2ccccc2)cc1)c1ccc2ccncc2c1]

Compound was prepared following the general procedure described above from isoquinoline-7-carboxylic acid (51.9 mg, 0.3 mmol). White solid (15.2 mg, yield 13%). ¹H NMR (600 MHz, CD₃OD) δ 9.78 (d, J=5.8 Hz, 1H), 8.96 (d, J=5.9 Hz, 1H), 8.66 (t, J=6.4 Hz, 1H), 8.56 (ddd, J=8.6, 6.8, 1.8 Hz, 1H), 8.41 (t, J=6.4 Hz, 1H), 8.34 (t, J=7.6 Hz, 1H), 7.89 (tt, J=10.1, 5.2 Hz, 2H), 7.75 (td, J=6.8, 2.3 Hz, 2H), 7.27-7.16 (m, 2H), 7.14-6.99 (m, 3H). t_{R}=4.34 min; HRMS m/z [M+H]⁺ calculated for C₂₂H₁₈N₃O₃S⁺ 404.1063, found 404.1056.

### Example 20

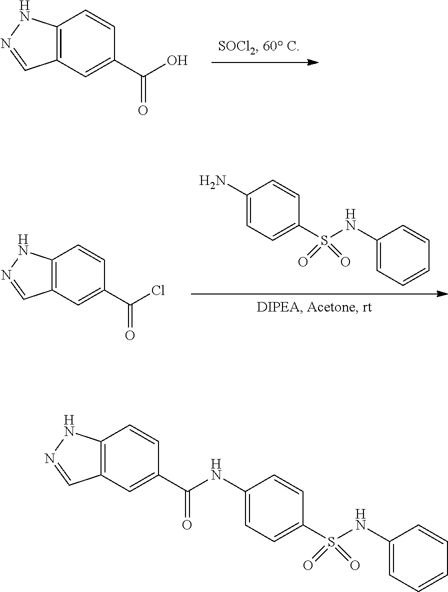 [SMILES: O=C(O)c1ccc2[nH]ncc2c1 ; "SOCl2, 60° C." ; O=C(Cl)c1ccc2[nH]ncc2c1 ; Nc1ccc(S(=O)(=O)Nc2ccccc2)cc1 ; "DIPEA, Acetone, rt" ; O=C(Nc1ccc(S(=O)(=O)Nc2ccccc2)cc1)c1ccc2[nH]ncc2c1]

Compound was prepared following the general procedure described above from 1H-indazole-5-carboxylic acid (48.6 mg, 0.3 mmol). White solid (15.2 mg, yield 13%). ¹H NMR (600 MHz, DMSO-d₆) δ 13.35 (s, 1H), 10.56 (s, 1H), 10.18 (s, 1H), 8.45 (d, J=7.1 Hz, 1H), 8.27 (s, 1H), 7.99-7.80 (m, 2H), 7.80-7.51 (m, 2H), 7.29-6.87 (m, 4H). t_{R}=4.37 min; HRMS m/z [M+H]⁺ calculated for C₂₀H₁₆N₂O₅S⁺ 410.0805, found 410.0812.

### Example 21

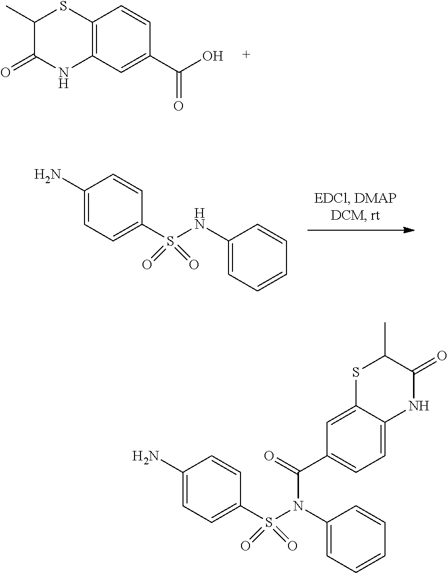 [SMILES: CC1Sc2ccc(C(=O)O)cc2NC1=O ; Nc1ccc(S(=O)(=O)Nc2ccccc2)cc1 ; "EDCl, DMAPDCM, rt" ; CC1Sc2cc(C(=O)N(c3ccccc3)S(=O)(=O)c3ccc(N)cc3)ccc2NC1=O]

To the solution of 2-methyl-3-oxo-3,4-dihydro-2H-benzo[b][1,4]thiazine-6-carboxylic acid (22.3 mg, 0.1 mmol) in DCM (3 mL) were added DMAP (4-amino-N-phenylbenzenesulfonamide) (24.8 mg, 0.1 mmol, 1.0 equiv), EDCI (1-ethyl-3-(3-dimethylaminopropyl)carbodiimide) (29 mg, 0.15 mmol, 1.5 equiv). After stirring overnight at room temperature, the resulting mixture was purified by preparative HPLC (10%-100% methanol/0.1% TFA in H₂O) to afford Example 21 as white solid (11.7 mg, yield 26%). ¹H NMR (600 MHz, DMSO-d₆) δ 10.60 (s, 1H), 7.48-7.38 (m, 1H), 7.31 (dt, J=4.8, 2.3 Hz, 3H), 7.19-7.08 (m, 2H), 7.06 (d, J=1.8 Hz, 1H), 6.94 (dd, J=8.1, 1.8 Hz, 1H), 6.64-6.52 (m, 2H), 6.30 (s, 2H), 3.64 (q, J=7.0 Hz, 1H), 1.20 (dd, J=7.0, 1.5 Hz, 3H). t_{R}=4.51 min; HRMS m/z [M+H]⁺ calculated for C₂₂H₂ON₃O₄S₂⁺ 454.0890, found 454.0887.

### Example 22

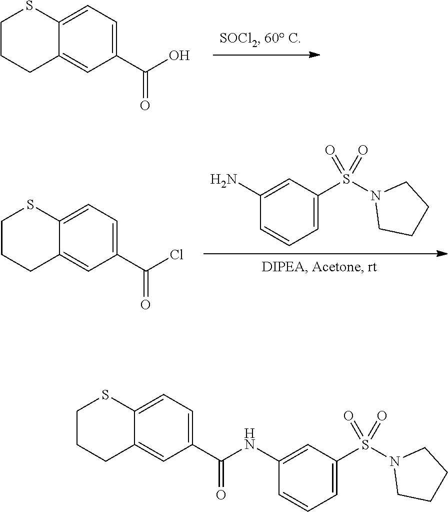

Compound was prepared following the general procedure described above from thiochromane-6-carboxylic acid (100 mg, 0.52 mmol) and 3-(pyrrolidin-1-ylsulfonyl)aniline (118 mg, 0.52 mmol). White solid (92.5 mg, yield 44%). ¹H NMR (600 MHz, DMSO-d₆) δ 10.40 (s, 1H), 8.27 (q, J=1.9 Hz, 1H), 8.09 (dd, J=6.5, 4.2 Hz, 1H), 7.74-7.63 (m, 2H), 7.58 (td, J=8.0, 2.0 Hz, 1H), 7.53-7.43 (m, 1H), 7.19 (dd, J=8.2, 2.0 Hz, 1H), 3.19-3.01 (m, 6H), 2.85 (t, J=6.1 Hz, 2H), 2.10-1.87 (m, 2H), 1.75-1.53 (m, 4H). t_{R}=4.83 min; HRMS m/z [M+H]⁺ calculated for C₂₀H₂₃N₂O₃S₂⁺ 403.1145, found 403.1098.

### Example 23

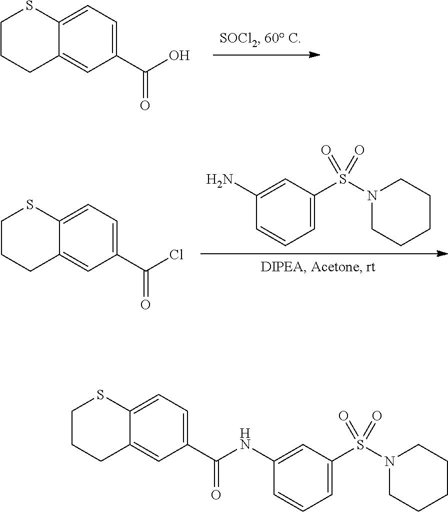

Compound was prepared following the general procedure described above from thiochromane-6-carboxylic acid (100 mg, 0.52 mmol) and 3-(piperidin-1-ylsulfonyl)aniline (125 mg, 0.52 mmol). White solid (118.2 mg, yield 55%). ¹H NMR (600 MHz, DMSO-d₆) δ 10.42 (s, 1H), 8.24-8.14 (m, 1H), 8.09 (d, J=8.2 Hz, 1H), 7.74-7.63 (m, 2H), 7.58 (td, J=8.0, 1.8 Hz, 1H), 7.40 (d, J=7.8 Hz, 1H), 7.18 (dd, J=8.2, 1.8 Hz, 1H), 3.15-3.03 (m, 2H), 2.86 (dt, J=24.3, 5.8 Hz, 6H), 2.07-1.92 (m, 2H), 1.60-1.41 (m, 4H), 1.41-1.25 (m, 2H). t_{R}=4.99 min; HRMS m/z [M+H]⁺ calculated for C₂₁H₂₅N₂O₃S₂⁺ 417.1301, found 417.1290.

### Example 24

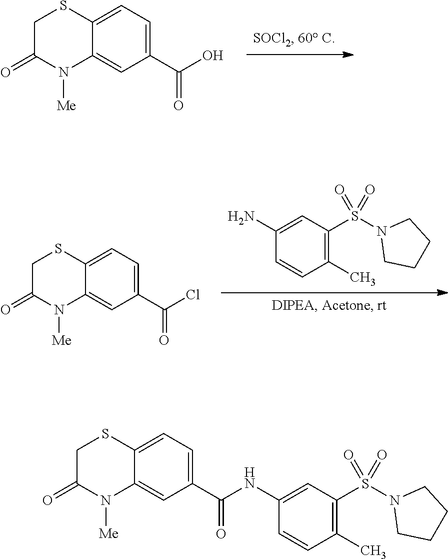

Compound was prepared following the general procedure described above from 4-methyl-3-oxo-3,4-dihydro-2H-benzo[b][1,4]thiazine-6-carboxylic acid (100 mg, 0.45 mmol) and 4-methyl-3-(pyrrolidin-1-ylsulfonyl)anilinene (108 mg, 0.45 mmol). White solid (92.8 mg, yield 46%). ¹H NMR (600 MHz, DMSO-d₆) δ 10.50 (s, 1H), 8.28 (d, J=2.5 Hz, 1H), 7.92 (dd, J=8.3, 2.3 Hz, 1H), 7.74 (d, J=1.8 Hz, 1H), 7.66 (dd, J=8.0, 1.8 Hz, 1H), 7.56 (dd, J=8.1, 1.5 Hz, 1H), 7.42 (d, J=8.3 Hz, 1H), 3.58 (s, 2H), 3.42 (s, 3H), 3.26-3.11 (m, 4H), 2.48 (q, J=2.0 Hz, 4H), 1.90-1.77 (m, 3H). t_{R}=4.62 min; HRMS m/z [M+H]⁺ calculated for C₂₁H₂₄N₂O₄S₂⁺ 446.1203, found 446.1096.

### Example 25

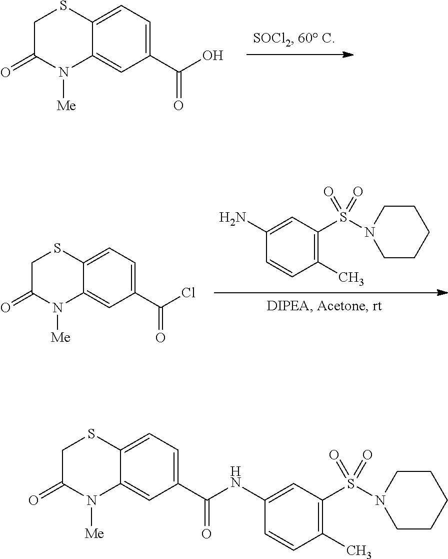

Compound was prepared following the general procedure described above from 4-methyl-3-oxo-3,4-dihydro-2H-benzo[b][1,4]thiazine-6-carboxylic acid (100 mg, 0.45 mmol) and 4-methyl-3-(piperidin-1-ylsulfonyl)aniline (114 mg, 0.45 mmol). White solid (92.8 mg, yield 46%). ¹H NMR (600 MHz, DMSO-d₆) δ 10.50 (s, 1H), 8.26 (s, 1H), 7.94 (dt, J=8.3, 2.2 Hz, 1H), 7.74 (s, 1H), 7.66 (d, J=8.1, 1.9 Hz, 1H), 7.56 (dd, J=8.1, 2.0 Hz, 1H), 7.41 (d, J=8.3 Hz, 1H), 3.57 (s, 2H), 3.42 (s, 3H), 3.05 (t, J=5.5 Hz, 4H), 1.52 (t, J=5.8 Hz, 6H), 1.44 (s, 3H). t_{R}=4.75 min; HRMS m/z [M+H]⁺ calculated for C₂₂H₂₆N₃O₄S₂⁺ 460.1359, found 460.1303.

### Example 26

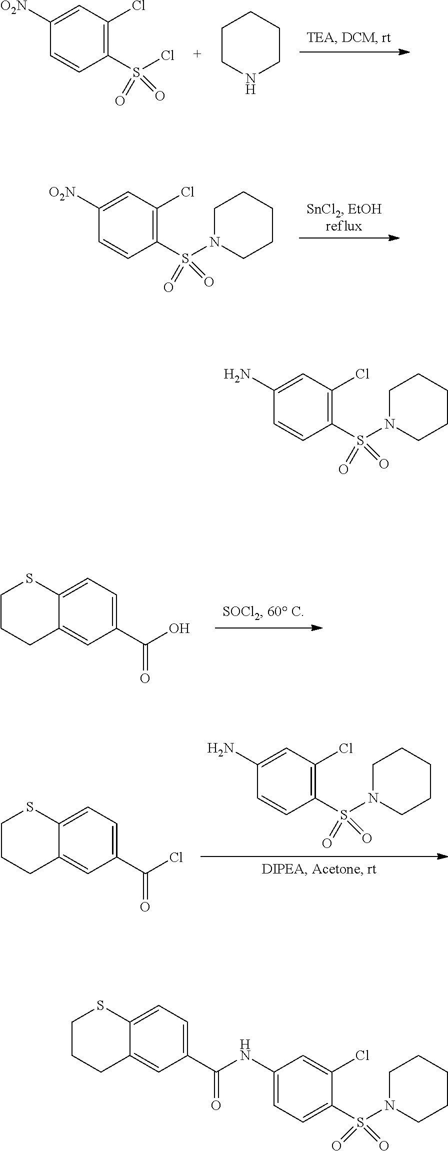

To the solution of 2-chloro-4-nitrobenzenesulfonyl chloride (128 mg, 0.5 mmol) in DCM (3 mL) was added piperidine (51 mg, 0.6 mmol, 1.2 equiv) and triethylamine (TEA, 152 mg, 1.5 mmol, 3.0 equiv). The reaction was stirring at room temperature for 3 h. Solvent was removed and the residue was purified by ISCO (Hexane/EtOAc=2:1) to obtain 1-((2-chloro-4-nitrophenyl)sulfonyl)piperidine (164 mg, yield 99%). This intermediate was dissolved in EtOH (5 mL). Tin chloride (SnCl₂, 337 mg, 1.5 mmol, 3 equiv) was added and the reaction was reflux overnight. Solvent was removed and the residue was purified by ISCO (DCM/MeOH=9:1) to yield intermediate 3-chloro-4-(piperidin-1-ylsulfonyl)aniline (140 mg, yield 98%). ESI-MS (m/z) [M+H]⁺: 275.08; Compound was prepared following the general procedure described above from thiochromane-6-carboxylic acid (97 mg, 0.5 mmol) and intermediate 3-chloro-4-(piperidin-1-ylsulfonyl)aniline (137 mg, 0.5 mmol). White solid (52.6 mg, yield 23%). ¹H NMR (600 MHz, DMSO-d₆) δ 10.48 (d, J=4.6 Hz, 1H), 8.43 (q, J=3.2 Hz, 1H), 8.16-8.00 (m, 1H), 7.74-7.51 (m, 3H), 7.18 (dt, J=7.8, 3.4 Hz, 1H), 3.21-3.10 (m, 4H), 3.07 (t, J=5.3 Hz, 2H), 2.84 (t, J=6.0 Hz, 2H), 2.02 (q, J=6.0 Hz, 2H), 1.56-1.37 (m, 6H). t_{R}=5.24 min; HRMS m/z [M+H]⁺ calculated for C₂₁H₂₄ClN₂O₃S₂⁺ 451.0911, found 451.0855.

### Example 27

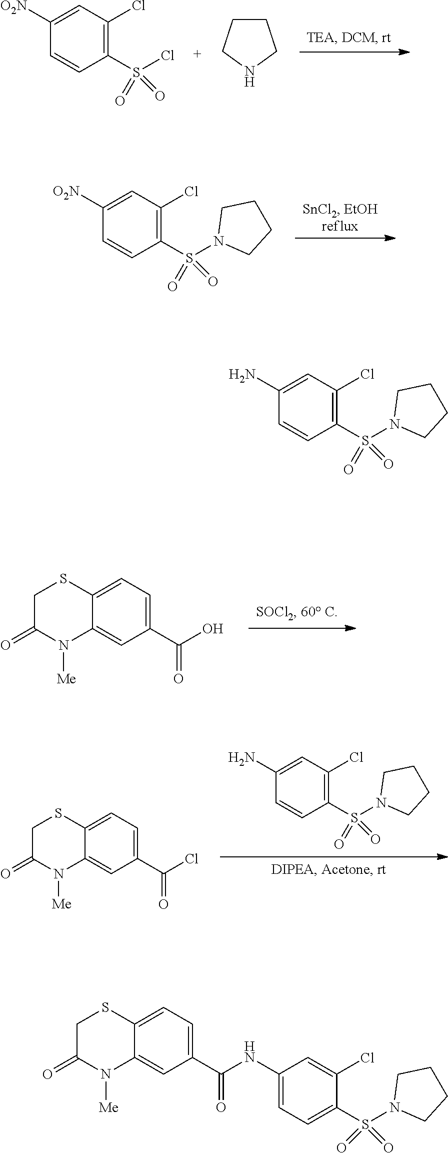

3-chloro-4-(pyrrolidin-1-ylsulfonyl)aniline was prepared following the general procedure for synthesizing 3-chloro-4-(piperidin-1-ylsulfonyl)aniline from 2-chloro-4-nitrobenzenesulfonyl chloride (128 mg, 0.5 mmol) and pyrrolidine (43 mg, 0.6 mmol, 1.2 equiv). White solid (125 mg, yield 98%). Compound was prepared following the general procedure described above from 4-methyl-3-oxo-3,4-dihydro-2H-benzo[b][1,4]thiazine-6-carboxylic acid (100 mg, 0.45 mmol) (112 mg, 0.5 mmol) and 3-chloro-4-(pyrrolidin-1-ylsulfonyl)aniline (130 mg, 0.5 mmol). White solid (43.5 mg, yield 19%). ¹H NMR (600 MHz, DMSO-d₆) δ 10.77 (s, 1H), 8.08 (d, J=2.1 Hz, 1H), 7.95-7.85 (m, 1H), 7.84-7.77 (m, 1H), 7.71-7.65 (m, 1H), 7.59 (dd, J=8.3, 1.9 Hz, 1H), 7.53 (dd, J=8.3, 2.7 Hz, 1H), 3.50 (s, 3H), 3.37 (s, 4H), 3.22 (s, 2H), 1.78-1.74 (m, 4H). t_{R}=4.65 min; HRMS m/z [M+H]⁺ calculated for C₂₀H₂₁ClN₃O₄S₂⁺ 466.0657, found 466.0559.

### Example 28

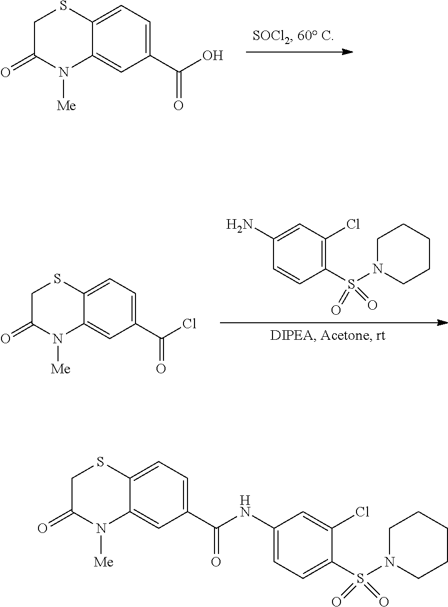 [SMILES: CN1C(=O)CSc2ccc(C(=O)O)cc21 ; "SOCl2, 60° C." ; CN1C(=O)CSc2ccc(C(=O)Cl)cc21 ; Nc1ccc(S(=O)(=O)N2CCCCC2)c(Cl)c1 ; "DIPEA, Acetone, rt" ; CN1C(=O)CSc2ccc(C(=O)Nc3ccc(S(=O)(=O)N4CCCCC4)c(Cl)c3)cc21]

Compound was prepared following the general procedure described above from 4-methyl-3-oxo-3,4-dihydro-2H-benzo[b][1,4]thiazine-6-carboxylic acid (100 mg, 0.45 mmol and 3-chloro-4-(piperidin-1-ylsulfonyl)aniline (137 mg, 0.5 mmol). White solid (25.8 mg, yield 11%). ¹H NMR (600 MHz, DMSO-d₆) δ 10.65 (s, 1H), 8.45 (d, J=2.6 Hz, 1H), 8.06 (dd, J=8.8, 2.6 Hz, 1H), 7.80-7.70 (m, 1H), 7.70-7.61 (m, 2H), 7.57 (d, J=8.0 Hz, 1H), 3.58 (s, 2H), 3.35 (s, 3H), 3.16 (t, J=5.4 Hz, 4H), 1.58-1.38 (m, 6H). t_{R}=4.83 min; HRMS m/z [M+H]⁺ calculated for C₂₁H₂₃ClN₃O₄S₂⁺ 480.0013, found 480.0022.

### Example 29

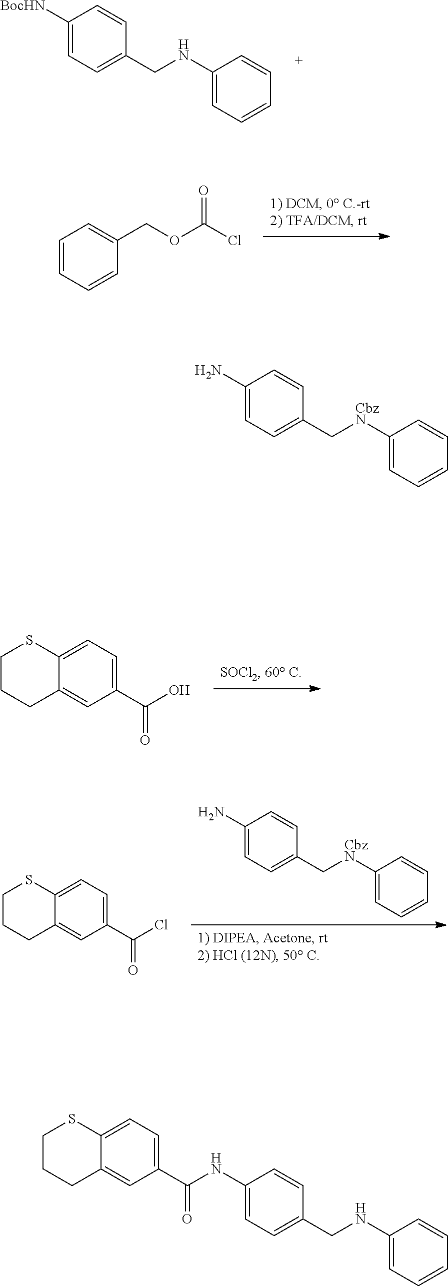

To the solution of tert-butyl (4-((phenylamino)methyl)phenyl)carbamate (240 mg, 0.81 mmol) in DCM (5 mL) was added benzyl carbonochloridate (165 mg, 0.97 mmol, 1.2 equiv) at 0° C. The reaction was warmed to room temperature and stirring overnight. Solvent was removed and the residue was purified by ISCO (Hexane/EtOAc=5:1) to 195 mg of intermediate (yield 56%). This intermediate was dissolved in DCM/TFA (3 mL/3 mL), and the resulting mixture was stirring for 30 mins. Solvent was evaporated and the crude product was dissolved in DMSO (1 mL). Thiochromane-6-carboxylic acid (58.2 mg, 0.3 mmol), EDCI (86.4 mg, 0.45 mmol, 1.5 equiv), HOAt (1-hydroxy-7-azabenzo-triazole) (61.2 mg, 0.45 mmol, 1.5 equiv), and NMM (N-Methylmorpholine) (90.9 mg, 0.9 mmol, 3.0 equiv) were added. The reaction was stirring overnight and purified by reverse phase ISCO: 70.8 mg. The white solid was dissolved in dioxane (1 mL) and HCl (12 N, a few drops) was added. The mixture was heated to 50° C. for 1 h. Sat. NaHCO₃ was added and extracted with EtOAc (3×10 mL), dried over Na₂SO₄. Solvent was removed and the residue was purified by reverse phase ISCO to obtain Example 29 as white solid (32.4 mg, yield 29%). ¹H NMR (600 MHz, DMSO-d₆) δ 10.06 (s, 1H), 7.71-7.58 (m, 4H), 7.30 (d, J=8.2 Hz, 2H), 7.15 (d, J=8.2 Hz, 1H), 7.07 (t, J=7.7 Hz, 2H), 6.62 (dd, J=28.0, 7.7 Hz, 3H), 4.23 (s, 2H), 3.15-2.99 (m, 2H), 2.83 (t, J=6.1 Hz, 2H), 2.01 (t, J=6.0 Hz, 2H). t_{R}=4.92 min; HRMS m/z [M+H]⁺ calculated for C₂₃H₂₃N₂OS⁺ 375.1526, found 375.1516.

### Example 30

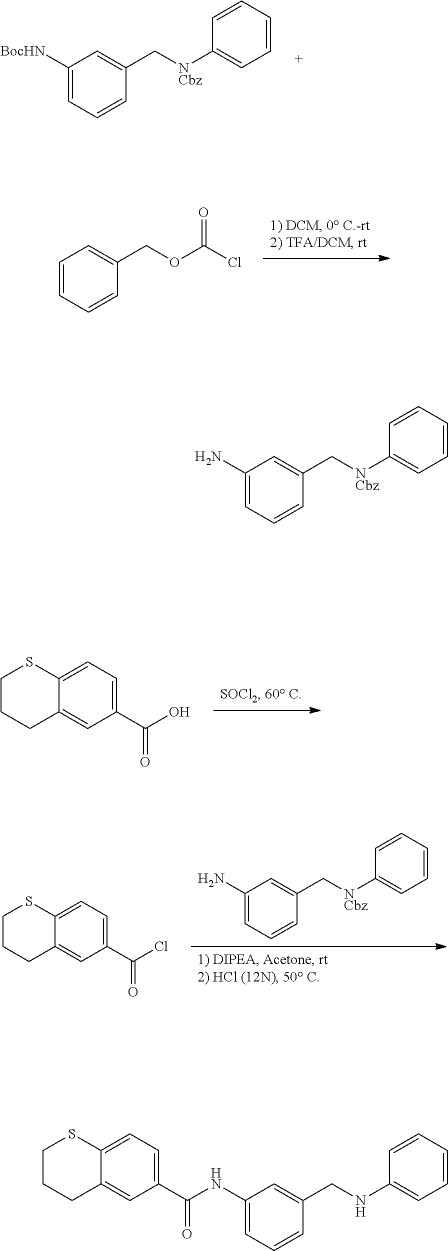

Compound was prepared following the general procedure described above from tert-butyl (3-((phenylamino)methyl)phenyl)carbamate (295 mg, 1 mmol). White solid (49.3 mg, yield 44%). ¹H NMR (600 MHz, DMSO-d₆) δ 10.08 (s, 1H), 7.75 (s, 1H), 7.70-7.55 (m, 3H), 7.26 (t, J=7.8 Hz, 1H), 7.19-6.94 (m, 4H), 6.66-6.45 (m, 3H), 4.24 (s, 2H), 3.06 (t, J=5.9 Hz, 2H), 2.83 (t, J=6.1 Hz, 2H), 2.01 (t, J=6.1 Hz, 2H)). t_{R}=4.98 min; HRMS m/z [M+H]⁺ calculated for C₂₃H₂₃N₂OS⁺ 375.1526, found 375.1546.

### Example 31

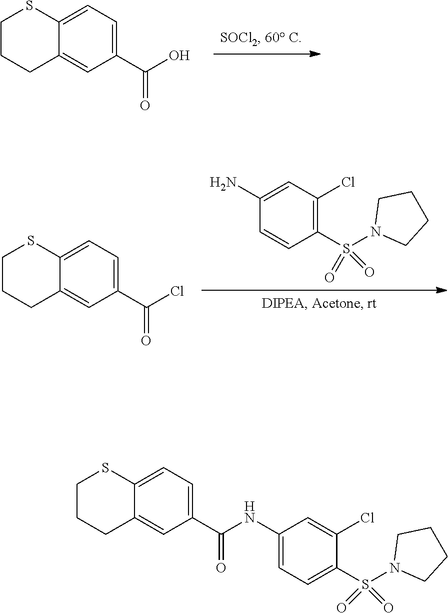

Compound was prepared following the general procedure described above from thiochromane-6-carboxylic acid (97 mg, 0.5 mmol) and 3-chloro-4-(pyrrolidin-1-ylsulfonyl)aniline (130 mg, 0.5 mmol). White solid (27.8 mg, yield 13%). ¹H NMR (600 MHz, DMSO-d₆) δ 10.49 (s, 1H), 8.45 (d, J=2.5 Hz, 1H), 8.05 (dt, J=8.9, 2.3 Hz, 1H), 7.71-7.57 (m, 3H), 7.18 (dd, J=8.2, 1.8 Hz, 1H), 3.33-3.23 (m, 4H), 3.13-3.01 (m, 2H), 2.84 (t, J=6.1 Hz, 2H), 2.01 (d, J=6.1 Hz, 2H), 1.83 (h, J=2.6 Hz, 4H). t_{R}=5.00 min; HRMS m/z [M+H]⁺ calculated for C₂₀H₂₂ClN₂O₃S₂⁺ 437.0755, found 437.0709.

### Typical Procedures for Examples 32-65

### Method A:

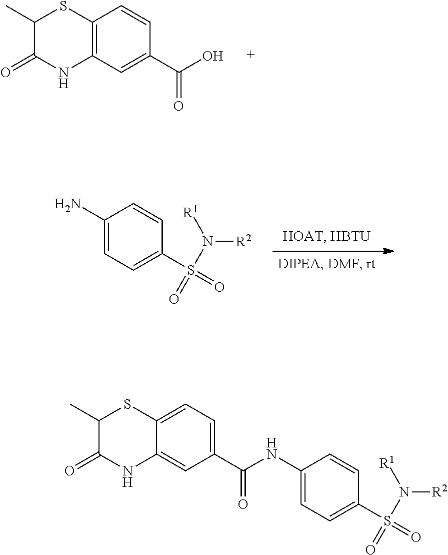

To a solution of 2-methyl-3-oxo-3,4-dihydro-2H-benzo[b][1,4]thiazine-6-carboxylic acid (22 mg, 0.1 mmol) in DMF (1 mL) was added amine (0.15 mmol), HOAT (20 mg, 0.15 mmol), HBTU (57 mg, 0.15 mmol) and DIPEA (0.052 mL, 0.3 mmol). The reaction mixture was stirred at rt overnight. Then the mixture was purified by prep-HPLC to give the desired product.

### Method B:

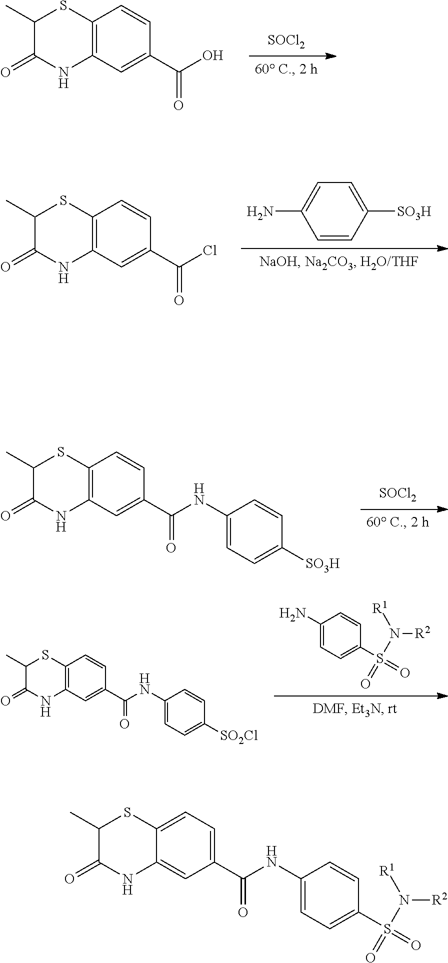

### Step 1. Synthesis of 2-methyl-3-oxo-3,4-dihydro-2H-benzo[b][1,4]thiazine-6-carbonyl chloride

2-methyl-3-oxo-3,4-dihydro-2H-benzo[b][1,4]thiazine-6-carboxylic acid (102 mg, 0.45 mmol) was dissolved in SOCl₂ (1 mL), then the reaction mixture was heated to 60° C. for 2 h. SOCl₂ was removed under reduced pressure and the residue was co-distilled with toluene/dichloromethane for 3 times to give the crude product. This product was used in the next step without further purification.

### Step 2. Synthesis of 4-(2-methyl-3-oxo-3,4-dihydro-2H-benzo[b][1,4]thiazine-6-carboxamido)benzenesulfonic acid

4-aminobenzenesulfonic acid (86 mg, 0.495 mmol) was dissolved in aq. NaOH (0.5 M, 0.6 mL) solution followed by added Na₂CO₃ (2 M, 0.25 mL). Then chloride from last step in THF (2 mL) was added dropwise. The reaction mixture was stirred at rt overnight. The mixture was acidized by 2N HCl, extracted with ethyl acetate (10 mL×3). The aqueous phase was distilled by rotary evaporator. The residue was purified by reverse chromatography to give the desired product 4-(2-methyl-3-oxo-3,4-dihydro-2H-benzo[b][1,4]thiazine-6-carboxamido)benzenesulfonic acid (151 mg, yield 89%) as a yellow solid. ESI: m/z=377.07 [M−H]⁻.

### Step 3. Synthesis of 4-(2-methyl-3-oxo-3,4-dihydro-2H-benzo[b][1,4]thiazine-6-carboxamido)benzenesulfonyl chloride

4-(2-methyl-3-oxo-3,4-dihydro-2H-benzo[b][1,4]thiazine-6-carboxamido)benzenesulfonic acid (150 mg, 0.4 mmol) was dissolved in SOCl₂ (1 mL), then the reaction mixture was heated to 60° C. for 2 h. SOCl₂ was removed under reduced pressure and the residue was co-distilled with toluene/dichloromethane for 3 times to give the crude product. This product was used immediately in the next step without further purification.

### Step 4. Synthesis of the Final Product

To a solution of 4-(2-methyl-3-oxo-3,4-dihydro-2H-benzo[b][1,4]thiazine-6-carboxamido)benzenesulfonyl chloride (40 mg, 0.1 mmol) in DMF (1 mL) was added Et₃N (0.042 mL, 0.3 mmol) and amine (0.15 mmol). The reaction mixture was stirred at rt overnight and purified by prep-HPLC to give the desired the product.

### Method C:

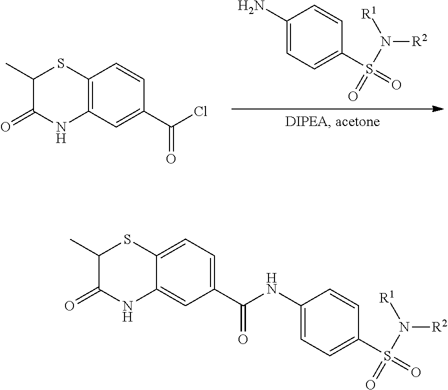

To a solution of amine (0.12 mmol) and DIPEA (0.087 mL, 0.5 mmol) in acetone (2 mL) was added the 2-methyl-3-oxo-3,4-dihydro-2H-benzo[b][1,4]thiazine-6-carbonyl chloride (24 mg, 0.1 mmol) in acetone (1 mL) by dropwise. The reaction mixture was stirred at rt overnight. Acetone was removed under reduced pressure and the residue was purified by ISCO to give the desired product.

### Example 32

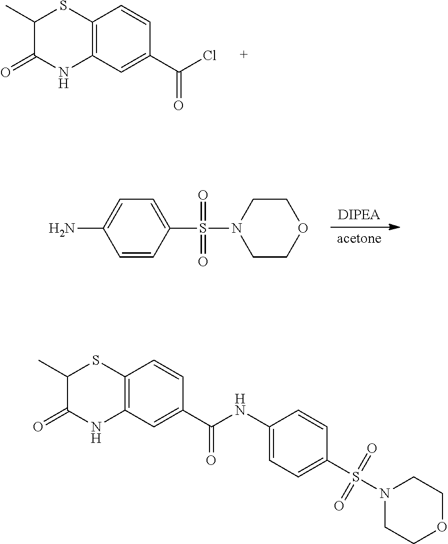 [SMILES: CC1Sc2ccc(C(=O)Cl)cc2NC1=O ; Nc1ccc(S(=O)(=O)N2CCOCC2)cc1 ; "DIPEA" ; "acetone" ; CC1Sc2ccc(C(=O)Nc3ccc(S(=O)(=O)N4CCOCC4)cc3)cc2NC1=O]

Example 32 was synthesized by method C. White solid, yield 60%. ¹H NMR (600 MHz, DMSO-d₆) δ 10.79 (s, 1H), 10.68 (s, 1H), 8.04 (d, J=8.4 Hz, 2H), 7.73 (d, J=8.4 Hz, 2H), 7.61 (d, J=8.4 Hz, 1H), 7.54 (s, 1H), 7.51 (d, J=8.4 Hz, 1H), 3.75 (q, J=7.2 Hz, 1H), 3.63 (t, J=4.2 Hz, 4H), 2.85 (d, J=4.2 Hz, 4H), 1.34 (d, J=7.2 Hz, 3H). ESI: m/z=448.10 [M+H]⁺.

### Example 33

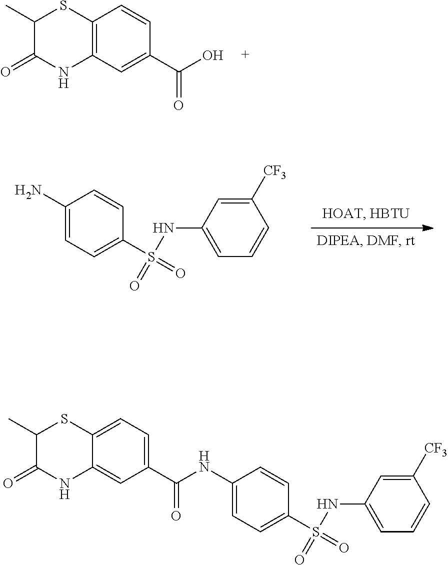

Example 33 was synthesized by method A. White solid, yield 52%. ¹H NMR (600 MHz, Methanol-d₄) δ 8.73 (d, J=3.0 Hz, 1H), 8.42 (d, J=8.4 Hz, 1H), 7.84 (d, J=8.4 Hz, 2H), 7.76 (d, J=8.4 Hz, 2H), 7.55-7.41 (m, 4H), 7.34 (t, J=7.2 Hz, 1H), 3.63 (q, J=7.2 Hz, 1H), 1.43 (d, J=7.2 Hz, 3H). ESI: m/z=522.06 [M+H]⁺.

### Example 34

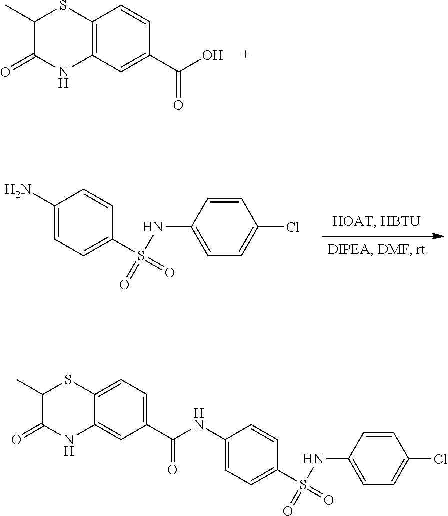

Example 34 was synthesized by method A. White solid, yield 27%. ¹H NMR (600 MHz, Methanol-d₄) δ 7.83 (d, J=8.4 Hz, 2H), 7.72 (d, J=8.4 Hz, 2H), 7.55 (dd, J=1.8, 8.4 Hz, 1H), 7.48 (s, 1H), 7.44 (d, J=7.8 Hz, 1H), 7.21 (d, J=8.4 Hz, 2H), 7.08 (d, J=8.4 Hz, 2H), 3.63 (q, J=7.2 Hz, 1H), 1.43 (d, J=7.2 Hz, 3H). ESI: m/z=488.04 [M+H]⁺.

### Example 35

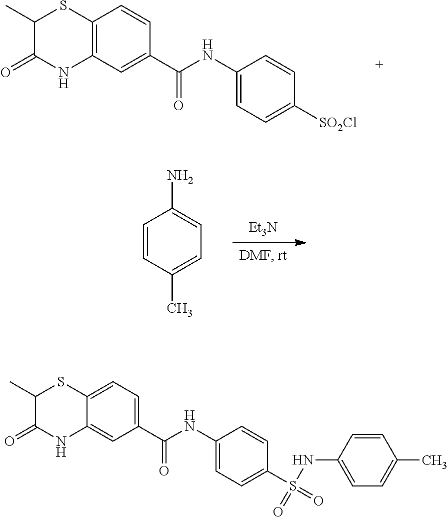

Example 35 was synthesized by method B. White solid, yield 52%. ¹H NMR (600 MHz, DMSO-d₆) δ 10.77 (s, 1H), 10.55 (s, 1H), 10.02 (s, 1H), 7.87 (d, J=9.0 Hz, 2H), 7.69 (d, J=9.0 Hz, 2H), 7.57 (dd, J=2.4, 8.4 Hz, 1H), 7.50-7.48 (m, 2H), 7.02 (d, J=9.0 Hz, 2H), 6.97 (d, J=9.0 Hz, 2H), 3.74 (q, J=7.2 Hz, 1H), 2.18 (s, 3H), 1.33 (d, J=7.2 Hz, 3H). ESI: m/z=468.09 [M+H]⁺.

### Example 36

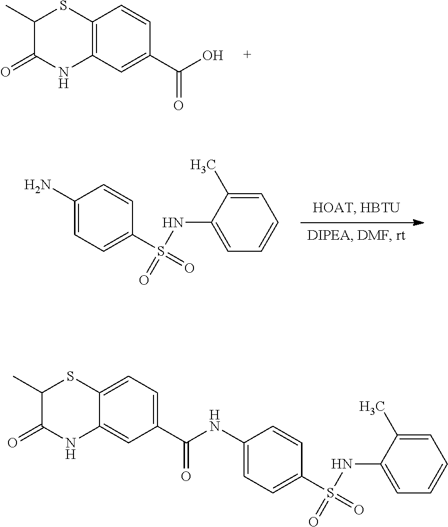

Example 36 was synthesized by method A. White solid, yield 32%. ¹H NMR (600 MHz, Methanol-d₄) δ 7.83 (d, J=9.0 Hz, 2H), 7.63 (d, J=9.0 Hz, 2H), 7.57 (dd, J=1.8, 7.8 Hz, 1H), 7.50 (s, 1H), 7.45 (d, J=7.8 Hz, 1H), 7.12-7.07 (m, 4H), 3.63 (q, J=7.2 Hz, 1H), 2.03 (s, 3H), 1.43 (d, J=7.2 Hz, 3H). ESI: m/z=468.10 [M+H]⁺.

### Example 37

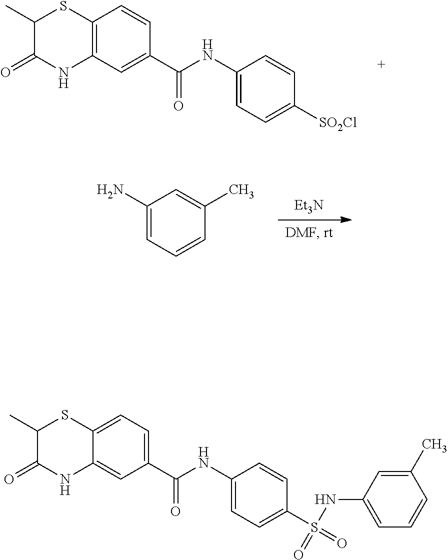

Example 37 was synthesized by method B. White solid, yield 61%. ¹H NMR (600 MHz, DMSO-d₆) δ 10.77 (s, 1H), 10.56 (s, 1H), 10.13 (s, 1H), 7.88 (d, J=11.4 Hz, 2H), 7.73 (d, J=11.4 Hz, 2H), 7.56 (dd, J=1.8, 7.8 Hz, 1H), 7.50-7.48 (m, 2H), 7.09 (t, J=7.2 Hz, 1H), 6.91-6.89 (m, 2H), 6.82 (d, J=7.8 Hz, 1H), 3.73 (q, J=7.2 Hz, 1H), 2.19 (s, 3H), 1.33 (d, J=7.2 Hz, 3H). ESI: m/z=468.09 [M+H]⁺.

### Example 38

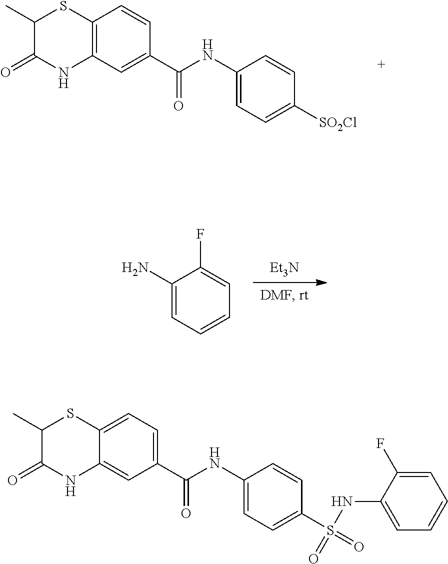 [SMILES: "Et3N" ; CC1Sc2ccc(C(=O)Nc3ccc(S(=O)(=O)Cl)cc3)cc2NC1=O ; Nc1ccccc1F ; "DMF, rt" ; CC1Sc2ccc(C(=O)Nc3ccc(S(=O)(=O)Nc4ccccc4F)cc3)cc2NC1=O]

Example 38 was synthesized by method B. White solid, yield 6%. ¹H NMR (600 MHz, DMSO-d₆) δ 10.78 (s, 1H), 10.60 (s, 1H), 10.08 (s, 1H), 7.91 (d, J=9.0 Hz, 2H), 7.70 (d, J=9.0 Hz, 2H), 7.60 (dd, J=1.8, 8.4 Hz, 1H), 7.52-7.50 (m, 2H), 7.26 (dt, J=1.8, 7.8 Hz, 1H), 7.19-7.11 (m, 3H), 3.75 (q, J=7.2 Hz, 1H), 1.34 (d, J=7.2 Hz, 3H). ESI: m/z=472.07 [M+H]⁺.

### Example 39

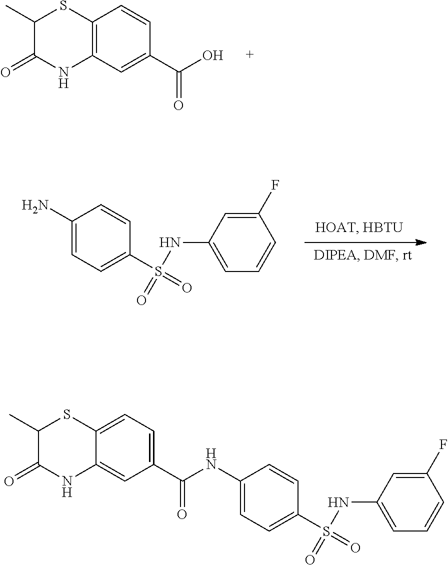

Example 39 was synthesized by method A. White solid, yield 38%. ¹H NMR (600 MHz, Methanol-d₄) δ 7.84 (d, J=8.4 Hz, 2H), 7.76 (d, J=8.4 Hz, 2H), 7.54 (dd, J=1.8, 7.8 Hz, 1H), 7.48 (s, 1H), 7.42 (d, J=7.8 Hz, 1H), 7.22-7.18 (m, 1H), 6.92-6.88 (m, 2H), 6.77-6.75 (m, 1H), 3.62 (q, J=7.2 Hz, 1H), 1.42 (d, J=7.2 Hz, 3H). ESI: m/z=472.07 [M+H]⁺.

### Example 40

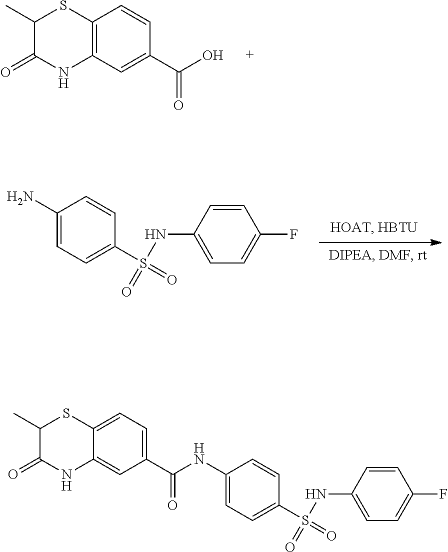

Example 40 was synthesized by method A. White solid, yield 36%. ¹H NMR (600 MHz, DMSO-d₆) δ 10.75 (s, 1H), 10.55 (s, 1H), 10.13 (s, 1H), 7.87 (d, J=8.4 Hz, 2H), 7.67 (d, J=8.4 Hz, 2H), 7.55 (d, J=8.4 Hz, 1H), 7.48 (s, 1H), 7.47 (d, J=8.4 Hz, 1H), 7.08-7.06 (m, 4H), 3.72 (q, J=6.6 Hz, 1H), 1.31 (d, J=6.6 Hz, 3H). ESI: m/z=472.08 [M+H]⁺.

### Example 41

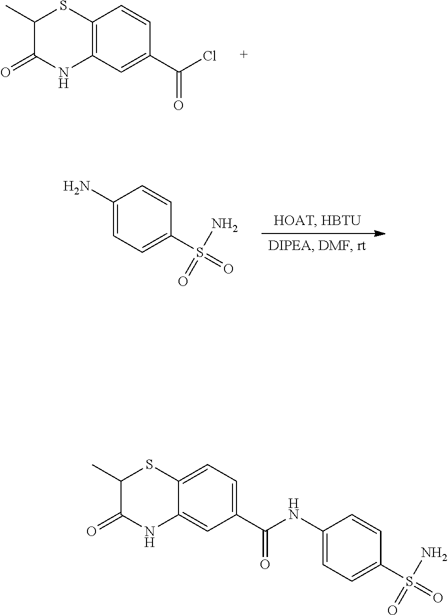

Example 41 was synthesized by method A. White solid, yield 19%. ¹H NMR (600 MHz, DMSO-d₆) δ 10.78 (s, 1H), 10.56 (s, 1H), 7.93 (d, J=8.4 Hz, 2H), 7.80 (d, J=8.4 Hz, 2H), 7.61 (d, J=8.4 Hz, 1H), 7.54 (s, 1H), 7.51 (d, J=8.4 Hz, 1H), 7.28 (s, 2H), 3.75 (q, J=7.2 Hz, 1H), 1.34 (d, J=7.2 Hz, 3H). ESI: m/z=378.05 [M+H]⁺.

### Example 42

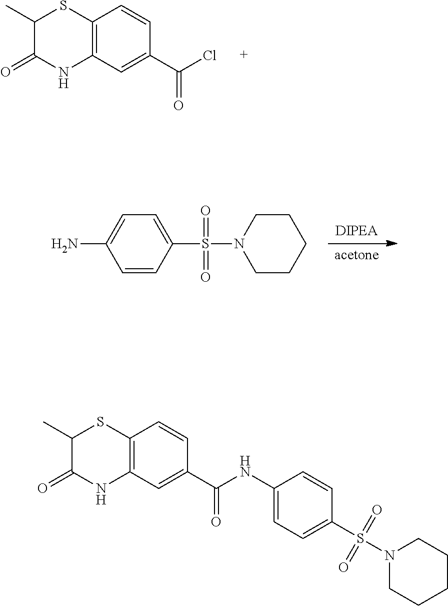 [SMILES: CC1Sc2ccc(C(=O)Cl)cc2NC1=O ; Nc1ccc(S(=O)(=O)N2CCCCC2)cc1 ; "DIPEA" ; "acetone" ; CC1Sc2ccc(C(=O)Nc3ccc(S(=O)(=O)N4CCCCC4)cc3)cc2NC1=O]

Example 42 was synthesized by method C. White solid, yield 65%. ¹H NMR (600 MHz, DMSO-d₆) δ 10.77 (s, 1H), 10.62 (s, 1H), 7.99 (d, J=9.0 Hz, 2H), 7.69 (d, J=9.0 Hz, 2H), 7.59 (dd, J=1.8, 8.4 Hz, 1H), 7.51 (d, J=1.8 Hz, 1H), 7.49 (d, J=7.8 Hz, 1H), 3.72 (q, J=7.2 Hz, 1H), 2.85 (t, J=4.8 Hz, 4H), 1.53-1.51 (m, 4H), 1.34-1.32 (m, 5H). ESI: m/z=446.12 [M+H]⁺.

### Example 43

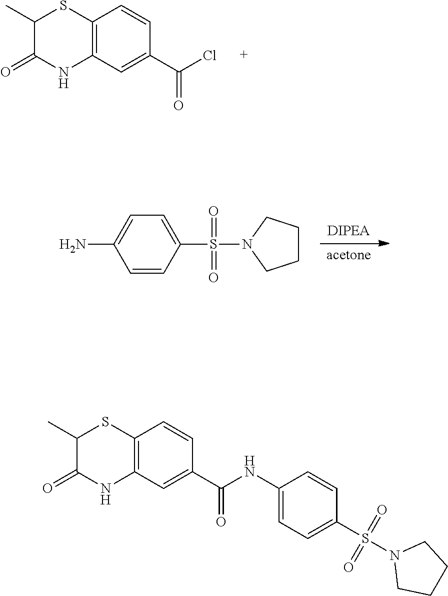 [SMILES: CC1Sc2ccc(C(=O)Cl)cc2NC1=O ; Nc1ccc(S(=O)(=O)N2CCCC2)cc1 ; "DIPEA" ; "acetone" ; CC1Sc2ccc(C(=O)Nc3ccc(S(=O)(=O)N4CCCC4)cc3)cc2NC1=O]

Example 43 was synthesized by method C. White solid, yield 67%. ¹H NMR (600 MHz, DMSO-d₆) δ 10.80 (s, 1H), 10.64 (s, 1H), 8.02 (d, J=9.0 Hz, 2H), 7.81 (d, J=9.0 Hz, 2H), 7.61 (dd, J=1.8, 8.4 Hz, 1H), 7.54 (d, J=1.8 Hz, 1H), 7.52 (d, J=7.8 Hz, 1H), 3.76 (q, J=7.2 Hz, 1H), 3.14 (t, J=6.6 Hz, 4H), 1.67-1.64 (m, 4H), 1.35 (d, J=7.2 Hz, 3H). ESI: m/z=432.10 [M+H]⁺.

### Example 44

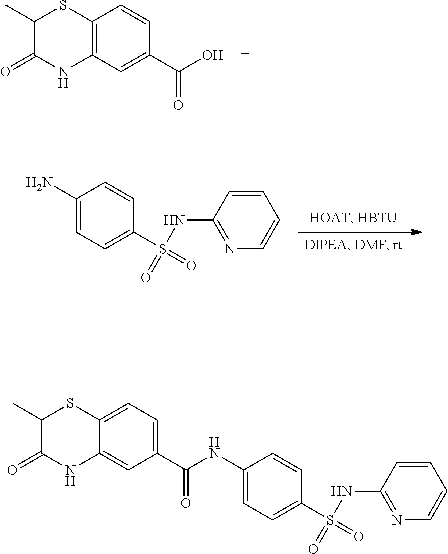

Example 44 was synthesized by method A. White solid, yield 33%. ¹H NMR (600 MHz, DMSO-d₆) δ 10.75 (s, 1H), 10.53 (s, 1H), 10.01 (s, 1H), 8.01 (s, 1H), 7.88 (d, J=8.4 Hz, 2H), 7.84 (d, J=8.4 Hz, 2H), 7.69 (t, J=7.2 Hz, 1H), 7.56 (dd, J=1.8, 7.8 Hz, 1H), 7.49 (d, J=1.8 Hz, 1H), 7.47 (d, J=7.8 Hz, 1H), 7.13-7.12 (m, 1H), 6.86 (s, 1H), 3.72 (q, J=7.2 Hz, 1H), 1.31 (d, J=7.2 Hz, 3H). ESI: m/z=455.08 [M+H]⁺.

### Example 45

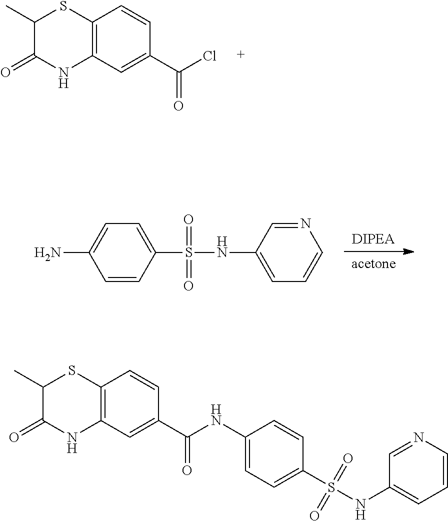 [SMILES: CC1Sc2ccc(C(=O)Cl)cc2NC1=O ; Nc1ccc(S(=O)(=O)Nc2cccnc2)cc1 ; "DIPEA" ; "acetone" ; CC1Sc2ccc(C(=O)Nc3ccc(S(=O)(=O)Nc4cccnc4)cc3)cc2NC1=O]

Example 45 was synthesized by method C. Yellow solid, yield 33%. ¹H NMR (600 MHz, CDCl₃) δ 9.04 (s, 1H), 8.53 (d, J=4.8 Hz, 1H), 7.69 (d, J=8.4 Hz, 1H), 7.65 (d, J=9.0 Hz, 2H), 7.32 (dd, J=4.8, 8.4 Hz, 1H), 7.10 (d, J=7.8 Hz, 1H), 7.03 (s, 1H), 7.00 (dd, J=1.8, 7.8 Hz, 1H), 6.65 (d, J=8.4 Hz, 2H), 3.50 (q, J=7.2 Hz, 1H), 1.43 (d, J=7.2 Hz, 3H). ESI: m/z=455.09 [M+H]⁺.

### Example 46

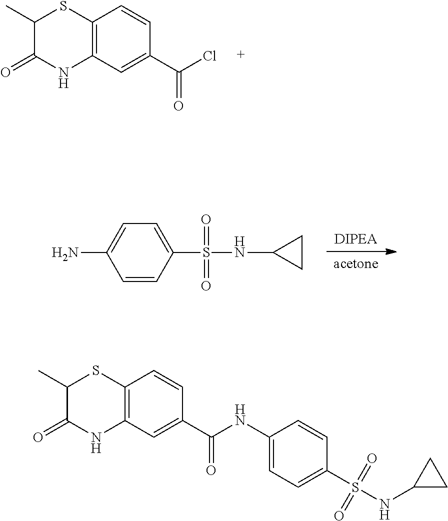 [SMILES: CC1Sc2ccc(C(=O)Cl)cc2NC1=O ; Nc1ccc(S(=O)(=O)NC2CC2)cc1 ; "DIPEA" ; "acetone" ; CC1Sc2ccc(C(=O)Nc3ccc(S(=O)(=O)NC4CC4)cc3)cc2NC1=O]

Example 46 was synthesized by method C. White solid, yield 70%. ¹H NMR (600 MHz, DMSO-d₆) δ 10.78 (s, 1H), 10.60 (s, 1H), 7.97 (d, J=7.8 Hz, 2H), 7.81 (s, 1H), 7.78 (d, J=7.8 Hz, 2H), 7.60 (d, J=7.8 Hz, 1H), 7.53 (s, 1H), 7.50 (d, J=7.8 Hz, 1H), 3.74 (q, J=7.2 Hz, 1H), 2.11-2.08 (m, 1H), 1.34 (d, J=7.2 Hz, 3H), 0.49-0.46 (m, 2H), 0.38-0.35 (m, 2H). ESI: m/z=418.08 [M+H]⁺.

### Example 47

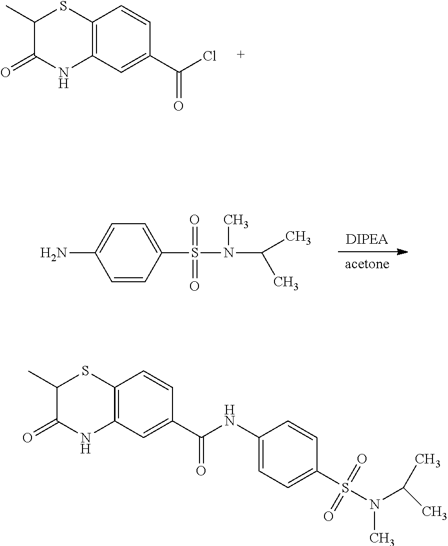 [SMILES: CC1Sc2ccc(C(=O)Cl)cc2NC1=O ; CC(C)N(C)S(=O)(=O)c1ccc(N)cc1 ; "DIPEA" ; "acetone" ; CC1Sc2ccc(C(=O)Nc3ccc(S(=O)(=O)N(C)C(C)C)cc3)cc2NC1=O]

Example 47 was synthesized by method C. Yellow solid, yield 37%. ¹H NMR (600 MHz, DMSO-d₆) δ 10.78 (s, 1H), 10.61 (s, 1H), 7.98 (d, J=8.4 Hz, 2H), 7.76 (d, J=8.4 Hz, 2H), 7.60 (dd, J=1.8, 7.8 Hz, 1H), 7.53 (d, J=1.8 Hz, 1H), 7.50 (d, J=8.4 Hz, 1H), 4.08-4.02 (m, 1H), 3.74 (q, J=7.2 Hz, 1H), 2.63 (s, 3H), 1.34 (d, J=7.2 Hz, 3H), 0.88 (d, J=6.6 Hz, 6H). ESI: m/z=434.11 [M+H]⁺.

### Example 48

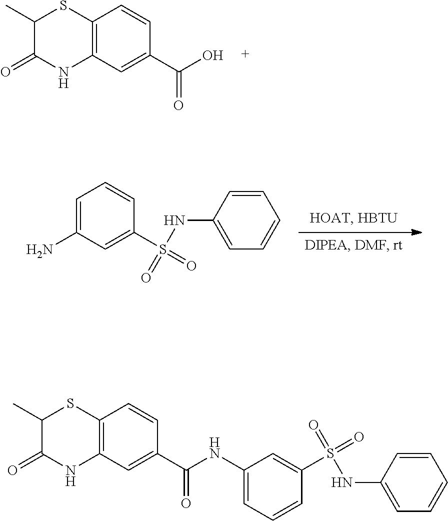

Example 48 was synthesized by method A. White solid, yield 82%. ¹H NMR (600 MHz, DMSO-d₆) δ 10.78 (s, 1H), 10.53 (s, 1H), 10.36 (s, 1H), 8.37 (t, J=1.8 Hz, 1H), 7.91 (d, J=9.0 Hz, 2H), 7.60 (dd, J=1.8, 8.4 Hz, 1H), 7.52-7.46 (m, 3H), 7.23 (t, J=7.8 Hz, 2H), 7.10 (d, J=8.4 Hz, 2H), 7.01 (t, J=7.8 Hz, 1H), 3.75 (q, J=6.6 Hz, 1H), 1.34 (d, J=6.6 Hz, 3H). ESI: m/z=454.08 [M+H]⁺.

### Example 49

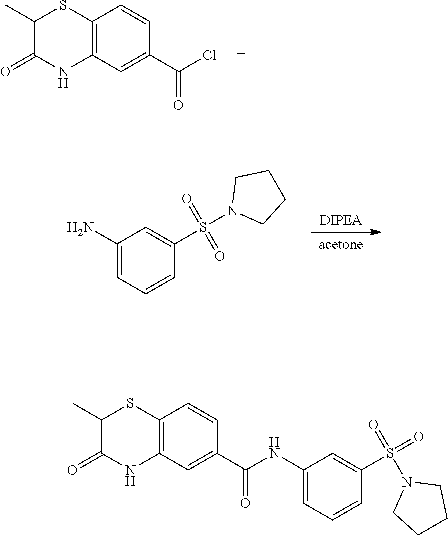 [SMILES: CC1Sc2ccc(C(=O)Cl)cc2NC1=O ; Nc1cccc(S(=O)(=O)N2CCCC2)c1 ; "DIPEA" ; "acetone" ; CC1Sc2ccc(C(=O)Nc3cccc(S(=O)(=O)N4CCCC4)c3)cc2NC1=O]

Example 49 was synthesized by method C. Yellow solid, yield 63%. ¹H NMR (600 MHz, CDCl₃) δ 9.17 (s, 1H), 8.93 (s, 1H), 8.74 (d, J=8.4 Hz, 1H), 8.01 (t, J=1.8 Hz, 1H), 7.75 (dd, J=1.8, 8.4 Hz, 1H), 7.66-7.59 (m, 3H), 7.44 (d, J=8.4 Hz, 1H), 3.59 (q, J=7.2 Hz, 1H), 3.26 (t, J=6.6 Hz, 4H), 1.72 (t, J=6.6 Hz, 4H), 1.51 (d, J=7.2 Hz, 3H). ESI: m/z=432.09 [M+H]⁺.

### Example 50

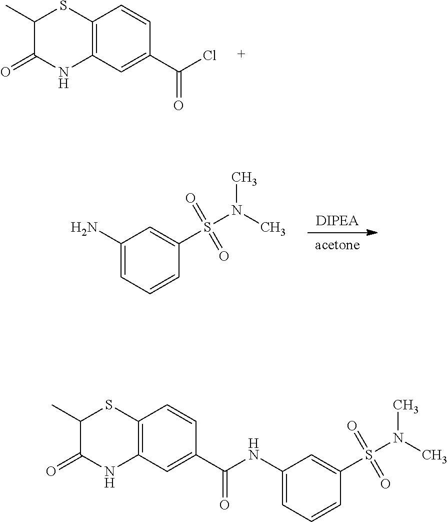 [SMILES: CC1Sc2ccc(C(=O)Cl)cc2NC1=O ; CN(C)S(=O)(=O)c1cccc(N)c1 ; "DIPEA" ; "acetone" ; CC1Sc2ccc(C(=O)Nc3cccc(S(=O)(=O)N(C)C)c3)cc2NC1=O]

Example 50 was synthesized by method C. Yellow solid, yield 64%. ¹H NMR (600 MHz, CDCl₃) δ 9.03 (s, 1H), 8.81 (s, 1H), 8.70 (dd, J=1.2, 8.4 Hz, 1H), 7.94 (t, J=1.8 Hz, 1H), 7.73 (dd, J=1.8, 8.4 Hz, 1H), 7.64-7.57 (m, 3H), 7.44 (d, J=7.8 Hz, 1H), 3.59 (q, J=7.2 Hz, 1H), 2.74 (s, 6H), 1.51 (d, J=7.2 Hz, 3H). ESI: m/z=406.10 [M+H]⁺.

### Example 51

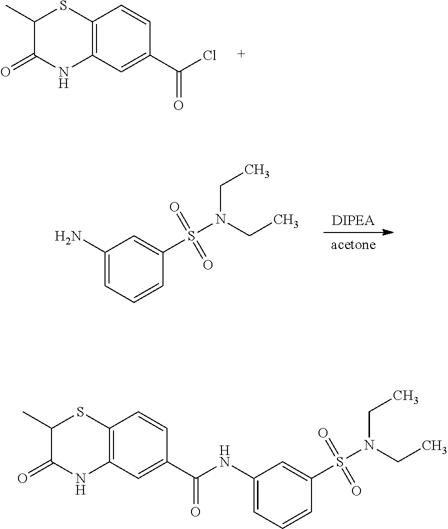 [SMILES: CC1Sc2ccc(C(=O)Cl)cc2NC1=O ; CCN(CC)S(=O)(=O)c1cccc(N)c1 ; "DIPEA" ; "acetone" ; CCN(CC)S(=O)(=O)c1cccc(NC(=O)c2ccc3c(c2)NC(=O)C(C)S3)c1]

Example 51 was synthesized by method C. Yellow solid, yield 62%. ¹H NMR (600 MHz, CDCl₃) δ 9.14 (s, 1H), 9.06 (s, 1H), 8.59 (d, J=8.4 Hz, 1H), 8.01 (t, J=1.8 Hz, 1H), 7.71 (dd, J=1.8, 7.8 Hz, 1H), 7.60-7.53 (m, 3H), 7.41 (d, J=7.8 Hz, 1H), 3.57 (q, J=7.2 Hz, 1H), 3.22 (q, J=7.2 Hz, 4H), 1.48 (d, J=7.2 Hz, 3H), 1.05 (t, J=7.2 Hz, 6H). ESI: m/z=434.12 [M+H]⁺.

### Example 52

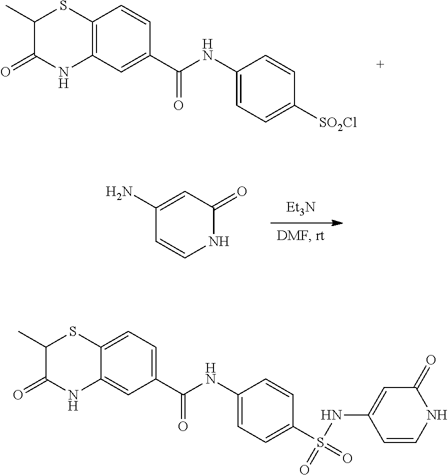 [SMILES: "Et3N" ; CC1Sc2ccc(C(=O)Nc3ccc(S(=O)(=O)Cl)cc3)cc2NC1=O ; Nc1cc[nH]c(=O)c1 ; "DMF, rt" ; CC1Sc2ccc(C(=O)Nc3ccc(S(=O)(=O)Nc4cc[nH]c(=O)c4)cc3)cc2NC1=O]

Example 52 was synthesized by method B. Yellow solid, yield 30%. ¹H NMR (600 MHz, DMSO-d₆) δ 10.78 (s, 1H), 10.72 (s, 1H), 10.28 (s, 1H), 8.01 (d, J=9.0 Hz, 2H), 7.89 (d, J=9.0 Hz, 2H), 7.65 (d, J=6.0 Hz, 1H), 7.60-7.57 (m, 2H), 7.52-7.49 (m, 2H), 6.42 (dd, J=1.8, 6.6 Hz, 1H), 6.18 (d, J=2.4 Hz, 1H), 3.73 (q, J=7.2 Hz, 1H), 1.32 (d, J=7.2 Hz, 3H). ESI: m/z=522.10 [M+H]⁺.

### Example 53

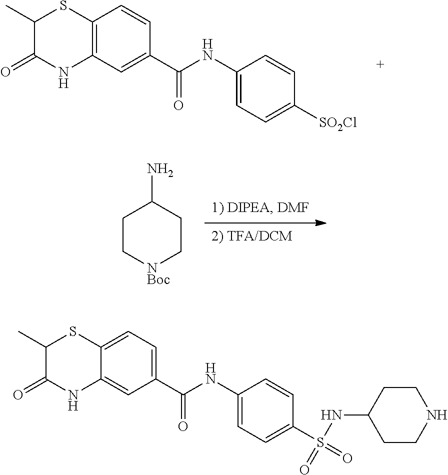

Example 53 was synthesized by method B. Yellow solid, yield 18%. ¹H NMR (600 MHz, DMSO-d₆) δ 10.77 (s, 1H), 10.61 (s, 1H), 7.95 (d, J=8.4 Hz, 2H), 7.87 (s, 1H), 7.78 (d, J=8.4 Hz, 2H), 7.58 (dd, J=1.8, 8.4 Hz, 1H), 7.51 (d, J=1.8 Hz, 1H), 7.49 (d, J=8.4 Hz, 1H), 3.73 (q, J=7.2 Hz, 1H), 3.28-3.24 (m, 1H), 3.16-3.13 (m, 2H), 2.90-2.85 (m, 2H), 1.73-1.70 (m, 2H), 1.53-1.47 (m, 2H), 1.32 (d, J=7.2 Hz, 3H). ESI: m/z=461.12 [M+H]⁺.

### Example 54

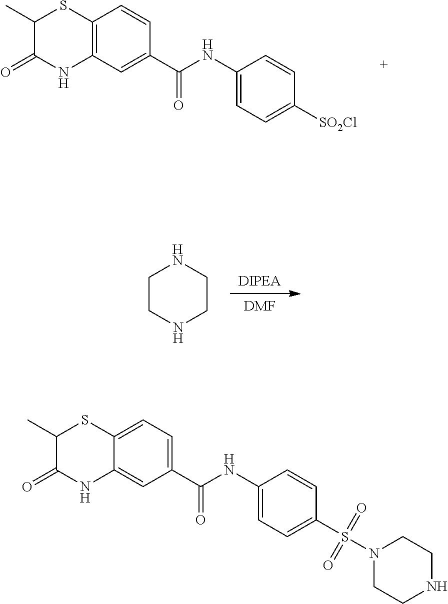

Example 54 was synthesized by method B. Yellow solid, yield 32%. ¹H NMR (600 MHz, DMSO-d₆) δ 10.78 (s, 1H), 10.71 (s, 1H), 8.05 (d, J=8.4 Hz, 2H), 7.75 (d, J=8.4 Hz, 2H), 7.60 (dd, J=1.8, 8.4 Hz, 1H), 7.54 (d, J=1.8 Hz, 1H), 7.49 (d, J=8.4 Hz, 1H), 3.73 (q, J=7.2 Hz, 1H), 3.12 (d, J=5.4 Hz, 4H), 3.03 (d, J=5.4 Hz, 4H), 1.32 (d, J=7.2 Hz, 3H). ESI: m/z=447.12 [M+H]⁺.

### Example 55

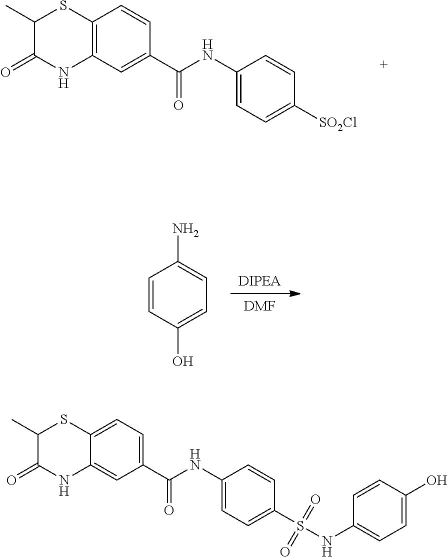

Example 55 was synthesized by method B. White solid, yield 48%. ¹H NMR (600 MHz, DMSO-d₆) δ 10.75 (s, 1H), 10.53 (s, 1H), 9.61 (s, 1H), 9.28 (s, 1H), 7.85 (d, J=9.0 Hz, 2H), 7.60 (d, J=9.0 Hz, 2H), 7.55 (dd, J=1.8, 8.4 Hz, 1H), 7.49 (d, J=1.8 Hz, 1H), 7.47 (d, J=8.4 Hz, 1H), 6.82 (d, J=9.0 Hz, 2H), 6.58 (m, J=9.0 Hz, 2H), 3.72 (q, J=7.2 Hz, 1H), 1.31 (d, J=7.2 Hz, 3H). ESI: m/z=470.10 [M+H]⁺.

### Example 56

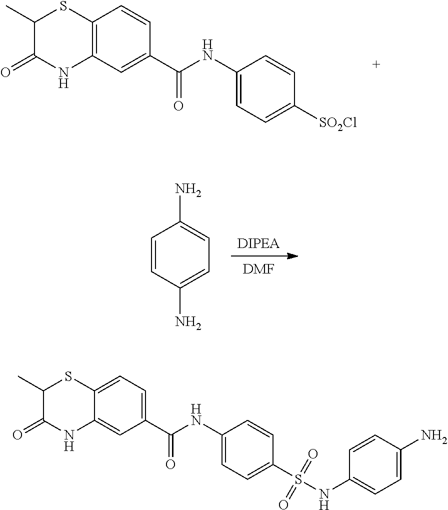

Example 56 was synthesized by method B. White solid, yield 27%. ¹H NMR (600 MHz, DMSO-d₆) δ 10.76 (s, 1H), 10.55 (s, 1H), 9.74 (s, 1H), 7.86 (d, J=8.4 Hz, 2H), 7.63 (d, J=8.4 Hz, 2H), 7.56 (dd, J=1.8, 8.4 Hz, 1H), 7.49 (d, J=1.8 Hz, 1H), 7.47 (d, J=8.4 Hz, 1H), 6.84 (d, J=9.0 Hz, 2H), 6.66 (d, J=9.0 Hz, 2H), 3.72 (q, J=7.2 Hz, 1H), 1.31 (d, J=7.2 Hz, 3H). ESI: m/z=469.10 [M+H]⁺.

### Example 57

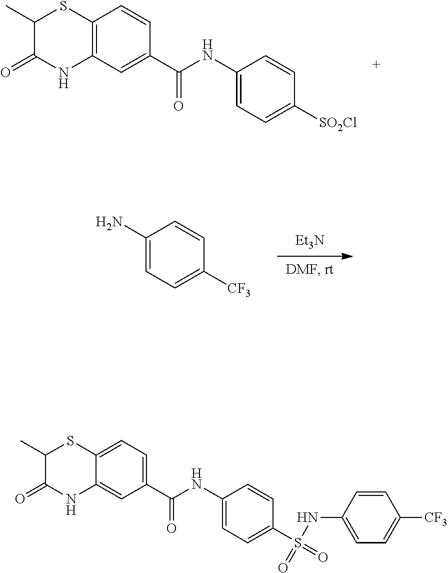 [SMILES: "Et3N" ; CC1Sc2ccc(C(=O)Nc3ccc(S(=O)(=O)Cl)cc3)cc2NC1=O ; Nc1ccc(C(F)(F)F)cc1 ; "DMF, rt" ; CC1Sc2ccc(C(=O)Nc3ccc(S(=O)(=O)Nc4ccc(C(F)(F)F)cc4)cc3)cc2NC1=O]

Example 57 was synthesized by method B. Yellow solid, yield 8%. ¹H NMR (600 MHz, DMSO-d₆) δ 10.80 (s, 1H), 10.75 (s, 1H), 10.58 (s, 1H), 7.91 (d, J=8.4 Hz, 2H), 7.79 (d, J=8.4 Hz, 2H), 7.58 (d, J=9.0 Hz, 2H), 7.55 (dd, J=1.8, 8.4 Hz, 1H), 7.48 (s, 1H), 7.47 (d, J=5.4 Hz, 1H), 7.27 (d, J=9.0 Hz, 2H), 3.72 (q, J=7.2 Hz, 1H), 1.31 (d, J=7.2 Hz, 3H). ESI: m/z=522.10 [M+H]⁺.

### Example 58

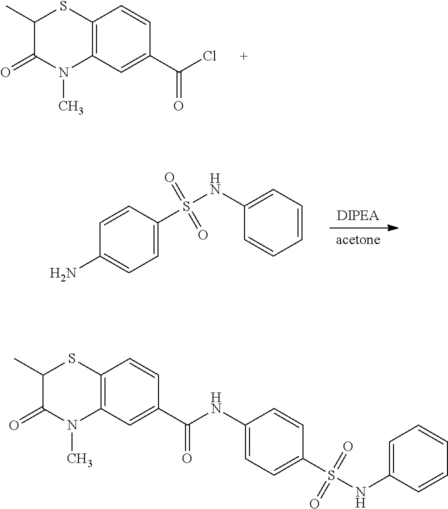 [SMILES: CC1Sc2ccc(C(=O)Cl)cc2N(C)C1=O ; Nc1ccc(S(=O)(=O)Nc2ccccc2)cc1 ; "DIPEA" ; "acetone" ; CC1Sc2ccc(C(=O)Nc3ccc(S(=O)(=O)Nc4ccccc4)cc3)cc2N(C)C1=O]

Example 58 was synthesized by method C. White solid, yield 32%. ¹H NMR (600 MHz, DMSO-d₆) δ 10.57 (s, 1H), 10.18 (s, 1H), 7.87 (d, J=9.0 Hz, 2H), 7.73 (d, J=9.0 Hz, 2H), 7.70 (d, J=1.8 Hz, 1H), 7.61 (dd, J=1.8, 8.4 Hz, 1H), 7.55 (d, J=8.4 Hz, 1H), 7.21 (t, J=8.4 Hz, 2H), 7.07 (d, J=8.4 Hz, 2H), 6.99 (t, J=7.8 Hz, 1H), 3.73 (q, J=7.2 Hz, 1H), 3.41 (s, 3H), 1.30 (d, J=7.2 Hz, 3H). ESI: m/z=468.10 [M+H]⁺.

### Example 59

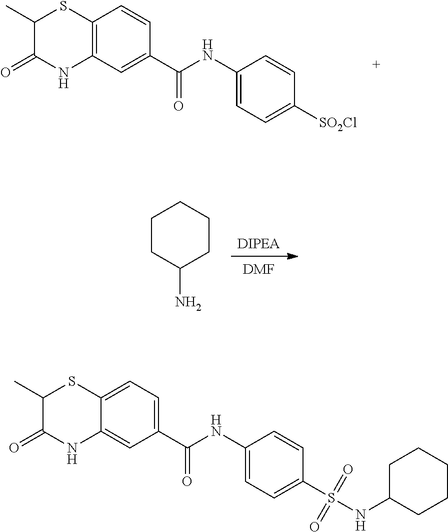

Example 59 was synthesized by method B. White solid, yield 48%. ¹H NMR (600 MHz, DMSO-d₆) δ 10.77 (s, 1H), 10.56 (s, 1H), 7.92 (d, J=9.0 Hz, 2H), 7.76 (d, J=9.0 Hz, 2H), 7.58 (dd, J=1.8, 7.8 Hz, 1H), 7.53-7.48 (m, 3H), 3.73 (q, J=7.2 Hz, 1H), 2.90-2.87 (m, 1H), 1.57-1.53 (m, 4H), 1.43-1.41 (m, 2H), 1.32 (d, J=7.2 Hz, 3H), 1.13-1.07 (m, 4H). ESI: m/z=460.15 [M+H]⁺.

### Example 60

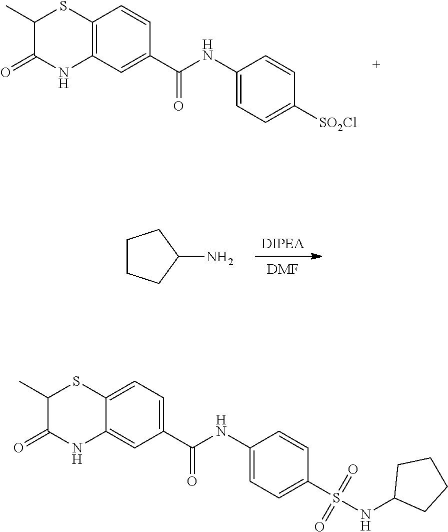

Example 60 was synthesized by method B. White solid, yield 53%. ¹H NMR (600 MHz, DMSO-d₆) δ 10.77 (s, 1H), 10.57 (s, 1H), 7.92 (d, J=9.0 Hz, 2H), 7.75 (d, J=9.0 Hz, 2H), 7.58 (dd, J=1.8, 7.8 Hz, 1H), 7.53-7.48 (m, 3H), 3.73 (q, J=7.2 Hz, 1H), 3.37-3.35 (m, 1H), 1.58-1.50 (m, 4H), 1.36-1.25 (m, 7H). ESI: m/z=446.12 [M+H]⁺.

### Example 61

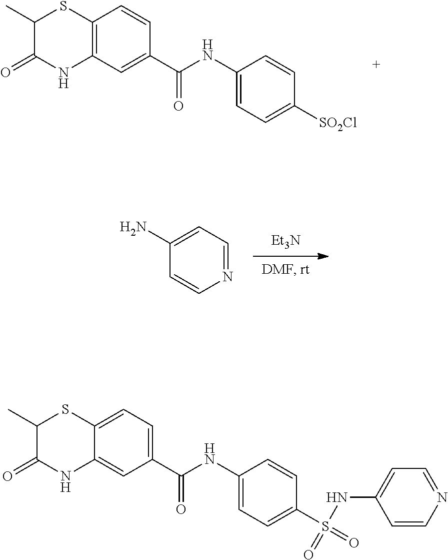 [SMILES: "Et3N" ; CC1Sc2ccc(C(=O)Nc3ccc(S(=O)(=O)Cl)cc3)cc2NC1=O ; Nc1ccncc1 ; "DMF, rt" ; CC1Sc2ccc(C(=O)Nc3ccc(S(=O)(=O)Nc4ccncc4)cc3)cc2NC1=O]

Example 61 was synthesized by method B. Yellow solid, yield 18%. ¹H NMR (600 MHz, DMSO-d₆) δ 10.76 (s, 1H), 10.60 (s, 1H), 10.28 (s, 1H), 8.29 (brs, 2H), 7.93 (d, J=9.0 Hz, 2H), 7.87 (d, J=9.0 Hz, 2H), 7.56 (dd, J=2.4, 8.4 Hz, 1H), 7.49 (s, 1H), 7.47 (d, J=8.4 Hz, 1H), 7.20 (brs, 2H), 3.72 (q, J=7.2 Hz, 1H), 1.31 (d, J=7.2 Hz, 3H). ESI: m/z=455.10 [M+H]⁺.

### Example 62

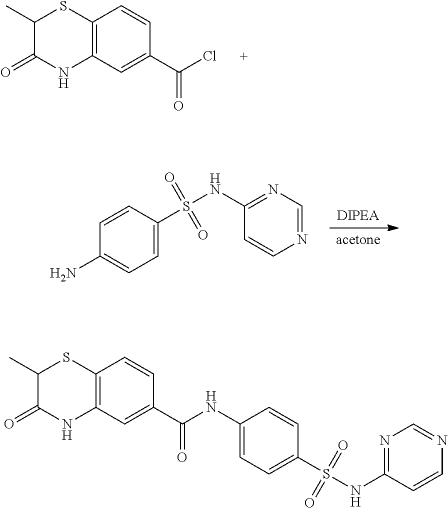 [SMILES: CC1Sc2ccc(C(=O)Cl)cc2NC1=O ; Nc1ccc(S(=O)(=O)Nc2ccncn2)cc1 ; "DIPEA" ; "acetone" ; CC1Sc2ccc(C(=O)Nc3ccc(S(=O)(=O)Nc4ccncn4)cc3)cc2NC1=O]

Example 62 was synthesized by method C. White solid, yield 18%. ¹H NMR (600 MHz, DMSO-d₆) δ 10.77 (s, 1H), 10.74 (s, 1H), 10.60 (s, 1H), 7.93 (s, 1H), 7.56 (d, J=9.0 Hz, 2H), 7.52 (d, J=1.8 Hz, 1H), 7.51-7.49 (m, 3H), 7.42 (d, J=8.4 Hz, 2H), 6.53 (brs, 1H), 3.73 (q, J=7.2 Hz, 1H), 1.32 (d, J=7.2 Hz, 3H). ESI: m/z=456.10 [M+H]⁺.

### Example 63

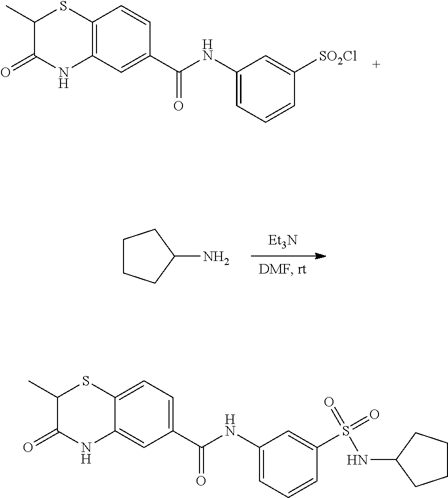 [SMILES: "Et3N" ; CC1Sc2ccc(C(=O)Nc3cccc(S(=O)(=O)Cl)c3)cc2NC1=O ; NC1CCCC1 ; "DMF, rt" ; CC1Sc2ccc(C(=O)Nc3cccc(S(=O)(=O)NC4CCCC4)c3)cc2NC1=O]

Example 63 was synthesized by method B. Yellow solid, yield 18%. ¹H NMR (600 MHz, DMSO-d₆) δ 10.77 (s, 1H), 10.52 (s, 1H), 8.31 (t, J=1.8 Hz, 1H), 7.94 (d, J=9.0 Hz, 1H), 7.66 (d, J=7.2 Hz, 1H), 7.47 (dd, J=1.8, 8.4 Hz, 1H), 7.56-7.48 (m, 3H), 6.52 (s, 1H), 3.73 (q, J=7.2 Hz, 1H), 3.34-3.33 (m, 1H), 1.62-1.56 (m, 2H), 1.53-1.50 (m, 2H), 1.37-1.28 (m, 7H). ESI: m/z=446.11 [M+H]⁺.

### Example 64

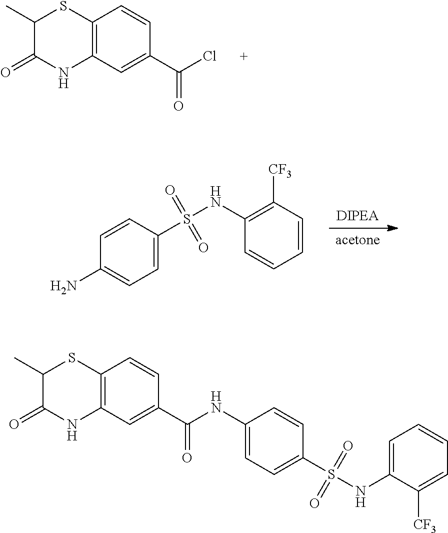 [SMILES: CC1Sc2ccc(C(=O)Cl)cc2NC1=O ; Nc1ccc(S(=O)(=O)Nc2ccccc2C(F)(F)F)cc1 ; "DIPEA" ; "acetone" ; CC1Sc2ccc(C(=O)Nc3ccc(S(=O)(=O)Nc4ccccc4C(F)(F)F)cc3)cc2NC1=O]

Example 64 was synthesized by method C. White solid, yield 52%. ¹H NMR (600 MHz, DMSO-d₆) δ 10.80 (s, 1H), 10.64 (s, 1H), 9.89 (s, 1H), 7.98 (d, J=9.0 Hz, 2H), 7.77 (d, J=9.0 Hz, 2H), 7.72 (d, J=7.2 Hz, 1H), 7.62 (dd, J=1.8, 7.8 Hz, 1H), 7.59 (t, J=7.8 Hz, 1H), 7.55 (d, J=1.8 Hz, 1H), 7.52 (d, J=8.4 Hz, 1H), 7.45 (t, J=7.2 Hz, 1H), 7.07 (d, J=7.8 Hz, 1H), 3.76 (q, J=7.2 Hz, 1H), 1.35 (d, J=7.2 Hz, 3H). ESI: m/z=522.10 [M+H]⁺.

### Example 65

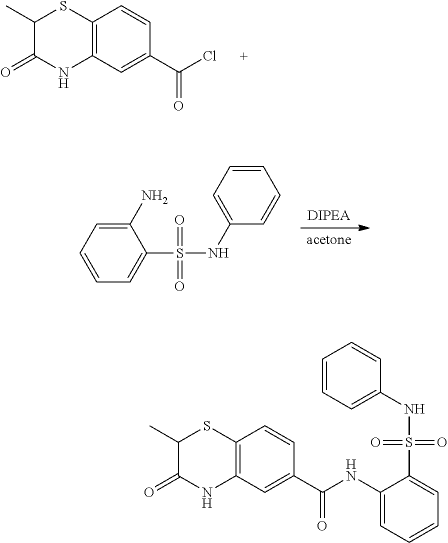 [SMILES: CC1Sc2ccc(C(=O)Cl)cc2NC1=O ; Nc1ccccc1S(=O)(=O)Nc1ccccc1 ; "DIPEA" ; "acetone" ; CC1Sc2ccc(C(=O)Nc3ccccc3S(=O)(=O)Nc3ccccc3)cc2NC1=O]

Example 65 was synthesized by method C. Yellow solid, yield 11%. ¹H NMR (600 MHz, DMSO-d₆) δ 10.84 (s, 1H), 10.47 (s, 1H), 10.04 (s, 1H), 8.30 (d, J=8.4 Hz, 1H), 7.73 (dd, J=1.8, 8.4 Hz, 1H), 7.62 (t, J=8.4 Hz, 1H), 7.54 (d, J=8.4 Hz, 1H), 7.51 (d, J=1.8 Hz, 1H), 7.42 (dd, J=1.8, 8.4 Hz, 1H), 7.24 (t, J=8.4 Hz, 1H), 7.15 (t, J=7.8 Hz, 2H), 7.02-7.00 (m, 3H), 3.75 (q, J=7.2 Hz, 1H), 1.34 (d, J=7.2 Hz, 3H). ESI: m/z=454.10 [M+H]⁺.

### General Synthetic Scheme and the Methods for the Synthesis of Examples 66-110

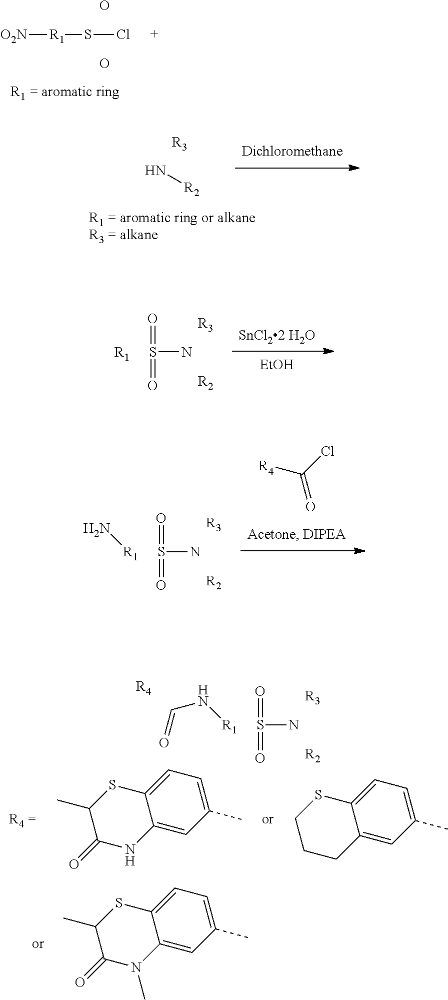

### Method D:

To a solution of anilines or amines (0.2 mmol) in dichloromethane (3 mL), was added sulfonyl chlorides (1 mmol) portionwise. The resulting solutions were stirred for 2 h and purified by flash chromatography on silica gel to give nitro-intermediates. The nitro-intermediates was dispersed in ethanol (5 mL) and tin (II) chloride dihydrate (0.6 mmol) was added. The resulting mixture was heated to reflux for 3 h. The solution was cooled to room temperature and filtered. The mother liquids were concentrated to dryness under reduced pressure. The residues and N,N-Diisopropylethylamine (1 mmol) was dispersed in acetone (3 mL) and to this mixture was added acyl chlorides (0.5 mmol). The resulting mixture was stirred overnight and filtered. The mother liquids were concentrated under reduced pressure and purified by prepare-HPLC to get final compounds.

### Example 66

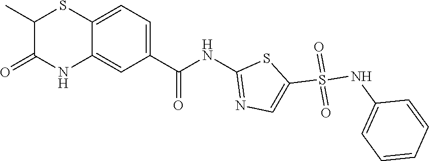 [SMILES: CC1Sc2ccc(C(=O)Nc3ncc(S(=O)(=O)Nc4ccccc4)s3)cc2NC1=O]

Materials 2-nitrothiazole-5-sulfonyl chloride, aniline and 2-methyl-3-oxo-3,4-dihydro-2H-benzo[b][1,4]thiazine-6-carbonyl chloride were used and followed method D to afford Example 66 (10 mg, yield 11%). H NMR (600 MHz, Methanol-d₄) δ 7.79 (s, 1H), 7.69 (s, 1H), 7.63 (d, J=8.1 Hz, 1H), 7.56 (s, 1H), 7.48 (d, J=8.3 Hz, 1H), 7.32-7.24 (m, 2H), 7.21-7.08 (m, 2H), 3.65 (q, J=7.0 Hz, 1H), 1.44 (d, J=7.0 Hz, 3H). MS (ESI) m/z 461.1 [M+H]⁺.

### Example 67

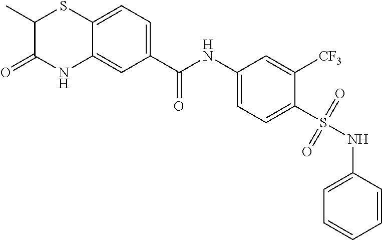 [SMILES: CC1Sc2ccc(C(=O)Nc3ccc(S(=O)(=O)Nc4ccccc4)c(C(F)(F)F)c3)cc2NC1=O]

Materials 4-nitro-2-(trifluoromethyl)benzenesulfonyl chloride, aniline and 2-methyl-3-oxo-3,4-dihydro-2H-benzo[b][1,4]thiazine-6-carbonyl chloride were used and followed method D to afford Example 67 (15 mg, yield 14%). ¹H NMR (600 MHz, Methanol-d₄) δ 8.37 (s, 1H), 8.06 (d, J=2.3 Hz, 2H), 7.60 (dd, J=8.1, 1.9 Hz, 1H), 7.52 (d, J=1.8 Hz, 1H), 7.47 (d, J=8.1 Hz, 1H), 7.24 (t, J=7.8 Hz, 2H), 7.14 (d, J=7.9 Hz, 2H), 7.07 (t, J=7.4 Hz, 1H), 3.66 (q, J=7.0 Hz, 1H), 1.46 (d, J=7.1 Hz, 3H). MS (ESI) m/z 522.2 [M+H]⁺.

### Example 68

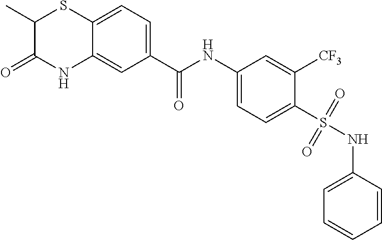 [SMILES: CC1Sc2ccc(C(=O)Nc3ccc(S(=O)(=O)Nc4ccccc4)c(C(F)(F)F)c3)cc2NC1=O]

Materials 2-chloro-4-nitrobenzenesulfonyl chloride, aniline and 2-methyl-3-oxo-3,4-dihydro-2H-benzo[b][1,4]thiazine-6-carbonyl chloride were used and followed method D to afford Example 68 (9 mg, yield 9%). ¹H NMR (600 MHz, Methanol-d₄) δ 8.06 (d, J=2.1 Hz, 1H), 8.01-7.94 (m, 1H), 7.68 (dd, J=8.8, 2.1 Hz, 1H), 7.55 (dd, J=8.1, 1.8 Hz, 1H), 7.47 (d, J=1.8 Hz, 1H), 7.44 (d, J=8.1 Hz, 1H), 7.21-7.16 (m, 2H), 7.16-7.11 (m, 2H), 7.06-6.97 (m, 1H), 3.63 (q, J=7.1 Hz, 1H), 1.43 (d, J=7.1 Hz, 3H). MS (ESI) m/z 488.1 [M+H]⁺.

### Example 69

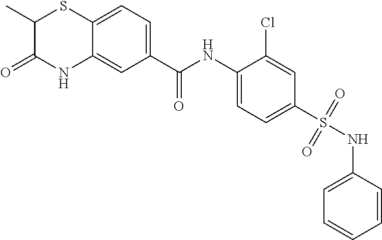 [SMILES: CC1Sc2ccc(C(=O)Nc3ccc(S(=O)(=O)Nc4ccccc4)cc3Cl)cc2NC1=O]

Materials 3-chloro-4-nitrobenzenesulfonyl chloride, aniline and 2-methyl-3-oxo-3,4-dihydro-2H-benzo[b][1,4]thiazine-6-carbonyl chloride were used and followed method D to afford Example 69 (13 mg, yield 13%). ¹H NMR (600 MHz, Methanol-d₄) δ 8.06 (d, J=2.1 Hz, 1H), 8.01-7.94 (m, 1H), 7.68 (dd, J=8.8, 2.1 Hz, 1H), 7.55 (dd, J=8.1, 1.8 Hz, 1H), 7.47 (d, J=1.8 Hz, 1H), 7.44 (d, J=8.1 Hz, 1H), 7.21-7.16 (m, 2H), 7.16-7.11 (m, 2H), 7.06-6.97 (m, 1H), 3.63 (q, J=7.1 Hz, 1H), 1.43 (d, J=7.1 Hz, 3H). MS (ESI) m/z 488.1 [M+H]⁺.

### Example 70

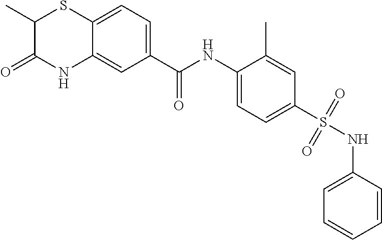 [SMILES: Cc1cc(S(=O)(=O)Nc2ccccc2)ccc1NC(=O)c1ccc2c(c1)NC(=O)C(C)S2]

Materials 3-methyl-4-nitrobenzenesulfonyl chloride, aniline and 2-methyl-3-oxo-3,4-dihydro-2H-benzo[b][1,4]thiazine-6-carbonyl chloride were used and followed method D to afford Example 70 (10 mg, yield 11%). ¹H NMR (600 MHz, Methanol-d₄) δ 7.68 (d, J=2.0 Hz, 1H), 7.64-7.60 (m, 1H), 7.60-7.54 (m, 2H), 7.53-7.49 (m, 1H), 7.46 (d, J=8.1 Hz, 1H), 7.21 (t, J=7.7 Hz, 2H), 7.14-7.08 (m, 2H), 7.07-7.03 (m, 1H), 3.67-3.61 (m, 1H), 2.31 (s, 3H), 1.44 (d, J=7.0 Hz, 3H). MS (ESI) m/z 468.1 [M+H]⁺.

### Example 71

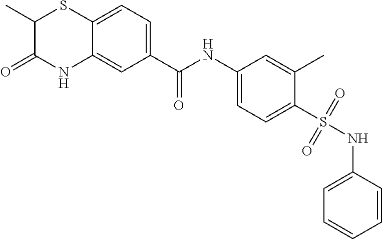 [SMILES: Cc1cc(NC(=O)c2ccc3c(c2)NC(=O)C(C)S3)ccc1S(=O)(=O)Nc1ccccc1]

Materials 2-methyl-4-nitrobenzenesulfonyl chloride, aniline and 2-methyl-3-oxo-3,4-dihydro-2H-benzo[b][1,4]thiazine-6-carbonyl chloride were used and followed method D to afford Example 71 (9 mg, yield 10%). ¹H NMR (600 MHz, Methanol-d₄) δ 7.89 (d, J=8.5 Hz, 1H), 7.71-7.61 (m, 2H), 7.56-7.53 (m, 1H), 7.47 (d, J=1.7 Hz, 1H), 7.44-7.41 (m, 1H), 7.21-7.14 (m, 2H), 7.09-7.04 (m, 2H), 7.00 (td, J=7.5, 1.2 Hz, 1H), 3.69-3.56 (m, 1H), 2.61 (s, 3H), 1.43 (dd, J=7.2, 1.0 Hz, 3H). MS (ESI) m/z 468.2 [M+H]⁺.

### Example 72

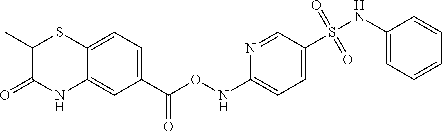 [SMILES: CC1Sc2ccc(C(=O)ONc3ccc(S(=O)(=O)Nc4ccccc4)cn3)cc2NC1=O]

Materials 6-nitropyridine-3-sulfonyl chloride, aniline and 2-methyl-3-oxo-3,4-dihydro-2H-benzo[b][1,4]thiazine-6-carbonyl chloride were used and followed method D to afford Example 72 (11 mg, yield 12%). ¹H NMR (600 MHz, Methanol-d₄) δ 8.16 (s, 1H), 7.76 (d, J=7.4 Hz, 1H), 7.66-7.57 (m, 2H), 7.41 (d, J=8.1, 5.3 Hz, 1H), 7.32-7.20 (m, 3H), 7.17-7.02 (m, 3H), 3.71-3.52 (m, 1H), 1.44-1.41 (m, 3H). MS (ESI) m/z 471.1 [M+H]⁺.

### Example 73

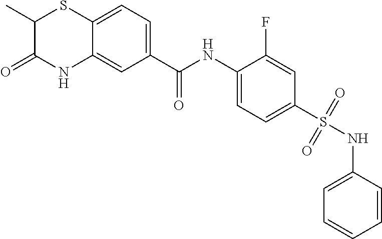 [SMILES: CC1Sc2ccc(C(=O)Nc3ccc(S(=O)(=O)Nc4ccccc4)cc3F)cc2NC1=O]

Materials 3-fluoro-4-nitrobenzenesulfonyl chloride, aniline and 2-methyl-3-oxo-3,4-dihydro-2H-benzo[b][1,4]thiazine-6-carbonyl chloride were used and followed method D to afford Example 73 (7, yield 7%). MS (ESI) m/z 472.2 [M+H]⁺.

### Example 74

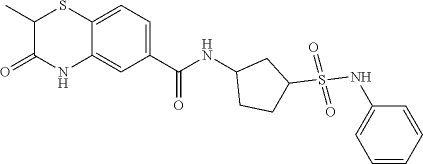 [SMILES: CC1Sc2ccc(C(=O)NC3CCC(S(=O)(=O)Nc4ccccc4)C3)cc2NC1=O]

Materials 5-nitrothiophene-2-sulfonyl chloride, aniline and 2-methyl-3-oxo-3,4-dihydro-2H-benzo[b][1,4]thiazine-6-carbonyl chloride were used and followed method D to afford Example 74 (11 mg, yield 12%). ¹H NMR (600 MHz, Methanol-d₄) δ 8.16 (s, 1H), 7.76 (d, J=7.4 Hz, 1H), 7.66-7.57 (m, 2H), 7.41 (d, J=8.1, 5.3 Hz, 1H), 7.32-7.20 (m, 3H), 7.17-7.02 (m, 3H), 3.71-3.52 (m, 1H), 1.44-1.41 (m, 3H). MS (ESI) m/z 460.2 [M+H]⁺.

### Example 75

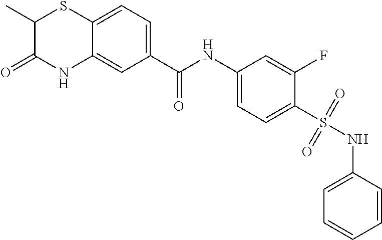 [SMILES: CC1Sc2ccc(C(=O)Nc3ccc(S(=O)(=O)Nc4ccccc4)c(F)c3)cc2NC1=O]

Materials 2-fluoro-4-nitrobenzenesulfonyl chloride, aniline and 2-methyl-3-oxo-3,4-dihydro-2H-benzo[b][1,4]thiazine-6-carbonyl chloride were used and followed method D to afford Example 75 (15 mg, yield 16%). ¹H NMR (600 MHz, Methanol-d₄) δ 7.85 (dd, J=12.8, 2.0 Hz, 1H), 7.75 (t, J=8.4 Hz, 1H), 7.54 (dd, J=8.1, 1.9 Hz, 1H), 7.50-7.46 (m, 2H), 7.44 (d, J=8.1 Hz, 1H), 7.26-7.16 (m, 2H), 7.16-7.10 (m, 2H), 7.07-6.97 (m, 1H), 3.63 (q, J=7.1 Hz, 1H), 1.43 (d, J=7.1 Hz, 3H). MS (ESI) m/z 472.1 [M+H]⁺.

### Example 76

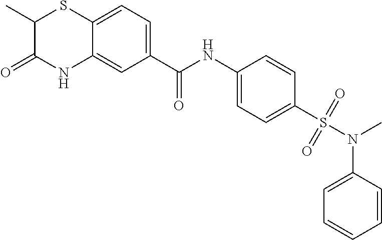 [SMILES: CC1Sc2ccc(C(=O)Nc3ccc(S(=O)(=O)N(C)c4ccccc4)cc3)cc2NC1=O]

Materials 4-nitrobenzenesulfonyl chloride, N-methyl aniline and 2-methyl-3-oxo-3,4-dihydro-2H-benzo[b][1,4]thiazine-6-carbonyl chloride were used and followed method D to afford Example 76 (7 mg, yield 7%). ¹H NMR (600 MHz, Methanol-d₄) δ 7.93-7.87 (m, 2H), 7.61 (dd, J=8.0, 1.9 Hz, 1H), 7.56-7.50 (m, 3H), 7.49 (d, J=8.1 Hz, 1H), 7.39-7.24 (m, 3H), 7.14 (dd, J=7.4, 1.9 Hz, 2H), 3.67 (q, J=7.1 Hz, 1H), 3.22 (s, 3H), 1.47 (d, J=7.1 Hz, 3H). MS (ESI) m/z 468.1 [M+H]⁺.

### Example 77

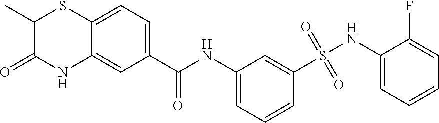 [SMILES: CC1Sc2ccc(C(=O)Nc3cccc(S(=O)(=O)Nc4ccccc4F)c3)cc2NC1=O]

Materials 3-nitrobenzenesulfonyl chloride, 2-fluoroaniline and 2-methyl-3-oxo-3,4-dihydro-2H-benzo[b][1,4]thiazine-6-carbonyl chloride were used and followed method D to afford Example 77 (13 mg, yield 14%). ¹H NMR (600 MHz, DMSO-d₆) δ 10.74 (s, 1H), 10.49 (s, 1H), 8.29 (s, 1H), 7.96 (d, J=9.0 Hz, 1H), 7.72-7.35 (m, 5H), 7.30-6.95 (m, 5H), 3.78-3.58 (m, 1H), 1.48-1.06 (m, 3H). MS (ESI) m/z 472.1 [M+H]⁺.

### Example 78

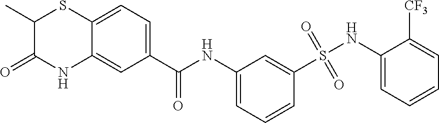 [SMILES: CC1Sc2ccc(C(=O)Nc3cccc(S(=O)(=O)Nc4ccccc4C(F)(F)F)c3)cc2NC1=O]

Materials 3-nitrobenzenesulfonyl chloride, 2-(trifluoromethyl)aniline and 2-methyl-3-oxo-3,4-dihydro-2H-benzo[b][1,4]thiazine-6-carbonyl chloride were used and followed method D to afford Example 78 (12 mg, yield 13%). ¹H NMR (600 MHz, DMSO-d₆) δ 10.75 (s, 1H), 10.54 (s, 1H), 10.01 (s, 1H), 8.47-8.17 (m, 1H), 8.02 (t, J=6.1 Hz, 1H), 7.70 (t, J=6.5 Hz, 1H), 7.67-7.25 (m, 7H), 7.14-6.90 (m, 1H), 3.78-3.65 (m, 1H), 1.32 (d, J=6.1 Hz, 3H). MS (ESI) m/z 522.1 [M+H]⁺.

### Example 79

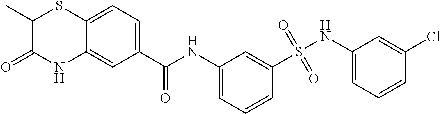 [SMILES: CC1Sc2ccc(C(=O)Nc3cccc(S(=O)(=O)Nc4cccc(Cl)c4)c3)cc2NC1=O]

Materials 3-nitrobenzenesulfonyl chloride, 3-chloroaniline and 2-methyl-3-oxo-3,4-dihydro-2H-benzo[b][1,4]thiazine-6-carbonyl chloride were used and followed method D to afford Example 79 (6 mg, yield 6%). ¹H NMR (600 MHz, Methanol-d₄) δ 8.34 (d, J=2.1 Hz, 1H), 7.88 (dt, J=8.1, 1.5 Hz, 1H), 7.61-7.53 (m, 2H), 7.54-7.49 (m, 2H), 7.46 (d, J=8.1 Hz, 1H), 7.24-7.15 (m, 2H), 7.07 (td, J=8.7, 2.1 Hz, 2H), 3.66 (q, J=7.1 Hz, 1H), 1.46 (d, J=7.1 Hz, 3H). MS (ESI) m/z 488.3 [M+H]⁺.

### Example 80

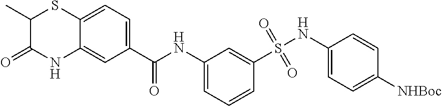 [SMILES: CC1Sc2ccc(C(=O)Nc3cccc(S(=O)(=O)Nc4ccc(NC(=O)OC(C)(C)C)cc4)c3)cc2NC1=O]

Materials 3-nitrobenzenesulfonyl chloride, tert-butyl (4-aminophenyl)carbamate and 2-methyl-3-oxo-3,4-dihydro-2H-benzo[b][1,4]thiazine-6-carbonyl chloride were used and followed method D to afford Example 80 (13 mg, yield 12%). ¹H NMR (800 MHz, Methanol-d₄) δ 8.22 (s, 1H), 7.89 (s, 1H), 7.59 (d, J=8.1 Hz, 1H), 7.54-7.44 (m, 4H), 7.27 (d, J=8.4 Hz, 2H), 7.02 (d, J=8.7 Hz, 2H), 3.67 (q, J=7.0 Hz, 1H), 1.62-1.41 (m, 12H). MS (ESI) m/z 569.1 [M+H]⁺.

### Example 81

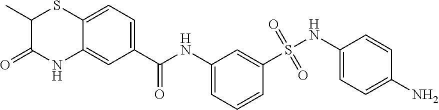 [SMILES: CC1Sc2ccc(C(=O)Nc3cccc(S(=O)(=O)Nc4ccc(N)cc4)c3)cc2NC1=O]

JJ095 was treated by trifluoroacetic acid (1 mL) and dichloromethane (1 mL) for 30 min and the volatile was concentrated into dryness to give Example 81 (yield 100%). ¹H NMR (500 MHz, Methanol-d₄) δ 8.27-8.00 (m, 1H), 7.97-7.68 (m, 1H), 7.62-7.27 (m, 5H), 6.96-6.79 (m, 2H), 6.74-6.48 (m, 2H), 3.59 (s, 1H), 1.40 (d, J=7.5 Hz, 3H). MS (ESI) m/z 469.1 [M+H]⁺.

### Example 82

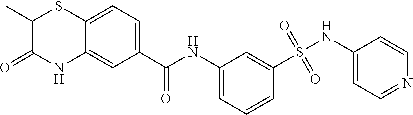 [SMILES: CC1Sc2ccc(C(=O)Nc3cccc(S(=O)(=O)Nc4ccncc4)c3)cc2NC1=O]

Materials 3-nitrobenzenesulfonyl chloride, pyridin-4-amine and 2-methyl-3-oxo-3,4-dihydro-2H-benzo[b][1,4]thiazine-6-carbonyl chloride were used and followed method D to afford Example 82 (11 mg, yield 12%). ¹H NMR (600 MHz, DMSO-d₆) δ 10.98-10.66 (m, 1H), 10.52 (s, 1H), 8.51-7.85 (m, 4H), 7.74-7.40 (m, 2H), 7.39-6.84 (m, 6H), 3.67-3.47 (m, 1H), 1.34 (d, 3H). MS (ESI) m/z 455.1 [M+H]⁺.

### Example 83

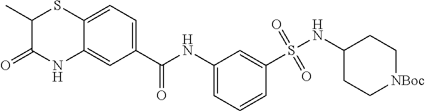 [SMILES: CC1Sc2ccc(C(=O)Nc3cccc(S(=O)(=O)NC4CCN(C(=O)OC(C)(C)C)CC4)c3)cc2NC1=O]

Materials 3-nitrobenzenesulfonyl chloride, tert-butyl 4-aminopiperidine-1-carboxylate and 2-methyl-3-oxo-3,4-dihydro-2H-benzo[b][1,4]thiazine-6-carbonyl chloride were used and followed method D to afford Example 83 (11 mg, yield 10%). ¹H NMR (800 MHz, Methanol-d₄) δ 8.40 (s, 1H), 7.88 (d, J=8.0 Hz, 1H), 7.67 (d, J=7.8 Hz, 1H), 7.62 (d, J=8.1 Hz, 1H), 7.61-7.54 (m, 2H), 7.49 (d, J=8.0 Hz, 1H), 3.93-3.88 (m, 2H), 3.67 (q, J=7.0 Hz, 1H), 3.32-3.27 (m, 1H), 2.88-2.86 (m, 2H), 1.77-1.70 (m, 2H), 1.50-1.43 (m, 12H), 1.41-1.33 (m, 2H). MS (ESI) m/z 561.2 [M+H]⁺.

### Example 84

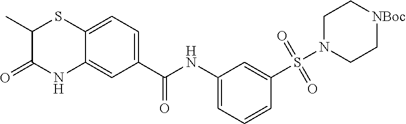 [SMILES: CC1Sc2ccc(C(=O)Nc3cccc(S(=O)(=O)N4CCN(C(=O)OC(C)(C)C)CC4)c3)cc2NC1=O]

Materials 3-nitrobenzenesulfonyl chloride, tert-butyl piperazine-1-carboxylate and 2-methyl-3-oxo-3,4-dihydro-2H-benzo[b][1,4]thiazine-6-carbonyl chloride were used and followed method D to afford Example 84 (14 mg, yield 13%). ¹H NMR (600 MHz, Methanol-d₄) δ 8.28 (dt, J=3.9, 1.9 Hz, 1H), 8.03-7.89 (m, 1H), 7.67-7.59 (m, 2H), 7.59-7.53 (m, 2H), 7.49 (d, J=8.1 Hz, 1H), 3.67 (q, J=7.1 Hz, 1H), 3.53 (s, 4H), 3.03 (t, J=5.1 Hz, 4H), 1.53-1.36 (m, 12H). MS (ESI) m/z 547.2 [M+H]⁺.

### Example 85

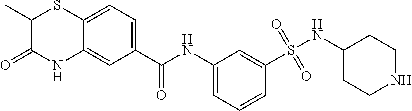 [SMILES: CC1Sc2ccc(C(=O)Nc3cccc(S(=O)(=O)NC4CCNCC4)c3)cc2NC1=O]

Example 83 was treated by trifluoroacetic acid (1 mL) and dichloromethane (1 mL) for 30 min and the volatile was concentrated into dryness to give Example 85 (yield 100%). ¹H NMR (600 MHz, Methanol-d₄) δ 8.44 (p, J=2.0 Hz, 1H), 7.83-7.75 (m, 1H), 7.66 (dt, J=7.9, 1.7 Hz, 1H), 7.61-7.54 (m, 2H), 7.52 (q, J=1.8 Hz, 1H), 7.47 (dd, J=8.1, 2.9 Hz, 1H), 3.68-3.61 (m, 1H), 3.48-3.39 (m, 1H), 3.37-3.31 (m, 2H), 3.05 (tt, J=13.5, 3.1 Hz, 2H), 2.00 (dt, J=14.5, 3.6 Hz, 2H), 1.71 (dtt, J=14.2, 11.0, 3.3 Hz, 2H), 1.45 (dd, J=7.2, 2.8 Hz, 3H). MS (ESI) m/z 461.2 [M+H]⁺.

### Example 86

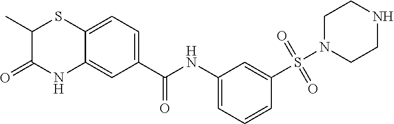 [SMILES: CC1Sc2ccc(C(=O)Nc3cccc(S(=O)(=O)N4CCNCC4)c3)cc2NC1=O]

Example 84 was treated by trifluoroacetic acid (1 mL) and dichloromethane (1 mL) for 30 min and the volatile was concentrated into dryness to give Example 86 (yield 100%). ¹H NMR (600 MHz, Methanol-d₄) δ 8.40-8.37 (m, 1H), 7.91 (d, J=8.1 Hz, 1H), 7.69-7.63 (m, 1H), 7.62-7.57 (m, 2H), 7.54-7.51 (m, 1H), 7.50-7.44 (m, 1H), 3.71-3.58 (m, 1H), 3.36-3.31 (m, 8H), 1.45-1.40 (m, 3H). MS (ESI) m/z 447.2 [M+H]⁺.

### Example 87

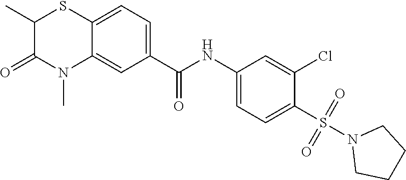 [SMILES: CC1Sc2ccc(C(=O)Nc3ccc(S(=O)(=O)N4CCCC4)c(Cl)c3)cc2N(C)C1=O]

Materials 2-chloro-4-nitrobenzenesulfonyl chloride, pyrrole and 2,4-dimethyl-3-oxo-3,4-dihydro-2H-benzo[b][1,4]thiazine-6-carbonyl chloride were used and followed method D to afford Example 87 (14 mg, yield 15%). ¹H NMR (800 MHz, Methanol-d₄) δ 8.52 (d, J=2.8 Hz, 1H), 8.04-7.98 (m, 1H), 7.84 (s, 1H), 7.70 (d, J=8.1 Hz, 1H), 7.63 (d, J=8.6 Hz, 1H), 7.56 (d, J=8.0 Hz, 1H), 3.69-3.64 (m, 1H), 3.56 (s, 3H), 3.47-3.44 (m, 4H), 2.00-1.94 (m, 4H), 1.45 (d, J=7.0 Hz, 3H). MS (ESI) m/z 480.2 [M+H]⁺.

### Example 88

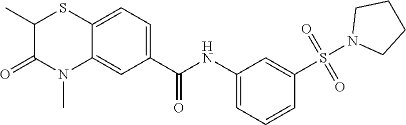 [SMILES: CC1Sc2ccc(C(=O)Nc3cccc(S(=O)(=O)N4CCCC4)c3)cc2N(C)C1=O]

Materials 3-nitrobenzenesulfonyl chloride, pyrrole and 2,4-dimethyl-3-oxo-3,4-dihydro-2H-benzo[b][1,4]thiazine-6-carbonyl chloride were used and followed method D to afford Example 88 (10 mg, yield 11%). ¹H NMR (800 MHz, Methanol-d₄) δ 8.36 (s, 1H), 8.04-7.98 (m, 1H), 7.85 (s, 1H), 7.71 (d, J=8.0 Hz, 1H), 7.64 (d, J=4.8 Hz, 2H), 7.57 (d, J=8.1 Hz, 1H), 3.67 (q, J=7.2 Hz, 1H), 3.57-3.53 (m, 4H), 1.86-1.74 (m, 4H), 1.46 (d, J=7.0 Hz, 3H). MS (ESI) m/z 446.1 [M+H]⁺.

### Example 89

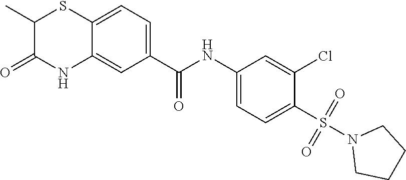 [SMILES: CC1Sc2ccc(C(=O)Nc3ccc(S(=O)(=O)N4CCCC4)c(Cl)c3)cc2NC1=O]

Materials 2-chloro-4-nitrobenzenesulfonyl chloride, pyrrole and 2-methyl-3-oxo-3,4-dihydro-2H-benzo[b][1,4]thiazine-6-carbonyl chloride were used and followed method D to afford Example 89 (17 mg, yield 18%). ¹H NMR (800 MHz, Methanol-d₄) δ 8.51 (d, J=2.8 Hz, 1H), 7.97 (dd, J=9.0, 2.8 Hz, 1H), 7.62 (dd, J=8.7, 5.2 Hz, 2H), 7.54 (s, 1H), 7.49 (d, J=8.1 Hz, 1H), 3.67 (q, J=7.1 Hz, 1H), 3.52-3.43 (m, 4H), 2.02-1.93 (m, 4H), 1.47 (d, J=7.0 Hz, 3H). MS (ESI) m/z 466.1 [M+H]⁺.

### Example 90

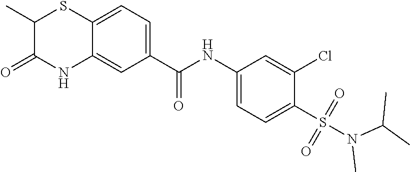 [SMILES: CC1Sc2ccc(C(=O)Nc3ccc(S(=O)(=O)N(C)C(C)C)c(Cl)c3)cc2NC1=O]

Materials 2-chloro-4-nitrobenzenesulfonyl chloride, N-methylpropan-2-amine and 2-methyl oxo-3,4-dihydro-2H-benzo[b][1,4]thiazine-6-carbonyl chloride were used and followed method D to afford Example 90 (13 mg, yield 14%). ¹H NMR (800 MHz, Methanol-d₄) δ 8.55 (d, J=2.7 Hz, 1H), 7.99 (dd, J=8.9, 2.8 Hz, 1H), 7.61 (t, J=8.9 Hz, 2H), 7.55 (s, 1H), 7.49 (d, J=8.0 Hz, 1H), 4.18-4.06 (m, 1H), 3.67 (q, J=7.0 Hz, 1H), 2.89 (s, 3H), 1.48 (d, J=7.0 Hz, 3H), 1.16 (d, J=6.7 Hz, 6H). MS (ESI) m/z 468.3 [M+H]⁺.

### Example 91

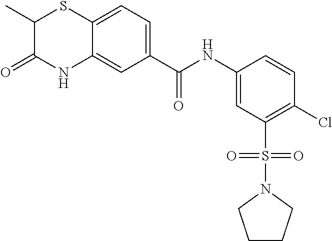 [SMILES: CC1Sc2ccc(C(=O)Nc3ccc(Cl)c(S(=O)(=O)N4CCCC4)c3)cc2NC1=O]

Materials 2-chloro-5-nitrobenzenesulfonyl chloride, pyrrole and 2-methyl-3-oxo-3,4-dihydro-2H-benzo[b][1,4]thiazine-6-carbonyl chloride were used and followed method D to afford Example 91 (10 mg, yield 11%). ¹H NMR (800 MHz, Methanol-d₄) δ 8.51 (d, J=2.7 Hz, 1H), 7.97 (dd, J=8.9, 2.7 Hz, 1H), 7.62 (dd, J=8.5, 5.2 Hz, 2H), 7.54 (s, 1H), 7.49 (d, J=8.1 Hz, 1H), 3.67 (q, J=7.0 Hz, 1H), 3.50-3.42 (m, 4H), 2.04-1.87 (m, 4H), 1.47 (d, J=7.1 Hz, 3H). MS (ESI) m/z 466.3 [M+H]⁺.

### Example 92

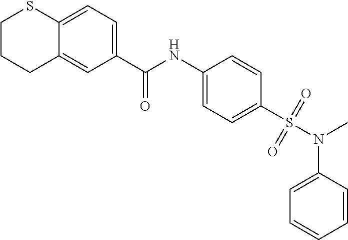 [SMILES: CN(c1ccccc1)S(=O)(=O)c1ccc(NC(=O)c2ccc3c(c2)CCCS3)cc1]

Materials 4-nitrobenzenesulfonyl chloride, N-methylaniline and thiochromane-6-carbonyl chloride were used and followed method D to afford Example 92 (9 mg, yield 10%). ¹H NMR (800 MHz, Methanol-d₄) δ 7.90 (d, J=8.4 Hz, 2H), 7.68-7.63 (m, 2H), 7.52 (d, J=8.4 Hz, 2H), 7.37-7.29 (m, 3H), 7.19 (d, J=8.2 Hz, 1H), 7.14 (d, J=7.8 Hz, 2H), 3.22 (s, 3H), 3.14-3.11 (m, 2H), 2.93 (t, J=6.1 Hz, 2H), 2.20-2.10 (m, 2H). MS (ESI) m/z 439.4 [M+H]⁺.

### Example 93

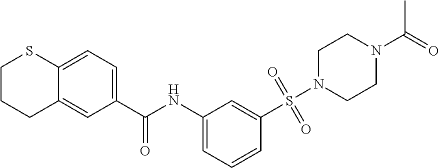 [SMILES: CC(=O)N1CCN(S(=O)(=O)c2cccc(NC(=O)c3ccc4c(c3)CCCS4)c2)CC1]

Materials 3-nitrobenzenesulfonyl chloride, 1-(piperazin-1-yl)ethan-1-one and thiochromane-6-carbonyl chloride were used and followed method D to afford Example 93 (8 mg, yield 9%). ¹H NMR (800 MHz, Methanol-d₄) δ 8.30 (s, 1H), 7.96 (d, J=8.1 Hz, 1H), 7.68 (s, 1H), 7.65 (d, J=8.2 Hz, 1H), 7.62 (t, J=7.9 Hz, 1H), 7.55 (d, J=7.8 Hz, 1H), 7.18 (d, J=8.1 Hz, 1H), 3.72-3.61 (m, 4H), 3.14-3.10 (m, 4H), 3.05 (s, 2H), 2.97-2.90 (m, 2H), 2.21-2.12 (m, 2H), 2.07 (s, 3H). MS (ESI) m/z 460.2 [M+H]⁺.

### Example 94

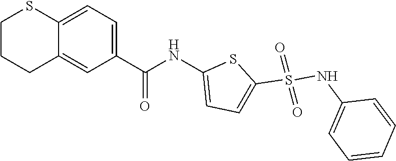 [SMILES: O=C(Nc1ccc(S(=O)(=O)Nc2ccccc2)s1)c1ccc2c(c1)CCCS2]

Materials 5-nitrothiophene-2-sulfonyl chloride, aniline and thiochromane-6-carbonyl chloride were used and followed method D to afford Example 94 (13 mg, yield 15%). ¹H NMR (800 MHz, Methanol-d₄) δ 7.70-7.58 (m, 2H), 7.32 (d, J=4.2 Hz, 1H), 7.26 (t, J=7.7 Hz, 2H), 7.21-7.15 (m, 3H), 7.10 (t, J=7.4 Hz, 1H), 6.77 (d, J=4.2 Hz, 1H), 3.16-3.07 (m, 2H), 2.92 (t, J=6.1 Hz, 2H), 2.15 (d, J=6.0 Hz, 2H). MS (ESI) m/z 431.2 [M+H]⁺.

### Example 95

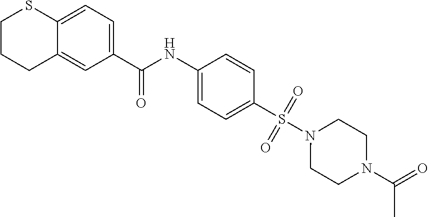 [SMILES: CC(=O)N1CCN(S(=O)(=O)c2ccc(NC(=O)c3ccc4c(c3)CCCS4)cc2)CC1]

Materials 4-nitrobenzenesulfonyl chloride, 1-(piperazin-1-yl)ethan-1-one and thiochromane carbonyl chloride were used and followed method D to afford Example 95 (10 mg, yield 11%). ¹H NMR (800 MHz, Methanol-d₄) δ 8.00 (d, J=8.4 Hz, 2H), 7.79 (d, J=8.5 Hz, 2H), 7.68 (s, 1H), 7.65 (d, J=8.3 Hz, 1H), 7.19 (d, J=8.2 Hz, 1H), 3.70-3.62 (m, 4H), 3.12 (t, J=5.9 Hz, 2H), 3.07 (t, J=5.0 Hz, 2H), 3.01 (t, J=5.2 Hz, 2H), 2.94 (t, J=6.0 Hz, 2H), 2.21-2.13 (m, 2H), 2.07 (s, 3H). MS (ESI) m/z 460.1 [M+H]⁺.

### Example 96

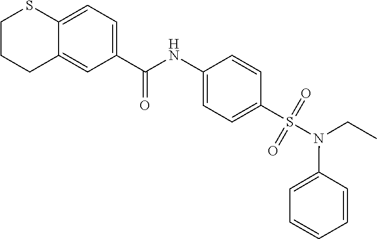 [SMILES: CCN(c1ccccc1)S(=O)(=O)c1ccc(NC(=O)c2ccc3c(c2)CCCS3)cc1]

Materials 4-nitrobenzenesulfonyl chloride, N-ethylaniline and thiochromane-6-carbonyl chloride were used and followed method D to afford Example 96 (9 mg, yield 10%). ¹H NMR (800 MHz, Methanol-d₄) δ 7.91 (d, J=8.4 Hz, 2H), 7.72-7.61 (m, 2H), 7.58 (d, J=8.4 Hz, 2H), 7.40-7.30 (m, 3H), 7.19 (d, J=8.2 Hz, 1H), 7.10-7.07 (m, 2H), 3.68 (q, J=7.1 Hz, 2H), 3.15-3.07 (m, 2H), 2.94 (t, J=6.1 Hz, 2H), 2.22-2.11 (m, 2H), 1.08 (t, J=7.1 Hz, 3H). MS (ESI) m/z 453.1 [M+H]⁺.

### Example 97

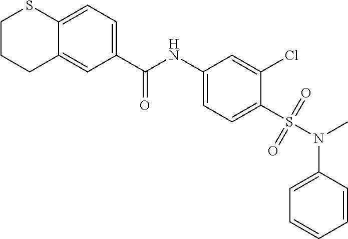 [SMILES: CN(c1ccccc1)S(=O)(=O)c1ccc(NC(=O)c2ccc3c(c2)CCCS3)cc1Cl]

Materials 2-chloro-4-nitrobenzenesulfonyl chloride, N-methylaniline and thiochromane-6-carbonyl chloride were used and followed method D to afford Example 97 (11 mg, yield 12%). ¹H NMR (800 MHz, Methanol-d₄) δ 8.15 (d, J=2.1 Hz, 1H), 7.79 (d, J=8.8 Hz, 1H), 7.70 (dd, J=9.1, 2.2 Hz, 1H), 7.66 (s, 1H), 7.64 (d, J=8.3 Hz, 1H), 7.35-7.30 (m, 2H), 7.29-7.24 (m, 3H), 7.19 (d, J=8.2 Hz, 1H), 3.42 (s, 3H), 3.15-3.04 (m, 2H), 2.93 (t, J=6.1 Hz, 2H), 2.16 (td, J=12.6, 12.0, 6.5 Hz, 2H). MS (ESI) m/z 473.1 [M+H]⁺.

### Example 98

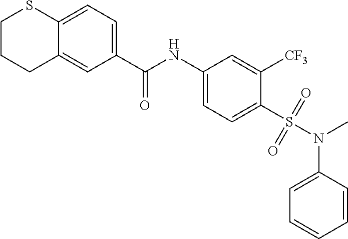 [SMILES: CN(c1ccccc1)S(=O)(=O)c1ccc(NC(=O)c2ccc3c(c2)CCCS3)cc1C(F)(F)F]

Materials 4-nitro-2-(trifluoromethyl)benzenesulfonyl chloride, N-methylaniline and thiochromane-6-carbonyl chloride were used and followed method D to afford Example 98 (10 mg, yield 11%). ¹H NMR (800 MHz, Methanol-d₄) δ 8.43 (s, 1H), 8.09 (d, J=8.8 Hz, 1H), 7.75 (d, J=8.8 Hz, 1H), 7.69 (s, 1H), 7.66 (d, J=8.2 Hz, 1H), 7.36 (t, J=7.6 Hz, 2H), 7.31 (t, J=7.3 Hz, 1H), 7.21 (s, 3H), 3.37 (s, 3H), 3.20-3.06 (m, 2H), 2.94 (t, J=6.1 Hz, 2H), 2.17-2.13 (m, 2H). MS (ESI) m/z 507.1 [M+H]⁺.

### Example 99

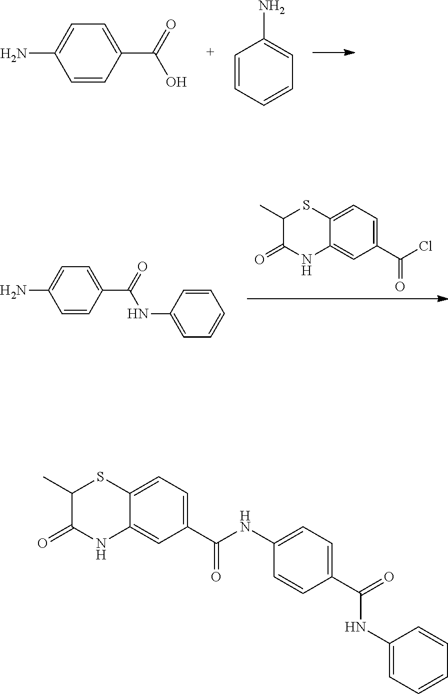 [SMILES: Nc1ccc(C(=O)O)cc1 ; Nc1ccccc1 ; Nc1ccc(C(=O)Nc2ccccc2)cc1 ; CC1Sc2ccc(C(=O)Cl)cc2NC1=O ; CC1Sc2ccc(C(=O)Nc3ccc(C(=O)Nc4ccccc4)cc3)cc2NC1=O]

To the solution of 4-aminobenzoic acid (27.4 mg, 0.2 mmol), EDCI (57.5 mg, 0.3 mmol), HOAt (40 mg, 0.3 mmol), NMM (40.4 mg, 0.4 mmol) in DMSO (5 mL), was added aniline (93.3 mg, 1 mmol). The resulting solution was stirred for 3 h and water (20 mL) was added, then extracted with ethyl acetate (10 mL) for 3 times. The organic phase was further washed with water (20 mL) and the organic phase was concentrated and purified by flash chromatography on silica gel (MeOH/DCM: 5-20%) to afford 4-amino-N-phenylbenzamide. MS (ESI) m/z 213.1 [M+H]⁺. 4-amino-N-phenylbenzamide was dissolved in acetone (5 mL) and DIPEA (130 mg, 1 mmol) was added. To the solution above was added 2-methyl-3-oxo-3,4-dihydro-2H-benzo[b][1,4]thiazine-6-carbonyl chloride (48 mg, 0.2 mmol). The resulting mixture was stirred overnight and concentrated, purified by pre-HPLC to afford Example 99 (20 mg, 25%). ¹H NMR (600 MHz, Methanol-d₄) δ 8.03-7.95 (m, 1H), 7.91-7.81 (m, 1H), 7.74-7.49 (m, 6H), 7.52-7.27 (m, 4H), 3.76-3.50 (m, 1H), 1.43 (d, J=7.2 Hz, 3H). MS (ESI) m/z 418.1 [M+H]⁺.

### Example 100

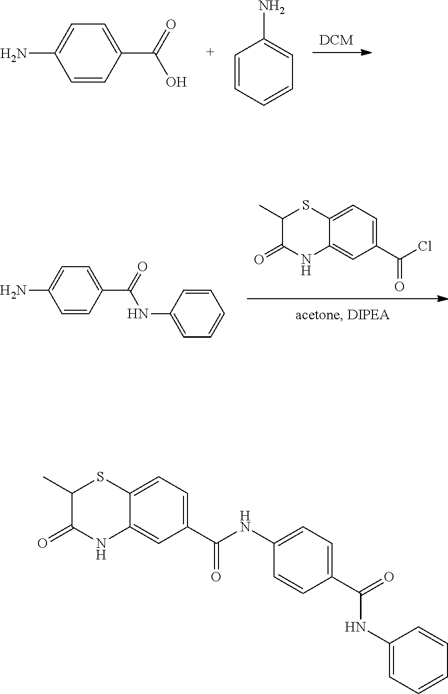

To a solution of benzene-1,4-diamine (108 mg, 1 mmol) in DCM (8 mL), was added benzenesulfonyl chloride (88 mg, 0.5 mmol). The resulting solution was stirred for 1 h, concentrated and purified by flash chromatography on silica gel (MeOH/DCM: 5-20%) to afford N-(4-aminophenyl)benzenesulfonamide (96 mg, 77%). MS (ESI) m/z 249.1 [M+H]⁺. The N-(4-aminophenyl)benzenesulfonamide (25 mg, 0.1 mmol) was dissolved in acetone (5 mL) and DIPEA (65 mg, 0.5 mmol) was added. To the solution above was added 2-methyl-3-oxo-3,4-dihydro-2H-benzo[b][1,4]thiazine-6-carbonyl chloride (24 mg, 0.1 mmol). The resulting mixture was stirred overnight, concentrated and purified by pre-HPLC to afford Example 100 (10 mg, 22%). ¹H NMR (600 MHz, Methanol-d₄) δ 7.74 (d, J=7.8 Hz, 1H), 7.65-7.32 (m, 9H), 7.07 (d, J=8.6 Hz, 2H), 3.63 (q, J=7.3 Hz, 1H), 1.50-1.35 (m, 3H). MS (ESI) m/z 454.2 [M+H]⁺.

### Method E:

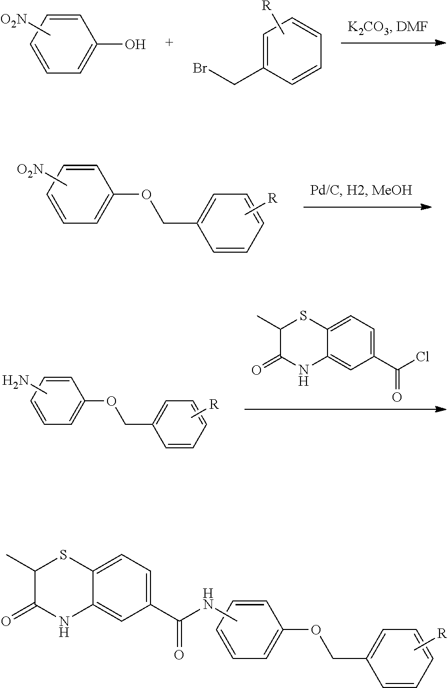

To a solution of 4-nitrophenol or 3-nitrophenol (28 mg, 0.2 mmol), K₂CO₃ (56 mg, 0.4 mmol) in DMF (5 mL), was added benzyl bromides (0.3 mmol). The resulting mixture was stirred for 2 h and water (10 mL) was added. The mixture was extracted with ethyl acetate for three times and the organic phase was further washed by water. The organic phase was concentrated and purified by flash chromatography on silica gel (hexane/ethyl acetate: 5-40%) to afford nitro-intermediates. The nitro-intermediate was dissolved in methanol (6 mL) and Pd/C (10%, 10 mg) was added. The solution was stirred under H₂ environment for 2 h and filtered. The mother liquid was concentrated and dissolved in acetone (5 mL). DIPEA (130 mg, 1 mmol) was added and followed by addition of 2-methyl-3-oxo-3,4-dihydro-2H-benzo[b][1,4]thiazine-6-carbonyl chloride (48 mg, 0.2 mmol). The resulting mixture was stirred overnight, concentrated and and purified by pre-HPLC to afford final compounds.

### Example 101

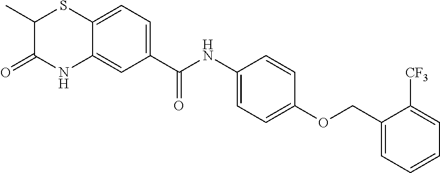 [SMILES: CC1Sc2ccc(C(=O)Nc3ccc(OCc4ccccc4C(F)(F)F)cc3)cc2NC1=O]

Materials 4-nitrophenol, 1-(bromomethyl)-2-(trifluoromethyl)benzene and 2-methyl-3-oxo-3,4-dihydro-2H-benzo[b][1,4]thiazine-6-carbonyl chloride were used and followed method E to afford Example 101 (13 mg, yield 14%). ¹H NMR (800 MHz, Methanol-d₄) δ 7.89-7.75 (m, 2H), 7.73-7.66 (m, 1H), 7.64-7.57 (m, 3H), 7.57-7.51 (m, 2H), 7.47 (d, J=8.1 Hz, 1H), 7.02 (d, J=8.8 Hz, 2H), 4.62 (s, 2H), 3.76-3.54 (m, 1H), 1.47 (d, J=7.0 Hz, 3H). MS (ESI) m/z 473.2 [M+H]⁺.

### Example 102

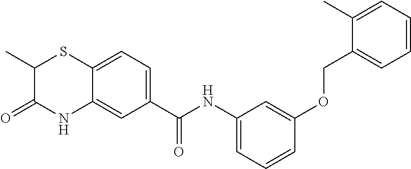 [SMILES: Cc1ccccc1COc1cccc(NC(=O)c2ccc3c(c2)NC(=O)C(C)S3)c1]

Materials 3-nitrophenol, 1-(bromomethyl)-2-methylbenzene and 2-methyl-3-oxo-3,4-dihydro-2H-benzo[b][1,4]thiazine-6-carbonyl chloride were used and followed method E to afford Example 102 (16 mg, yield 20%). ¹H NMR (800 MHz, Methanol-d₄) δ 7.59 (d, J=8.1 Hz, 1H), 7.54-7.46 (m, 3H), 7.43 (d, J=7.5 Hz, 1H), 7.32-7.19 (m, 5H), 6.87-6.84 (m, 1H), 5.12 (s, 2H), 3.66 (q, J=7.0 Hz, 1H), 2.41 (s, 3H), 1.47 (d, J=7.0 Hz, 3H). MS (ESI) m/z 419.1 [M+H]⁺.

### Example 103

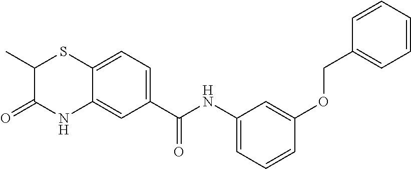 [SMILES: CC1Sc2ccc(C(=O)Nc3cccc(OCc4ccccc4)c3)cc2NC1=O]

Materials 3-nitrophenol, benzyl bromide and 2-methyl-3-oxo-3,4-dihydro-2H-benzo[b][1,4]thiazine-6-carbonyl chloride were used and followed method E to afford Example 103 (14 mg, yield 18%). ¹H NMR (800 MHz, Methanol-d₄) δ 7.59 (d, J=8.0 Hz, 1H), 7.52 (s, 1H), 7.51-7.46 (m, 4H), 7.40 (t, J=7.4 Hz, 2H), 7.35-7.31 (m, 1H), 7.31-7.23 (m, 2H), 6.83 (d, J=8.1 Hz, 1H), 5.13 (s, 2H), 3.66 (q, J=6.9 Hz, 1H), 1.47 (d, J=7.1 Hz, 3H). MS (ESI) m/z 404.1 [M+H]⁺.

### Example 104

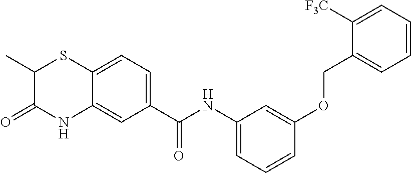 [SMILES: CC1Sc2ccc(C(=O)Nc3cccc(OCc4ccccc4C(F)(F)F)c3)cc2NC1=O]

Materials 3-nitrophenol, 1-(bromomethyl)-2-(trifluoromethyl)benzene and 2-methyl-3-oxo-3,4-dihydro-2H-benzo[b][1,4]thiazine-6-carbonyl chloride were used and followed method E to afford Example 104 (15 mg, yield 16%). ¹H NMR (800 MHz, Methanol-d₄) δ 7.83-7.76 (m, 2H), 7.70-7.66 (m, 1H), 7.59 (d, J=7.9 Hz, 1H), 7.53 (d, J=19.2 Hz, 3H), 7.48 (d, J=8.1 Hz, 1H), 7.31 (d, J=4.8 Hz, 2H), 6.83-6.80 (m, 1H), 5.31 (s, 2H), 3.67 (d, J=6.7 Hz, 1H), 1.47 (d, J=7.0 Hz, 3H). MS (ESI) m/z 473.1 [M+H]⁺.

### Example 105

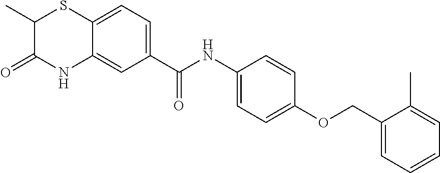 [SMILES: Cc1ccccc1COc1ccc(NC(=O)c2ccc3c(c2)NC(=O)C(C)S3)cc1]

Materials 4-nitrophenol, 1-(bromomethyl)-2-methylbenzene and 2-methyl-3-oxo-3,4-dihydro-2H-benzo[b][1,4]thiazine-6-carbonyl chloride were used and followed method E to afford Example 105 (11 mg, yield 13%). ¹H NMR (800 MHz, Methanol-d₄) δ 7.59 (d, J=8.4 Hz, 2H), 7.52 (s, 1H), 7.47 (d, J=7.8 Hz, 1H), 7.42 (d, J=7.6 Hz, 1H), 7.25 (d, J=7.6 Hz, 2H), 7.21 (d, J=6.8 Hz, 1H), 7.05 (d, J=8.8 Hz, 2H), 6.94 (d, J=8.6 Hz, 1H), 5.11 (s, 2H), 3.66 (q, J=6.8 Hz, 1H), 2.41 (s, 3H), 1.47 (d, J=7.1 Hz, 3H). MS (ESI) m/z 419.1 [M+H]⁺.

### Method F

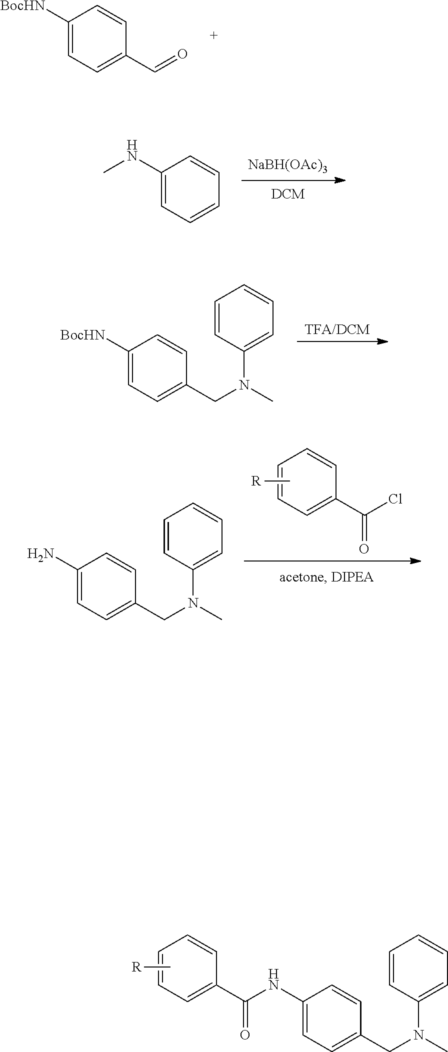

To a solution of tert-butyl (4-formylphenyl)carbamate (321 mg, 3 mmol), N-methylaniline in DCM (10 mL), was added NaBH(OAc)₃ (1.2 g, 6 mmol). The resulting mixture was stirred overnight and extracted with DCM/H₂O. Organic phase was concentrated and purified by flash chromatography on silica gel (hexane/ethyl acetate: 5-40%) to afford tert-butyl (4-((methyl(phenyl)amino)methyl)phenyl)carbamate (400 mg, 42%). MS (ESI) m/z 313.2 [M+H]⁺. tert-butyl (4-((methyl(phenyl)amino)methyl)phenyl)carbamate (31.2 mg, 0.1 mmol) was treated by TFA/DCM for 30 min and the volatile was removed. The residue above was dissolved DCM (3 mL) and TEA (50 mg, 0.5 mmol) was added. Then acyl chloride (0.1 mmol) was added. The resulting solution was stirred for 20 min and the volatile was removed. The residue was purified by pre-HPLC to afford final compounds.

### Example 106

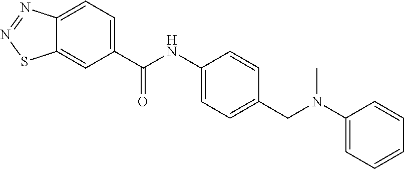 [SMILES: CN(Cc1ccc(NC(=O)c2ccc3nnsc3c2)cc1)c1ccccc1]

Benzo[d][1,2,3]thiadiazole-6-carbonyl chloride was used and followed method F to afford Example 106 (14 mg, yield 20%). ¹H NMR (800 MHz, Methanol-d₄) δ 9.37-9.18 (m, 1H), 8.48-8.36 (m, 1H), 8.33 (dd, J=15.4, 8.4 Hz, 1H), 7.88-7.70 (m, 3H), 7.50 (d, J=8.2 Hz, 1H), 7.30 (t, J=7.9 Hz, 4H), 6.99 (d, J=56.0 Hz, 1H), 4.65 (s, 2H), 3.16 (s, 3H). MS (ESI) m/z 375.1 [M+H]⁺.

### Example 107

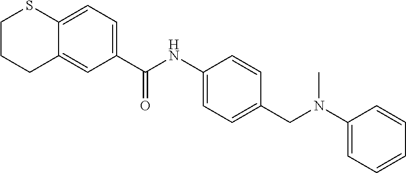 [SMILES: CN(Cc1ccc(NC(=O)c2ccc3c(c2)CCCS3)cc1)c1ccccc1]

Thiochromane-6-carbonyl chloride was used and followed method F to afford Example 107 (12 mg, yield 15%). ¹H NMR (800 MHz, Methanol-d₄) δ 7.69-7.59 (m, 3H), 7.31-7.22 (m, 5H), 7.17 (d, J=8.2 Hz, 1H), 6.93 (d, J=60.1 Hz, 3H), 4.61 (s, 2H), 3.34 (s, 3H), 3.16-3.07 (m, 2H), 2.96-2.85 (m, 2H), 2.16-2.13 (m, 2H). MS (ESI) m/z 389.2 [M+H]⁺.

### Example 108

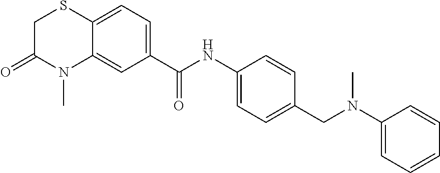 [SMILES: CN(Cc1ccc(NC(=O)c2ccc3c(c2)N(C)C(=O)CS3)cc1)c1ccccc1]

4-methyl-3-oxo-3,4-dihydro-2H-benzo[b][1,4]thiazine-6-carbonyl chloride was used and followed method F to afford the example (12 mg, yield 14%). ¹H NMR (800 MHz, Methanol-d₄) δ 7.80 (s, 1H), 7.72-7.62 (m, 3H), 7.56 (t, J=7.3 Hz, 2H), 7.41-7.35 (m, 2H), 7.27 (d, J=8.2 Hz, 2H), 7.19-7.11 (m, 2H), 4.68 (s, 2H), 3.54 (s, 3H), 3.33 (q, J=3.2 Hz, 3H), 3.22 (s, 2H). MS (ESI) m/z 434.2 [M+H]⁺.

### Example 109

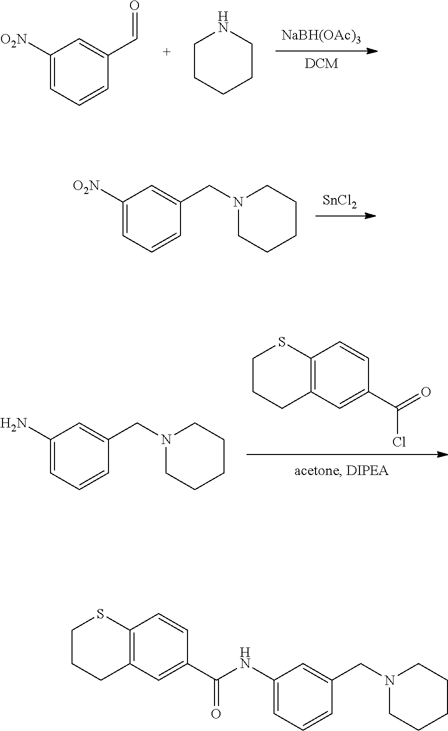

To a solution of 3-nitrobenzaldehyde (30 mg, 0.2 mmol), piperidine (25 mg, 03 mmol) in DCM (5 mL), was added NaBH(OAc)₃ (88 mg, 0.4 mmol). The resulting mixture was stirred overnight and extracted with DCM/H₂O. Organic phase was concentrated and purified by flash chromatography on silica gel (hexane/ethyl acetate: 5-40%) to afford 1-(3-nitrobenzyl)piperidine (25 mg, 57%). MS (ESI) m/z 221.2 [M+H]⁺. To the solution of 1-(3-nitrobenzyl)piperidine (25 mg, 0.11 mmol) in ethanol (6 mL), was added Tin (II) chloride dihydrate (90 mg, 0.4 mmol). The mixture was heated to reflux for 3 h, cooled to room temperature and filtered. The mother liquid was concentrated. The resulting residue was dispersed in acetone (5 mL) and DIPEA (65 mg, 0.5 mmol) was added. thiochromane-6-carbonyl chloride (21.2 mg, 0.1 mmol) was added and the mixture was stirred overnight. The volatile was removed and the residue was purified by pre-HPLC to afford the example (10 mg, 27%). ¹H NMR (800 MHz, Methanol-d₄) δ 8.02 (s, 1H), 7.70-7.64 (m, 3H), 7.51 (t, J=7.8 Hz, 1H), 7.30 (d, J=7.6 Hz, 1H), 7.19 (d, J=8.1 Hz, 1H), 4.32 (s, 2H), 3.52 (d, J=12.4 Hz, 3H), 3.17-3.09 (m, 2H), 3.02 (td, J=12.8, 3.1 Hz, 2H), 2.94 (t, J=6.1 Hz, 2H), 2.16 (p, J=6.1 Hz, 2H), 1.98 (d, J=14.5 Hz, 2H), 1.87 (d, J=12.7 Hz, 1H), 1.82-1.74 (m, 2H), 1.55 (qt, J=12.9, 3.8 Hz, 1H), 1.40 (td, J=7.1, 3.6 Hz, 1H). MS (ESI) m/z 367.2 [M+H]⁺.

### Example 110

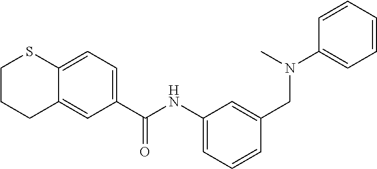 [SMILES: CN(Cc1cccc(NC(=O)c2ccc3c(c2)CCCS3)c1)c1ccccc1]

The example was prepared as described above. (11 mg, 29%). ¹H NMR (800 MHz, Methanol-d₄) δ 7.68-7.56 (m, 4H), 7.33-7.24 (m, 3H), 7.15 (d, J=8.2 Hz, 1H), 7.02 (d, J=7.6 Hz, 1H), 6.99-6.94 (m, 2H), 6.88 (s, 1H), 4.62 (s, 2H), 3.40-3.35 (s, 3H), 3.14 (s, 3H), 3.11-3.07 (m, 2H), 2.93-2.86 (m, 2H), 2.19-2.11 (m, 2H). MS (ESI) m/z 389.2 [M+H]⁺.

### General Synthetic Scheme for the Synthesis of Examples 111-121

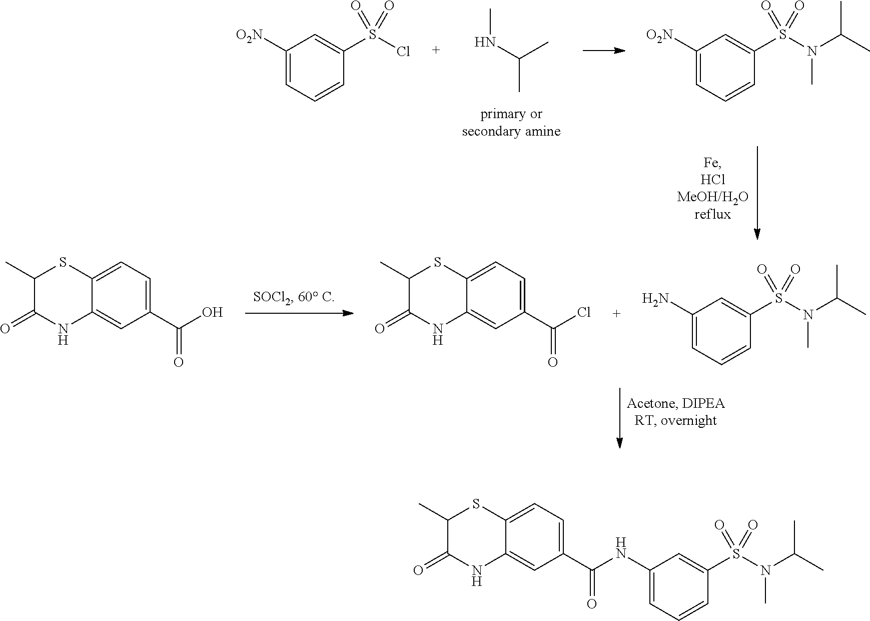

### Example 111

To a stirring mixture of 3-Nitro-benzenesulfonylChloride was added N-methyl-isopropyl amine. The resulting Sulfonamide then reduced to its corresponding amine by heating with Fe powder, HCl in MeOH/H₂O mixture. After work-up the reaction mixture purified by Flash Column Chromatography and obtained amine is reacted with the reacted with 2-methyl-3-oxo-3,4-dihydro-2H-benzo[b][1,4]thiazine-6-carbonyl chloride, which was obtained as described before, amine (1.0 equiv) and DIPEA (N,N-diisopropylethylamine) (3.0 equiv) were added. The reaction was stirring at room temperature overnight in acetone. The solvent was evaporated and the mixture was purified with the reverse phase ISCO to obtain a white solid. MS (ESI) m/z 434 [M+H]⁺.

Examples 113 to 122 are synthesized according to the general chemical route shown above.

### Example 112

Synthesized according to the general chemical route shown above and described above. MS (ESI) m/z 420 [M+H]⁺.

### Example 113

Synthesized according to the general chemical route shown above and described above. MS (ESI) m/z 473 [M+H]⁺.

### Example 114

Synthesized according to the general chemical route shown above. MS (ESI) m/z 488 [M+H]⁺.

### Example 115

Synthesized according to the general chemical route shown above. MS (ESI) m/z 472 [M+H]⁺.

### Example 116

Synthesized according to the general chemical route shown above. MS (ESI) m/z 522 [M+H]⁺.

### Example 117

Synthesized according to the general chemical route shown above. MS (ESI) m/z 468 [M+H]⁺.

### Example 118

Synthesized according to the general chemical route shown above. MS (ESI) m/z 418 [M+H]⁺.

### Example 119

Synthesized according to the general chemical route shown above. MS (ESI) m/z 522 [M+H]⁺.

### Example 120

Synthesized according to the general chemical route shown above. MS (ESI) m/z 468 [M+H]⁺.

### Example 121

Synthesized according to the general chemical route shown above. MS (ESI) m/z 468 [M+H]⁺.

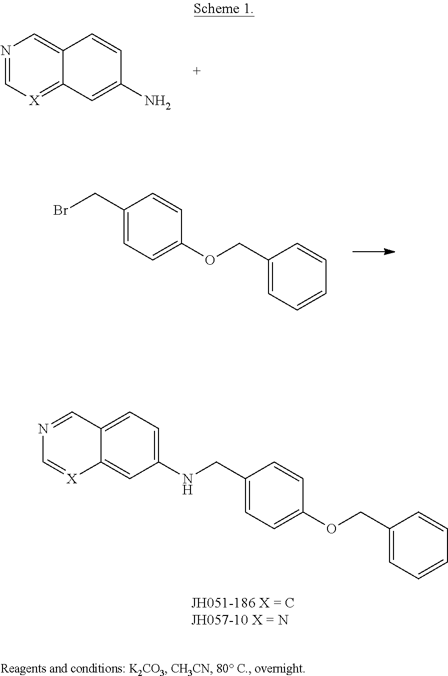

### Example 122

### N-(4-(benzyloxy)benzyl)isoquinolin-6-amine

To a solution of isoquinolin-6-amine (50 mg, 0.35 mmol) and 1-(benzyloxy)-4-(bromomethyl)benzene (105 mg, 0.38 mmol) in CH₃CN (2 mL) was added K₂CO₃ (145 mg, 1.05 mmol). The reaction mixture was heated to 80° C. overnight. The reaction was monitored by UPLC. Upon completion, the reaction mixture was purified by preparative HPLC to give Example 122 (68 g, 0.2 mmol, 57% yield) as white solid. ¹H NMR (800 MHz, DMSO-d₆) δ 9.40 (s, 1H), 8.24 (d, J=7.1 Hz, 1H), 8.03 (d, J=9.1 Hz, 1H), 7.80 (d, J=7.2 Hz, 1H), 7.51-7.45 (m, 3H), 7.42 (d, J=7.6 Hz, 2H), 7.37 (t, J=7.5 Hz, 2H), 7.31 (t, J=7.2 Hz, 1H), 7.28 (dd, J=9.4, 2.1 Hz, 1H), 7.05 (d, J=8.3 Hz, 2H), 6.88 (d, J=2.2 Hz, 1H), 5.54 (s, 2H), 5.10 (s, 2H). MS (ESI) m/z 341.20 [M+H]⁺

### Example 123

Following the similar experimental procedure to obtain compound.

### N-(4-(benzyloxy)benzyl)quinazolin-7-amine

52%, yellow solid. ¹H NMR (800 MHz, DMSO-d₆) δ 9.38 (s, 1H), 9.11 (s, 1H), 8.09 (s, 1H), 7.99 (d, J=9.1 Hz, 1H), 7.48 (d, J=8.3 Hz, 2H), 7.42 (d, J=7.6 Hz, 2H), 7.38 (t, J=7.4 Hz, 2H), 7.32 (t, J=7.2 Hz, 1H), 7.29 (dd, J=9.2, 2.0 Hz, 1H), 7.07 (d, J=8.4 Hz, 2H), 6.90 (s, 1H), 5.48 (s, 2H), 5.11 (s, 2H). MS (ESI) m/z 342.25 [M+H]⁺

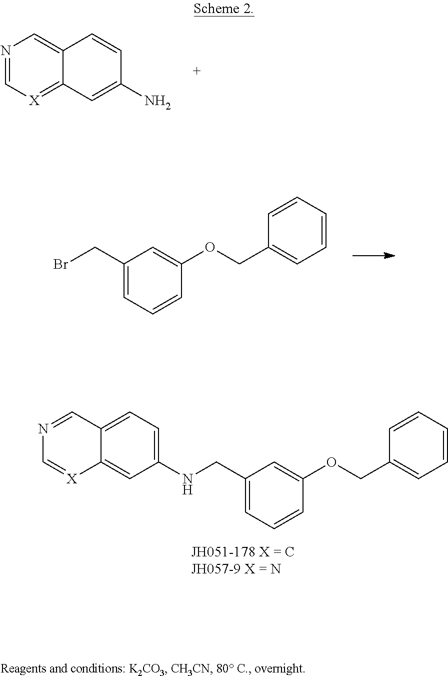

### Example 124

### N-(3-(benzyloxy)benzyl)isoquinolin-6-amine

To a solution of isoquinolin-6-amine (50 mg, 0.35 mmol) and 1-(benzyloxy)-3-(bromomethyl)benzene (105 mg, 0.38 mmol) in CH₃CN (2 mL) was added K₂CO₃ (145 mg, 1.05 mmol). The reaction mixture was heated to 80° C. overnight. The reaction was monitored by UPLC. Upon completion, the reaction mixture was purified by preparative HPLC to give Example 124 (61 g, 0.18 mmol, 51% yield) as yellow solid. ¹H NMR (800 MHz, DMSO-d₆) δ 9.45 (s, 1H), 8.26 (d, J=7.1 Hz, 1H), 8.04 (d, J=9.1 Hz, 1H), 7.79 (d, J=7.0 Hz, 1H), 7.58 (s, 2H), 7.40 (d, J=7.5 Hz, 2H), 7.33 (q, J=8.6, 8.1 Hz, 3H), 7.28 (t, J=7.3 Hz, 1H), 7.19 (s, 1H), 7.07 (d, J=7.7 Hz, 1H), 7.03 (dd, J=8.4, 2.6 Hz, 1H), 6.91 (d, J=2.1 Hz, 1H), 5.59 (s, 2H), 5.09 (s, 2H). MS (ESI) m/z 341.20 [M+H]⁺

### Example 125

A similar experimental procedure above is used to obtain the compound.

### N-(3-(benzyloxy)benzyl)quinazolin-7-amine

50%, white solid. ¹H NMR (600 MHz, Methanol-d₄) δ 9.11 (s, 1H), 8.88 (s, 1H), 7.91 (d, J=9.0 Hz, 1H), 7.36 (d, J=7.3 Hz, 3H), 7.30 (dt, J=15.7, 4.7 Hz, 3H), 7.23 (t, J=7.3 Hz, 1H), 7.09-7.02 (m, 3H), 6.99 (s, 1H), 5.50 (s, 2H), 5.09 (s, 2H). MS (ESI) m/z 342.25 [M+H]⁺

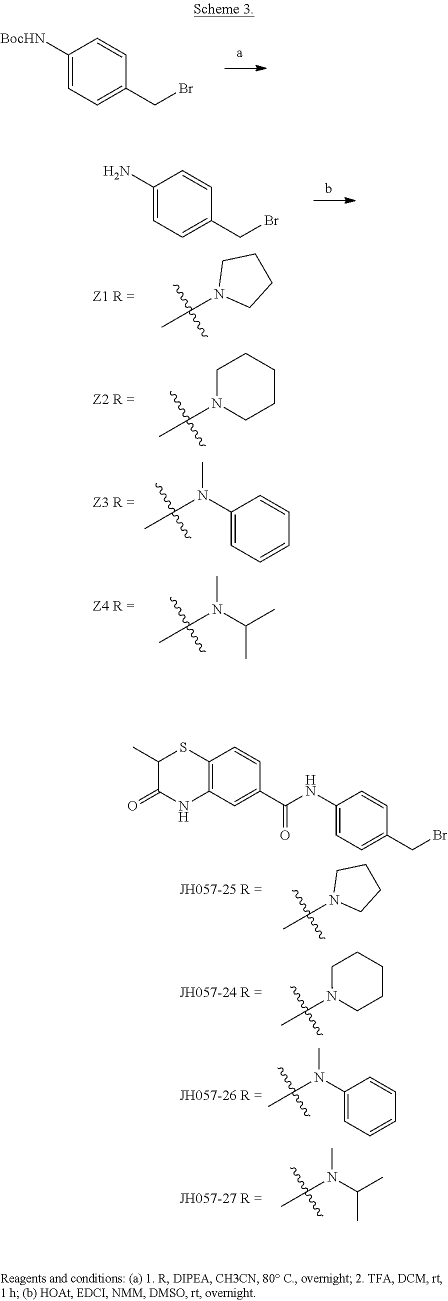

### Intermediate Z1

### 4-(pyrrolidin-1-ylmethyl)aniline

To a solution of tert-butyl (4-(bromomethyl)phenyl)carbamate (50 mg, 0.18 mmol) and pyrrolidine (16 μL, 0.19 mmol) in CH₃CN (2 mL) was added DIPEA (92 μL, 0.53 mmol). The reaction mixture was heated to 80° C. overnight. The reaction was monitored by UPLC. Upon completion, the reaction mixture was purified by preparative HPLC to give intermediate. The intermediate was dissolved in DCM (0.5 mL), then addition of TFA (0.5 mL). The mixture was stirred at room temperature for 1 h. The reaction was monitored by UPLC. Upon completion, the reaction mixture was purified by preparative HPLC to give Z1 (18 mg, 0.1 mmol, 56% yield) as yellow solid.

### Example 126

### 2-methyl-3-oxo-N-(4-(piperidin-1-ylmethyl)phenyl)-3,4-dihydro-2H-benzo[b][1,4]thiazine-6-carboxamide

50%, white solid. ¹H NMR (800 MHz, DMSO-d₆) δ 10.78 (s, 1H), 10.42 (s, 1H), 7.84 (d, J=8.1 Hz, 2H), 7.59 (d, J=8.2 Hz, 1H), 7.53 (s, 1H), 7.50 (d, J=8.0 Hz, 1H), 7.47 (d, J=8.2 Hz, 2H), 4.24 (d, J=4.4 Hz, 2H), 3.74 (q, J=7.0 Hz, 1H), 2.86 (dt, J=19.9, 10.2 Hz, 2H), 1.81 (d, J=14.3 Hz, 2H), 1.72-1.66 (m, 2H), 1.62 (d, J=13.8 Hz, 2H), 1.42-1.29 (m, 5H). MS (ESI) m/z 396.24 [M+H]⁺

### Examples 127

### 2-methyl-3-oxo-N-(4-(pyrrolidin-1-ylmethyl)phenyl)-3,4-dihydro-2H-benzo[b][1,4]thiazine-6-carboxamide

To a solution of Z1 (47 mg, 0.27 mmol) and 2-methyl-3-oxo-3,4-dihydro-2H-benzo[b][1,4]thiazine-6-carboxylic acid (60 mg, 0.27 mmol) in DMSO (2 mL) were added HOAt (55 mg, 0.41 mmol), EDCI (80 mg, 0.41 mmol), and NMM (89 μL, 0.81 mmol). The reaction mixture was stirred at room temperature overnight. The reaction was monitored by UPLC. Upon completion, the reaction mixture was purified by preparative HPLC to give Example 127 (57 g, 0.15 mmol, 56% yield) as white solid. ¹H NMR (600 MHz, Methanol-d₄) δ 7.83 (d, J=8.4 Hz, 2H), 7.58 (dd, J=8.1, 2.0 Hz, 1H), 7.51 (d, J=7.7 Hz, 3H), 7.45 (d, J=8.1 Hz, 1H), 4.36 (s, 2H), 3.64 (q, J=7.1 Hz, 1H), 3.50 (t, J=7.3 Hz, 2H), 3.19 (dt, J=12.8, 7.4 Hz, 2H), 2.18 (q, J=7.6 Hz, 2H), 2.01 (q, J=6.6 Hz, 2H), 1.44 (d, J=7.1 Hz, 3H). MS (ESI) m/z 382.34 [M+H]⁺

### Example 128

### 2-methyl-N-(4-((methyl(phenyl)amino)methyl)phenyl)-3-oxo-3,4-dihydro-2H-benzo[b][1,4]thiazine-6-carboxamide

53%, white solid. ¹H NMR (800 MHz, DMSO-d₆) δ 10.74 (s, 1H), 10.22 (s, 1H), 7.66 (d, J=8.2 Hz, 2H), 7.56 (d, J=8.1 Hz, 1H), 7.50 (s, 1H), 7.47 (d, J=8.1 Hz, 1H), 7.18 (d, J=8.2 Hz, 2H), 7.14 (t, J=7.8 Hz, 2H), 6.72 (d, J=8.2 Hz, 2H), 6.61 (t, J=7.2 Hz, 1H), 4.52 (s, 2H), 2.99 (s, 3H), 1.33 (d, J=7.0 Hz, 3H). MS (ESI) m/z 416.94 [M+H]⁺

### Example 129

### N-(4-((isopropyl(methyl)amino)methyl)phenyl)-2-methyl-3-oxo-3,4-dihydro-2H-benzo[b][1,4]thiazine-6-carboxamide

56%, white solid. ¹H NMR (600 MHz, Methanol-d₄) δ 7.89-7.80 (m, 2H), 7.58 (dd, J=8.1, 2.0 Hz, 1H), 7.54-7.49 (m, 3H), 7.44 (d, J=8.1 Hz, 1H), 4.43 (d, J=13.0 Hz, 1H), 4.16 (d, J=13.1 Hz, 1H), 3.64 (dq, J=16.3, 7.0 Hz, 2H), 2.71 (s, 3H), 1.42 (td, J=9.9, 8.5, 5.5 Hz, 9H). MS (ESI) m/z 384.34 [M+H]⁺

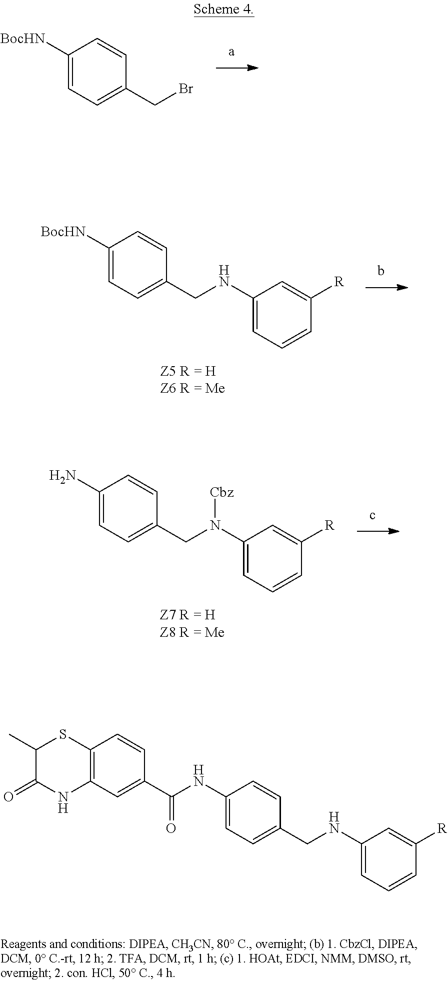

### Intermediate Z5

### tert-butyl (4-((phenylamino)methyl)phenyl)carbamate

To a solution of tert-butyl (4-(bromomethyl)phenyl)carbamate (200 mg, 0.7 mmol) and DIPEA (77 μL, 0.84 mmol) in CH₃CN (5 mL) was added aniline (0.37 mL, 2.1 mmol). The reaction mixture was heated to 80° C. overnight. The reaction was monitored by UPLC. Upon completion, the reaction mixture was purified by reverse column to give Z5 (119 mg, 0.4 mmol, 57% yield) as yellow solid.

Intermediate Z7

### benzyl (4-aminobenzyl)(phenyl)carbamate

To a solution of Z5 (100 mg, 0.34 mmol) in DCM (2 mL) was added DIPEA (175 μL, 1.0 mmol) and CbzCl (58 μL, 0.41 mmol) at 0° C. The reaction mixture was stirred at room temperature overnight. The reaction was monitored by UPLC. Upon completion, the reaction mixture was purified by reverse column to give 60 mg intermediate. The intermediate was dissolved in DCM (0.5 mL), then addition of TFA (0.5 mL). The mixture was stirred at room temperature for 1 h. The reaction was monitored by UPLC. Upon completion, the reaction mixture was purified by preparative HPLC to give intermediate Z7 (50 mg, 0.15 mmol, 44% yield) as yellow solid.

### Example 130

### 2-methyl-3-oxo-N-(4-((phenylamino)methyl)phenyl)-3,4-dihydro-2H-benzo[b][1,4]thiazine-6-carboxamide

To a solution of Z7 (50 mg, 0.15 mmol) and 2-methyl-3-oxo-3,4-dihydro-2H-benzo[b][1,4]thiazine-6-carboxylic acid (34 mg, 0.15 mmol) in DMSO (5 mL) were added HOAt (31 mg, 0.23 mmol), EDCI (45 mg, 0.23 mmol), and NMM (49 μL, 0.45 mmol). The reaction mixture was stirred at room temperature overnight. The reaction was monitored by UPLC. Upon completion, the reaction mixture was purified by preparative HPLC to give intermediate. The intermediate was dissolved in con. HCl (1 mL) and then was heated to 50° C. for 5 h. The reaction was monitored by UPLC. Upon completion, the reaction mixture was purified by preparative HPLC to give Example 130 (20 mg, 0.05 mmol, 33% yield) as yellow solid. ¹H NMR (600 MHz, Chloroform-d) δ 7.61 (d, J=7.9 Hz, 2H), 7.48 (d, J=8.1 Hz, 1H), 7.40 (s, 1H), 7.36 (d, J=8.1 Hz, 1H), 7.32 (d, J=8.1 Hz, 2H), 7.23 (t, J=7.7 Hz, 2H), 6.91 (t, J=7.4 Hz, 1H), 6.84 (d, J=7.9 Hz, 2H), 4.31 (s, 2H), 3.63-3.49 (m, 1H), 1.46 (d, J=7.0 Hz, 3H). MS (ESI) m/z 402.19 [M+H]⁺

### Example 131: Synthesis of JH057-43 (JJ131)

Following the similar experimental procedure above.

### 2-methyl-3-oxo-N-(4-((m-tolylamino)methyl)phenyl)-3,4-dihydro-2H-benzo[b][1,4]thiazine-6-carboxamide

56%, white solid. ¹H NMR (800 MHz, DMSO-d₆) δ 10.75 (s, 1H), 10.22 (s, 1H), 7.68 (d, J=8.3 Hz, 2H), 7.57 (d, J=8.1 Hz, 1H), 7.52 (s, 1H), 7.47 (d, J=8.0 Hz, 1H), 7.32 (d, J=8.2 Hz, 2H), 6.94 (t, J=7.7 Hz, 1H), 6.46 (s, 1H), 6.40 (dd, J=17.9, 7.8 Hz, 2H), 4.23 (s, 2H), 3.76-3.71 (m, 1H), 2.16 (s, 3H), 1.34 (d, J=7.0 Hz, 3H). MS (ESI) m/z 418.41 [M+H]⁺

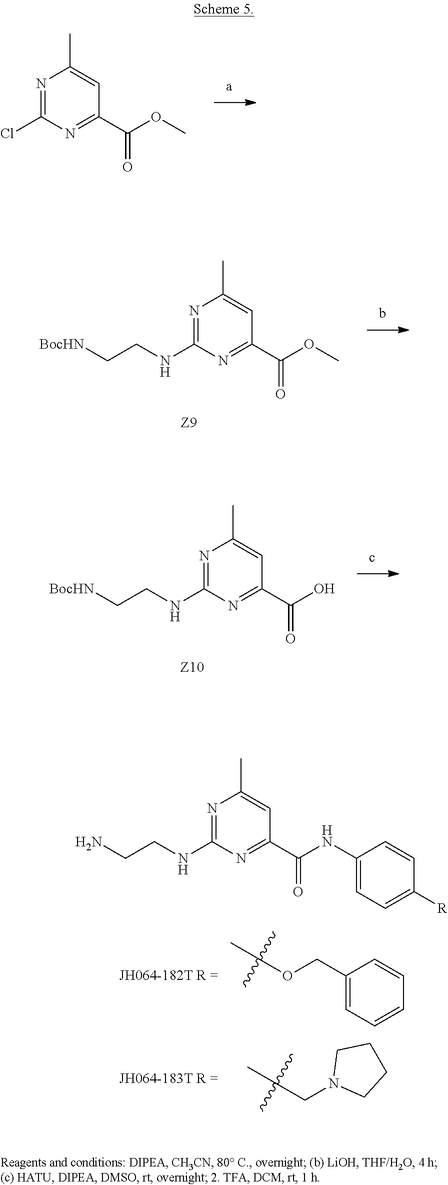

### Intermediate Z9

### methyl 2-((2-((tert-butoxycarbonyl)amino)ethyl)amino)-6-methylpyrimidine-4-carboxylate

To a solution of methyl 2-chloro-6-methylpyrimidine-4-carboxylate (200 mg, 1.08 mmol) and DIPEA (0.56 mL, 3.23 mmol) in CH₃CN (5 mL) was added tert-butyl (2-aminoethyl)carbamate (259 mg, 1.62 mmol). The reaction mixture was heated to 80° C. overnight. The reaction was monitored by UPLC. Upon completion, the reaction mixture was purified by reverse column to give Z9 (248 mg, 0.8 mmol, 74% yield) as white solid.

### Intermediate Z10

### 2-((2-((tert-butoxycarbonyl)amino)ethyl)amino)-6-methylpyrimidine-4-carboxylic acid

To a solution of Z9 (200 mg, 0.65 mmol) in THF (2 mL) and H₂O (1 mL) was added LiOH (47 mg, 1.95 mmol). The reaction mixture was stirred at room temperature overnight. The reaction was monitored by UPLC. Upon completion, the reaction mixture was purified by reverse column to give Z10 (178 mg, 0.6 mmol, 92% yield) as white solid.

### Example 132

### 2-((2-aminoethyl)amino)-N-(4-(benzyloxy)phenyl)-6-methylpyrimidine-4-carboxamide

To a solution of Z10 (50 mg, 0.17 mmol) and 4-(benzyloxy)aniline (34 mg, 0.17 mmol) in DMSO (2 mL) were added HATU (97 mg, 0.26 mmol) and DIPEA (89 μL, 0.51 mmol). The reaction mixture was stirred at room temperature overnight. The reaction was monitored by UPLC. Upon completion, the reaction mixture was purified by preparative HPLC to give intermediate. The intermediate was dissolved in DCM (0.5 mL), then addition of TFA (0.5 mL). The mixture was stirred at room temperature for 1 h. The reaction was monitored by UPLC. Upon completion, the reaction mixture was purified by preparative HPLC to give Example 132 (38 mg, 0.1 mmol, 59% yield) as yellow solid. ¹H NMR (800 MHz, DMSO-d₆) δ 10.11 (s, 1H), 7.95 (d, J=28.4 Hz, 3H), 7.75-7.66 (m, 2H), 7.45 (d, J=7.5 Hz, 2H), 7.39 (t, J=7.5 Hz, 2H), 7.33 (t, J=7.4 Hz, 1H), 7.16 (s, 1H), 7.04 (d, J=8.5 Hz, 2H), 5.11 (s, 2H), 3.77-3.62 (m, 2H), 3.04 (p, J=5.7 Hz, 3H), 2.38 (s, 2H). MS (ESI) m/z 378.33 [M+H]⁺

### Example 133

Following the similar experimental procedure described above.

### 2-((2-aminoethyl)amino)-6-methyl-N-(4-(pyrrolidin-1-ylmethyl)phenyl)pyrimidine-4-carboxamide

46%, white solid. ¹H NMR (800 MHz, Methanol-d₄) δ 7.90 (d, J=8.1 Hz, 2H), 7.54 (d, J=8.1 Hz, 2H), 7.28 (s, 1H), 4.37 (s, 2H), 3.80 (s, 2H), 3.50 (t, J=9.6 Hz, 2H), 3.23 (t, J=6.0 Hz, 2H), 3.19 (q, J=8.9, 8.1 Hz, 2H), 2.47 (s, 3H), 2.18 (d, J=7.9 Hz, 2H), 2.09-1.91 (m, 2H). MS (ESI) m/z 355.59 [M+H]⁺

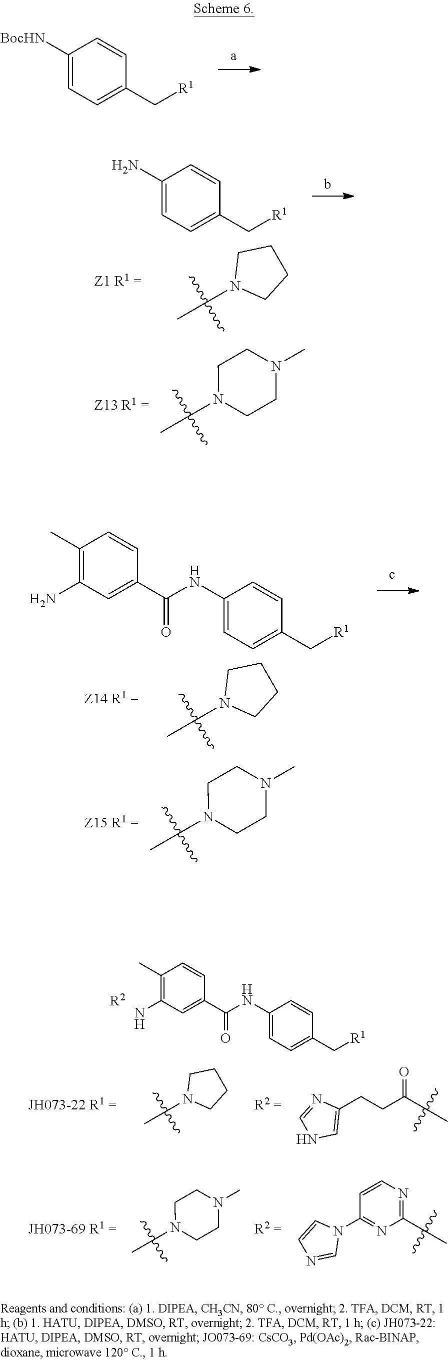

Following the similar experimental procedure of intermediate Z1 in scheme 3 to obtain intermediate Z13.

### Intermediate Z14

### 3-amino-4-methyl-N-(4-(pyrrolidin-1-ylmethyl)phenyl)benzamide

To a solution of Z1 (50 mg, 0.3 mmol) and 3-((tert-butoxycarbonyl)amino)-4-methylbenzoic acid (75 mg, 0.3 mmol) in DMSO (2 mL) were added HATU (171 mg, 0.45 mmol) and DIPEA (157 μL, 0.9 mmol). The reaction mixture was stirred at room temperature overnight. The reaction was monitored by UPLC. Upon completion, the reaction mixture was purified by preparative HPLC to give intermediate. The intermediate was dissolved in DCM (0.5 mL), then addition of TFA (0.5 mL). The mixture was stirred at room temperature for 1 h. The reaction was monitored by UPLC. Upon completion, the reaction mixture was purified by preparative HPLC to give Z14 (43 mg, 0.14 mmol, 47% yield) as yellow solid.

### Example 134

### 3-(3-(1H-imidazol-4-yl)propanamido)-4-methyl-N-(4-(pyrrolidin-1-ylmethyl)phenyl)benzamide

To a solution of Z14 (40 mg, 0.13 mmol) and 3-(1H-imidazol-4-yl)propanoic acid (18 mg, 0.13 mmol) in DMSO (2 mL) were added HATU (76 mg, 0.2 mmol) and DIPEA (70 μL, 0.4 mmol). The reaction mixture was stirred at room temperature overnight. The reaction was monitored by UPLC. Upon completion, the reaction mixture was purified by preparative HPLC to give Example 134 (43 mg, 0.14 mmol, 47% yield) as yellow solid. MS (ESI) [M+H]⁺=432.49.

### Example 135

### 3-((4-(1H-imidazol-1-yl)pyrimidin-2-yl)amino)-4-methyl-N-(4-((4-methylpiperazin-1-yl)methyl)phenyl)benzamide

A solution of Z15 (68 mg, 0.2 mmol), 2-bromo-4-(1H-imidazol-1-yl)pyrimidine (45 mg, 0.2 mmol), Pd(OAc)₂ (5 mg, 0.02 mmol), cesium carbonate (130 mg, 0.4 mmol), Rac-BINAP (13 mg, 0.02 mmol) in dioxane was heated to 120° C., under microwave for 0.5 h, After cooling to room temperature, the mixture was poured in water and extracted with EtOAc. The combined organic layer was washed with brine and concentrated. The resulting residue was purified by preparative HPLC to give the compound JH073-69 as white solid (29 mg, 0.06 mmol, 30% yield). ¹H NMR (800 MHz, Methanol-d₄) δ 8.59 (d, J=5.4 Hz, 1H), 8.26 (s, 1H), 8.19 (s, 1H), 7.72 (t, J=8.6 Hz, 4H), 7.56-7.51 (m, 1H), 7.44 (d, J=8.0 Hz, 1H), 7.39 (d, J=8.0 Hz, 2H), 7.21 (d, J=5.4 Hz, 1H), 3.77 (s, 3H), 2.87-2.83 (m, 6H), 2.52-2.48 (m, 4H), 2.40 (s, 3H).

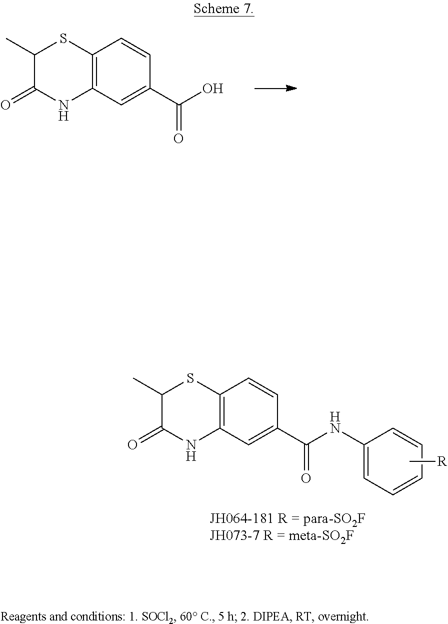

### Example 136

### 4-(2-methyl-3-oxo-3,4-dihydro-2H-benzo[b][1,4]thiazine-6-carboxamido)benzenesulfonyl fluoride

To a solution of 2-methyl-3-oxo-3,4-dihydro-2H-benzo[b][1,4]thiazine-6-carboxylic acid (100 mg, 0.45 mmol) was added SOCl₂ (2 mL) at 0° C. Then the reaction mixture was heated to 60° C. for 5 h. The reaction was evaporated to give intermediate. The intermediate was dissolved in acetone (1 mL), then addition of DIPEA (0.24 mL, 1.35 mmol) and 4-fluorobenzenesulfonyl fluoride (80 mg, 0.45 mmol) at 0° C. The mixture was stirred at room temperature for 3 h. The reaction was monitored by UPLC. Upon completion, the reaction mixture was purified by reverse column to give JH064-181 (91 mg, 0.24 mmol, 53% yield) as white solid. ¹H NMR (800 MHz, DMSO-d₆) δ 10.88 (s, 1H), 10.80 (s, 1H), 8.16 (d, J=8.8 Hz, 2H), 8.13 (d, J=8.8 Hz, 2H), 7.63 (d, J=8.1 Hz, 1H), 7.55 (s, 1H), 7.52 (d, J=8.0 Hz, 1H), 3.75 (q, J=7.0 Hz, 1H), 1.34 (d, J=7.0 Hz, 3H). MS (ESI) m/z 379.23 [M+H]⁺

Followed the similar experimental procedure to obtain compound JH073-7.

### Example 137

### 3-(2-methyl-3-oxo-3,4-dihydro-2H-benzo[b][1,4]thiazine-6-carboxamido)benzenesulfonyl fluoride

46%, white solid. ¹H NMR (800 MHz, DMSO-d₆) δ 10.81 (s, 1H), 10.77 (s, 1H), 8.65 (s, 1H), 8.26 (d, J=8.3 Hz, 1H), 7.85 (d, J=7.9 Hz, 1H), 7.79 (t, J=8.0 Hz, 1H), 7.64 (d, J=8.0 Hz, 1H), 7.56 (s, 1H), 7.52 (d, J=8.0 Hz, 1H), 3.75 (q, J=7.1 Hz, 1H), 1.34 (dd, J=7.1, 3.4 Hz, 3H). MS (ESI) m/z 381.08 [M+H]⁺

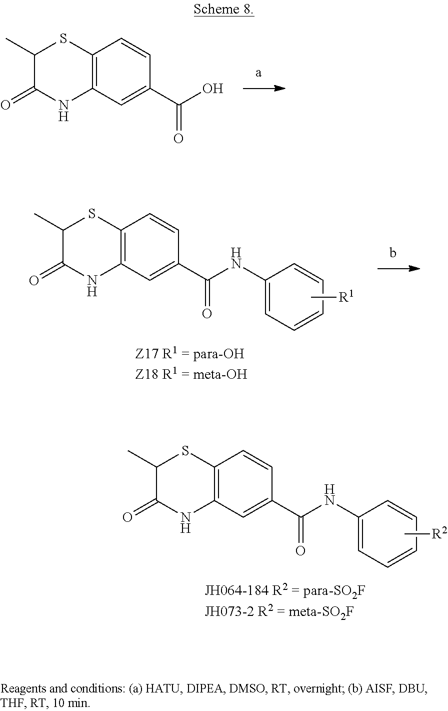

### Intermediate Z17

### N-(4-hydroxyphenyl)-2-methyl-3-oxo-3,4-dihydro-2H-benzo[b][1,4]thiazine-6-carboxamide

To a solution of 2-methyl-3-oxo-3,4-dihydro-2H-benzo[b][1,4]thiazine-6-carboxylic acid (100 mg, 0.45 mmol) and 4-aminophenol (49 mg, 0.45 mmol) in DMSO (2 mL) were added HATU (0.26 g, 0.68 mmol) and DIPEA (235 μL, 1.35 mmol). The reaction mixture was stirred at room temperature overnight. The reaction was monitored by UPLC. Upon completion, the reaction mixture was purified by reverse column to give Z17 (75 mg, 0.24 mmol, 53% yield) as yellow solid. ¹H NMR (800 MHz, DMSO-d₆) δ 10.73 (s, 1H), 10.02 (s, 1H), 9.26 (s, 1H), 7.56 (d, J=8.2 Hz, 1H), 7.50 (d, J=9.3 Hz, 2H), 7.45 (d, J=8.0 Hz, 1H), 6.73 (d, J=8.6 Hz, 1H), 3.72 (q, J=6.9 Hz, 1H), 1.34 (d, J=7.0 Hz, 3H). MS (ESI) m/z 434 [M+H]⁺

### Example 138

### 4-(2-methyl-3-oxo-3,4-dihydro-2H-benzo[b][1,4]thiazine-6-carboxamido)phenyl sulfurofluoridate

To a solution of Z17 (50 mg, 0.16 mmol) and AISF (60 mg, 0.19 mmol) in THF (2 mL) was added DBU (53 μL, 0.35 mmol). The reaction mixture was stirred at room temperature for 10 min. The reaction was monitored by UPLC. Upon completion, the reaction mixture was diluted with EtOAc and washed with 0.5N HCl and brine. The organic layer was evaporated and purified by flash column (EtOAc/Hexane=0-25%) to give JH064-184 (32 mg, 0.08 mmol, 50% yield) as yellow solid. ¹H NMR (800 MHz, DMSO-d₆) δ 10.78 (s, 1H), 10.53 (s, 1H), 8.10-7.87 (m, 2H), 7.60 (t, J=8.1 Hz, 3H), 7.54 (s, 1H), 7.51 (d, J=8.0 Hz, 1H), 3.74 (q, J=6.9 Hz, 1H), 1.34 (d, J=7.0 Hz, 3H). MS (ESI) m/z 397.08 [M+H]⁺

Following the similar experimental procedure to obtain compound JH073-2.

### Example 139: Synthesis of JH073-2 (JJ155)

### 3-(2-methyl-3-oxo-3,4-dihydro-2H-benzo[b][1,4]thiazine-6-carboxamido)phenyl sulfurofluoridate

48%, white solid. ¹H NMR (800 MHz, DMSO-d₆) δ 10.79 (s, 1H), 10.62 (s, 1H), 8.09 (s, 1H), 7.84 (d, J=8.3 Hz, 1H), 7.61 (d, J=8.1 Hz, 1H), 7.58 (t, J=8.2 Hz, 1H), 7.54 (s, 1H), 7.51 (d, J=8.0 Hz, 1H), 7.32 (dd, J=8.3, 2.6 Hz, 1H), 3.74 (q, J=6.9 Hz, 1H), 1.34 (d, J=7.1 Hz, 3H). MS (ESI) m/z 397.18 [M+H]⁺

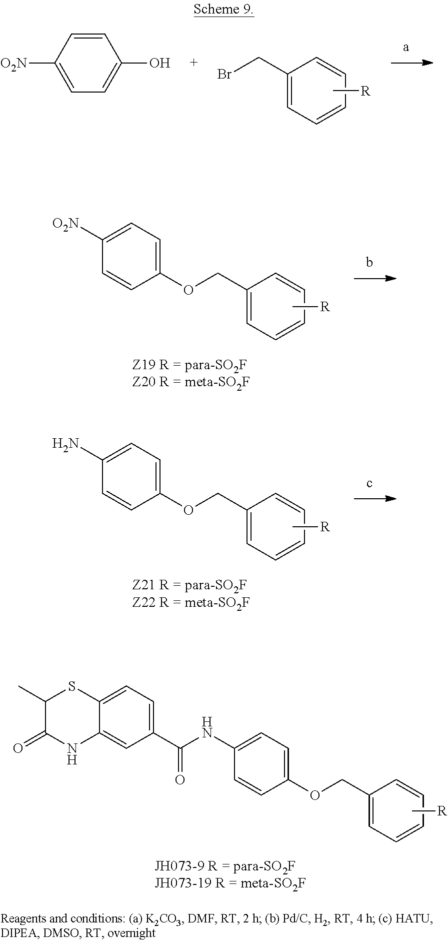

### Intermediate Z19

### 4-((4-nitrophenoxy)methyl)benzenesulfonyl fluoride

To a solution of 4-nitrophenol (100 mg, 0.72 mmol) and 4-(bromomethyl)benzenesulfonyl fluoride (182 mg, 0.72 mmol) in DMF (2 mL) was added K₂CO₃ (149 mg, 1.08 mmol). The reaction mixture was stirred at room temperature for 2 h. The reaction was monitored by UPLC. Upon completion, the reaction mixture was purified by reverse column to give Z19 (137 mg, 0.44 mmol, 61% yield) as white solid.

### Intermediate Z21

### 4-((4-aminophenoxy)methyl)benzenesulfonyl fluoride

To a solution of Z19 (100 mg, 0.32 mmol) in MeOH (2 mL) was added Pd/C (20 mg). The reaction mixture was filled with hydrogen and stirred at room temperature for 4 h. The reaction was monitored by UPLC. Upon completion, the reaction mixture was filtered to give Z21 (79 mg, 0.28 mmol, 88% yield) as white solid.

### Example 140: Synthesis of JH073-9 (JJ157)

### 4-((4-(2-methyl-3-oxo-3,4-dihydro-2H-benzo[b][1,4]thiazine-6-carboxamido)phenoxy)methyl)benzenesulfonyl fluoride

To a solution of Z21 (50 mg, 0.18 mmol) and 2-methyl-3-oxo-3,4-dihydro-2H-benzo[b][1,4]thiazine-6-carboxylic acid (40 mg, 0.18 mmol) in DMSO (2 mL) were added HATU (103 mg, 0.27 mmol) and DIPEA (94 μL, 0.54 mmol). The reaction mixture was stirred at room temperature overnight. The reaction was monitored by UPLC. Upon completion, the reaction mixture was purified by reverse column to give JH073-9 (49 mg, 0.1 mmol, 56% yield) as yellow solid. ¹H NMR (800 MHz, DMSO-d₆) δ 10.74 (s, 1H), 10.16 (s, 1H), 8.17 (d, J=8.1 Hz, 2H), 7.84 (d, J=8.1 Hz, 2H), 7.67 (d, J=8.8 Hz, 2H), 7.57 (d, J=8.0 Hz, 1H), 7.51 (s, 1H), 7.47 (d, J=8.0 Hz, 1H), 7.12-6.96 (m, 2H), 5.32 (s, 2H), 3.73 (q, J=7.0 Hz, 1H), 1.34 (d, J=7.0 Hz, 3H). MS (ESI) m/z 487.11 [M+H]⁺

### Example 141

Following the similar experimental procedure above compound JH073-19 is obtained.

### 3-((4-(2-methyl-3-oxo-3,4-dihydro-2H-benzo[b][1,4]thiazine-6-carboxamido)phenoxy)methyl)benzenesulfonyl fluoride

49%, white solid. ¹H NMR (800 MHz, DMSO-d₆) δ 10.75 (s, 1H), 10.16 (s, 1H), 8.20 (s, 1H), 8.11 (d, J=7.9 Hz, 1H), 8.03 (d, J=7.7 Hz, 1H), 7.83 (t, J=7.8 Hz, 1H), 7.67 (d, J=8.8 Hz, 2H), 7.57 (d, J=8.1 Hz, 1H), 7.52 (s, 1H), 7.47 (d, J=8.0 Hz, 1H), 7.04 (d, J=8.7 Hz, 2H), 5.28 (s, 2H), 3.73 (q, J=7.0 Hz, 1H), 1.34 (d, J=7.1 Hz, 3H). MS (ESI) m/z 487.16 [M+H]⁺

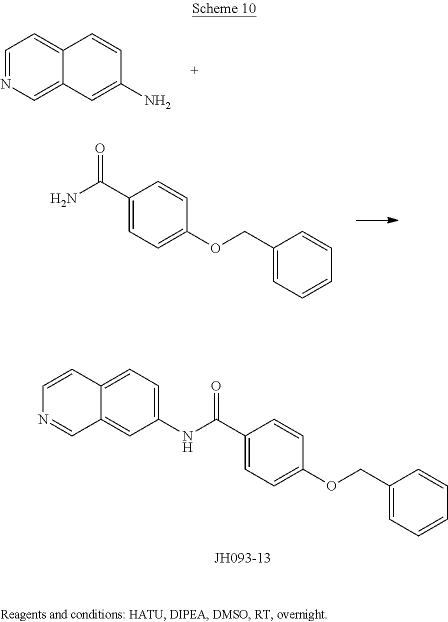 [SMILES: Nc1ccc2ccncc2c1 ; NC(=O)c1ccc(OCc2ccccc2)cc1 ; O=C(Nc1ccc2ccncc2c1)c1ccc(OCc2ccccc2)cc1]

### Example 142: Synthesis of JH093-13 (JJ187)

### 4-(benzyloxy)-N-(isoquinolin-7-yl)benzamide

To a solution of isoquinolin-7-amine (50 mg, 0.35 mmol) and 4-(benzyloxy)benzoic acid (79 mg, 0.35 mmol) in DMSO (2 mL) were added HATU (0.57 g, 0.53 mmol) and DIPEA (183 μL, 1.05 mmol). The reaction mixture was stirred at room temperature overnight. The reaction was monitored by UPLC. Upon completion, the reaction mixture was purified by reverse column to give JH093-13 (64 mg, 0.18 mmol, 56% yield) as yellow solid. ¹H NMR (800 MHz, DMSO-d₆) δ 10.78 (s, 1H), 9.09 (d, J=5.0 Hz, 1H), 8.87 (s, 1H), 8.82 (d, J=8.2 Hz, 1H), 8.20 (d, J=8.9 Hz, 1H), 8.11 (d, J=8.9 Hz, 1H), 8.03 (d, J=8.4 Hz, 2H), 7.77 (dd, J=8.2, 4.9 Hz, 1H), 7.48 (d, J=7.5 Hz, 2H), 7.41 (t, J=7.5 Hz, 2H), 7.35 (t, J=7.4 Hz, 1H), 7.19 (d, J=8.4 Hz, 2H), 5.23 (s, 2H). MS (ESI) m/z 355.24 [M+H]⁺

### Example 143

JH093-20 is obtained using the same experimental procedure for JH093-13. MS (ESI) m/z 356.41 [M+H]⁺

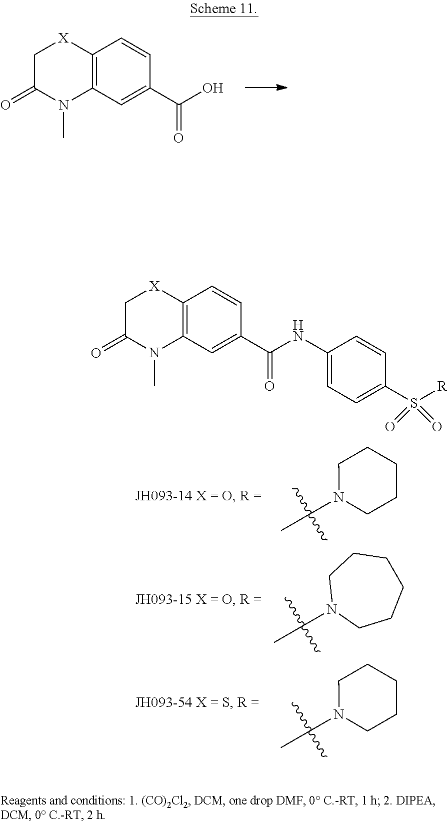

### Example 144: Synthesis of JH093-14 (JJ188)

### 4-methyl-3-oxo-N-(4-(piperidin-1-ylsulfonyl)phenyl)-3,4-dihydro-2H-benzo[b][1,4]oxazine-6-carboxamide

To a solution of 4-methyl-3-oxo-3,4-dihydro-2H-benzo[b][1,4]oxazine-6-carboxylic acid (60 mg, 0.29 mmol) in DCM (2 mL) and one drop DMF was added (CO)₂Cl₂ (2 mL) at 0° C. Then the reaction mixture was stirred at room temperature for 1 h. The reaction was evaporated to give intermediate. The intermediate was dissolved in DCM (2 mL), then addition of META (0.15 mL, 0.87 mmol) and 4-(piperidin-1-ylsulfonyl)aniline (70 mg, 0.29 mmol) at 0° C. The mixture was stirred at room temperature for 2 h. The reaction was monitored by HPLC. Upon completion, the reaction mixture was purified by reverse column to give JH093-14 (51 mg, 0.12 mmol, 41% yield) as white solid. ¹H NMR (800 MHz, DMSO-d₆) δ 10.57 (s, 1H), 8.03 (d, J=8.4 Hz, 2H), 7.72 (d, J=8.4 Hz, 2H), 7.70 (d, J=6.7 Hz, 2H), 7.15 (d, J=8.9 Hz, 1H), 4.76 (s, 2H), 3.36 (s, 2H), 2.87 (t, J=5.5 Hz, 4H), 2.08 (s, 1H), 1.54 (p, J=5.6 Hz, 4H), 1.35 (p, J=6.3, 5.8 Hz, 2H). MS (ESI) m/z 430.27 [M+H]⁺

Followed a similar experimental procedure to obtain compounds JH093-15 and JH093-54.

### Example 145: Synthesis of JH093-15 (JJ189)

### N-(4-(azepan-1-ylsulfonyl)phenyl)-4-methyl-3-oxo-3,4-dihydro-2H-benzo[b][1,4]oxazine-6-carboxamide

42%, white solid. ¹H NMR (800 MHz, DMSO-d₆) δ 10.52 (s, 1H), 7.99 (d, J=8.4 Hz, 2H), 7.77 (d, J=8.4 Hz, 2H), 7.73-7.64 (m, 2H), 7.14 (d, J=8.8 Hz, 1H), 4.76 (s, 2H), 3.36 (s, 3H), 3.20 (t, J=6.0 Hz, 4H), 1.61 (q, J=5.4 Hz, 4H), 1.49 (p, J=3.0 Hz, 4H). MS (ESI) m/z 444.22 [M+H]⁺

### Example 146: Synthesis of JH093-54 (JJ216)

### 4-methyl-3-oxo-N-(4-(piperidin-1-ylsulfonyl)phenyl)-3,4-dihydro-2H-benzo[b][1,4]thiazine-6-carboxamide

52%, white solid. ¹H NMR (800 MHz, DMSO-d₆) δ 10.62 (s, 1H), 7.99 (d, J=8.4 Hz, 2H), 7.78 (d, J=8.4 Hz, 2H), 7.75 (s, 1H), 7.67 (d, J=8.0 Hz, 1H), 7.59 (d, J=7.9 Hz, 1H), 3.60 (s, 2H), 3.43 (s, 3H), 3.20 (t, J=6.0 Hz, 2H), 1.62 (t, J=5.3 Hz, 4H), 1.49 (p, J=3.0 Hz, 4H). MS (ESI) m/z 460.17 [M+H]⁺

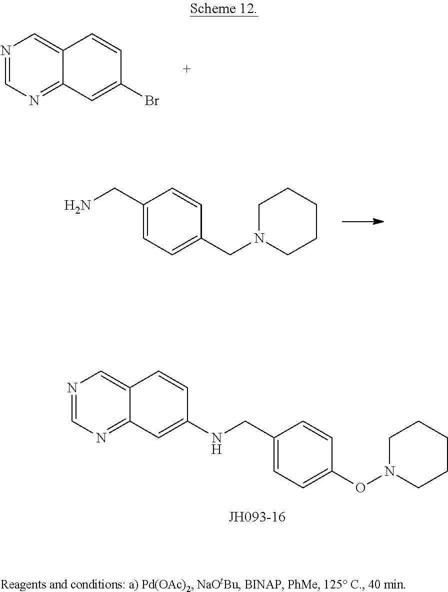 [SMILES: Brc1ccc2cncnc2c1 ; NCc1ccc(CN2CCCCC2)cc1 ; c1ncc2ccc(NCc3ccc(ON4CCCCC4)cc3)cc2n1]

### Example 147: Synthesis of JH093-16 (JJ190)

### N-(4-(piperidin-1-ylmethyl)benzyl)quinazolin-7-amine

A solution of 7-bromoquinazoline (59 mg, 0.28 mmol), (4-(piperidin-1-ylmethyl)phenyl)methanamine (57 mg, 0.28 mmol), palladium(II) acetate (3 mg), sodium tert-butoxide (40 mg, 0.42 mmol) and (+/−)-2,2′-Bis(diphenylphosphino)-1,1′-binaphthyl (17 mg, 0.028 mmol) in PhMe (6 mL) under nitrogen atmosphere was heated to 125° C. by microwave for 40 min. After cooling to room temperature, the reaction mixture was purified by reverse phase column to give JH093-16 (40 mg, 0.12 mmol, 43% yield) as white solid. ¹H NMR (800 MHz, DMSO-d₆) δ 8.34 (s, 1H), 8.06 (s, 1H), 7.18 (d, J=9.2 Hz, 1H), 6.72 (q, J=8.1 Hz, 4H), 6.61 (dd, J=9.3, 2.2 Hz, 1H), 6.07 (s, 1H), 3.87 (s, 2H), 3.47 (s, 2H), 2.62 (d, J=12.3 Hz, 2H), 2.13 (td, J=12.7, 3.1 Hz, 2H), 1.10 (dt, J=15.1, 3.6 Hz, 2H), 1.01 (dt, J=13.5, 3.8 Hz, 1H), 0.96-0.88 (m, 2H), 0.71-0.65 (m, 1H). MS (ESI) m/z 333.09 [M+H]⁺

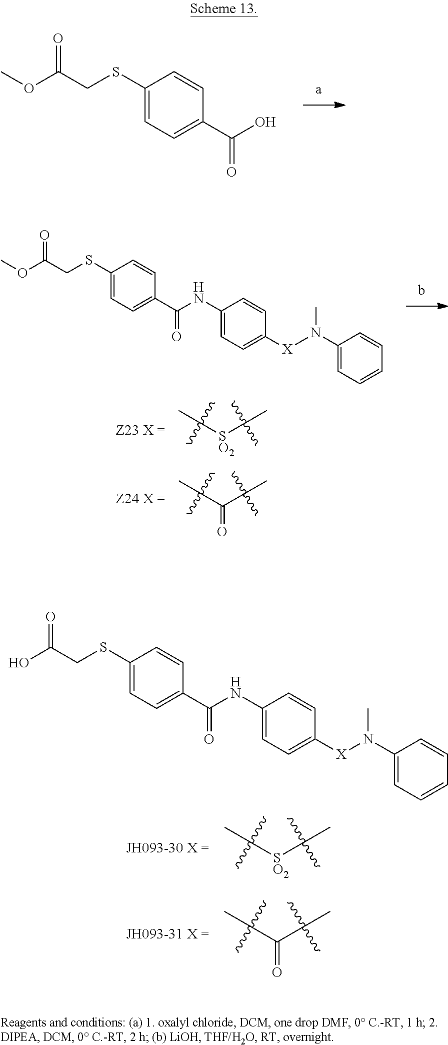

### Intermediate Z23

### methyl 2-((4-((4-(N-methyl-N-phenylsulfamoyl)phenyl)carbamoyl)phenyl)thio)acetate

To a solution of 4-((2-methoxy-2-oxoethyl)thio)benzoic acid (100 mg, 0.44 mmol) in DCM were added oxalyl chloride (57 μL, 0.66 mmol) and 1 drop of DMF. The mixture was stirred at room temperature for 2 h, then the solvent was removed by evaporation to the intermediate. The intermediate was dissolved in acetone, then addition of 4-amino-N-methyl-N-phenylbenzenesulfonamide (115 mg, 0.44 mmol) and DIPEA (115 uL, 0.66 mmol) in an ice bath. The mixture was stirred at RT for 1 h. Then quenched with N₄Cl, the mixture was extracted with ethyl acetate. The combined organic extracts were washed with brine; dried over sodium sulfate, concentrated. The resulting residue was purified by silica gel flash chromatography to give the compound Z23 (71 mg, 0.15 mmol, 34% yield) as yellow solid.

### Example 148: Synthesis of JH093-30 (JJ200)

### 2-((4-((4-(N-methyl-N-phenylsulfamoyl)phenyl)carbamoyl)phenyl)thio)acetic acid

To a solution of Z23 (50 mg, 0.11 mmol) in THF (2 mL) and H₂O (1 mL) was added LiOH (5 mg, 0.22 mmol). The reaction mixture was stirred at room temperature overnight. The reaction was monitored by UPLC. Upon completion, the reaction mixture was purified by reverse column to give JH093-30 (36 mg, 0.08 mmol, 73% yield) as yellow solid. ¹H NMR (800 MHz, DMSO-d₆) δ 10.58 (d, J=5.3 Hz, 1H), 7.97 (d, J=8.4 Hz, 2H), 7.91 (d, J=8.1 Hz, 2H), 7.49 (d, J=8.3 Hz, 1H), 7.45 (d, J=8.2 Hz, 2H), 7.35 (t, J=7.6 Hz, 2H), 7.29 (t, J=7.3 Hz, 1H), 7.12 (d, J=7.8 Hz, 2H), 3.95 (d, J=4.1 Hz, 2H), 3.14 (d, J=3.3 Hz, 3H). MS (ESI) m/z 457.07 [M+H]⁺

### Example 149: Synthesis of JH093-31 (JJ201)

Following the similar experimental procedure to obtain compound JH093-31.

### 2-((4-((4-(methyl(phenyl)carbamoyl)phenyl)carbamoyl)phenyl)thio)acetic acid

52%, white solid. ¹H NMR (800 MHz, DMSO-d₆) δ 10.24 (s, 1H), 7.84 (d, J=8.2 Hz, 2H), 7.60 (d, J=8.4 Hz, 2H), 7.42 (d, J=8.2 Hz, 2H), 7.28 (t, J=7.6 Hz, 2H), 7.23 (d, J=8.3 Hz, 1H), 7.16 (t, J=8.0 Hz, 3H), 3.93 (s, 2H). MS (ESI) m/z 421.39 [M+H]⁺

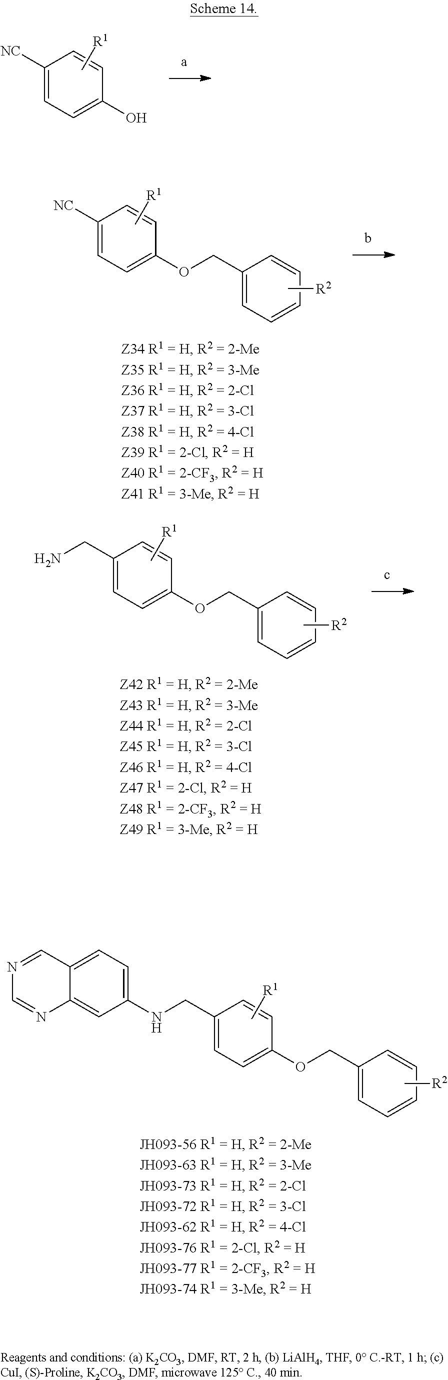

### Intermediate Z34

### 4-((2-methylbenzyl)oxy)benzonitrile

A solution of 4-cyanophenol (100 mg, 0.84 mmol) and 1-(bromomethyl)-2-methylbenzene (155 mg, 0.84 mmol) in 5 mL of DMF was treated with K₂CO₃ (139 mg, 1.0 mmol). The resulting mixture was stirred overnight at RT. After the reaction was completed, the reaction mixture was poured into ice water, aqueous phase was extracted with ethyl acetate. The combined organic phase was washed with brine twice, dried and concentrated. The resulting residue was purified by silica gel flash chromatography to give the compound Z34 (120 mg, 0.54 mmol, 64% yield) as yellow solid.

### Intermediate Z42

### (4-((2-methylbenzyl)oxy)phenyl)methanamine

Z34 (120 mg, 0.54 mmol) was added to a solution of LiAlH₄ (62 mg, 1.62 mmol) in anhydrous THF at 0° C., the resulting mixture was stirred at RT for 18 h. Then the mixture was cooled in ice bath and quenched with water, and 2N NaOH. The white suspension was diluted with water and extracted with ethyl acetate. The combined organic extracts were washed with brine, dried over sodium sulfate, and concentrated. The resulting residue was purified by reverse phase column to give the compound Z42 (77 mg, 0.34 mmol, 63% yield) as yellow solid.

### Example 150: Synthesis of JH093-56 (JJ217)

### N-(4-((2-methylbenzyl)oxy)benzyl)quinazolin-7-amine

A solution of 7-bromoquinazoline (40 mg, 0.2 mmol), Z42 (45 mg, 0.2 mmol), CuI (4 mg), K₂CO₃ (55 mg, 0.4 mmol) and (S)-(−)-Proline (5 mg, 0.04 mmol) in DMF (1 mL) under nitrogen atmosphere was heated to 125° C. by microwave for 40 min. The reaction mixture was purified by reverse phase column to give the compound JH093-56 (36 mg, 0.1 mmol, 50% yield) as yellow solid. ¹H NMR (800 MHz, DMSO-d₆) δ 9.29 (s, 1H), 9.00 (s, 1H), 8.58 (s, 1H), 7.95 (d, J=9.1 Hz, 1H), 7.40-7.36 (m, 2H), 7.36-7.33 (m, 2H), 7.22 (dt, J=14.4, 7.3 Hz, 2H), 7.18 (t, J=7.4 Hz, 1H), 7.03 (d, J=8.2 Hz, 2H), 6.81 (s, 1H), 5.06 (s, 2H), 4.48 (s, 2H), 2.30 (s, 3H). MS (ESI) m/z 356.19 [M+H]⁺

Followed the same experimental procedure to obtain compound JH093-51, 56, 62, 63, 72 and 73.

### Example 151: Synthesis of JH093-51 (JJ215)

### N-(4-(cyclohexylmethoxy)benzyl)quinazolin-7-amine

52%, white solid. ¹H NMR (800 MHz, DMSO-d₆) δ 9.15 (s, 1H), 8.92 (s, 1H), 8.26 (s, 1H), 7.88 (d, J=9.1 Hz, 1H), 7.30 (d, J=8.3 Hz, 3H), 6.91 (d, J=8.3 Hz, 2H), 6.74 (s, 1H), 4.43 (s, 2H), 3.74 (d, J=6.4 Hz, 2H), 1.78 (dd, J=12.8, 3.8 Hz, 2H), 1.69 (dq, J=12.4, 4.5, 3.6 Hz, 3H), 1.63 (dt, J=12.7, 3.8 Hz, 1H), 1.23 (qt, J=12.5, 3.5 Hz, 2H), 1.15 (qt, J=12.5, 3.4 Hz, 1H), 1.01 (qd, J=12.4, 3.6 Hz, 2H). MS (ESI) m/z 348.24 [M+H]⁺

### Example 152: Synthesis of JH093-62 (JJ219)

### N-(4-((4-chlorobenzyl)oxy)benzyl)quinazolin-7-amine

54%, white solid. ¹H NMR (800 MHz, DMSO-d₆) δ 9.15 (s, 1H), 8.38 (s, 1H), 7.92 (d, J=9.1 Hz, 1H), 7.44 (q, J=8.3 Hz, 3H), 7.33 (t, J=9.5 Hz, 3H), 7.25-7.16 (m, 1H), 7.00 (d, J=8.3 Hz, 2H), 6.76 (s, 1H), 5.08 (s, 2H), 4.45 (s, 2H). MS (ESI) m/z 376.18 [M+H]⁺

### Example 153: Synthesis of JH093-63 (JJ220)

### N-(4-((3-methylbenzyl)oxy)benzyl)quinazolin-7-amine

48%, white solid. ¹H NMR (800 MHz, DMSO-d₆) δ 9.09-8.93 (m, 1H), 8.27 (s, 1H), 7.89 (d, J=9.1 Hz, 1H), 7.31 (t, J=10.4 Hz, 3H), 7.25 (dd, J=15.2, 7.7 Hz, 2H), 7.21 (d, J=7.5 Hz, 1H), 7.13 (d, J=7.5 Hz, 1H), 7.00 (d, J=8.3 Hz, 2H), 6.75 (s, 1H), 5.03 (s, 2H), 4.44 (s, 2H), 2.30 (s, 3H). MS (ESI) m/z 356.29 [M+H]⁺

### Example 154: Synthesis of JH093-72 (JJ221)

### N-(4-((3-chlorobenzyl)oxy)benzyl)quinazolin-7-amine

47%, white solid. ¹H NMR (800 MHz, DMSO-d₆) δ 9.30 (d, J=48.1 Hz, 1H), 8.99 (s, 1H), 8.43 (s, 1H), 7.92 (d, J=9.1 Hz, 1H), 7.49 (s, 1H), 7.43-7.36 (m, 2H), 7.33 (d, J=8.4 Hz, 3H), 7.01 (d, J=8.4 Hz, 2H), 6.78 (s, 1H), 5.11 (s, 2H), 4.46 (s, 2H). MS (ESI) m/z 376.18 [M+H]⁺

### Example 155: Synthesis of JH093-73 (JJ222)

### N-(4-((2-chlorobenzyl)oxy)benzyl)quinazolin-7-amine

42%, white solid. ¹H NMR (800 MHz, DMSO-d₆) δ 9.27 (dt, J=38.5, 13.9 Hz, 1H), 8.98 (s, 1H), 8.59 (d, J=14.8 Hz, 1H), 7.95 (d, J=9.2 Hz, 1H), 7.57 (d, J=7.0 Hz, 1H), 7.50 (t, J=6.9 Hz, 1H), 7.43-7.30 (m, 5H), 7.02 (dd, J=14.8, 8.4 Hz, 2H), 6.81 (d, J=7.3 Hz, 1H), 5.14 (s, 2H), 4.49 (s, 2H). MS (ESI) m/z 376.38 [M+H]⁺

### Example 156: Synthesis of JH093-74 (JJ223)

### N-(4-(benzyloxy)-2-methylbenzyl)quinazolin-7-amine

40%, white solid. ¹H NMR (800 MHz, DMSO-d₆) δ 9.06 (s, 1H), 8.51 (s, 1H), 7.95 (d, J=9.1 Hz, 1H), 7.47 (d, J=7.5 Hz, 2H), 7.37 (d, J=9.0 Hz, 1H), 7.32 (t, J=7.5 Hz, 2H), 7.27 (t, J=7.3 Hz, 1H), 7.16 (d, J=7.6 Hz, 1H), 6.99 (s, 1H), 6.77 (s, 1H), 6.75 (d, J=7.7 Hz, 1H), 5.18 (s, 2H), 4.48 (s, 2H), 2.28 (s, 3H). ESI [M+H]⁺: 356.24.

### Example 157: Synthesis of JH093-76 (JJ224)

### N-(4-(benzyloxy)-3-chlorobenzyl)quinazolin-7-amine

46%, white solid. ¹H NMR (800 MHz, DMSO-d₆) δ 9.19-8.75 (m, 1H), 8.01 (s, 1H), 7.85 (d, J=9.0 Hz, 1H), 7.48 (s, 1H), 7.45 (d, J=7.7 Hz, 2H), 7.39 (t, J=7.5 Hz, 2H), 7.33 (t, J=7.8 Hz, 2H), 7.26 (d, J=9.0 Hz, 1H), 7.22 (d, J=8.5 Hz, 1H), 6.71 (s, 1H), 5.19 (s, 2H), 4.43 (d, J=4.7 Hz, 2H). ESI [M+H]⁺: 376.1.

### Example 158: Synthesis of JH093-77 (JJ225)

### N-(4-(benzyloxy)-3-(trifluoromethyl)benzyl)quinazolin-7-amine

48%, white solid. ¹H NMR (800 MHz, DMSO-d₆) δ 8.97 (s, 1H), 8.28 (s, 1H), 7.91 (d, J=9.1 Hz, 1H), 7.70 (s, 1H), 7.65 (d, J=8.6 Hz, 1H), 7.43 (d, J=7.7 Hz, 2H), 7.39 (t, J=7.5 Hz, 2H), 7.34 (d, J=8.9 Hz, 1H), 7.33-7.28 (m, 2H), 6.77 (s, 1H), 5.26 (s, 2H), 4.52 (s, 2H). ESI [M+H]⁺: 410.22.

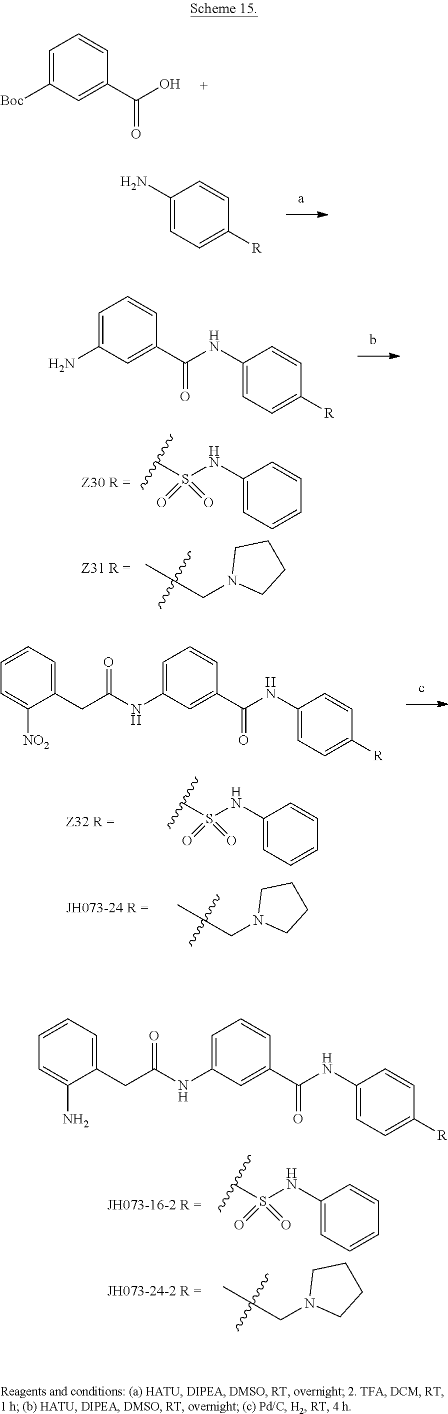

### Intermediate Z30

### 3-amino-N-(4-(N-phenylsulfamoyl)phenyl)benzamide

To a solution of 3-((tert-butoxycarbonyl)amino)benzoic acid (50 mg, 0.21 mmol) and 4-amino-N-phenylbenzenesulfonamide (52 mg, 0.21 mmol) in DMSO (2 mL) were added HATU (120 mg, 0.32 mmol) and DIPEA (110 μL, 0.63 mmol). The reaction mixture was stirred at room temperature overnight. The reaction was monitored by UPLC. Upon completion, the reaction mixture was purified by reserve column to give intermediate. The intermediate was dissolved in DCM (0.5 mL), then addition of TFA (0.5 mL). The mixture was stirred at room temperature for 1 h. The reaction was monitored by UPLC. Upon completion, the reaction mixture was purified by preparative HPLC to give Z30 (40 mg, 0.11 mmol, 58% yield) as yellow solid.

### Intermediate Z32

### 3-(2-(2-nitrophenyl)acetamido)-N-(4-(N-phenylsulfamoyl)phenyl)benzamide

To a solution of Z30 (40 mg, 0.11 mmol) and 2-(2-nitrophenyl)acetic acid (20 mg, 0.11 mmol) in DMSO (2 mL) were added HATU (63 mg, 0.17 mmol) and DIPEA (58 μL, 0.33 mmol). The reaction mixture was stirred at room temperature overnight. The reaction was monitored by UPLC. Upon completion, the reaction mixture was purified by preparative HPLC to give Z32 (32 mg, 0.06 mmol, 55% yield) as yellow solid.

Following the similar experimental procedures of compound Z32 to obtain compound JH073-24.

### Example 159: Synthesis of JH073-24 (JJ162)

### 3-(2-(2-nitrophenyl)acetamido)-N-(4-(pyrrolidin-1-ylmethyl)phenyl)benzamide

50% yield, white solid. ¹H NMR (800 MHz, DMSO-d₆) δ 10.55 (s, 1H), 10.44 (s, 1H), 10.24 (dt, J=13.4, 6.3 Hz, 1H), 8.14 (s, 1H), 8.07 (d, J=8.1 Hz, 1H), 7.84 (d, J=8.2 Hz, 2H), 7.78 (d, J=8.1 Hz, 1H), 7.72 (t, J=7.5 Hz, 1H), 7.64 (d, J=7.7 Hz, 1H), 7.61-7.55 (m, 2H), 7.47 (dd, J=17.8, 8.2 Hz, 2H), 4.31 (d, J=4.5 Hz, 2H), 4.18 (s, 2H), 3.37 (dt, J=11.7, 5.4 Hz, 2H), 3.08 (dq, J=14.3, 7.4 Hz, 2H), 2.02 (t, J=7.5 Hz, 2H), 1.85 (q, J=6.8, 6.3 Hz, 2H). ESI [M+H]⁺: 459.32.

### Example 160

### 3-(2-(2-aminophenyl)acetamido)-N-(4-(N-phenylsulfamoyl)phenyl)benzamide

To a solution of Z32 (30 mg, 0.06 mmol) in MeOH (2 mL) was added Pd/C (6 mg). The reaction mixture was filled with hydrogen and stirred at room temperature for 4 h. The reaction was monitored by UPLC. Upon completion, the reaction mixture was filtered to give JH073-16-2 (20 mg, 0.04 mmol, 67% yield) as white solid. ¹H NMR (800 MHz, DMSO-d₆) δ 10.59 (s, 1H), 10.37 (s, 1H), 10.20 (s, 1H), 8.08 (s, 1H), 7.90 (d, J=8.5 Hz, 2H), 7.84 (d, J=8.2 Hz, 1H), 7.73 (d, J=8.5 Hz, 2H), 7.60 (d, J=7.7 Hz, 1H), 7.46 (t, J=7.9 Hz, 1H), 7.23 (t, J=7.7 Hz, 2H), 7.09 (d, J=8.1 Hz, 2H), 7.07 (d, J=7.5 Hz, 1H), 7.01 (t, J=7.4 Hz, 1H), 6.96 (t, J=7.6 Hz, 1H), 6.67 (d, J=7.9 Hz, 1H), 6.55 (t, J=7.3 Hz, 1H), 5.10 (s, 2H), 3.52 (s, 2H). ESI [M+H]=: 501.46.

Following the similar experimental procedure of compound JH073-16-2 to obtain compound JH073-24-2 from compound Z32.

### Example 161

### 3-(2-(2-aminophenyl)acetamido)-N-(4-(pyrrolidin-1-ylmethyl)phenyl)benzamide

60% yield, white solid. ¹H NMR (800 MHz, DMSO-d₆) δ 10.42 (s, 1H), 10.02 (s, 1H), 8.15 (s, 1H), 7.83 (dd, J=21.6, 8.6 Hz, 2H), 7.63 (d, J=7.8 Hz, 1H), 7.55-7.39 (m, 2H), 7.10 (d, J=7.8 Hz, 1H), 6.98 (t, J=7.6 Hz, 1H), 6.71 (d, J=7.9 Hz, 1H), 6.58 (dt, J=14.5, 7.5 Hz, 1H), 4.31 (d, J=4.1 Hz, 2H), 3.55 (s, 2H), 3.09 (dq, J=14.1, 7.3, 6.7 Hz, 2H), 2.02 (q, J=7.5 Hz, 2H), 1.85 (p, J=7.4, 6.6 Hz, 2H). ESI [M+H]⁺: 459.32

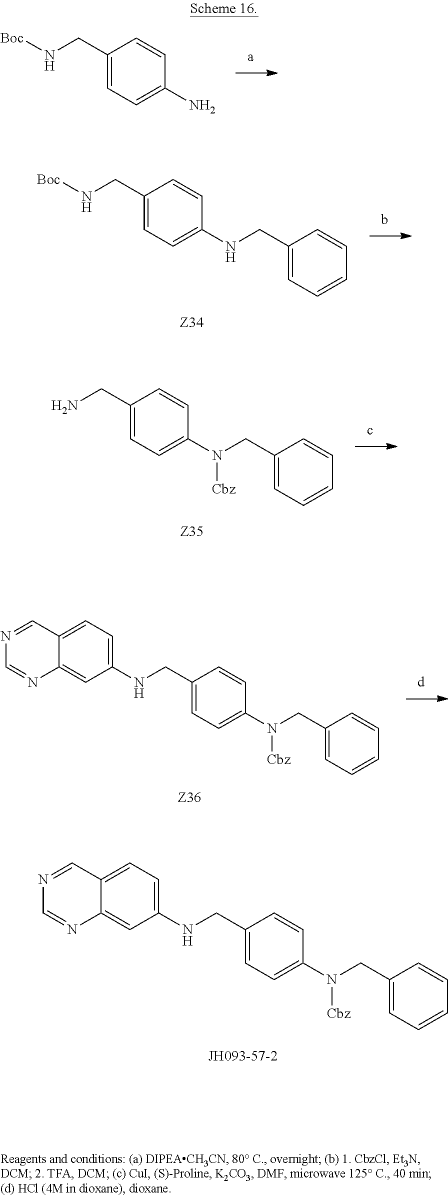 [SMILES: CC(C)(C)OC(=O)NCc1ccc(N)cc1 ; CC(C)(C)OC(=O)NCc1ccc(NCc2ccccc2)cc1 ; NCc1ccc(N(Cc2ccccc2)C(=O)OCc2ccccc2)cc1 ; O=C(OCc1ccccc1)N(Cc1ccccc1)c1ccc(CNc2ccc3cncnc3c2)cc1 ; O=C(OCc1ccccc1)N(Cc1ccccc1)c1ccc(CNc2ccc3cncnc3c2)cc1]

Compound Z35 was made by similar procedures as compound Z27 of scheme 4. Compound Z36 was made by similar procedure as compound JH093-56 of scheme 14.

### Example 162: Synthesis of JH093-57-2

### N-(4-(benzylamino)benzyl)quinazolin-7-amine

To the solution of compound Z36 (50 mg, 0.1 mmol) in dioxane were treated with HCl (4M in dioxane, 0.5 mL). The resulting mixture was heated to 55° C. for 4 h. After cooling to room temperature, the mixture was purified by reverse phase column to give JH093-57-2 (20 mg, 0.06 mmol, 60% yield) as yellow solid.

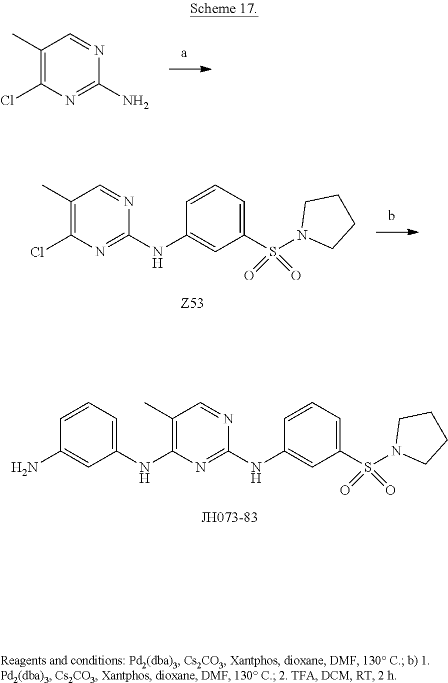

### Intermediate Z53

### 4-chloro-5-methyl-N-(3-(pyrrolidin-1-ylsulfonyl)phenyl)pyrimidin-2-amine

A solution of 4-chloro-5-methylpyrimidin-2-amine (27 mg, 0.2 mmol), 1-((3-Bromophenyl)sulfonyl)pyrrolidine (58 mg, 0.2 mmol), Pd₂(dba)₃ (9 mg, 0.01 mmol), cesium carbonate (130 mg, 0.4 mmol), xantphos (12 mg, 0.02 mmol) in dioxane was heated to 130° C. under microwave for 0.5 h. After cooling to room temperature, the mixture was poured in water and extracted with EtOAc. The combined organic layer was washed with brine and concentrated. The resulting residue was purified by silica gel flash chromatography to give the compound Z37 as white solid (20 mg, 0.06 mmol, 30% yield).

### Example 163: Synthesis of JH073-83 (JJ166)

### N⁴-(3-aminophenyl)-5-methyl-N²-(3-(pyrrolidin-1-ylsulfonyl)phenyl)pyrimidine-2,4-diamine

A solution of Z37 (71 mg, 0.2 mmol), tert-butyl (3-aminophenyl)carbamate (42 mg, 0.2 mmol), Pd₂(dba)₃ (9 mg, 0.01 mmol), cesium carbonate (130 mg, 0.4 mmol), xantphos (12 mg, 0.02 mmol) in dioxane was heated to 130° C. under microwave for 0.5 h. After cooling to room temperature, the mixture was poured in water and extracted with EtOAc. The combined organic layer was washed with brine and concentrated. The resulting residue was purified by silica gel flash chromatography to give the 30 mg intermediate. Then the intermediate was dissolved in DCM (1 mL) and addition of TFA (1 mL), the mixture was stirred at room temperature for 2 h. The reaction was monitored by UPLC. Upon completion, the reaction mixture was evaporated and purified by preparative HPLC to give JH073-83 (20 mg, 0.05 mmol, 25% yield) as white solid. ¹H NMR (800 MHz, DMSO-d₆) δ 10.20 (s, 1H), 9.34 (s, 1H), 8.09 (d, J=8.2 Hz, 1H), 7.93 (s, 1H), 7.76 (s, 1H), 7.44 (t, J=7.9 Hz, 1H), 7.41 (d, J=7.9 Hz, 1H), 7.16 (t, J=7.9 Hz, 1H), 7.00 (s, 1H), 6.94 (s, 1H), 6.64 (d, J=8.1 Hz, 1H), 3.18-3.03 (m, 4H), 2.15 (s, 3H), 1.63 (d, J=6.8 Hz, 4H). ESI [M+H]⁺: 425.4.

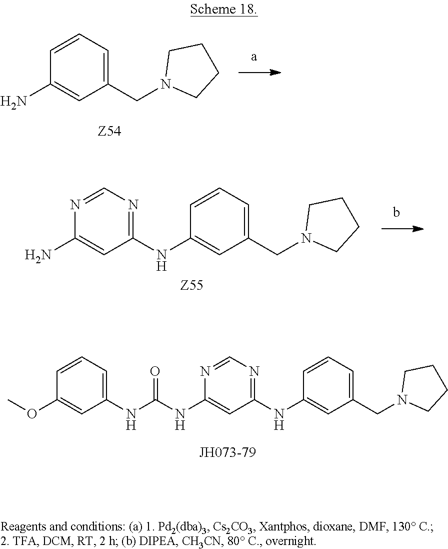 [SMILES: Nc1cccc(CN2CCCC2)c1 ; Nc1cc(Nc2cccc(CN3CCCC3)c2)ncn1 ; COc1cccc(NC(=O)Nc2cc(Nc3cccc(CN4CCCC4)c3)ncn2)c1]

Following the similar experimental procedure of intermediate Z1 in scheme 3 to obtain intermediate Z54. Following the similar experimental procedure of compound JH073-83 in scheme 17 to obtain compound Z55.

### Example 164: JH073-69 (JJ165)

### 1-(3-methoxyphenyl)-3-(6-((3-(pyrrolidin-1-ylmethyl)phenyl)amino)pyrimidin-4-yl)urea

To a solution of Z55 (50 mg, 0.2 mmol) and DIPEA (105 μL, 0.6 mmol) in CH₃CN (5 mL) was added 1-isocyanato-3-methoxybenzene (30 mg, 0.2 mmol). The reaction mixture was heated to 80° C. overnight. The reaction was monitored by UPLC. Upon completion, the reaction mixture was purified by reverse column to give JH073-79 (119 mg, 0.1 mmol, 50% yield) as yellow solid. MS (ESI) [M+H]⁺ 419.48.

### Example 165

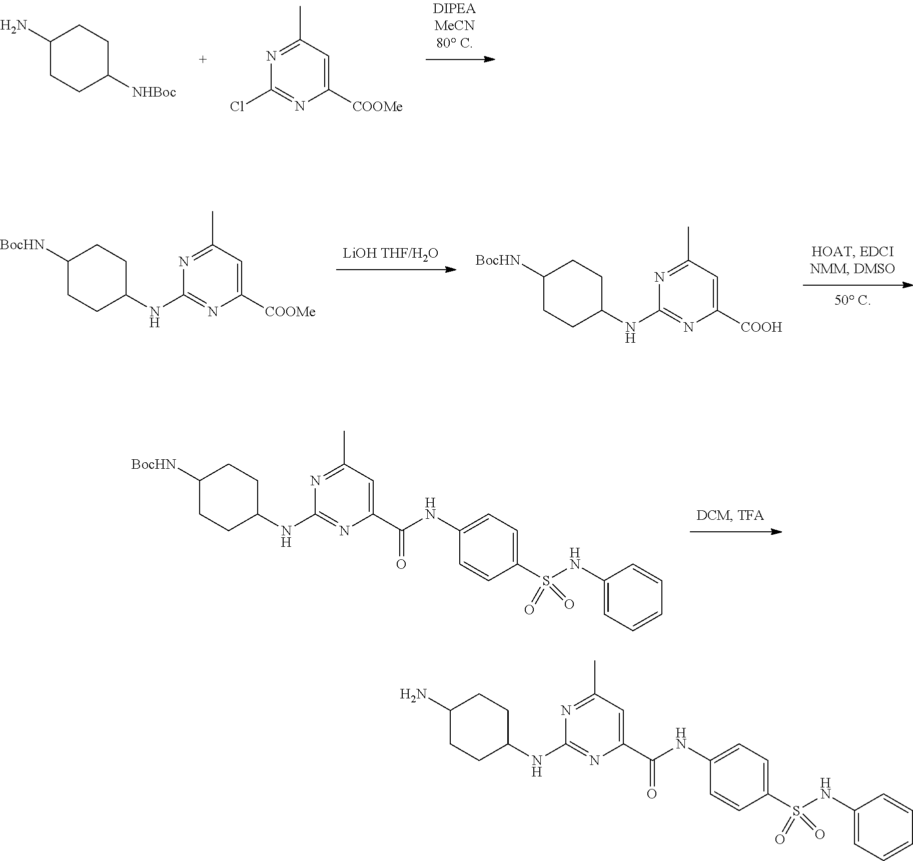 [SMILES: CC(C)(C)OC(=O)NC1CCC(N)CC1 ; COC(=O)c1cc(C)nc(Cl)n1 ; COC(=O)c1cc(C)nc(NC2CCC(NC(=O)OC(C)(C)C)CC2)n1 ; Cc1cc(C(=O)O)nc(NC2CCC(NC(=O)OC(C)(C)C)CC2)n1 ; Cc1cc(C(=O)Nc2ccc(S(=O)(=O)Nc3ccccc3)cc2)nc(NC2CCC(NC(=O)OC(C)(C)C)CC2)n1 ; Cc1cc(C(=O)Nc2ccc(S(=O)(=O)Nc3ccccc3)cc2)nc(NC2CCC(N)CC2)n1]

A mixture of tert-butyl (4-aminocyclohexyl)carbamate (0.2 mmol), methyl 2-chloro-6-methylpyrimidine-4-carboxylate (0.2 mmol) and DIPEA (0.4 mmol) in MeCN was stirred at 80° C. for 10 h. The reaction was monitored by LC-MS and upon completion; the solvent was evaporated under reduced pressure to obtain methyl 2-chloro-6-methylpyrimidine-4-carboxylate. This intermediate was used for the next step without further purification.

A solution of 2-chloro-6-methylpyrimidine-4-carboxylate and LiOH (1 mmol) in 3 mL THF and H₂O was stirred at room temperature overnight. The reaction was monitored by LC-MS and upon completion; the reaction mixture was purified by reverse column chromatography to give 2-((4-((tert-butoxycarbonyl)amino)cyclohexyl)amino)-6-methylpyrimidine-4-carboxylic acid. Yield is 90% for two steps.

A mixture of 2-((4-((tert-butoxycarbonyl)amino)cyclohexyl)amino)-6-methylpyrimidine-4-carboxylic acid, 4-amino-N-phenylbenzenesulfonamide (0.2 mmol), HOAT (0.4 mmol), EDCI (0.4 mmol) and NMM (0.6 mmol) in 2 mL DMSO was stirred at room temperature overnight. The mixture was purified by pre-HPLC to get tert-butyl (4-((4-methyl-6-((4-(N-phenylsulfamoyl)phenyl)carbamoyl)pyrimidin-2-yl)amino)cyclohexyl)carbamate. Then the ester compound was stirred in TFA/DCM (1 mL/2 mL) for 2 h. After purified by pre-HPLC, FM-74052 (JJ132) was obtained with a yield of 45%.

¹H NMR (800 MHz, Methanol-d4) δ 7.88 (d, J=8.4 Hz, 2H), 7.77 (d, J=8.6 Hz, 2H), 7.24 (t, J=7.7 Hz, 2H), 7.19 (s, 1H), 7.12 (d, J=8.0 Hz, 2H), 7.08 (t, J=7.4 Hz, 1H), 4.08-3.92 (m, 1H), 3.20-3.14 (m, 1H), 2.45 (s, 3H), 2.25-2.21 (m, 2H), 2.16-2.12 (m, 2H), 1.65-1.58 (m, 2H), 1.50-1.43 (m, 2H). MS (ESI) m/z 481.3 [M+H]⁺.

### Example 166

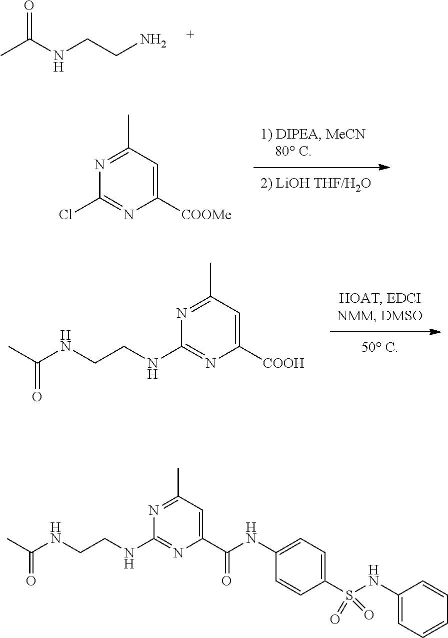

A mixture of N-(2-aminoethyl)acetamide (0.2 mmol), methyl 2-chloro-6-methylpyrimidine-4-carboxylate (0.2 mmol) and DIPEA (0.4 mmol) in MeCN was stirred at 80° C. for 10 h. The reaction was monitored by LC-MS and upon completion; the solvent was evaporated under reduced pressure to obtain methyl 2-((2-acetamidoethyl)amino)-6-methylpyrimidine-4-carboxylate. This intermediate was used for the next step without further purification.

A solution of previous compound and LiOH (1 mmol) in 3 mL THF and H₂O was stirred at room temperature overnight. The reaction was monitored by LC-MS and upon completion; the reaction mixture was purified by reverse column chromatography to give 2-((2-acetamidoethyl)amino)-6-methylpyrimidine-4-carboxylic acid. Yield is 83% for two steps.

A mixture of 2-((2-acetamidoethyl)amino)-6-methylpyrimidine-4-carboxylic acid, 4-amino-N-phenylbenzenesulfonamide (0.2 mmol), HOAT (0.4 mmol), EDCI (0.4 mmol) and NMM (0.6 mmol) in 2 mL DMSO was stirred at room temperature overnight. The mixture was purified by pre-HPLC to get FM-74056 (JJ133) with a yield of 40%.

¹H NMR (800 MHz, Methanol-d4) δ 8.09-8.01 (m, 2H), 7.77 (d, J=8.6 Hz, 2H), 7.27-7.21 (m, 3H), 7.13 (d, J=8.0 Hz, 2H), 7.08 (t, J=7.4 Hz, 1H), 4.67-4.58 (m, 2H), 3.68-3.61 (m, 2H), 2.44 (s, 3H), 1.91 (s, 3H). MS (ESI) m/z 469.3 [M+H]⁺.

### Example 167

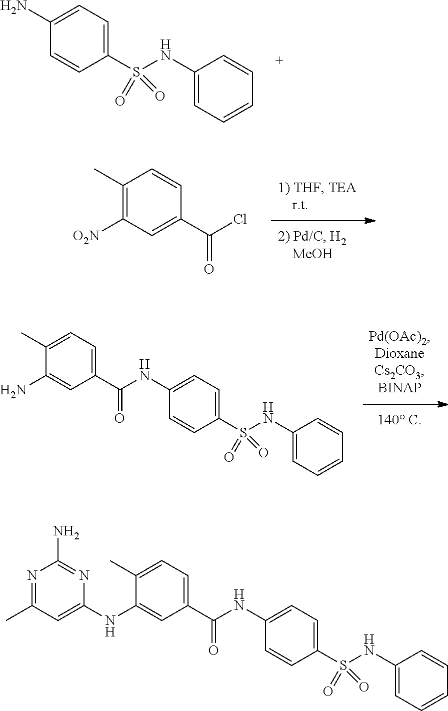 [SMILES: Nc1ccc(S(=O)(=O)Nc2ccccc2)cc1 ; Cc1ccc(C(=O)Cl)cc1[N+](=O)[O-] ; Cc1ccc(C(=O)Nc2ccc(S(=O)(=O)Nc3ccccc3)cc2)cc1N ; Cc1cc(Nc2cc(C(=O)Nc3ccc(S(=O)(=O)Nc4ccccc4)cc3)ccc2C)nc(N)n1]

A mixture of 4-amino-N-phenylbenzenesulfonamide (0.2 mmol), 4-methyl-3-nitrobenzoyl chloride (0.2 mmol) and TEA (0.2 mmol) in THF was stirred at room temperature for 3 h. The reaction was monitored by LC-MS and upon completion; the solvent was evaporated under reduced pressure to obtain 4-methyl-3-nitro-N-(4-(N-phenylsulfamoyl)phenyl)benzamide. This intermediate was used for the next step without further purification.

To a solution of 4-methyl-3-nitro-N-(4-(N-phenylsulfamoyl)phenyl)benzamide in 10 mL MeOH was added Pd/C (10 wt %). Exchanged with H₂ for three times before stirred at room temperature overnight. The reaction was monitored by LC-MS and upon completion; the reaction mixture was purified by reverse column chromatography to give 3-amino-4-methyl-N-(4-(N-phenylsulfamoyl)phenyl)benzamide with 75% yield for two steps.

A mixture of 3-amino-4-methyl-N-(4-(N-phenylsulfamoyl)phenyl)benzamide, 4-chloro-6-methylpyrimidin-2-amine (0.2 mmol), Pd(OAc)₂ (0.02 mmol), BINAP (0.02 mmol) and Cs₂CO₃ (0.4 mmol) in 2 mL Dioxane was stirred at 140° C. overnight. The mixture was purified by pre-HPLC to get FM-74059 (JJ134) with 77% yield.

¹H NMR (400 MHz, Methanol-d4) δ 7.88-7.80 (m, 3H), 7.77-7.69 (m, 3H), 7.26-7.18 (m, 3H), 7.13-7.03 (m, 4H), 2.37-2.33 (m, 6H). MS (ESI) m/z 489.2 [M+H]⁺.

### Example 168: Synthesis of FM-74028 (JJ135)

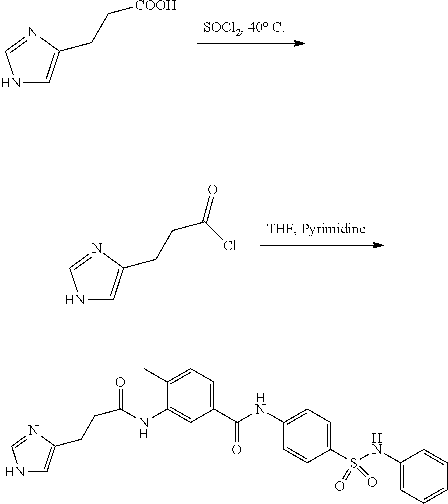 [SMILES: O=C(O)CCc1c[nH]cn1 ; O=C(Cl)CCc1c[nH]cn1 ; Cc1ccc(C(=O)Nc2ccc(S(=O)(=O)Nc3ccccc3)cc2)cc1NC(=O)CCc1c[nH]cn1]

A mixture of 3-(1H-imidazol-4-yl)propanoic acid (0.2 mmol) in 1 mL SOCl₂ was stirred at 40° C. for 3 h. The reaction was monitored by LC-MS and upon completion; the solvent was evaporated under reduced pressure to obtain 3-(1H-imidazol-4-yl)propanoyl chloride.

To a solution of 3-(1H-imidazol-4-yl)propanoyl chloride in 3 mL Acetone was added 3-amino-4-methyl-N-(4-(N-phenylsulfamoyl)phenyl)benzamide (0.2 mmol) and pyrimidine (0.2 mmol). The reaction was monitored by LC-MS and upon completion; the reaction mixture was purified by reverse column chromatography to give FM-74028 (JJ135) with 75% yield for two steps.

¹H NMR (800 MHz, Acetone-d6) δ 9.96 (s, 1H), 9.35 (s, 1H), 8.97 (s, 1H), 8.81 (s, 1H), 8.24 (s, 1H), 8.23 (s, 1H), 8.00 (d, J=8.5 Hz, 2H), 7.78 (d, J=8.6 Hz, 2H), 7.69 (d, J=8.0 Hz, 1H), 7.46 (s, 1H), 7.33 (d, J=7.9 Hz, 1H), 7.30-7.23 (m, 4H), 7.08 (t, J=7.0 Hz, 1H), 3.20 (t, J=7.5 Hz, 2H), 2.98 (t, J=7.3 Hz, 2H), 2.30 (s, 3H). MS (ESI) m/z 504.2 [M+H]⁺.

### Example 169: Synthesis of FM-71146 (JJ136)

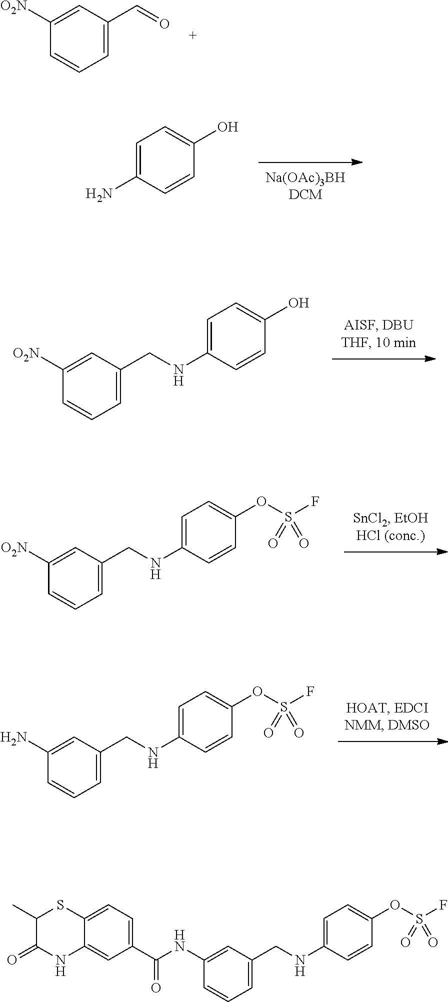 [SMILES: O=Cc1cccc([N+](=O)[O-])c1 ; Nc1ccc(O)cc1 ; O=[N+]([O-])c1cccc(CNc2ccc(O)cc2)c1 ; O=[N+]([O-])c1cccc(CNc2ccc(OS(=O)(=O)F)cc2)c1 ; Nc1cccc(CNc2ccc(OS(=O)(=O)F)cc2)c1 ; CC1Sc2ccc(C(=O)Nc3cccc(CNc4ccc(OS(=O)(=O)F)cc4)c3)cc2NC1=O]

To a solution of 3-nitrobenzaldehyde (0.2 mmol) and 4-aminophenolin (0.2 mmol) in 3 mL DCM was added Na(OAc)₃BH (0.4 mmol). The solution was stirred at room temperature for 3 h. The mixture was purified by chromatography to give 4-((3-nitrobenzyl)amino)phenol with 85% yield.

To a solution of 4-((3-nitrobenzyl)amino)phenol (0.1 mmol) in THF was added AISF (0.1 mmol) and DBU (0.15 mmol). The solution was stirred at room temperature for 10 min. The mixture was purified by chromatography to give 4-((3-nitrobenzyl)amino)phenyl sulfurofluoridate with 61% yield.

To a solution of 4-((3-nitrobenzyl)amino)phenyl sulfurofluoridate (0.1 mmol) and SnCl₂ (0.25 mmol) in 5 mL EtOH was added 1 drop of concentrated HCl. The solution was stirred at 80° C. for 3 h. The mixture was purified by reverse column chromatography to give 4-((3-aminobenzyl)amino)phenyl sulfurofluoridate with 90% yield.

A mixture of 2-methyl-3-oxo-3,4-dihydro-2H-benzo[b][1,4]thiazine-6-carboxylic acid (0.2 mmol), 4-((3-aminobenzyl)amino)phenyl sulfurofluoridate (0.2 mmol), HOAT (0.4 mmol), EDCI (0.4 mmol) and NMM (0.6 mmol) in 2 mL DMSO was stirred at room temperature overnight. Upon completion, water was added to the mixture and the water phase was extracted with EA for 3 times. The organic phase was dried with Na₂SO₄. The mixture was purified by chromatography to get FM-71146 (JJ136) with a yield of 40%.

¹H NMR (800 MHz, Acetone-d6) δ 9.72 (s, 1H), 9.59 (s, 1H), 7.88 (s, 1H), 7.76 (s, 1H), 7.75 (s, 1H), 7.71-7.61 (m, 2H), 7.50-7.44 (m, 1H), 7.34 (t, J=7.8 Hz, 1H), 7.24 (d, J=8.8 Hz, 2H), 7.18 (d, J=7.6 Hz, 1H), 6.78 (d, J=9.0 Hz, 2H), 4.42 (s, 2H), 3.74-3.65 (m, 1H), 1.49-1.43 (m, 3H). MS (ESI) m/z 502.1 [M+H]⁺.

### Example 170: Synthesis of FM-74015 (JJ137)

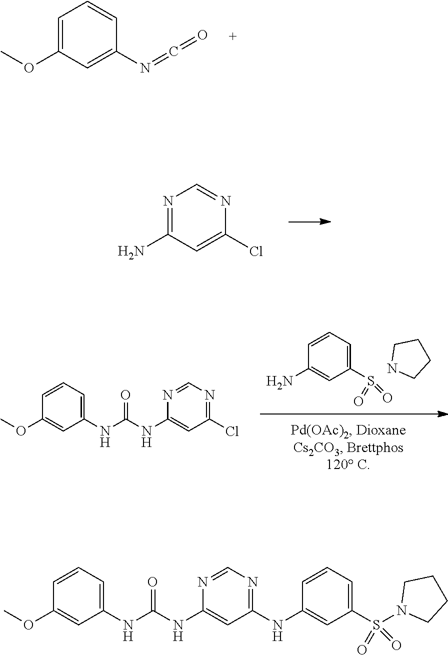

To a solution of 6-chloropyrimidin-4-amine (1.0 mmol) in 4 mL DCM was added 1-isocyanato-3-methoxybenzene (1.0 mmol) dropwise. The mixture was stirred at room temperature for 24 h and then was purified by reverse column chromatography to get 1-(6-chloropyrimidin-4-yl)-3-(3-methoxyphenyl)urea with a yield of 15%.

A mixture of 1-(6-chloropyrimidin-4-yl)-3-(3-methoxyphenyl)urea (0.2 mmol), 3-(pyrrolidin-1-ylsulfonyl)aniline (0.2 mmol), Pd(OAc)₂ (0.02 mmol), Brettphos (0.02 mmol) and Cs₂CO₃ (0.4 mmol) in 2 mL Dioxane was stirred at 120° C. overnight. The mixture was purified by pre-HPLC to get FM-74015 (JJ137) with 56% yield.

¹H NMR (800 MHz, Acetone-d6) δ 10.89-10.53 (m, 1H), 10.02-9.54 (m, 1H), 8.67 (s, 1H), 8.30 (s, 1H), 8.29 (s, 1H), 8.03-7.97 (m, 1H), 7.64 (t, J=7.9 Hz, 1H), 7.60-7.56 (m, 1H), 7.39 (s, 1H), 7.24 (t, J=8.1 Hz, 1H), 7.11 (d, J=8.0 Hz, 1H), 6.96-6.86 (m, 1H), 6.67 (d, J=8.2 Hz, 1H), 3.81 (s, 3H), 3.31-3.26 (m, 4H), 1.81-1.75 (m, 4H). MS (ESI) m/z 469.3 [M+H]+.

### Example 171: Synthesis of FM-71140 (JJ138)

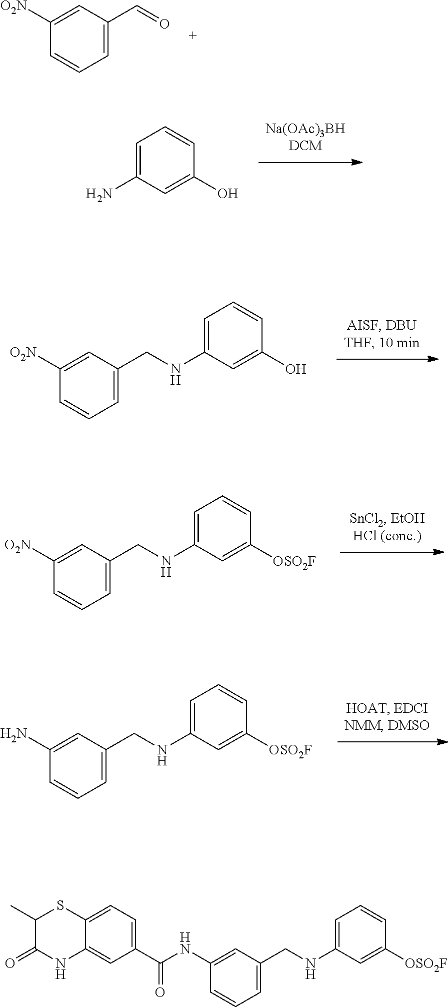

FM-71140 (JJ138) was prepared as the procedure of JJ136. ¹H NMR (800 MHz, Acetone-d6) δ 9.72 (s, 1H), 9.59 (s, 1H), 7.89 (s, 1H), 7.76 (d, J=7.9 Hz, 1H), 7.70 (s, 1H), 7.64 (d, J=8.1 Hz, 1H), 7.46 (d, J=8.0 Hz, 1H), 7.34 (t, J=7.8 Hz, 1H), 7.28 (t, J=8.2 Hz, 1H), 7.19 (d, J=7.6 Hz, 1H), 6.83-6.78 (m, 1H), 6.76 (s, 1H), 6.70-6.65 (m, 1H), 6.23-6.20 (m, 1H), 4.44 (s, 2H), 3.70 (q, J=7.0 Hz, 1H), 1.45 (d, J=7.0 Hz, 3H). MS (ESI) m/z 502.2 [M+H]⁺.

### Example 172: Synthesis of FM-71170 (JJ139)

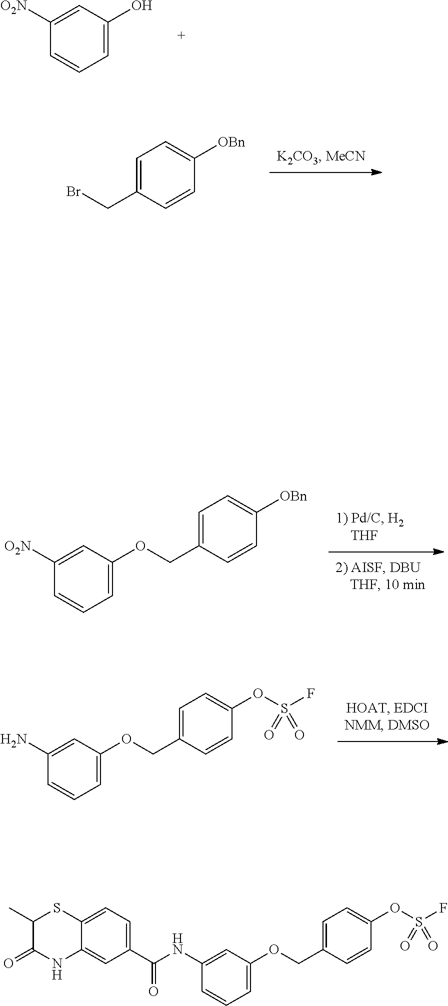 [SMILES: O=[N+]([O-])c1cccc(O)c1 ; BrCc1ccc(OCc2ccccc2)cc1 ; O=[N+]([O-])c1cccc(OCc2ccc(OCc3ccccc3)cc2)c1 ; Nc1cccc(OCc2ccc(OS(=O)(=O)F)cc2)c1 ; CC1Sc2ccc(C(=O)Nc3cccc(OCc4ccc(OS(=O)(=O)F)cc4)c3)cc2NC1=O]

A mixture of 3-nitrophenol (1.0 mmol), 1-(benzyloxy)-4-(bromomethyl)benzene (1.0 mmol) and K₂CO₃ (2.0 mmol) in 4 mL MeCN was stirred at room temperature for 12 h. Upon completion, the mixture was purified by chromatography to get 1-((4-(benzyloxy)benzyl)oxy)-3-nitrobenzene with 59% yield.

To a solution of 1-((4-(benzyloxy)benzyl)oxy)-3-nitrobenzene (0.2 mmol) in 5 mL THF was added Pd/C (10 wt %). Exchanged with H₂ for three times before stirred at room temperature overnight. The reaction was monitored by LC-MS and upon completion; the solid was filtered and the solvent was removed under reduced pressure to give crude product which can be used without further purification. This crude product was dissolved in THF. AISF (0.2 mmol) and DBU (0.3 mmol) was added to this solution. The solution was stirred at room temperature for 10 min. The mixture was purified by chromatography to give 4-((3-aminophenoxy)methyl)phenyl sulfurofluoridate with 57% yield.

A mixture of 2-methyl-3-oxo-3,4-dihydro-2H-benzo[b][1,4]thiazine-6-carboxylic acid (0.2 mmol), 4-((3-aminophenoxy)methyl)phenyl sulfurofluoridate (0.2 mmol), HOAT (0.4 mmol), EDCI (0.4 mmol) and NMM (0.6 mmol) in 2 mL DMSO was stirred at room temperature overnight. Upon completion, water was added to the mixture and the water phase was extracted with EA for 3 times. The organic phase was dried with Na₂SO₄. The mixture was purified by chromatography to get FM-71170 (JJ139) with 55% yield.

¹H NMR (800 MHz, Acetone-d6) δ 9.74 (s, 1H), 9.58 (s, 1H), 7.79-7.73 (m, 2H), 7.70 (s, 1H), 7.69-7.67 (m, 1H), 7.63 (d, J=8.1 Hz, 1H), 7.61 (d, J=8.3 Hz, 1H), 7.55-7.51 (m, 1H), 7.50-7.45 (m, 1H), 7.40 (d, J=8.1 Hz, 1H), 7.29 (t, J=8.1 Hz, 1H), 6.84-6.80 (m, 1H), 5.25 (s, 2H), 3.70 (q, J=7.1 Hz, 1H), 1.46 (d, J=7.0 Hz, 3H). MS (ESI) m/z 503.2 [M+H]⁺.

### Example 173: Synthesis of FM-71150 (JJ140)

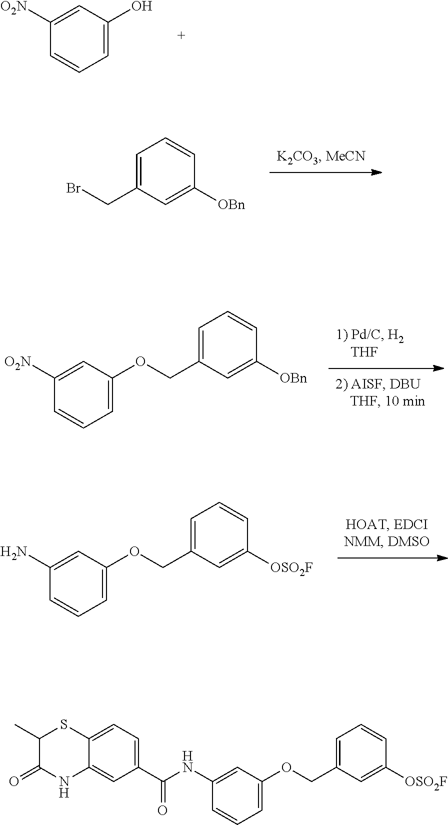

FM-71150 (JJ140) was prepared as the procedure of JJ139.

¹H NMR (800 MHz, Acetone-d6) δ 9.74 (s, 1H), 9.59 (s, 1H), 7.77-7.75 (m, 1H), 7.73-7.67 (m, 4H), 7.64 (d, J=8.1 Hz, 1H), 7.55-7.52 (m, 1H), 7.47 (d, J=8.0 Hz, 1H), 7.41 (d, J=8.2 Hz, 1H), 7.30 (t, J=8.1 Hz, 1H), 6.85-6.82 (m, 1H), 5.28 (s, 2H), 3.70 (q, J=7.0 Hz, 1H), 1.46 (d, J=7.0 Hz, 3H). MS (ESI) m/z 503.0 [M+H]⁺.

### Example 174: Synthesis of FM-74044 (JJ141)

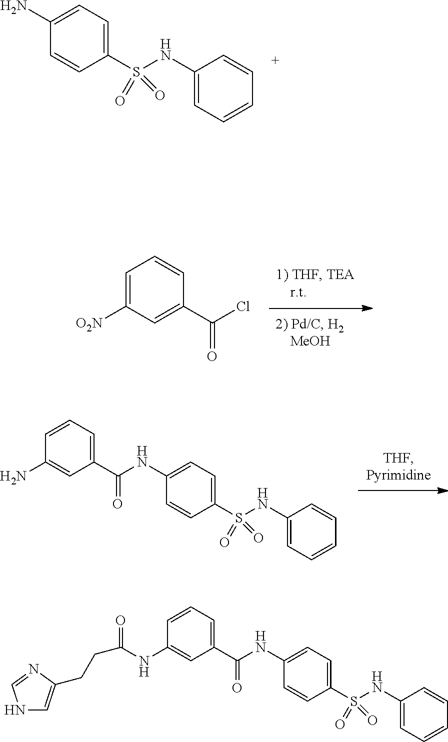 [SMILES: Nc1ccc(S(=O)(=O)Nc2ccccc2)cc1 ; O=C(Cl)c1cccc([N+](=O)[O-])c1 ; Nc1cccc(C(=O)Nc2ccc(S(=O)(=O)Nc3ccccc3)cc2)c1 ; O=C(CCc1c[nH]cn1)Nc1cccc(C(=O)Nc2ccc(S(=O)(=O)Nc3ccccc3)cc2)c1]

FM-74044 (JJ141) was prepared as the procedure of JJ134 and JJ135.

¹H NMR (800 MHz, Acetone-d6) δ 10.23 (s, 1H), 10.02 (s, 1H), 8.97 (s, 1H), 8.76 (s, 1H), 8.43-8.38 (m, 2H), 8.06 (d, J=8.4 Hz, 2H), 7.94 (d, J=8.2 Hz, 1H), 7.78 (d, J=8.6 Hz, 2H), 7.66 (d, J=7.7 Hz, 1H), 7.46-7.41 (m, 2H), 7.33-7.22 (m, 4H), 7.08 (t, J=6.9 Hz, 1H), 3.21-3.15 (m, 2H), 2.93-2.88 (m, 2H). MS (ESI) m/z 490.3 [M+H]⁺.

### Example 175: Synthesis of FM-74069 (JJ142)

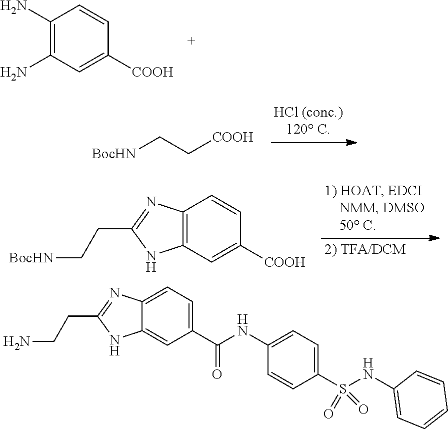

To a sealed tube was added 3-((tert-butoxycarbonyl)amino)propanoic acid (4.0 mmol), 3,4-diaminobenzoic acid (1.0 mmol) and 2 mL concentrated HCl. The mixture was stirred at 120° C. for 16 h. Upon completion, the mixture was purified by reverse column chromatography to get 2-(2-((tert-butoxycarbonyl)amino)ethyl)-1H-benzo[d]imidazole-6-carboxylic acid with 21% yield.

A mixture of 2-(2-((tert-butoxycarbonyl)amino)ethyl)-1H-benzo[d]imidazole-6-carboxylic acid (0.2 mmol), 4-amino-N-phenylbenzenesulfonamide (0.2 mmol), HOAT (0.4 mmol), EDCI (0.4 mmol) and NMM (0.6 mmol) in 2 mL DMSO was stirred at 50° C. overnight. Upon completion, the mixture was purified by reverse column chromatography to get FM-74069 (JJ142) with 41% yield.

¹H NMR (400 MHz, DMSO-d6) δ 10.56 (s, 1H), 10.19 (s, 1H), 9.06 (s, 1H), 8.20 (s, 1H), 7.97-7.90 (m, 2H), 7.84 (d, J=8.5 Hz, 1H), 7.80-7.70 (m, 2H), 7.65 (d, J=8.4 Hz, 1H), 7.29-7.18 (m, 2H), 7.09 (d, J=7.9 Hz, 2H), 7.01 (t, J=7.4 Hz, 1H), 3.22 (t, J=7.1 Hz, 2H), 3.14-3.03 (m, 4H). MS (ESI) m/z 436.2 [M+H]⁺.

### Example 176: Synthesis of FM-89068 (JJ167)

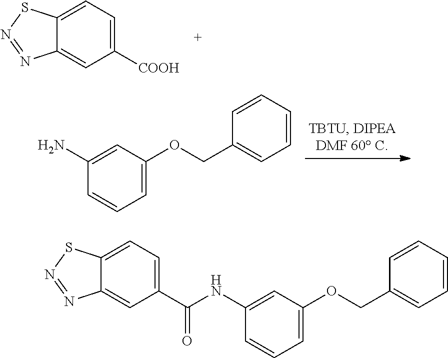

A mixture of benzo[d][1,2,3]thiadiazole-5-carboxylic acid (0.2 mmol), 3-(benzyloxy)aniline (0.2 mmol), DIPEA (0.4 mmol) and TBTU (0.4 mmol) in 1 mL DMF was stirred at 60° C. for 6 h. Upon completion, the mixture was purified by pre-HPLC to get FM-89068 (JJ167) with 44% yield.

¹H NMR (600 MHz, DMSO-d6) δ 10.59 (s, 1H), 9.32 (s, 1H), 8.59-8.53 (m, 1H), 8.35-8.30 (m, 1H), 7.63 (s, 1H), 7.51-7.46 (m, 2H), 7.44-7.38 (m, 3H), 7.36-7.34 (m, 1H), 7.34-7.27 (m, 1H), 6.84-6.79 (m, 1H), 5.13 (s, 2H). MS (ESI) m/z 362.2 [M+H]⁺.

### Example 177: Synthesis of FM-89073 (JJ169)

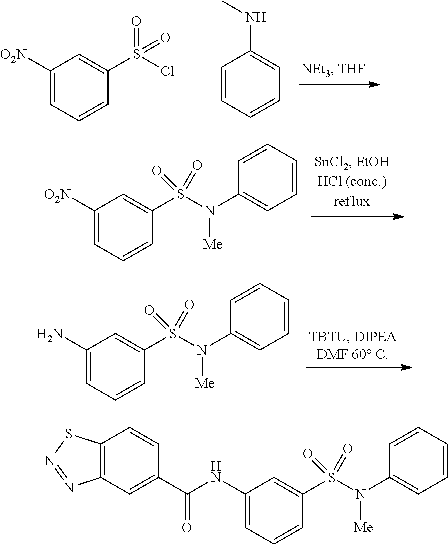

A mixture of 3-nitrobenzenesulfonyl chloride (1.0 mmol), N-methylaniline (1.0 mmol) and NEt₃ (1.2 mmol) in 10 mL THF was stirred at room temperature for 3 h. Upon completion, the mixture was purified by chromatography to get N-methyl-3-nitro-N-phenylbenzenesulfonamide with 95% yield.

To a solution of N-methyl-3-nitro-N-phenylbenzenesulfonamide (0.2 mmol) and SnCl₂ (0.5 mmol) in 10 mL EtOH was added 0.5 mL concentrated HCl. The solution was stirred at 80° C. for 3 h. The mixture was purified by reverse column chromatography to give 3-amino-N-methyl-N-phenylbenzenesulfonamide with 90% yield.

A mixture of 3-amino-N-methyl-N-phenylbenzenesulfonamide (0.2 mmol), DIPEA (0.4 mmol), benzo[d][1,2,3]thiadiazole-5-carboxylic acid (0.2 mmol) and TBTU (0.4 mmol) in 1 mL DMF was stirred at 60° C. for 6 h. Upon completion, the mixture was purified by pre-HPLC to get FM-89073 (JJ169) with 56% yield.

¹H NMR (600 MHz, DMSO-d6) δ 10.91 (s, 1H), 9.37 (s, 1H), 8.58 (d, J=8.5 Hz, 1H), 8.34 (d, J=8.6 Hz, 1H), 8.23-8.18 (m, 2H), 7.60 (t, J=8.0 Hz, 1H), 7.40-7.33 (m, 2H), 7.32-7.27 (m, 1H), 7.21 (d, J=8.0 Hz, 1H), 7.17-7.14 (m, 2H), 3.21 (d, J=1.7 Hz, 3H). MS (ESI) m/z 425.2 [M+H]⁺.

### Example 178: Synthesis of FM-89075 (JJ170)

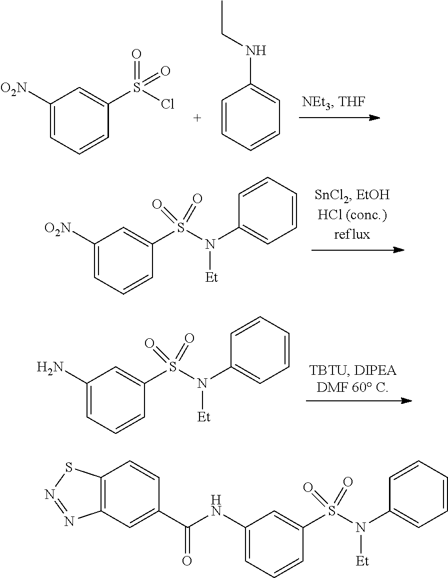

FM-89075 (JJ170) was prepared as the procedure of JJ169.

¹H NMR (600 MHz, DMSO-d6) δ 10.91 (s, 1H), 9.39-9.36 (m, 1H), 8.58 (d, J=8.5 Hz, 1H), 8.35 (dd, J=8.5, 1.6 Hz, 1H), 8.25 (t, J=2.0 Hz, 1H), 8.22-8.18 (m, 1H), 7.61 (t, J=8.0 Hz, 1H), 7.42-7.37 (m, 2H), 7.37-7.32 (m, 1H), 7.30 (d, J=7.8 Hz, 1H), 7.13-7.09 (m, 2H), 3.66 (q, J=7.1 Hz, 2H), 1.00 (t, J=7.1 Hz, 3H). MS (ESI) m/z 439.1 [M+H]⁺.

### Example 179: Synthesis of FM-89076 (JJ192)

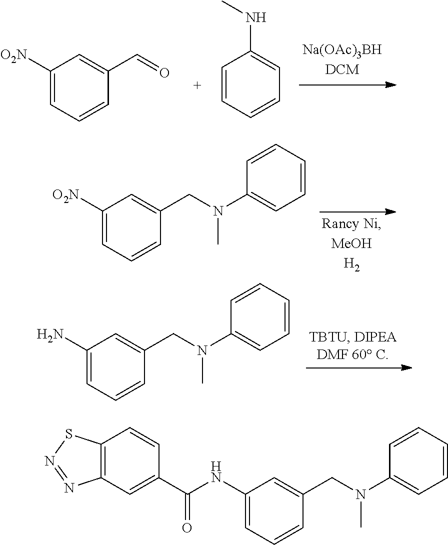

To a solution of 3-nitrobenzaldehyde (0.2 mmol) and N-methylaniline (0.2 mmol) in 3 mL DCM was added Na(OAc)₃BH (0.4 mmol). The solution was stirred at room temperature for 3 h. The mixture was purified by chromatography to give N-methyl-N-(3-nitrobenzyl)aniline with 85% yield.

To a solution of N-methyl-N-(3-nitrobenzyl)aniline in 10 mL MeOH was added Raney Ni. Exchanged the air with H₂ for three times before stirred at room temperature overnight. The reaction was monitored by LC-MS and upon completion. The solid was filtered and the solvent was removed under reduced pressure to give crude product without further purification.

A mixture of benzo[d][1,2,3]thiadiazole-5-carboxylic acid (0.2 mmol), N-(3-aminobenzyl)-N-methylaniline (0.2 mmol), DIPEA (0.4 mmol) and TBTU (0.4 mmol) in 1 mL DMF was stirred at 60° C. for 6 h. Upon completion, the mixture was purified by pre-HPLC to get FM-89076 (JJ192) with 60% yield.

¹H NMR (800 MHz, Methanol-d₄) δ 9.21 (s, 1H), 8.39 (d, J=8.4 Hz, 1H), 8.29 (d, J=8.5 Hz, 1H), 7.69 (d, J=8.0 Hz, 1H), 7.67 (s, 1H), 7.36 (t, J=7.7 Hz, 1H), 7.18 (t, J=7.8 Hz, 2H), 7.10 (d, J=7.6 Hz, 1H), 6.80 (d, J=8.2 Hz, 2H), 6.67 (t, J=7.3 Hz, 1H), 4.60 (s, 2H), 3.08 (s, 3H).

MS (ESI) m/z 375.2 [M+H]⁺.

### Example 180: Synthesis of FM-89078 (JJ193)

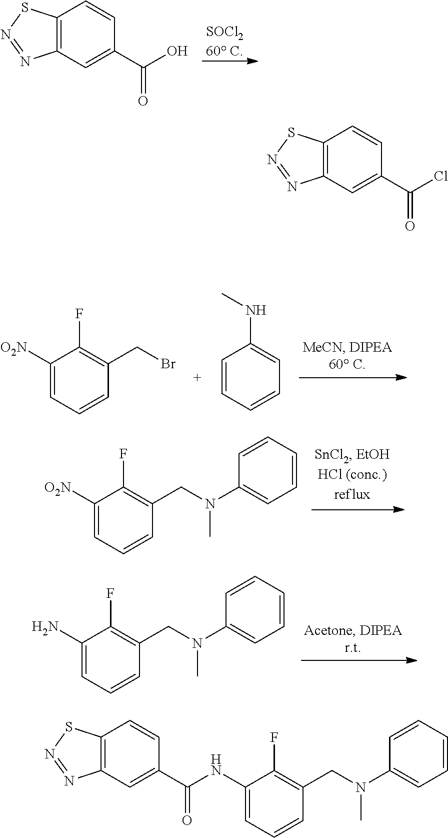

A mixture of benzo[d][1,2,3]thiadiazole-5-carboxylic acid (0.2 mmol) in 1 mL SOCl₂ was stirred at 60° C. for 3 h. The reaction was monitored by LC-MS and upon completion; the solvent was evaporated under reduced pressure to obtain crude benzo[d][1,2,3]thiadiazole-5-carbonyl chloride without further purification.

A mixture of 1-(bromomethyl)-2-fluoro-3-nitrobenzene (0.2 mmol), N-methylaniline (0.2 mmol) and DIPEA (0.2 mmol) in 2 mL MeCN was stirred at 60° C. for 6 h. The mixture was purified by chromatography to obtain N-(2-fluoro-3-nitrobenzyl)-N-methylaniline with 85% yield.

To a solution of N-(2-fluoro-3-nitrobenzyl)-N-methylaniline (0.1 mmol) and SnCl₂ (0.25 mmol) in 5 mL EtOH was added 1 drop of concentrated HCl. The solution was stirred at 80° C. for 3 h. The mixture was purified by reverse column chromatography to obtain N-(3-amino-2-fluorobenzyl)-N-methylaniline with 90% yield.

To a solution of benzo[d][1,2,3]thiadiazole-5-carbonyl chloride (0.1 mmol) and N-(3-amino-2-fluorobenzyl)-N-methylaniline (0.1 mmol) in 2 mL Acetone was added DIPEA (0.1 mmol). The mixture was stirred at room temperature for 3 h. The mixture was purified by reverse column chromatography to obtain FM-89078 (JJ193) with 92% yield.

¹H NMR (800 MHz, Acetone-d6) δ 9.71 (s, 1H), 9.37 (s, 1H), 8.57 (d, J=8.4 Hz, 1H), 8.44 (d, J=8.4 Hz, 1H), 8.07-7.99 (m, 1H), 7.26-7.16 (m, 3H), 7.08 (t, J=7.2 Hz, 1H), 6.82 (d, J=8.2 Hz, 2H), 6.69 (t, J=7.2 Hz, 1H), 4.70 (s, 2H), 3.11 (s, 3H). MS (ESI) m/z 393.2 [M+H]⁺.

### Example 181: Synthesis of FM-89079 (JJ194)

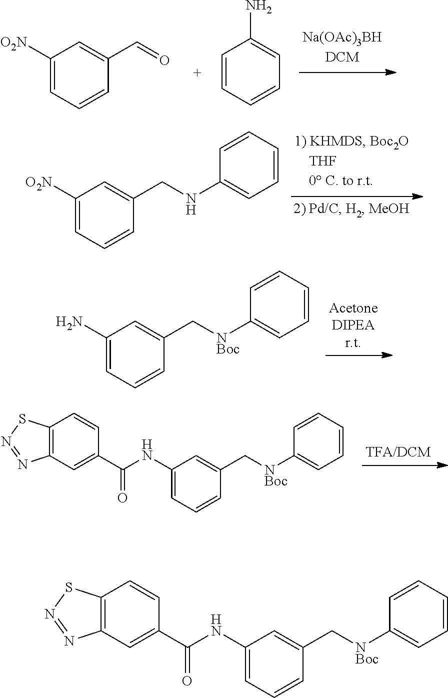

The preparation of FM-89079 (JJ194) was similar with that of JJ193 and JJ192.

The solution of N-(3-nitrobenzyl)aniline (0.2 mmol) in 4 mL THF was cooled to 0° C. To this solution was added KHMDS (0.3 mmol) dropwise. After 30 min, Boc₂O (0.2 mmol) was added to the mixture. The solution was stirred at room temperature for 3 h. the mixture was purified by chromatography to obtain tert-butyl (3-nitrobenzyl)(phenyl)carbamate with 40% yield.

¹H NMR (800 MHz, Acetone-d6) δ 10.00 (s, 1H), 9.33 (s, 1H), 8.65 (s, 1H), 8.55 (d, J=8.4 Hz, 1H), 8.43 (d, J=8.4 Hz, 1H), 8.27 (d, J=8.5 Hz, 1H), 8.09 (s, 1H), 7.88 (t, J=9.1 Hz, 1H), 7.38 (t, J=7.7 Hz, 1H), 7.30 (s, 2H), 7.26-7.18 (m, 2H), 7.13 (t, J=7.3 Hz, 1H), 5.32 (s, 2H). MS (ESI) m/z 361.2 [M+H]⁺.

### Example 182: Synthesis of FM-89102 (JJ195)

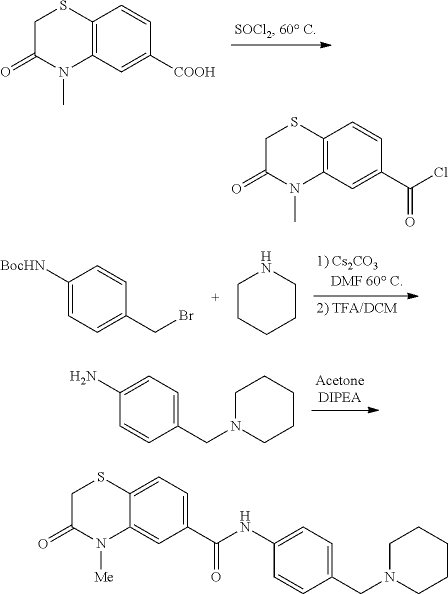

The preparation of 4-methyl-3-oxo-3,4-dihydro-2H-benzo[b][1,4]thiazine-6-carbonyl chloride was the same with that of benzo[d][1,2,3]thiadiazole-5-carbonyl chloride.

A mixture of tert-butyl (4-(bromomethyl)phenyl)carbamate (0.2 mmol), piperidine (0.2 mmol) and Cs₂CO₃ in 2 mL DMF was stirred at 60° C. for 6 h. Upon completion, water was added and then the mixture was extracted by EA for 3 times. The organic phase was dried with Na₂SO₄. The crude product can be used without further purification.

Then the preparation of FM-89102 (JJ195) was similar with that of JJ193.

¹H NMR (800 MHz, DMSO-d6) δ 10.46 (s, 1H), 7.87 (d, J=8.2 Hz, 2H), 7.75 (s, 1H), 7.67 (d, J=8.1 Hz, 1H), 7.60 (d, J=7.9 Hz, 1H), 7.50 (d, J=8.2 Hz, 2H), 4.26 (d, J=5.3 Hz, 2H), 3.61 (s, 2H), 3.18 (s, 3H), 2.91-2.84 (m, 2H), 1.85-1.81 (m, 3H), 1.72-1.68 (m, 1H), 1.63 (d, J=13.4 Hz, 3H), 1.41-1.33 (m, 1H). MS (ESI) m/z 396.3 [M+H]⁺.

### Example 183: Synthesis of FM-89103 (JJ196)

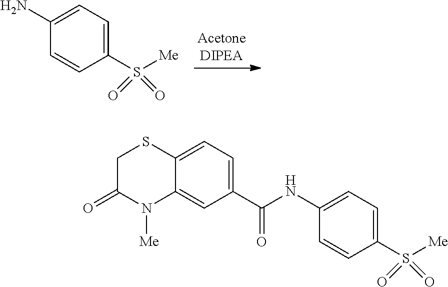 [SMILES: CS(=O)(=O)c1ccc(N)cc1 ; CN1C(=O)CSc2ccc(C(=O)Nc3ccc(S(C)(=O)=O)cc3)cc21]

The preparation of FM-89103 (JJ196) was similar with that of JJ193.

¹H NMR (800 MHz, DMSO-d6) δ 10.69 (s, 1H), 8.05 (d, J=8.5 Hz, 2H), 7.94 (d, J=8.5 Hz, 2H), 7.77 (s, 1H), 7.70 (d, J=7.9 Hz, 1H), 7.62 (d, J=8.1 Hz, 1H), 3.61 (s, 2H), 3.45 (s, 3H), 3.20 (s, 3H). MS (ESI) m/z 377.1 [M+H]⁺.

### Example 184: Synthesis of FM-89107 (JJ197)

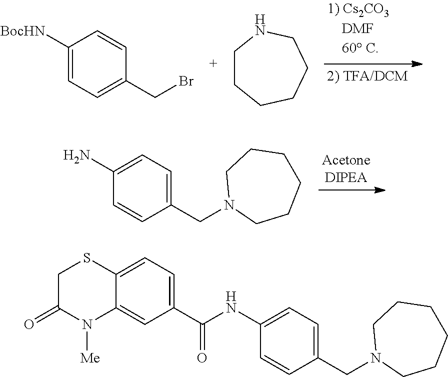

The preparation of FM-89107 (JJ197) was similar with that of JJ195

¹H NMR (800 MHz, DMSO-d6) δ 10.46 (s, 1H), 7.86 (d, J=8.1 Hz, 2H), 7.75 (d, J=5.5 Hz, 1H), 7.67 (d, J=7.9 Hz, 1H), 7.59 (dd, J=18.9, 8.1 Hz, 1H), 7.53 (d, J=8.2 Hz, 2H), 4.33-4.30 (m, 2H), 3.61 (s, 2H), 3.45 (s, 3H), 3.11-3.05 (m, 4H), 1.90-1.84 (m, 2H), 1.79-1.70 (m, 2H), 1.68-1.56 (m, 4H). MS (ESI) m/z 410.3 [M+H]⁺.

### Example 185: Synthesis of FM-89109 (JJ198)

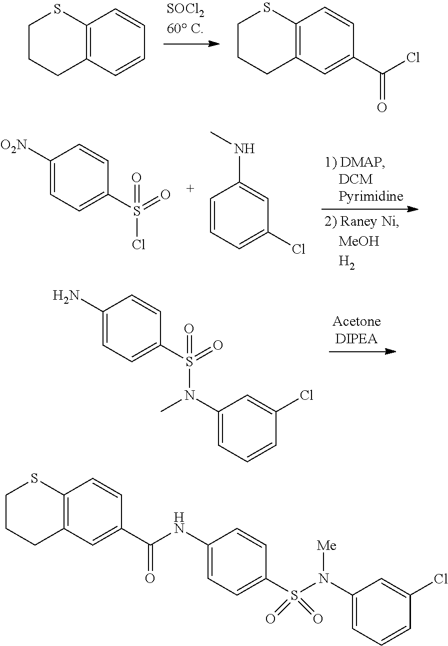

The preparation of thiochromane-6-carbonyl chloride was the same with that of 4-methyl-3-oxo-3,4-dihydro-2H-benzo[b][1,4]thiazine-6-carbonyl chloride.

A mixture of 4-nitrobenzenesulfonyl chloride (1.0 mmol), 3-chloro-N-methylaniline (1.0 mmol), DMAP (0.1 mmol) and Pyrimidine (1 mmol) in 2 mL DCM was stirred at room temperature for 6 h. Upon completion, the solvent was evaporated under reduced pressure to obtain crude product. The crude product can be used without further purification.

The crude product was dissolved in 5 mL MeOH and then Raney Ni was added. Exchange the air with H₂ for 3 times, the mixture was stirred at room temperature for 6 h. The mixture was purified by reverse column chromatograph to obtain 4-amino-N-(3-chlorophenyl)-N-methylbenzenesulfonamide with 57% yield.

To a solution of 4-amino-N-(3-chlorophenyl)-N-methylbenzenesulfonamide (0.2 mmol) and thiochromane-6-carbonyl chloride (0.2 mmol) in 3 mL Acetone was added DIPEA (0.2 mmol). The mixture was stirred at room temperature for 3 h. Upon completion, the mixture was purified by reverse column chromatograph to obtain FM-89109 (JJ198) with 78% yield.

¹H NMR (800 MHz, DMSO-d6) δ 10.51 (s, 1H), 7.99 (d, J=8.5 Hz, 2H), 7.70 (s, 1H), 7.68 (d, J=8.4 Hz, 1H), 7.52 (d, J=8.5 Hz, 2H), 7.41-7.36 (m, 2H), 7.24 (s, 1H), 7.22 (d, J=8.2 Hz, 1H), 7.12 (d, J=7.4 Hz, 1H), 3.15 (s, 3H), 3.13-3.07 (m, 2H), 2.87 (t, J=6.1 Hz, 2H), 2.06-2.04 (m, 2H). MS (ESI) m/z 473.1[M+H]⁺.

### Example 186: Synthesis of FM-89100 (JJ228)

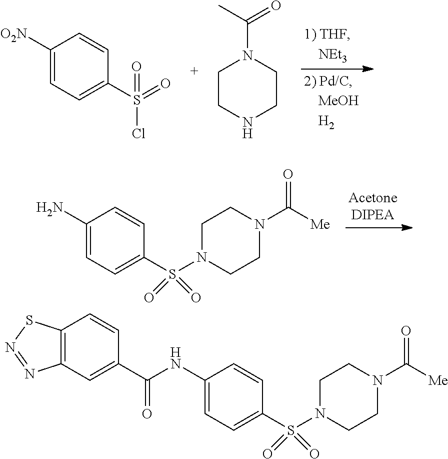

The preparation of FM-89100 (JJ228) was similar with that of JJ198.

¹H NMR (800 MHz, DMSO-d6) δ 11.04 (s, 1H), 9.38 (s, 1H), 8.59 (d, J=8.4 Hz, 1H), 8.34 (d, J=8.4 Hz, 1H), 8.14 (d, J=8.4 Hz, 2H), 7.79 (d, J=8.4 Hz, 2H), 3.56-3.50 (m, 4H), 2.97-2.92 (m, 2H), 2.88 (t, J=5.4 Hz, 2H), 1.95 (s, 3H). MS (ESI) m/z 446.2 [M+H]⁺.

### Example 187: Synthesis of FM-89110 (JJ229)

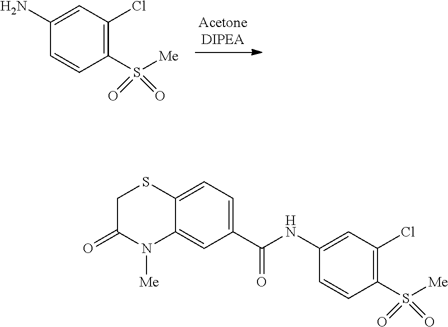 [SMILES: CS(=O)(=O)c1ccc(N)cc1Cl ; CN1C(=O)CSc2ccc(C(=O)Nc3ccc(S(C)(=O)=O)c(Cl)c3)cc21]

The preparation of FM-89110 (JJ229) was similar with that of JJ198.

¹H NMR (800 MHz, DMSO-d6) δ 10.80 (s, 1H), 8.21 (s, 1H), 8.06 (d, J=8.7 Hz, 1H), 7.99 (d, J=8.4 Hz, 1H), 7.77 (s, 1H), 7.69 (d, J=8.0 Hz, 1H), 7.63 (d, J=8.0 Hz, 1H), 3.62 (s, 2H), 3.45 (s, 3H), 3.20-3.17 (m, 3H). MS (ESI) m/z 411.1 [M+H]⁺.

### Example 188: Synthesis of FM-89112 (JJ230)

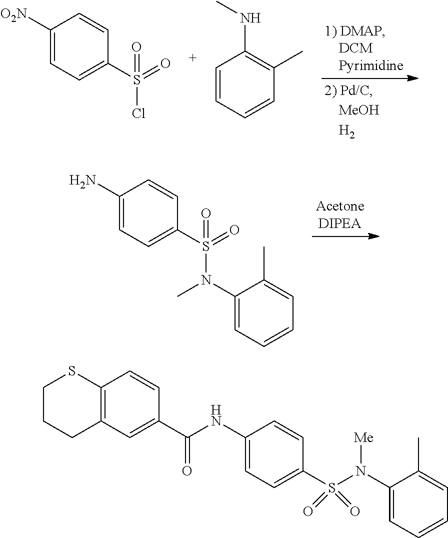

The preparation of FM-89112 (JJ230) was similar with that of JJ198.

¹H NMR (800 MHz, DMSO-d6) δ 10.53 (s, 1H), 8.03 (d, J=8.6 Hz, 2H), 7.72 (s, 1H), 7.70 (d, J=8.6 Hz, 1H), 7.66 (d, J=8.6 Hz, 2H), 7.33 (d, J=7.5 Hz, 1H), 7.26 (d, J=7.6 Hz, 1H), 7.23 (d, J=8.0 Hz, 1H), 7.13 (s, 1H), 6.64 (d, J=8.0 Hz, 1H), 3.14-3.10 (m, 2H), 3.09 (s, 3H), 2.89 (t, J=6.0 Hz, 2H), 2.32 (s, 3H), 2.06 (s, 2H). MS (ESI) m/z 453.1 [M+H]⁺.

### Example 189: Synthesis of FM-89113 (JJ231)

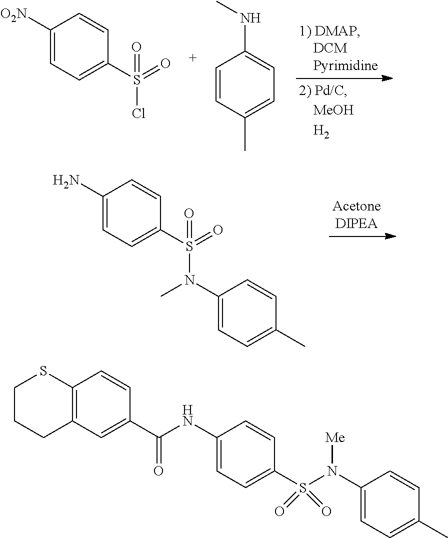

The preparation of FM-89113 (JJ231) was similar with that of JJ198.

¹H NMR (800 MHz, DMSO-d6) δ 10.49 (s, 1H), 7.96 (d, J=8.5 Hz, 2H), 7.70 (s, 1H), 7.68 (d, J=8.4 Hz, 1H), 7.49 (d, J=8.5 Hz, 2H), 7.22 (d, J=8.2 Hz, 1H), 7.15 (d, J=8.0 Hz, 2H), 6.99 (d, J=8.0 Hz, 2H), 3.12-3.09 (m, 5H), 2.89-2.85 (m, 2H), 2.30 (s, 3H), 2.08-2.02 (m, 2H). MS (ESI) m/z 453.1 [M+H]⁺.

### Example 190: Synthesis of FM-89114 (JJ232)

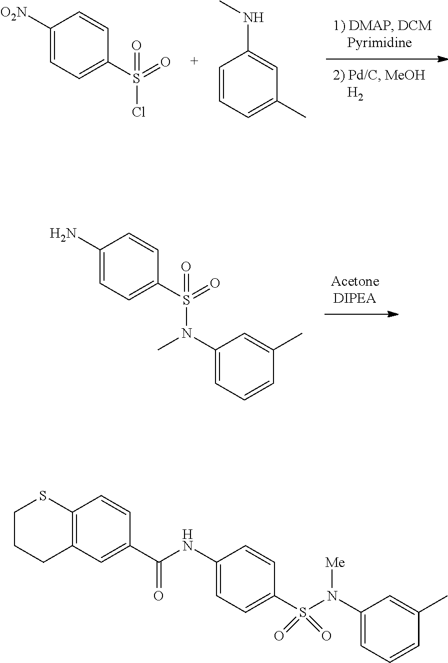

The preparation of FM-89114 (JJ232) was similar with that of JJ198.

¹H NMR (800 MHz, DMSO-d6) δ 10.49 (s, 1H), 7.97 (d, J=8.5 Hz, 2H), 7.70 (s, 1H), 7.68 (d, J=8.5 Hz, 1H), 7.51 (d, J=8.5 Hz, 2H), 7.23 (t, J=9.1 Hz, 2H), 7.12 (d, J=7.5 Hz, 1H), 6.98 (s, 1H), 6.87 (d, J=8.1 Hz, 1H), 3.14-3.09 (m, 5H), 2.89-2.86 (m, 2H), 2.28 (s, 3H), 2.10-2.01 (m, 2H). MS (ESI) m/z 453.1 [M+H]⁺.

### Example 191: Synthesis of FM-89115 (JJ233)

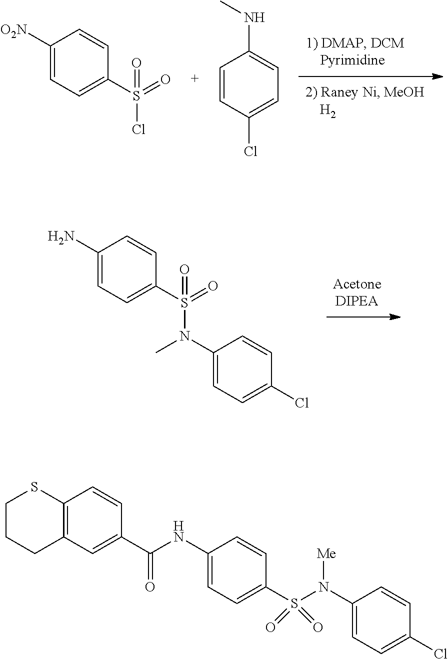

The preparation of FM-89115 (JJ233) was similar with that of JJ198.

¹H NMR (800 MHz, DMSO-d6) δ 10.50 (s, 1H), 7.98 (d, J=8.5 Hz, 2H), 7.70 (s, 1H), 7.68 (d, J=8.4 Hz, 1H), 7.50 (d, J=8.4 Hz, 2H), 7.43 (d, J=8.5 Hz, 2H), 7.22 (d, J=8.1 Hz, 1H), 7.16 (d, J=8.5 Hz, 2H), 3.13 (s, 3H), 3.12-3.09 (m, 2H), 2.87 (t, J=6.1 Hz, 2H), 2.06 (d, J=6.3 Hz, 2H). MS (ESI) m/z 473.0739 [M+H]⁺.

### Example 192: Synthesis of FM-89118 (JJ234)

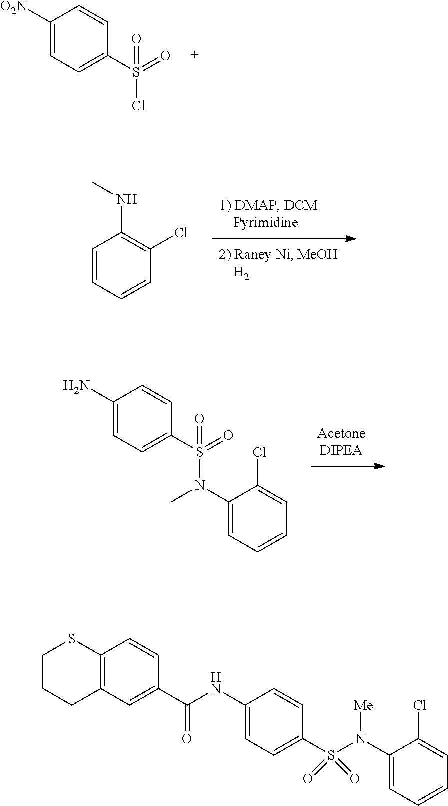

The preparation of FM-89118 (JJ234) was similar with that of JJ198.

¹H NMR (800 MHz, DMSO-d6) δ 10.53 (s, 1H), 8.04 (d, J=8.6 Hz, 2H), 7.73-7.68 (m, 4H), 7.58 (d, J=7.9 Hz, 1H), 7.40 (t, J=7.7 Hz, 1H), 7.35 (t, J=7.6 Hz, 1H), 7.23 (d, J=8.0 Hz, 1H), 7.07 (d, J=7.7 Hz, 1H), 3.14-3.09 (m, 5H), 2.89-2.87 (m, 2H), 2.06 (s, 2H). MS (ESI) m/z 473.1 [M+H]⁺.

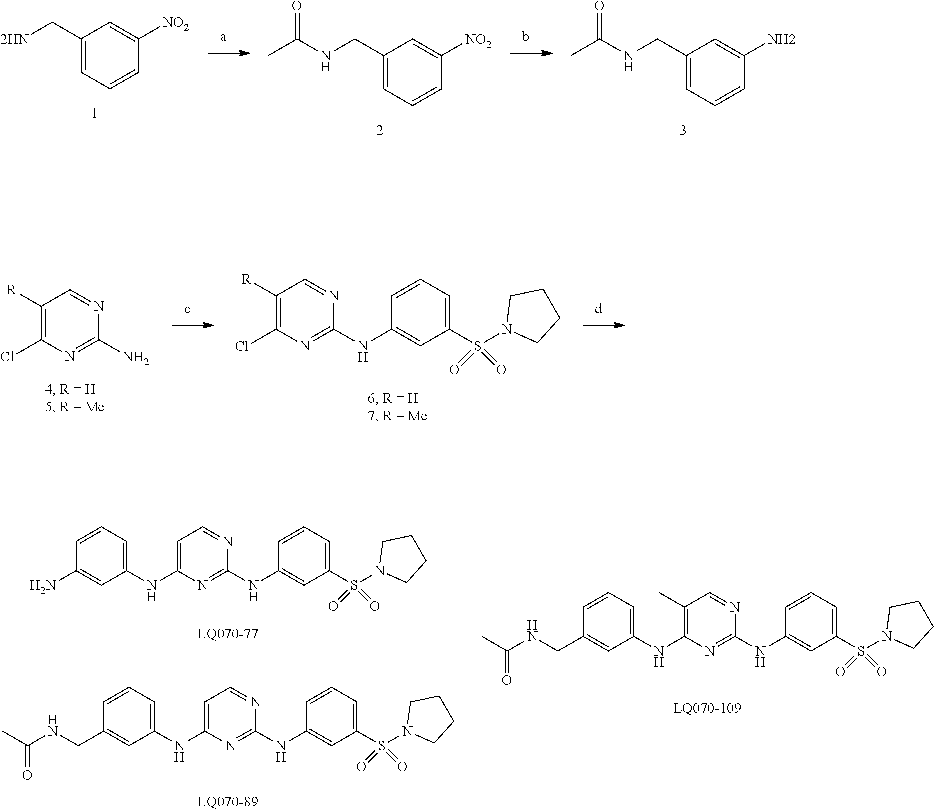

Reagents and Conditions: a) Et₃N, DCM, 0° C., 87%; b) Pd/C, H₂, MeOH; c) Pd₂(dba)₃, Cs₂CO₃, Xantphos, dioxane, DMF, 130° C., 30%; d) Pd₂(dba)₃, Cs₂CO₃, Xantphos, dioxane, DMF, 135° C.

### N-(3-nitrobenzyl)acetamide (2)

3-Nitrobenzylamine hydrochloride (500 mg, 2.6 mmol) and Et₃N (902 μL, 6.5 mmol) were dissolved in dichloromethane and treated with acetyl chloride (223 μL, 3.2 mmol) at 0° C. After being stirred 1 h at 0° C. the mixture was washed with sodium bicarbonate solution, dried over sodium sulfate and concentrated. The resulting residue was purified by silica gel flash chromatography to yield the title compound as yellow solid (438 mg, 87%). MS (ESI): m/z 195.1 [M+H]⁺.

### N-(3-aminobenzyl)acetamide (3)

10% Pd on carbon (50 mg) was added to a solution of N-(3-nitrobenzyl)acetamide (530 mg, 2.7 mmol) in MeOH, and the mixture was stirred under H₂ atmosphere overnight. The catalyst was removed by filtration through a pad of celite, the solvent was removed in vacuo and the residue was used in next step without further purification. ¹H NMR (800 MHz, Methanol-d₄) δ 7.07 (t, J=7.8 Hz, 1H), 6.67 (s, 1H), 6.63 (t, J=8.5 Hz, 2H), 4.27 (s, 2H), 2.00 (s, 3H). MS (ESI): m/z 165.2 [M+H]⁺.

### 4-Chloro-N-(3-(pyrrolidin-1-ylsulfonyl)phenyl)pyrimidin-2-amine (6)

A solution of 2-Amino-4-chloropyrimidine (27 mg, 0.2 mmol), 1-((3-Bromophenyl)sulfonyl)pyrrolidine (60 mg, 0.2 mmol), Pd₂(dba)₃ (9 mg, 0.01 mmol), cesium carbonate (130 mg, 0.4 mmol), xantphos (12 mg, 0.02 mmol) in dioxane was heated to 130° C. under microwave for 0.5 h. After cooling to room temperature, the mixture was poured in water and extracted with EtOAc. The combined organic layer was washed with brine and concentrated in vacuo. The resulting residue was purified by silica gel flash chromatography to give the compound as white solid (20 mg, 30%). MS (ESI): m/z 339.1 [M+H]⁺.

### 4-Chloro-5-methyl-N-(3-(pyrrolidin-1-ylsulfonyl)phenyl)pyrimidin-2-amine (7)

Compound 7 was prepared using same procedures as preparing compound 6 from 4-Chloro methylpyrimidin-2-amine and 1-((3-Bromophenyl)sulfonyl)pyrrolidine. Yield: 34%. MS (ESI): m/z 353.1 [M+H]⁺.

### Example 193: Synthesis of LQ070-77 (JJ143)

### N⁴-(3-aminophenyl)-N²-(3-(pyrrolidin-1-ylsulfonyl)phenyl)pyrimidine-2,4-diamine

Compound LQ070-77 was prepared using same procedures as preparing compound 6 from compound 6 and m-Phenylenediamine. Yield: 20%. ¹H NMR (500 MHz, Methanol-d₄) δ 8.01-7.98 (m, 1H), 7.93 (d, J=7.1 Hz, 1H), 7.86 (d, J=8.0 Hz, 1H), 7.74-7.71 (m, 1H), 7.65 (t, J=7.9 Hz, 1H), 7.55-7.51 (m, 1H), 7.42 (t, J=8.1 Hz, 1H), 7.38 (s, 1H), 7.10-7.06 (m, 1H), 6.52 (d, J=7.2 Hz, 1H), 3.25-3.20 (m, 5H), 1.77-1.73 (m, 4H). MS (ESI): m/z 411.3[M+H]⁺. HRMS m/z [M+H]⁺ calcd for C₂₀H₂₃N₆O₂S⁺ 411.1598, found 411.2184.

### Example 194: Synthesis of LQ070-89 (JJ146)

### N-(3-((2-((3-(pyrrolidin-1-ylsulfonyl)phenyl)amino)pyrimidin-4-yl)amino)benzyl)acetamide

Compound LQ070-89 was prepared using same procedures as preparing compound 6 from compound 6 and Compound 3. Yield: 25%. ¹H NMR (500 MHz, Methanol-d₄) δ 7.97-7.83 (m, 3H), 7.72-7.68 (m, 1H), 7.62 (t, J=8.0 Hz, 1H), 7.53-7.30 (m, 3H), 7.19-7.13 (m, 1H), 6.46 (d, J=7.2 Hz, 1H), 4.31 (s, 2H), 3.35-3.31 (m, 1H), 2.00 (s, 3H), 1.77-1.67 (m, 5H). MS (ESI): m/z 467.4 [M+H]⁺. HRMS m/z [M+H]⁺ calcd for C₂₃H₂₇N₆O₃S⁺ 467.1860, found 467.2792.

### Example 195

### N-(3-((5-methyl-2-((3-(pyrrolidin-1-ylsulfonyl)phenyl)amino)pyrimidin-4-yl)amino)benzyl)acetamide

Compound LQ070-109 was prepared using same procedures as preparing compound 6 from compound 7 and Compound 3. Yield: 30%. ¹H NMR (500 MHz, Methanol-d₄) δ 7.88-7.85 (m, 1H), 7.78-7.76 (m, 1H), 7.69 (t, J=1.9 Hz, 1H), 7.60-7.57 (m, 1H), 7.46-7.38 (m, 4H), 7.26-7.24 (m, 1H), 4.36 (s, 2H), 3.24-3.16 (m, 4H), 2.28-2.24 (m, 3H), 1.98 (s, 3H), 1.77-1.68 (m, 4H). MS (ESI): m/z 481.3 [M+H]⁺. HRMS m/z [M+H]⁺ calcd for C₂₄H₂₉N₆O₃S⁺ 481.2016, found 481.3400.

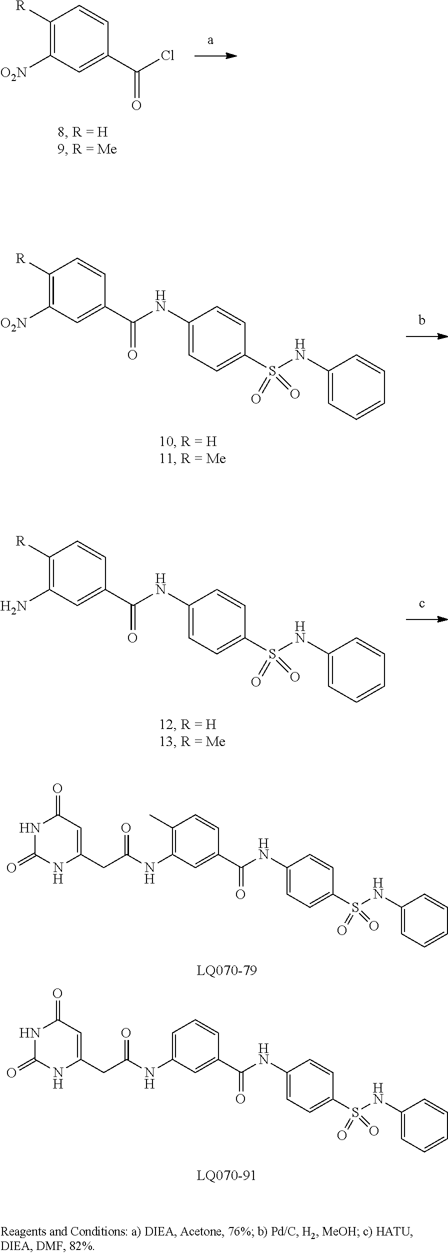

### 3-Nitro-N-(4-(N-phenylsulfamoyl)phenyl)benzamide (10)

To a stirred solution of 4-Amino-n-phenyl-benzenesulfonamide (50 mg, 0.2 mmol) and DIEA (66 μL, 0.4 mmol) in acetone was added 3-Nitrobenzoyl Chloride (32 mg, 0.2 mmol) at 0° C. The resulting mixture was warmed to room temperature to stirred for 1 h and quenched with NH₄Cl solution, then the mixture was extracted with ethyl acetate (5 mL×3). The combined organic extracts were washed with brine, dried over sodium sulfate, concentrated. The resulting residue was purified by silica gel flash chromatography to give the compound as white solid (60 mg, 76%). MS (ESI): m/z 398.2 [M+H]⁺.

### 4-Methyl-3-nitro-N-(4-(N-phenylsulfamoyl)phenyl)benzamide (11)

Compound 11 was prepared using same procedures as preparing compound 10 from 4-methyl-3-nitrobenzoyl chloride and 4-Amino-n-phenyl-benzenesulfonamide. Yield: 80%. MS (ESI): m/z 412.9 [M+H]⁺.

### 3-Amino-N-(4-(N-phenylsulfamoyl)phenyl)benzamide (12)

Compound 12 was prepared using same procedures as preparing compound 3 from Compound 10. MS (ESI): m/z 368.2 [M+H]⁺.

### 3-Amino-4-methyl-N-(4-(N-phenylsulfamoyl)phenyl)benzamide (13)

Compound 12 was prepared using same procedures as preparing compound 3 from Compound 11. MS (ESI): m/z 382.3 [M+H]⁺.

### Example 196: Synthesis of LQ070-79 (JJ144)

### 3-(2-(2,6-dioxo-1,2,3,6-tetrahydropyrimidin-4-yl)acetamido)-4-methyl-N-(4-(N-phenylsulfamoyl)phenyl)benzamide

To the solution of 2-(2,6-dioxo-1,2,3,6-tetrahydropyrimidin-4-yl)acetic acid (51 mg, 0.3 mmol) in DCM were treated with compound 13 (113 mg, 0.3 mmol), HATU (125 mg, 0.33 mmol) and TEA (62 μL, 0.45 mmol). After being stirring overnight at room temperature, the resulting mixture was purified by reverse phase column to afford the compound 32 as yellow solid. Yield: 82%. ¹H NMR (800 MHz, Methanol-d₄) δ 11.84 (s, 1H), 11.78 (s, 1H), 11.34 (s, 1H), 11.02 (d, J=2.9 Hz, 1H), 10.49 (s, 1H), 8.82 (d, J=10.7 Hz, 1H), 8.73 (d, J=8.1 Hz, 2H), 8.55 (t, J=9.4 Hz, 3H), 8.21 (d, J=7.9 Hz, 1H), 8.05 (t, J=7.7 Hz, 2H), 7.92 (d, J=7.9 Hz, 2H), 7.84 (t, J=7.5 Hz, 1H), 6.32 (s, 1H), 4.36 (s, 3H), 3.32 (s, 2H). MS (ESI): m/z 534.0 [M+H]⁺. HRMS m/z [M+H]⁺ calcd for C₂₆H₂₄N₅O₆S⁺ 534.1442, found 534.1428.

### Example 197: Synthesis of LQ070-91 (JJ147)

### 3-(2-(2,6-dioxo-1,2,3,6-tetrahydropyrimidin-4-yl)acetamido)-N-(4-(N-phenylsulfamoyl)phenyl)benzamide

Compound LQ070-91 was prepared using same procedures as preparing compound LQ070-79 from Compound 12. ¹H NMR (500 MHz, DMSO-d₆) δ 11.00 (s, 1H), 10.89 (s, 1H), 10.59 (s, 1H), 10.36 (s, 1H), 10.19 (s, 1H), 8.08 (s, 1H), 7.91 (d, J=8.8 Hz, 2H), 7.84 (d, J=8.0 Hz, 1H), 7.74 (d, J=8.8 Hz, 2H), 7.65 (d, J=7.7 Hz, 1H), 7.50 (t, J=7.9 Hz, 1H), 7.24 (t, J=7.8 Hz, 2H), 7.10 (d, J=8.0 Hz, 2H), 7.03 (t, J=7.4 Hz, 1H), 5.47 (s, 1H), 3.52 (s, 2H). MS (ESI): m/z 520.1 [M+H]⁺. HRMS m/z [M+H]⁺ calcd for C₂₅H₂₂N₅O₆S⁺ 520.1285, found 520.1274.

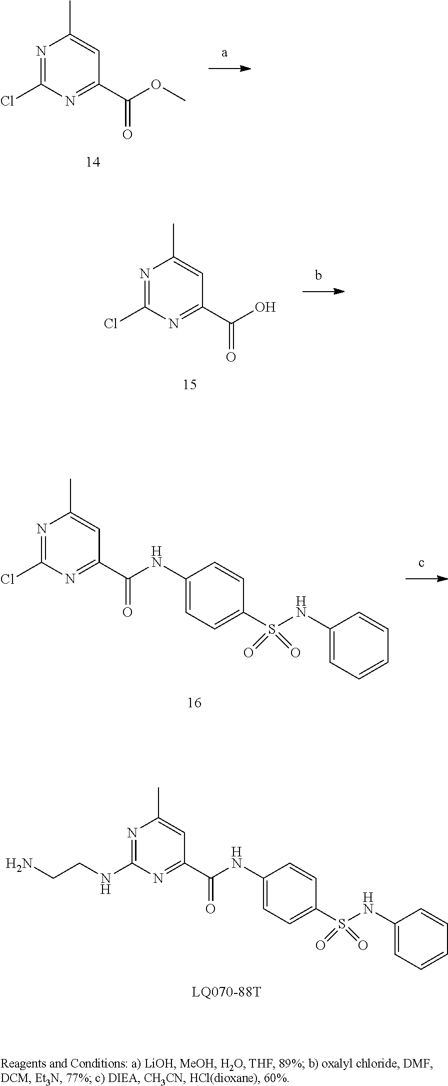 [SMILES: COC(=O)c1cc(C)nc(Cl)n1 ; Cc1cc(C(=O)O)nc(Cl)n1 ; Cc1cc(C(=O)Nc2ccc(S(=O)(=O)Nc3ccccc3)cc2)nc(Cl)n1 ; Cc1cc(C(=O)Nc2ccc(S(=O)(=O)Nc3ccccc3)cc2)nc(NCCN)n1]

### 2-Chloro-6-methylpyrimidine-4-carboxylic acid (15)

To a solution of commercial available methyl 2-Chloro-6-methylpyrimidine-4-carboxylate (50 mg, 0.27 mmol) in 0.5 m L MeOH, 0.5 mL H₂O, and 0.5 mL THF, LiOH (8 mg, 0.3 mmol) was added. The mixture was stirred at RT overnight. Then the mixture was purified by reverse phase column to afford white solid (41 mg, 89%). MS (ESI): m/z 173.0 [M+H]⁺.

### 2-Chloro-6-methyl-N-(4-(N-phenylsulfamoyl)phenyl)pyrimidine-4-carboxamide (16)

To a solution of 2-chloro-6-methylpyrimidine-4-carboxylic acid (15, 83 mg, 0.48 mmol) in DCM were added oxalyl chloride (61 μL, 0.72 mmol) and 1 drop of DMF. The mixture was stirred at room temperature for 2 h, the solvent was removed by evaporation which was used in next step. To a solution of 4-Amino-n-phenyl-benzenesulfonamide (120 mg, 0.48 mmol) and DIEA (200 μL, 1.2 mmol) in acetone was added a solution of acid chloride in DCM dropwise in an ice bath. The mixture was stirred at RT for 1 h. Then quenched with NH₄Cl, the mixture was extracted with ethyl acetate. The combined organic extracts were washed with brine, dried over sodium sulfate, concentrated. The resulting residue was purified by silica gel flash chromatography to give the compound as yellow solid (150 mg, 77%) MS (ESI): m/z 403.1 [M+H]⁺.

### Example 198

### 2-((2-Aminoethyl)amino)-6-methyl-N-(4-(N-phenylsulfamoyl)phenyl)pyrimidine-4-carboxamide

To a solution of Compound 16 (85 mg, 0.21 mmol) and DIEA (42 μL, 0.25 mmol) in acetonitrile tert-Butyl (2-aminoethyl)carbamate (34 mg, 0.21 mmol) was added, and the solution was refluxed for 3 h. After cooling down, the mixture was diluted with ethyl acetate, washed with brine, dried over anhydrous sodium sulfate and then concentrated. The residue was dissolved in HCl (4M in dioxane). The resulting mixture was stirred for 30 min. Then it was concentrated and purified by preparative HPLC to yield the compound. Yield: 60%. ¹H NMR (500 MHz, DMSO-d6) δ 10.23 (s, 1H), 7.96-7.91 (m, 2H), 7.81-7.76 (m, 2H), 7.23 (t, J=7.8 Hz, 2H), 7.16 (s, 1H), 7.10 (d, J=8.0 Hz, 2H), 7.03 (t, J=7.4 Hz, 1H), 3.03 (q, J=6.0 Hz, 2H), 2.40 (s, 3H). MS (ESI): m/z 427.3 [M+H]⁺. HRMS m/z [M+H]⁺ calcd for C₂₀H₂₃N₆O₃S⁺ 427.1547, found 427.2736.

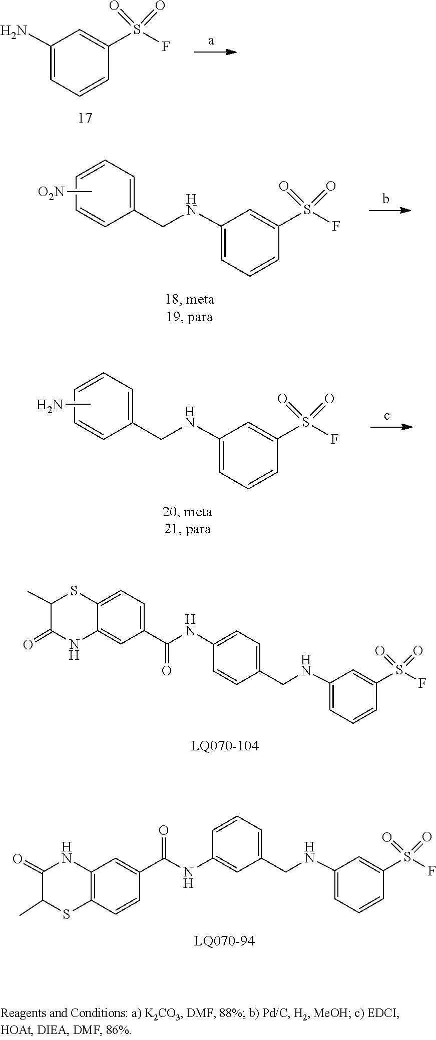

### 3-((3-Nitrobenzyl)amino)benzenesulfonyl fluoride (18)

A solution of 3-Nitrobenzyl bromide (50 mg, 0.23 mmol) and 3-aminobenzene-1-sulfonyl fluoride (41 mg, 0.23 mmol) in 20 mL of DMF was treated with K₂CO₃ (35 mg, 0.25 mmol). The resulting mixture was stirred overnight at RT. After the reaction was completed, the reaction mixture was poured into ice water, aqueous phase was extracted with ethyl acetate. The combined organic phase was washed with brine twice, dried and concentrated. The resulting residue was purified by silica gel flash chromatography to give the compound as yellow oil (63 mg, 88%) MS (ESI): m/z 311.1 [M+H]⁺.

### 3-((4-Nitrobenzyl)amino)benzenesulfonyl fluoride (19)

Compound 19 was prepared using same procedures as preparing compound 18 from 4-Nitrobenzyl bromide. MS (ESI): m/z 311.1 [M+H]⁺.

### 3-((3-Aminobenzyl)amino)benzenesulfonyl fluoride (20)

Compound 20 was prepared using same procedures as preparing compound 3 from Compound 18. MS (ESI): m/z 281.2 [M+H]⁺.

### 3-((4-Aminobenzyl)amino)benzenesulfonyl fluoride (21)

Compound 21 was prepared using same procedures as preparing compound 3 from Compound 19. MS (ESI): m/z 281.2 [M+H]⁺.

### Example 199: Synthesis of LQ070-94 (JJ148)

### 3-((3-(2-Methyl-3-oxo-3,4-dihydro-2H-benzo[b][1,4]thiazine-6-carboxamido)benzyl)amino)benzenesulfonyl fluoride

2-Methyl-3-oxo-3,4-dihydro-2H-1,4-benzothiazine-6-carboxylic acid (46 mg, 0.2 mmol) added to a solution of Compound 20 (58 mg, 0.2 mmol), EDCI (50 mg, 0.26 mmol), HOAt (35 mg, 0.26 mmol), DIEA (60 μL, 0.3 mmol) in 5 mL CH₂Cl₂, the resulting mixture was stirred overnight. Then the organic phase was washed with water and brine, dried over anhydrous Na₂SO₄ and concentrated, the resulting residue was purified by reverse phase column. Yield: 86%. ¹H NMR (500 MHz, DMSO-d₆) δ 10.75 (s, 1H), 10.26 (s, 1H), 7.79 (s, 1H), 7.66 (d, J=8.1 Hz, 1H), 7.58 (d, J=8.1 Hz, 1H), 7.52 (s, 1H), 7.45 (dd, J=22.4, 8.0 Hz, 2H), 7.33 (t, J=7.8 Hz, 1H), 7.22-7.15 (m, 3H), 7.09 (dd, J=26.0, 8.0 Hz, 2H), 4.37 (d, J=5.9 Hz, 2H), 3.74 (q, J=6.9 Hz, 1H), 1.35 (d, J=7.0 Hz, 3H). MS (ESI): m/z 486.2 [M+H]⁺. HRMS m/z [M+H]⁺ calcd for C₂₃H₂₁FN₃O₄S₂⁺ 486.0952, found 486.0937.

### Example 200

### 3-((4-(2-Methyl-3-oxo-3,4-dihydro-2H-benzo[b][1,4]thiazine-6-carboxamido)benzyl)amino)benzenesulfonyl fluoride

Compound LQ070-104 was prepared using same procedures as preparing compound LQ070-104 from Compound 21. ¹H NMR (500 MHz, DMSO-d₆) δ 10.75 (s, 1H), 10.24 (s, 1H), 7.72 (d, J=8.4 Hz, 2H), 7.59 (dd, J=8.2, 1.8 Hz, 1H), 7.53 (d, J=1.8 Hz, 1H), 7.50-7.42 (m, 2H), 7.35 (d, J=8.3 Hz, 2H), 7.20-7.06 (m, 4H), 4.34 (d, J=5.8 Hz, 2H), 3.74 (q, J=7.0 Hz, 1H), 1.35 (d, J=7.0 Hz, 3H). MS (ESI): m/z 486.1 [M+H]⁺. HRMS m/z [M+H]⁺ calcd for C₂₃H₂₁FN₃O₄S₂⁺ 486.0952, found 486.0933.

### Example 201: Synthesis of LQ086-5 (JJ182)

### N-(4-(N-phenylsulfamoyl)phenyl)-[1,2,4]triazolo[4,3-a]pyridine-6-carboxamide

Compound LQ086-5 was prepared using same procedures as preparing compound 16 from [1,2,4]Triazolo[4,3-a]pyridine-6-carboxylic acid and 4-amino-N-phenylbenzene-1-sulfonamide. ¹H NMR (800 MHz, Methanol-d₄) δ 10.01 (s, 1H), 9.73 (s, 1H), 8.37 (d, J=9.6 Hz, 1H), 8.31 (d, J=8.4 Hz, 2H), 8.20-8.14 (m, 4H), 8.12-8.09 (m, 2H), 8.06 (d, J=9.7 Hz, 1H), 7.46 (d, J=8.4 Hz, 2H). HRMS m/z [M+H]⁺ calcd for C₁₉H₁₆N₅O₃S⁺ 394.0968, found 394.1635.

### Example 202: Synthesis of LQ086-6 (JJ183)

### 4-methyl-3-oxo-N-(4-(piperidin-1-ylsulfonyl)phenyl)-3,4-dihydro-2H-benzo[b][1,4]thiazine-6-carboxamide

Compound LQ086-6 was prepared using same procedures as preparing compound 16 from 4-methyl-3-oxo-3,4-dihydro-2H-1,4-benzothiazine-6-carboxylic acid and 4-(piperidine-1-sulfonyl)aniline. HRMS m/z [M+H]⁺ calcd for C₂₁H₂₄N₃O₄S₂⁺ 446.1203, found 446.1194.

### Example 203: Synthesis of LQ086-7 (JJ184)

### N-(4-(phenoxymethyl)benzyl)quinazolin-7-amine

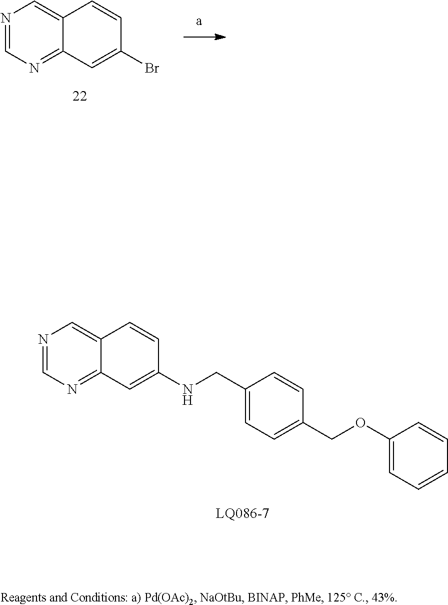 [SMILES: Brc1ccc2cncnc2c1 ; c1ccc(OCc2ccc(CNc3ccc4cncnc4c3)cc2)cc1]

A solution of 7-bromoquinazoline (59 mg, 0.28 mmol), (4-(phenoxymethyl)phenyl)methanamine (60 mg, 0.28 mmol), palladium(II) acetate (3 mg), sodium tert-butoxide (40 mg, 0.42 mmol) and (+/−)-2,2′-Bis(diphenylphosphino)-1,1′-binaphthyl (17 mg, 0.028 mmol) in PhMe (6 mL) under nitrogen atmosphere was heated to 125° C. by microwave for 40 min. After cooling to room temperature, the reaction mixture was purified by reverse phase column. Yield: 43%. ¹H NMR (800 MHz, Methanol-d₄) δ 9.12 (s, 1H), 8.87 (s, 1H), 7.96 (d, J=9.2 Hz, 1H), 7.52-7.42 (m, 4H), 7.39 (d, J=9.2 Hz, 1H), 7.27 (t, J=7.9 Hz, 2H), 6.98 (d, J=8.0 Hz, 2H), 6.94 (t, J=7.4 Hz, 1H), 6.89 (s, 1H), 5.09 (s, 2H), 4.63 (s, 2H). MS (ESI): m/z 342.2 [M+H]⁺. HRMS m/z [M+H]⁺ calcd for C₂₂H₂₀N₃O⁺ 342.1601, found 342.3477.

### Example 204: Synthesis of LQ086-8 (JJ185)

### N-(4-(azepan-1-ylmethyl)benzyl)quinazolin-7-amine

Compound LQ086-8 was prepared using same procedures as preparing compound LQ086-7 from 7-bromoquinazoline and 4-Azepan-1-ylmethyl-benzylamine. ¹H NMR (600 MHz, Methanol-d₄) δ 9.17 (s, 1H), 8.89 (s, 1H), 8.01 (d, J=9.2 Hz, 1H), 7.57-7.54 (m, 4H), 7.42 (dd, J=9.2, 2.2 Hz, 1H), 6.89 (s, 1H), 4.70 (s, 2H), 4.36 (s, 2H), 3.47-3.41 (m, 2H), 3.18 (t, J=11.7 Hz, 2H), 2.01-1.91 (m, 2H), 1.88-1.81 (m, 2H), 1.79-1.70 (m, 4H). MS (ESI): m/z 347.2 [M+H]⁺. HRMS m/z [M+H]⁺ calcd for C₁₉H₁₆N₅O₃S⁺ 347.2230, found 347.2204.

### Example 205: Synthesis of LQ086-10 (JJ186)

### N-(4-(N-methyl-N-phenylsulfamoyl)phenyl)-[1,2,4]triazolo[4,3-a]pyridine-6-carboxamide

Compound LQ086-10 was prepared using same procedures as preparing compound 16 from [1,2,4]Triazolo[4,3-a]pyridine-6-carboxylic acid and 4-amino-N-methyl-N-phenylbenzenesulfonamide. ¹H NMR (800 MHz, DMSO-d₆) δ 10.85 (s, 1H), 9.45 (s, 1H), 9.30 (s, 1H), 7.96 (d, J=8.3 Hz, 2H), 7.92 (d, J=9.5 Hz, 1H), 7.86 (d, J=9.6 Hz, 1H), 7.55 (d, J=8.3 Hz, 1H), 7.37 (t, J=7.6 Hz, 2H), 7.30 (t, J=7.4 Hz, 1H), 7.13 (d, J=7.8 Hz, 2H), 3.16 (s, 3H). MS (ESI): m/z 408.1 [M+H]⁺. HRMS m/z [M+H]⁺ calcd for C₂₀H₁₈N₅O₃S⁺ 408.1125, found 408.1382.

### Example 206: Synthesis of LQ086-11 (JJ210)

### N-(4-(benzyl(methyl)amino)benzyl)quinolin-7-amine

Compound LQ086-11 was prepared using same procedures as preparing compound LQ086-7 from 7-Bromoquinoline and 4-(aminomethyl)-N-benzyl-N-methylaniline. ¹H NMR (600 MHz, Methanol-d₄) δ 8.96 (s, 1H), 8.80 (s, 1H), 7.74 (d, J=9.0 Hz, 1H), 7.30-7.25 (m, 2H), 7.24-7.16 (m, 6H), 6.79-6.73 (m, 3H), 4.53 (s, 2H), 4.36 (s, 2H), 3.00 (s, 3H). HRMS m/z [M+H]⁺. calcd for C₂₄H₂₄N₃⁺ 354.1965, found 354.1854.

### Example 207: Synthesis of LQ086-12 (JJ211)

### N-(4-(benzyl(methyl)amino)benzyl)quinazolin-7-amine

Compound LQ086-12 was prepared using same procedures as preparing compound LQ086-7 from 7-bromoquinazoline and 4-(aminomethyl)-N-benzyl-N-methylaniline. ¹H NMR (600 MHz, Methanol-d₄) δ 8.96 (s, 1H), 8.80 (s, 1H), 7.74 (d, J=9.0 Hz, 1H), 7.30-7.16 (m, 9H), 6.79-6.73 (m, 4H), 4.53 (s, 2H), 4.36 (s, 2H), 3.00 (s, 3H). MS (ESI): m/z 355.2 [M+H]⁺. HRMS m/z [M+H]⁺ calcd for C₂₃H₂₃N₄⁺ 355.1917, found 355.2153.

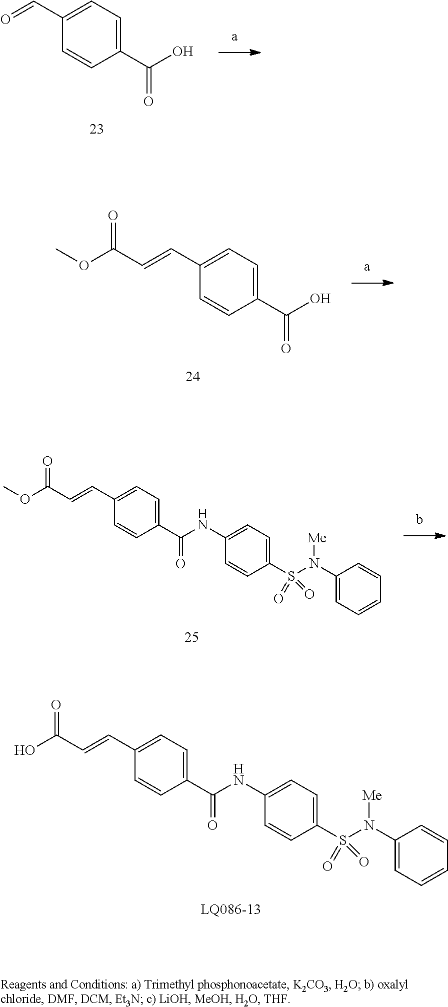 [SMILES: O=Cc1ccc(C(=O)O)cc1 ; COC(=O)/C=C/c1ccc(C(=O)O)cc1 ; COC(=O)/C=C/c1ccc(C(=O)Nc2ccc(S(=O)(=O)N(C)c3ccccc3)cc2)cc1 ; CN(c1ccccc1)S(=O)(=O)c1ccc(NC(=O)c2ccc(/C=C/C(=O)O)cc2)cc1]

### (E)-4-(3-methoxy-3-oxoprop-1-en-1-yl)benzoic acid (24)

4-Formylbenzoic acid (400 mg, 2.7 mmol) and K₂CO₃ (1.1 g, 8 mmol) were dissolved in water, cooled to ice bath. Trimethyl phosphonoacetate was charged dropwise. The reaction was then warmed and stirred at RT for 1.5 h before acidifying to pH=2. The resulting precipitate was filtered and dried, which was used in next step without further purification.

### Methyl (E)-3-(4-((4-(N-methyl-N-phenylsulfamoyl)phenyl)carbamoyl)phenyl)acrylate (25)

Compound 24 was prepared using same procedures as preparing compound 16 from Compound 24 and 4-amino-N-phenylbenzene-1-sulfonamide. MS (ESI): m/z 451.1 [M+H]⁺.

### Example 208: Synthesis of LQ086-22 (JJ213)

### (E)-3-(4-((4-(N-methyl-N-phenylsulfamoyl)phenyl)carbamoyl)phenyl)acrylic acid

Compound LQ086-13 was prepared using same procedures as preparing compound 15 from Compound 25. ¹H NMR (800 MHz, DMSO-d₆) δ 12.58 (s, 1H), 10.70 (s, 1H), 8.00 (t, J=8.9 Hz, 3H), 7.89 (d, J=7.9 Hz, 2H), 7.68 (d, J=16.1 Hz, 1H), 7.51 (d, J=8.4 Hz, 1H), 7.36 (t, J=7.7 Hz, 2H), 7.30 (t, J=7.4 Hz, 1H), 7.13 (d, J=7.8 Hz, 2H), 6.70 (d, J=16.0 Hz, 1H), 3.16 (s, 3H). MS (ESI): m/z 435.4 [M−H]⁻. HRMS m/z [M+H]⁺ calcd for C₂₃H₂₁N₂O₅S⁺ 437.1166, found 437.1148.

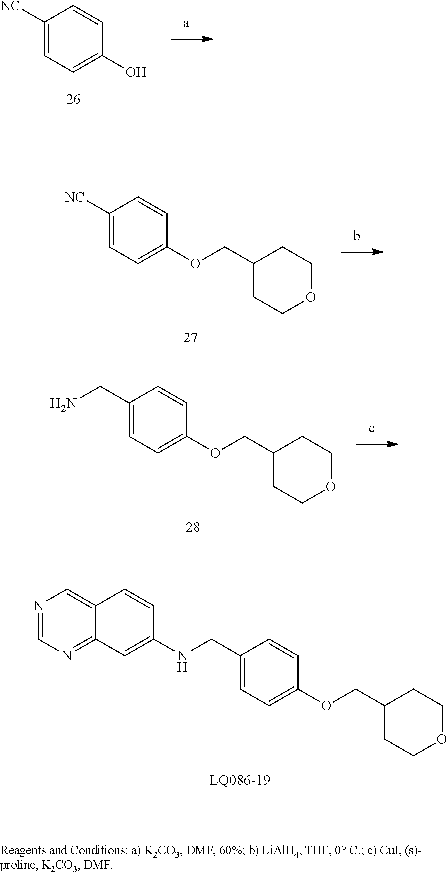 [SMILES: N#Cc1ccc(O)cc1 ; N#Cc1ccc(OCC2CCOCC2)cc1 ; NCc1ccc(OCC2CCOCC2)cc1 ; c1ncc2ccc(NCc3ccc(OCC4CCOCC4)cc3)cc2n1]

### 4-((Tetrahydro-2H-pyran-4-yl)methoxy)benzonitrile (27)

A solution of 4-Cyanophenol (0.49 g, 4.1 mmol) and 4-(Bromomethyl)tetrahydropyran (0.73 g, 4.1 mmol) in 20 mL of DMF was treated with K₂CO₃ (0.67 g, 4.9 mmol). The resulting mixture was stirred overnight at RT. After the reaction was completed, the reaction mixture was poured into ice water, aqueous phase was extracted with ethyl acetate. The combined organic phase was washed with brine twice, dried and concentrated. The resulting residue was purified by silica gel flash chromatography to give the compound as yellow solid (530 mg, 60%)

### (4-((Tetrahydro-2H-pyran-4-yl)methoxy)phenyl)methanamine (28)

4-((tetrahydro-2H-pyran-4-yl)methoxy)benzonitrile (27, 530 mg, 2.4 mmol) was added to a solution of LiAlH₄ (278 mg, 7.3 mmol) in anhydrous THF at 0° C., the resulting mixture was stirred at RT for 18 h. Then cool the reaction flask in ice bath and quenched with water, 2N NaOH. The white suspension was dilute with water and extracted with ethyl acetate. The combined organic extracts were washed with brine, dried over sodium sulfate, concentrated. The resulting residue was purified by reverse phase column. MS (ESI): m/z 205 [M+H−NH₃]⁺.

### Example 209: Synthesis of LQ086-19 (JJ212)

### N-(4-((tetrahydro-2H-pyran-4-yl)methoxy)benzyl)quinazolin-7-amine

A solution of 7-bromoquinazoline (40 mg, 0.2 mmol), (4-((tetrahydro-2H-pyran-4-yl)methoxy)phenyl)methanamine (28, 44 mg, 0.2 mmol), CuI (4 mg), K₂CO₃ (55 mg, 0.4 mmol) and (S)-(−)-Proline (5 mg, 0.04 mmol) in DMF (1 mL) under nitrogen atmosphere was heated to 125° C. by microwave for 40 min. After cooling to room temperature, the reaction mixture was purified by reverse phase column. Yield: 50%. ¹H NMR (600 MHz, Methanol-d₄) δ 9.19 (s, 1H), 8.90 (s, 1H), 7.98 (d, J=9.2 Hz, 1H), 7.40 (dd, J=9.1, 2.1 Hz, 1H), 7.36-7.31 (m, 2H), 6.96-6.89 (m, 3H), 4.54 (s, 2H), 4.01-3.95 (m, 2H), 3.83 (d, J=6.3 Hz, 2H), 3.46 (td, J=11.9, 2.2 Hz, 2H), 2.10-2.00 (m, 1H), 1.79-1.72 (m, 2H), 1.49-1.39 (m, 2H). MS (ESI): m/z 350.2 [M+H]⁺. HRMS m/z [M+H]⁺ calcd for C₂₁H₂₄N₃O₂⁺ 350.1863, found 350.3608.

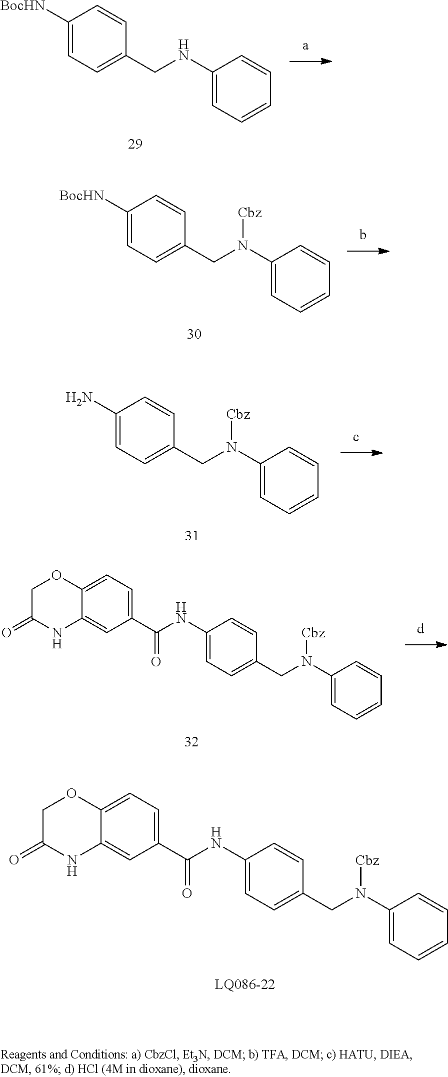 [SMILES: CC(C)(C)OC(=O)Nc1ccc(CNc2ccccc2)cc1 ; CC(C)(C)OC(=O)Nc1ccc(CN(C(=O)OCc2ccccc2)c2ccccc2)cc1 ; Nc1ccc(CN(C(=O)OCc2ccccc2)c2ccccc2)cc1 ; O=C1COc2ccc(C(=O)Nc3ccc(CN(C(=O)OCc4ccccc4)c4ccccc4)cc3)cc2N1 ; O=C1COc2ccc(C(=O)Nc3ccc(CN(C(=O)OCc4ccccc4)c4ccccc4)cc3)cc2N1]

### Benzyl (4-((tert-butoxycarbonyl)amino)benzyl)(phenyl)carbamate (30)

To a stirred solution of tert-butyl (4-((phenylamino)methyl)phenyl)carbamate (290 mg, 0.93 mmol) and triethylamine (195 μL, 1.5 mmol) in DCM was added benzyl chloroformate (150 μL, 1.1 mmol) at 0° C. The resulting mixture was warmed to room temperature to stirred for 3 h and then quenched with NH₄Cl, The mixture was extracted with DCM twice. The combined organic extracts were washed with brine, dried over anhydrous sodium sulfate, filtered and concentrated. The resulting residue was purified by flash column chromatography. ¹H NMR (600 MHz, Methanol-d₄) δ 7.37-7.27 (m, 4H), 7.26-7.21 (m, 3H), 7.18-7.12 (m, 3H), 6.86-6.74 (m, 2H), 4.67 (s, 2H), 4.63 (s, 2H), 1.52 (s, 9H). MS (ESI): m/z 455.3 [M+H]⁺.

### Benzyl (4-aminobenzyl)(phenyl)carbamate (31)

Compound 30 was treated with dichloromethane and trifluoroacetic acid for 1 h. After removal of the solvents, the resulting residue was purified by reverse phase column. MS (ESI): m/z 333.2 [M+H]⁺.

### Benzyl (4-(3-oxo-3,4-dihydro-2H-benzo[b][1,4]oxazine-6-carboxamido)benzyl)(phenyl)carbamate (32)

To the solution of 3-oxo-3,4-dihydro-2H-benzo[b][1,4]oxazine-6-carboxylic acid (23 mg, 0.12 mmol) in DCM were treated with compound 31 (40 mg, 0.12 mmol), HATU (49 mg, 0.13 mmol) and DIEA (24 μL, 0.14 mmol). After being sitting overnight at room temperature, the resulting mixture was purified by reverse phase column to afford the compound 32 as yellow solid (37 mg, 61%) MS (ESI): m/z 508.3 [M+H]⁺.

### Examples 210: Synthesis of LQ086-22 (JJ213)

### 3-oxo-N-(4-((phenylamino)methyl)phenyl)-3,4-dihydro-2H-benzo[b][1,4]oxazine-6-carboxamide

To the solution of compound 32 (37 mg, 0.07 mmol) in dioxane were treated with HCl (4M in dioxane, 0.5 mL). The resulting mixture was heated to 55° C. for 4 h. After cool to room temperature, the mixture was purified by reverse phase column. ¹H NMR (600 MHz, Methanol-d₄) δ 9.05 (dd, J=8.6, 2.8 Hz, 2H), 8.92 (d, J=2.1 Hz, 1H), 8.91-8.86 (m, 1H), 8.65 (d, J=8.1 Hz, 2H), 8.39 (t, J=7.7 Hz, 2H), 8.30 (d, J=8.3 Hz, 1H), 8.03 (d, J=7.9 Hz, 2H), 7.94 (t, J=7.3 Hz, 1H), 5.93 (s, 2H), 5.64 (s, 2H).

### Example 211: Synthesis of LQ086-24 (JJ214)

### N-(4-((phenylamino)methyl)phenyl)-[1,2,4]triazolo[4,3-a]pyridine-6-carboxamide

Compound LQ086-24 was prepared using same procedures as preparing compound LQ086-22 from [1,2,4]Triazolo[4,3-a]pyridine-6-carboxylic acid. ¹H NMR (800 MHz, Methanol-d₄) δ 7.76 (s, 1H), 7.63 (s, 1H), 6.43 (d, J=9.6 Hz, 1H), 6.27 (d, J=9.6 Hz, 1H), 6.16 (d, J=8.0 Hz, 2H), 5.89-5.85 (m, 2H), 5.83-5.75 (m, 3H), 5.70 (d, J=7.8 Hz, 2H), 2.96 (s, 2H). MS (ESI): m/z 344.1 [M+H]⁺. HRMS m/z [M+H]⁺ calcd for C₂₀H₁₈N₅O⁺ 344.1506, found 344.2108.

### Example 212: Synthesis of LQ086-29 (JJ246)

### N-(4-(benzyloxy)-2-(trifluoromethyl)benzyl)quinazolin-7-amine

Compound LQ086-29 was prepared using same procedures as preparing compound LQ086-19 from 4-hydroxy-2-(trifluoromethyl)benzonitrile and benzyl bromide. ¹H NMR (800 MHz, Methanol-d₄) δ 7.61 (s, 1H), 7.35 (s, 1H), 6.43 (d, J=9.1 Hz, 1H), 5.97 (d, J=8.6 Hz, 1H), 5.90 (d, J=7.6 Hz, 2H), 5.87-5.81 (m, 4H), 5.80-5.76 (m, 1H), 5.69 (d, J=8.7 Hz, 1H), 5.27 (s, 1H), 3.62 (s, 2H), 3.14 (s, 2H). MS (ESI): m/z 410.3 [M+H]⁺. HRMS m/z [M+H]⁺ calcd for C₂₃H₁₉F₃N₃O⁺ 410.1475, found 410.2605.

### Example 213: Synthesis of LQ086-30 (JJ247)

### N-(4-(benzyloxy)-3-methylbenzyl)quinazolin-7-amine

Compound LQ086-30 was prepared using same procedures as preparing compound LQ086-19 from 4-Hydroxy-3-methylbenzonitrile and benzyl bromide. ¹H NMR (800 MHz, Methanol-d₄) δ 7.55 (s, 1H), 7.31 (s, 1H), 6.37 (d, J=9.1 Hz, 1H), 5.88 (d, J=7.5 Hz, 2H), 5.83-5.78 (m, 3H), 5.77-5.73 (m, 1H), 5.65 (s, 1H), 5.63 (d, J=8.4 Hz, 1H), 5.40 (d, J=8.3 Hz, 1H), 5.33 (s, 1H), 3.54 (s, 2H), 2.93 (s, 2H), 0.70 (s, 3H). MS (ESI): m/z 356.2 [M+H]⁺. HRMS m/z [M+H]⁺ calcd for C₂₃H₂₂N₃O⁺ 356.1757, found 356.3466.

### Example 214

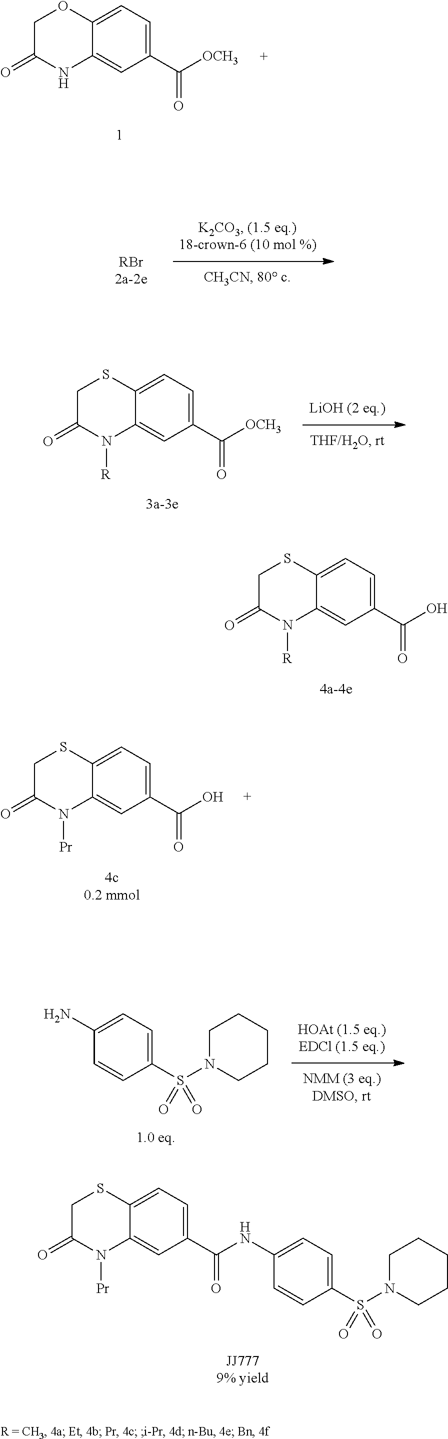

A mixture of carboxylate 1 (446.5 mg, 2 mmol), alkyl chloride 2a-2f (2 mmol), K₂CO₃ (3 mmol), and 18-crown-6 (52.9 mg, 0.2 mmol) in MeCN (5 mL) was stirred at 80° C. for 16 h. The resulting mixture was filtered, and the filtrate was evaporated in vacuo to dryness. The crude residue was dissolved in CH₂Cl₂ (50 mL), and the solution was washed successively with 5% aqueous KCl (50 mL) and water (2×50 mL), dried over Na₂SO₄, and evaporated in vacuo. The obtained residue was purified via ISCO (silica gel, 0-20% EA/Hexane) to afford the title compounds 3a-3f in 41-92% yield (3a: 335.0 mg, 70%, LCMS m/z=238.2244 [M+H]⁺; 3b: 419.9 mg, 83%, LCMS m/z=252.2185 [M+H]⁺; 3c: 429.8 mg, 80%, LCMS m/z=266.1631 [M+H]⁺; 3d: 223.7 mg, 41%, LCMS m/z=266.2630 [M+H]⁺; 3e: 484.2 mg, 86%, LCMS m/z=280.2079 [M+H]⁺; 3f: 605.8 mg, 92%, LCMS m/z=314.1470 [M+H]⁺).

A solution of ester 3a-f (1.0 equiv) and LiOH (2.0 equiv) in a mixture of water and THF (0.15 M, v/v (THF:H₂O)=2:1) was stirred at rt 2 h. The reaction mixture was acidified with 1N HCl until pH3 was reached and then was purified via ISCO (C-18, 50 g, MeOH/H₂O) to afford acids 4a-4f in 70-80% yield (4a: 250.5 mg, 79%, LCMS m/z=224.1807 [M+H]⁺; 4b: 309.1 mg, 78%, LCMS m/z=238.1244 [M+H]⁺; 4c: 389.5 mg, 96%, LCMS m/z=252.1685 [M+H]⁺; 4d: 128.9 mg, 61%, LCMS m/z=252.2185 [M+H]⁺; 4e: 254.0 mg, 55%, LCMS m/z=266.1631 [M+H]⁺; 4f: 526.8 mg, 91%, LCMS m/z=300.1513 [M+H]⁺).

HOAt (1.5 equiv), EDCI (1.5 equiv) and NMM (3.0 equiv) were added to a solution of acid (51.1 mg, 0.2 mmol) and amide (48.6 mg, 0.2 mmol) in DMSO (1 mL). After stirring at rt for 16 h, the mixture was subject to preparative HPLC purification to afford compound JJ177: 8.5 mg, 9% yield. ¹H NMR (600 MHz, Methanol-d₄) δ 8.01-7.95 (m, 2H), 7.85 (d, J=1.8 Hz, 1H), 7.79-7.74 (m, 2H), 7.67 (dd, J=8.0, 1.8 Hz, 1H), 7.57 (d, J=8.1 Hz, 1H), 4.10 (t, J=7.4 Hz, 2H), 3.50 (s, 2H), 2.99 (t, J=5.6 Hz, 4H), 1.64 (p, J=6.6, 5.8 Hz, 4H), 1.48-1.42 (m, 2H), 1.29 (s, 2H), 0.94 (t, J=7.4 Hz, 3H). ESI m/z=474.1 [M+H]⁺.

### Example 215: Synthesis of YH87-046 (JJ178)

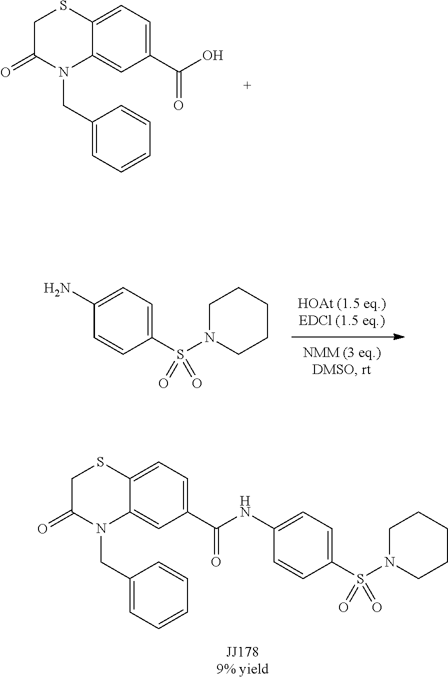 [SMILES: O=C(O)c1ccc2c(c1)N(Cc1ccccc1)C(=O)CS2 ; Nc1ccc(S(=O)(=O)N2CCCCC2)cc1 ; O=C(Nc1ccc(S(=O)(=O)N2CCCCC2)cc1)c1ccc2c(c1)N(Cc1ccccc1)C(=O)CS2]

HOAt (1.5 equiv), EDCI (1.5 equiv) and NMM (3.0 equiv) were added to a solution of acid (62.8 mg, 0.2 mmol) and amide (49.5 mg, 0.2 mmol) in DMSO (1 mL). After stirring at rt for 16 h, the mixture was subject to preparative HPLC purification to afford compound JJ178: 9.2 mg, 9% yield. ¹H NMR (800 MHz, Methanol-d₄) δ 7.91 (d, J=8.3 Hz, 2H), 7.77 (s, 1H), 7.73 (d, J=8.3 Hz, 2H), 7.60 (d, J=8.1 Hz, 1H), 7.54 (d, J=8.0 Hz, 1H), 7.29 (t, J=7.6 Hz, 2H), 7.26 (d, J=7.7 Hz, 2H), 7.21 (t, J=7.4 Hz, 1H), 5.37 (s, 2H), 3.65 (s, 2H), 2.98 (t, J=5.5 Hz, 4H), 1.64 (t, J=5.8 Hz, 4H), 1.47-1.42 (m, 2H). ESI m/z=522.4 [M+H]⁺.

### Example 216

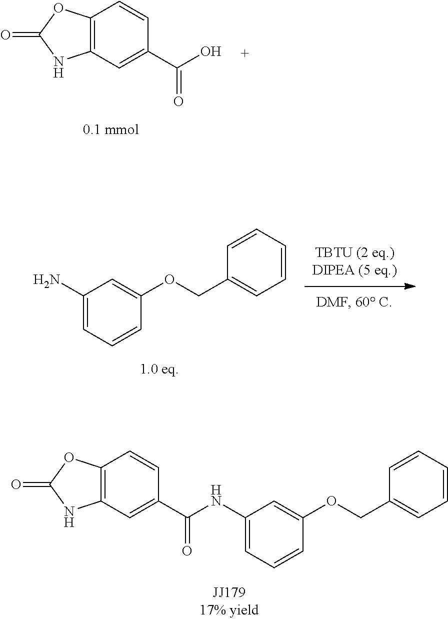 [SMILES: O=C(O)c1ccc2oc(=O)[nH]c2c1 ; Nc1cccc(OCc2ccccc2)c1 ; O=C(Nc1cccc(OCc2ccccc2)c1)c1ccc2oc(=O)[nH]c2c1]

TBTU (2.0 equiv) and DIPEA (5 equiv) were added to a solution of acid (17.7 mg, 0.1 mmol) and amide (23.4 mg, 0.1 mmol, 1.0 equiv) in DMF (1 mL). After stirring at rt for 16 h, the mixture was subject to preparative HPLC purification to afford compound JJ179: 5.9 mg, 17% yield. ¹H NMR (600 MHz, Methanol-d₄) δ 7.74 (dd, J=8.5, 1.9 Hz, 1H), 7.65 (d, J=1.8 Hz, 1H), 7.49-7.43 (m, 3H), 7.38 (t, J=7.5 Hz, 2H), 7.32 (t, J=8.6 Hz, 2H), 7.29-7.21 (m, 2H), 6.81 (d, J=7.8 Hz, 1H), 5.11 (s, 2H). ESI m/z=361.2 [M+H]⁺.

### Example 217

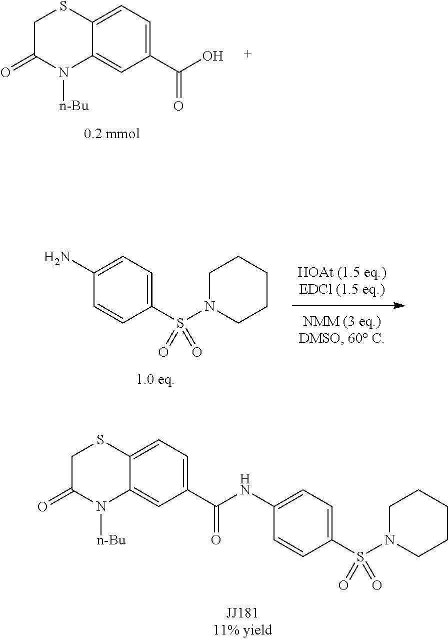

HOAt (1.5 equiv), EDCI (1.5 equiv) and NMM (3.0 equiv) were added to a solution of acid (54.7 mg, 0.2 mmol) and amide (47.8 mg, 0.2 mmol) in DMSO (1 mL). After stirring at rt for 16 h, the mixture was subject to preparative HPLC purification to afford compound JJ181: 10.2 mg, 11% yield. ¹H NMR (800 MHz, Methanol-d₄) δ 7.98 (d, J=8.4 Hz, 2H), 7.86 (s, 1H), 7.76 (d, J=8.3 Hz, 2H), 7.67 (d, J=8.0 Hz, 1H), 7.57 (d, J=8.0 Hz, 1H), 4.14 (t, J=7.5 Hz, 2H), 3.49 (s, 2H), 2.99 (t, J=5.5 Hz, 4H), 1.67-1.58 (m, 6H), 1.45 (t, J=6.1 Hz, 2H), 1.40-1.34 (m, 2H), 0.95 (t, J=7.4 Hz, 3H). ESI m/z=488.1 [M+H]⁺.

### Example 218

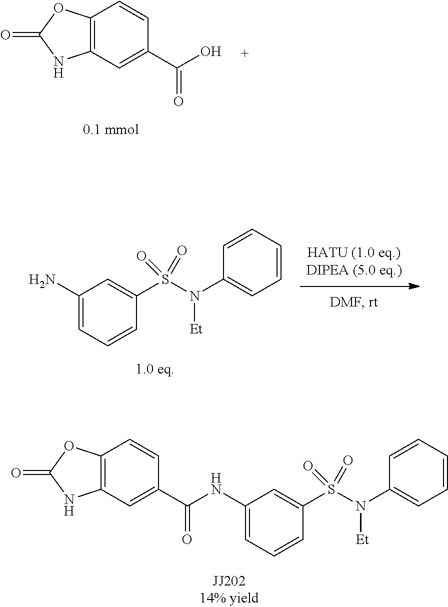 [SMILES: O=C(O)c1ccc2oc(=O)[nH]c2c1 ; CCN(c1ccccc1)S(=O)(=O)c1cccc(N)c1 ; CCN(c1ccccc1)S(=O)(=O)c1cccc(NC(=O)c2ccc3oc(=O)[nH]c3c2)c1]

HATU (1.0 equiv) and DIPEA (5.0 equiv) were added to a solution of acid (23.5 mg, 0.1 mmol) and amide (29.2 mg, 0.1 mmol, 1.0 equiv) in DMF (1 mL). After stirring at rt for 16 h, the mixture was purified via ISCO (C-18, 50 g, MeOH/H₂O) to afford the impure JJ202: 30.4 mg. Then the impure compound was purified via ISCO (silica gel, 12 g, 0-20%-50% EA/Hexane) to afford JJ202: 6.7 mg, 14% yield. ¹H NMR (600 MHz, Acetone-d₆) δ 7.37-7.28 (m, 4H), 7.19 (t, J=8.0 Hz, 2H), 7.12-7.04 (m, 3H), 6.91 (dd, J=8.2, 1.9 Hz, 2H), 6.78 (d, J=7.7 Hz, 1H), 3.63 (q, J=7.1 Hz, 2H), 1.01 (t, J=7.1 Hz, 3H). ESI m/z=437.1 [M]⁺.

### Example 219

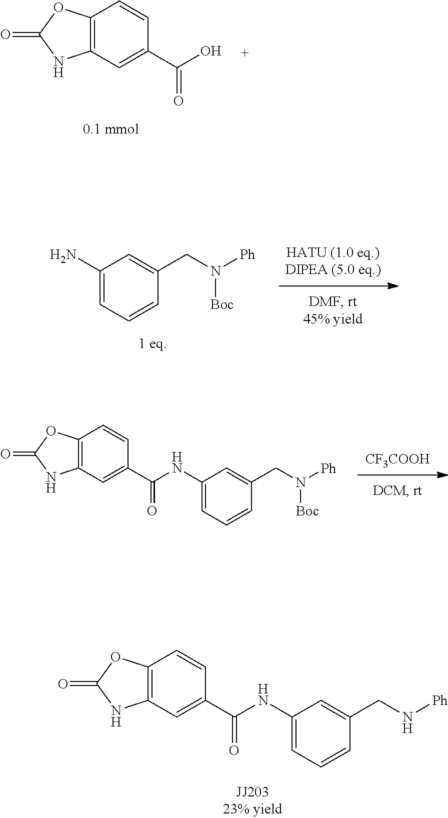 [SMILES: O=C(O)c1ccc2oc(=O)[nH]c2c1 ; CC(C)(C)OC(=O)N(Cc1cccc(N)c1)c1ccccc1 ; CC(C)(C)OC(=O)N(Cc1cccc(NC(=O)c2ccc3oc(=O)[nH]c3c2)c1)c1ccccc1 ; "CF3COOH" ; O=C(Nc1cccc(CNc2ccccc2)c1)c1ccc2oc(=O)[nH]c2c1]

HATU (1.0 equiv) and DIPEA (5.0 equiv) were added to a solution of acid (18.2 mg, 0.1 mmol) and amide (30.4 mg, 0.1 mmol, 1.0 equiv) in DMF (1 mL). After stirring at rt for 16 h, the mixture was purified via ISCO (C-18, 50 g, MeOH/H₂O) to afford the intermediate: 21.1 mg, 45% yield. Then the intermediate was treated with CF₃COOH to remove the Boc to afford the product JJ203: 8.5 mg, 23% yield. ¹H NMR (600 MHz, Acetone-d₆) δ 7.89-7.68 (m, 4H), 7.34-7.26 (m, 2H), 7.16 (d, J=7.5 Hz, 1H), 7.09 (d, J=8.0 Hz, 2H), 6.70 (d, J=8.1 Hz, 2H), 6.61 (d, J=7.8 Hz, 1H), 4.37 (s, 2H). ESI m/z=360.2 [M+H]⁺.

### Example 220

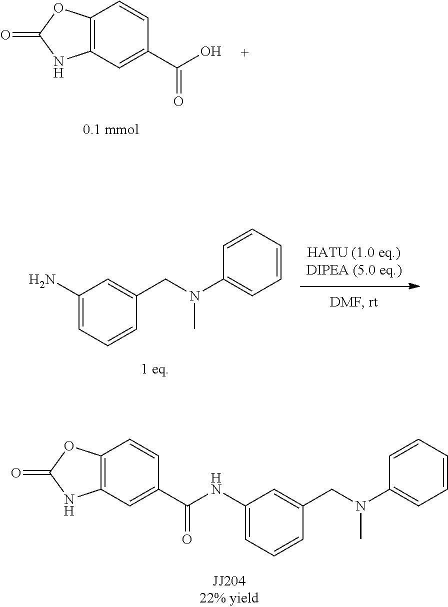 [SMILES: O=C(O)c1ccc2oc(=O)[nH]c2c1 ; CN(Cc1cccc(N)c1)c1ccccc1 ; CN(Cc1cccc(NC(=O)c2ccc3oc(=O)[nH]c3c2)c1)c1ccccc1]

HATU (1.0 equiv) and DIPEA (5.0 equiv) were added to a solution of acid (17.8 mg, 0.1 mmol) and amide (22.5 mg, 0.1 mmol, 1.0 equiv) in DMF (1 mL). After stirring at rt for 16 h, the mixture was subject to preparative HPLC purification to afford compound JJ204: 8.7 mg, 22% yield. ¹H NMR (600 MHz, Acetone-d₆) δ 9.59 (s, 1H), 7.78 (td, J=11.2, 2.4 Hz, 2H), 7.71 (d, J=1.8 Hz, 1H), 7.67 (s, 1H), 7.33-7.27 (m, 2H), 7.18-7.13 (m, 2H), 7.01 (d, J=7.6 Hz, 1H), 6.76 (d, J=10.4 Hz, 2H), 6.63 (d, J=9.6 Hz, 1H), 4.56 (s, 2H), 3.06 (s, 3H). ESI m/z=374.4 [M+H]⁺.

### Example 221

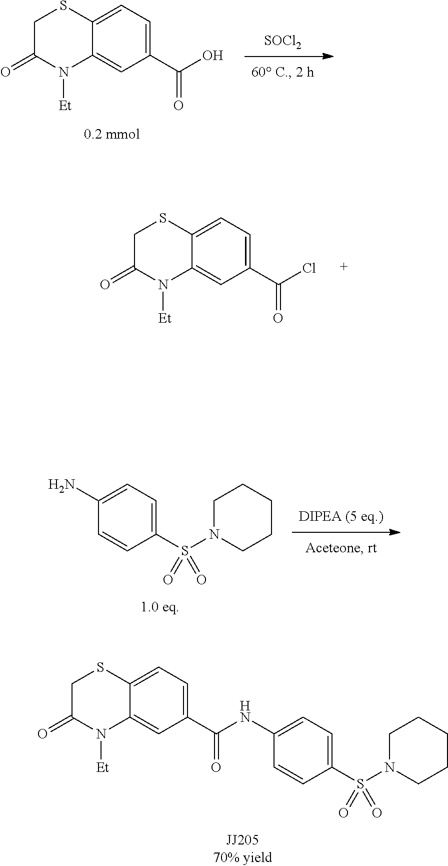 [SMILES: CCN1C(=O)CSc2ccc(C(=O)O)cc21 ; "SOCl2" ; CCN1C(=O)CSc2ccc(C(=O)Cl)cc21 ; Nc1ccc(S(=O)(=O)N2CCCCC2)cc1 ; "DIPEA (5 eq.)" ; CCN1C(=O)CSc2ccc(C(=O)Nc3ccc(S(=O)(=O)N4CCCCC4)cc3)cc21]

A solution of acid (47.9 mg, 0.2 mmol) in thionyl chloride (1 ml) was heated at 60° C. for 2 h. The solvent was removed under reduced pressure and then the residue was used directly in the next step. Amine (44.8 mg, 0.2 mmol) was added to a solution of carbonyl chloride and DIPEA (5.0 equiv) in acetone (1 ml), After stirring overnight at rt, the reaction mixture was then concentrated in vacuo and the residue was purified via ISCO (C-18, 50 g, MeOH/H₂O) to give JJ205: 60.3 mg, 70% yield. ¹H NMR (600 MHz, Acetone-d₆) δ 9.98 (s, 1H), 8.07 (d, J=9.0 Hz, 2H), 7.87 (s, 1H), 7.74 (d, J=8.4 Hz, 2H), 7.69 (dd, J=8.1, 1.7 Hz, 1H), 7.53 (d, J=8.0 Hz, 1H), 4.11 (q, J=7.1 Hz, 2H), 3.50 (s, 2H), 2.97-2.93 (m, 4H), 1.64-1.57 (m, 4H), 1.45-1.38 (m, 2H), 1.24 (t, J=7.1 Hz, 3H). ESI m/z=460.4 [M+H]⁺.

### Example 222

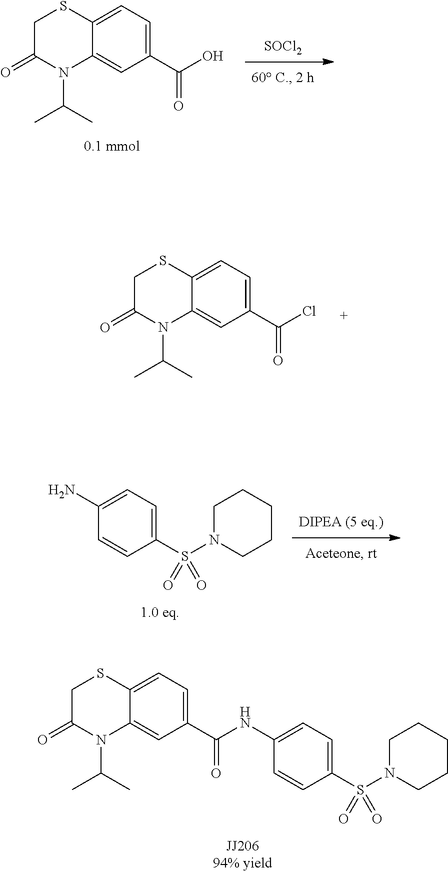 [SMILES: CC(C)N1C(=O)CSc2ccc(C(=O)O)cc21 ; "SOCl2" ; CC(C)N1C(=O)CSc2ccc(C(=O)Cl)cc21 ; Nc1ccc(S(=O)(=O)N2CCCCC2)cc1 ; "DIPEA (5 eq.)" ; CC(C)N1C(=O)CSc2ccc(C(=O)Nc3ccc(S(=O)(=O)N4CCCCC4)cc3)cc21]

Same to the preparation of JJ205, the reaction of acid (27.4 mg, 0.1 mmol), amine (21.3 mg, 0.1 mmol, 1.0 equiv) and DIPEA (5.0 equiv) in acetone afford JJ206: 39.3 mg, 945 yield. ¹H NMR (600 MHz, Acetone-d₆) δ 9.99 (s, 1H), 8.06 (d, J=9.1 Hz, 2H), 7.92 (s, 1H), 7.74 (d, J=6.6 Hz, 2H), 7.70 (dd, J=8.1, 1.8 Hz, 1H), 7.56 (d, J=8.1 Hz, 1H), 4.68-4.60 (m, 1H), 3.40 (s, 2H), 2.95 (t, J=5.5 Hz, 4H), 1.65-1.57 (m, 4H), 1.54 (d, J=6.9 Hz, 6H), 1.45-1.38 (m, 2H). ESI m/z=474.2 [M+H]⁺.

### Example 223

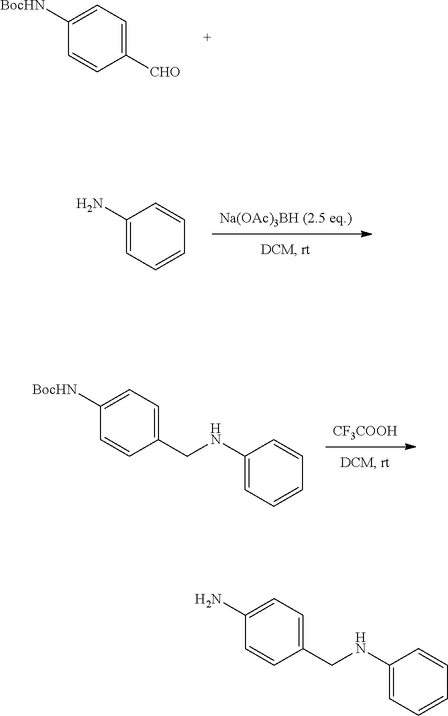 [SMILES: CC(C)(C)OC(=O)Nc1ccc(C=O)cc1 ; Nc1ccccc1 ; "Na(OAc)3BH (2.5 eq.)" ; CC(C)(C)OC(=O)Nc1ccc(CNc2ccccc2)cc1 ; "CF3COOH" ; Nc1ccc(CNc2ccccc2)cc1]

Aldehyde (0.443.3 g, 2.4 mmol), aniline (0.1863 g, 0.2 mL, 2.0 mmol), and sodium triacetoxyhydroborate (1.0570 g, 5.0 mmol) were combined in dichloromethane (10 mL). The reaction mixture was stirred at ambient temperature for 4 hours, diluted with dichloromethane, washed with saturated sodium bicarbonate solution and brine. The organic layer was dried with anhydrous sodium sulfate, filtered and concentrated. The residue was purified by ISCO (silica gel, 2-4% MeOH/Dichloromethane) to afford the intermediate tert-butyl (4-((phenylamino)methyl)phenyl)carbamate (0.5311 g, 89% yield, LCMS m/z=150.0725 [½M+H]⁺). Then the intermediate was treated with CF₃COOH to remove the Boc to afford the product N-(4-aminobenzyl)aniline (LCMS m/z=199.2931 [M+H]⁺) and used directly in the next step.

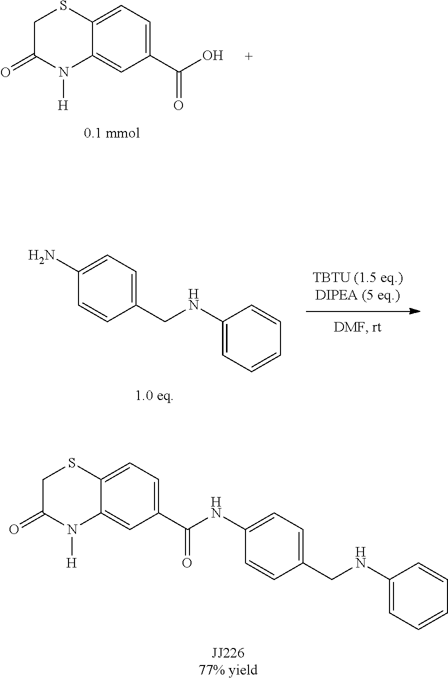 [SMILES: O=C1CSc2ccc(C(=O)O)cc2N1 ; Nc1ccc(CNc2ccccc2)cc1 ; O=C1CSc2ccc(C(=O)Nc3ccc(CNc4ccccc4)cc3)cc2N1]

TBTU (1.5 equiv) and DIPEA (5.0 equiv) were added to a solution of acid (20.9 mg, 0.1 mmol) and amide (19.6 mg, 0.1 mmol, 1.0 equiv) in DMF (2 mL). After stirring at rt for 16 h, the mixture was subject to HPLC purification to afford compound JJ226: 29.7 mg, 77% yield. ¹H NMR (600 MHz, Acetone-d₆) δ 8.59-8.54 (m, 2H), 8.49 (d, J=1.9 Hz, 1H), 8.40 (dd, J=8.1, 1.9 Hz, 1H), 8.23 (d, J=8.1 Hz, 1H), 8.17 (d, J=8.4 Hz, 2H), 7.89-7.82 (m, 2H), 7.47-7.42 (m, 2H), 7.35 (t, J=7.3 Hz, 1H), 5.12 (s, 2H), 4.30 (s, 2H). ESI m/z=388.3 [M−H]⁻, 297.1280 [M−PhNH₂]⁺.

### Example 224

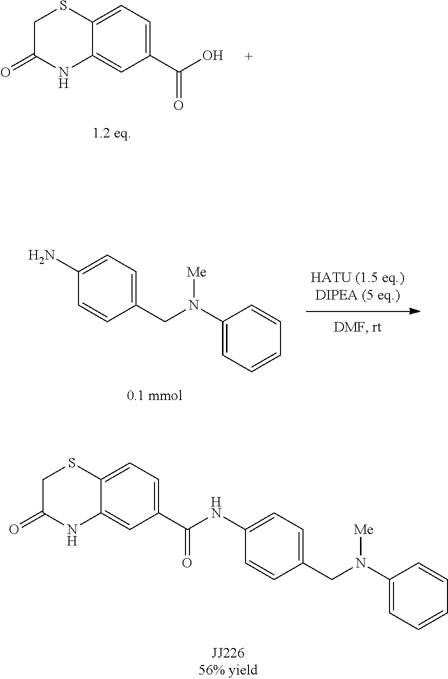 [SMILES: O=C1CSc2ccc(C(=O)O)cc2N1 ; CN(Cc1ccc(N)cc1)c1ccccc1 ; CN(Cc1ccc(NC(=O)c2ccc3c(c2)NC(=O)CS3)cc1)c1ccccc1]

HATU (1.0 equiv) and DIPEA (5.0 equiv) were added to a solution of acid (25.1 mg, 0.12 mmol, 1.2 equiv) and amide (21.6 mg, 0.1 mmol) in DMF (2 mL). After stirring at rt for 16 h, the mixture was subject to HPLC purification to afford compound JJ227: 22.8 mg, 56% yield. ¹H NMR (600 MHz, Methanol-d₄) δ 7.61 (d, J=9.0 Hz, 2H), 7.48 (dd, J=8.1, 1.9 Hz, 1H), 7.43 (d, J=8.0 Hz, 3H), 7.37 (d, J=8.1 Hz, 1H), 7.32 (d, J=8.1 Hz, 3H), 7.17 (d, J=8.5 Hz, 2H), 4.67 (s, 2H), 3.42 (s, 2H), 3.26 (s, 3H). ESI m/z=404.2 [M+H]⁺.

### Example 225

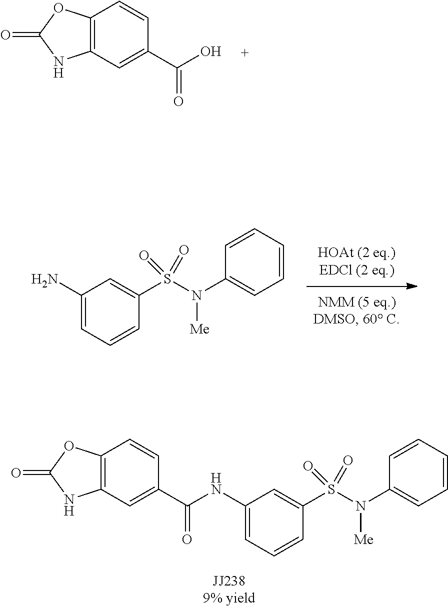 [SMILES: O=C(O)c1ccc2oc(=O)[nH]c2c1 ; CN(c1ccccc1)S(=O)(=O)c1cccc(N)c1 ; CN(c1ccccc1)S(=O)(=O)c1cccc(NC(=O)c2ccc3oc(=O)[nH]c3c2)c1]

HOAt (2.0 equiv), EDCI (2.0 equiv) and NMM (5.0 equiv) were added to a solution of acid (21.7 mg, 0.1 mmol) and amide (26.6 mg, 0.1 mmol, 1.0 equiv) in DMF (2 mL). After stirring at rt for 16 h, the mixture was subject to HPLC purification to afford compound JJ238: 3.8 mg, 9% yield. ¹H NMR (600 MHz, Acetone-d₆) δ 8.22-8.15 (m, 2H), 8.10-8.02 (m, 1H), 7.86-7.77 (m, 2H), 7.51 (t, J=7.9 Hz, 1H), 7.34 (d, J=8.0 Hz, 3H), 7.29 (q, J=7.4 Hz, 1H), 7.16 (dd, J=12.9, 7.7 Hz, 2H), 3.25 (s, 3H). LCMS m/z=424.0 [M+H]⁺.

### Example 226

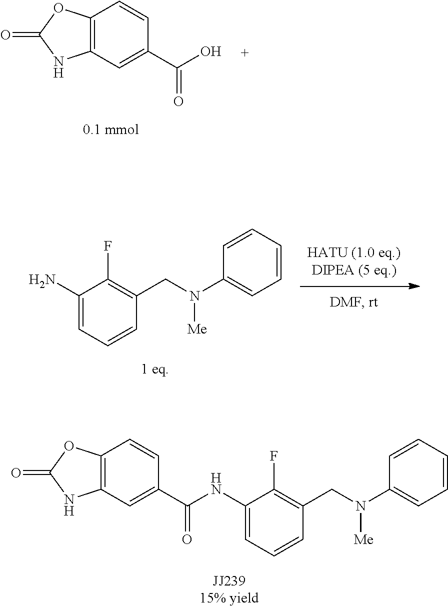 [SMILES: O=C(O)c1ccc2oc(=O)[nH]c2c1 ; CN(Cc1cccc(N)c1F)c1ccccc1 ; CN(Cc1cccc(NC(=O)c2ccc3oc(=O)[nH]c3c2)c1F)c1ccccc1]

HATU (1.0 equiv) and DIPEA (5.0 equiv) were added to a solution of acid (24.0 mg, 0.1 mmol) and amide (21.2 mg, 0.1 mmol, 1.0 equiv) in DMF (2 mL). After stirring at rt for 16 h, the mixture was subject to HPLC purification to afford compound JJ239: 5.9 mg, 15% yield. ¹H NMR (600 MHz, Methanol-d₄) δ 7.76 (dd, J=8.4, 1.8 Hz, 1H), 7.70-7.65 (m, 1H), 7.62 (t, J=10.4 Hz, 1H), 7.36-7.28 (m, 3H), 7.13 (t, J=7.8 Hz, 1H), 7.10-7.03 (m, 3H), 6.99 (t, J=7.6 Hz, 1H), 4.73 (s, 2H), 3.18 (s, 3H). ESI m/z=392.2 [M+H]⁺.

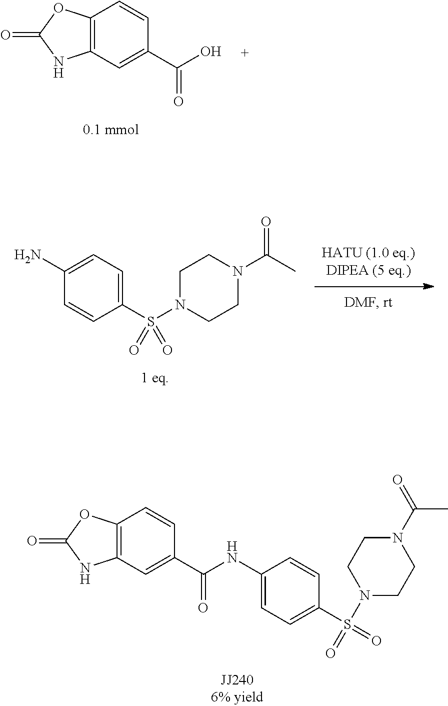 [SMILES: O=C(O)c1ccc2oc(=O)[nH]c2c1 ; CC(=O)N1CCN(S(=O)(=O)c2ccc(N)cc2)CC1 ; CC(=O)N1CCN(S(=O)(=O)c2ccc(NC(=O)c3ccc4oc(=O)[nH]c4c3)cc2)CC1]

### Example 227

HATU (1.0 equiv) and DIPEA (5.0 equiv) were added to a solution of acid (24.4 mg, 0.1 mmol) and amide (27.7 mg, 0.1 mmol, 1.0 equiv) in DMF (2 mL). After stirring at rt for 16 h, the mixture was subject to HPLC purification to afford compound JJ240: 2.8 mg, 6% yield. ¹H NMR (600 MHz, Acetone-d₆) δ 8.04 (d, J=8.7 Hz, 1H), 7.80-7.62 (m, 4H), 7.27 (d, J=8.4 Hz, 2H), 3.85-3.77 (m, 4H), 3.58-3.49 (m, 4H), 1.92 (s, 3H). ESI m/z=445.1 [M+H]⁺.

### Example 228

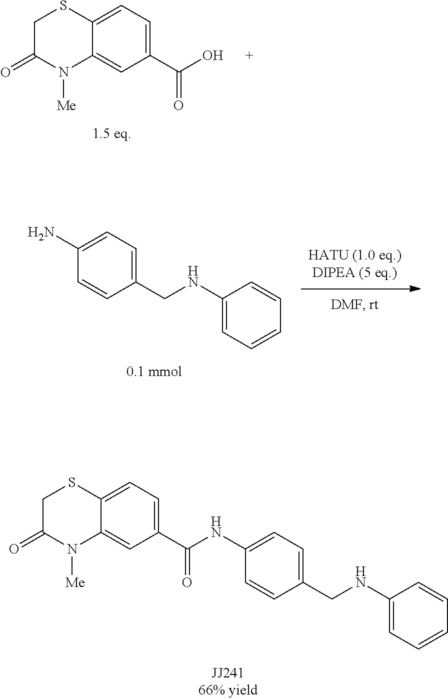 [SMILES: CN1C(=O)CSc2ccc(C(=O)O)cc21 ; Nc1ccc(CNc2ccccc2)cc1 ; CN1C(=O)CSc2ccc(C(=O)Nc3ccc(CNc4ccccc4)cc3)cc21]

HATU (1.0 equiv) and DIPEA (5.0 equiv) were added to a solution of acid (34.0 mg, 0.15 mmol, 1.5 eq.) and amide (20.4 mg, 0.1 mmol) in DMF (2 mL). After stirring at rt for 16 h, the mixture was subject to HPLC purification to afford compound JJ241: 27.3 mg, 66% yield. ¹H NMR (600 MHz, Acetone-d₆) δ 7.85-7.74 (m, 3H), 7.66 (dd, J=8.1, 1.9 Hz, 1H), 7.51 (d, J=7.8 Hz, 1H), 7.38 (d, J=8.4 Hz, 2H), 7.16 (d, J=7.2 Hz, 2H), 6.85 (d, J=8.0 Hz, 2H), 6.76 (t, J=7.2 Hz, 1H), 4.41 (s, 2H), 3.51 (s, 2H), 3.46 (s, 3H). ESI m/z=402.2 [M−H]⁻, 311.0979 [M−PhNH₂]⁺.

### Example 229

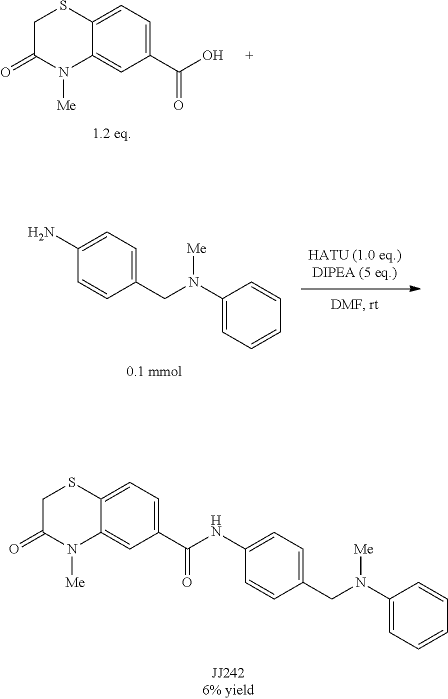 [SMILES: CN1C(=O)CSc2ccc(C(=O)O)cc21 ; CN(Cc1ccc(N)cc1)c1ccccc1 ; CN(Cc1ccc(NC(=O)c2ccc3c(c2)N(C)C(=O)CS3)cc1)c1ccccc1]

HATU (1.0 equiv) and DIPEA (5.0 equiv) were added to a solution of acid (27.4 mg, 0.12 mmol, 1.2 eq.) and amide (19.2 mg, 0.1 mmol) in DMF (2 mL). After stirring at rt for 16 h, the mixture was subject to HPLC purification to afford compound JJ242: 2.2 mg, 6% yield. ¹H NMR (600 MHz, Acetone-d₆) δ 7.56-7.48 (m, 1H), 7.31 (d, J=7.7 Hz, 3H), 7.24-7.18 (m, 5H), 7.06 (s, 2H), 7.02 (dd, J=7.9, 1.7 Hz, 1H), 3.89 (s, 2H), 3.43 (s, 3H), 3.40 (s, 3H), 3.11 (s, 2H). ESI m/z=418.2 [M+H]⁺.

### Example 230

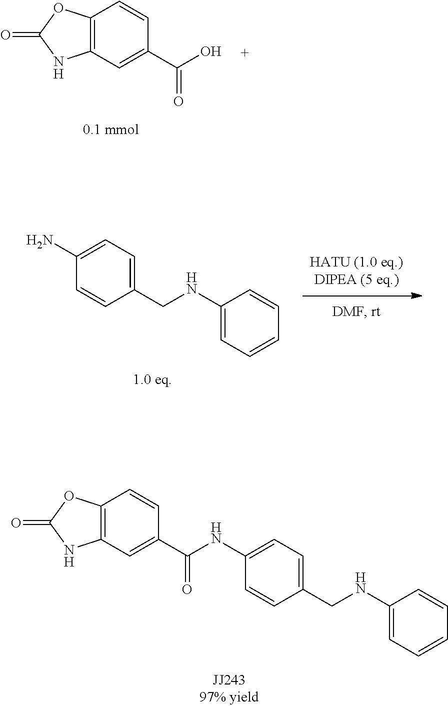 [SMILES: O=C(O)c1ccc2oc(=O)[nH]c2c1 ; Nc1ccc(CNc2ccccc2)cc1 ; O=C(Nc1ccc(CNc2ccccc2)cc1)c1ccc2oc(=O)[nH]c2c1]

HATU (1.0 equiv) and DIPEA (5.0 equiv) were added to a solution of acid (19.3 mg, 0.1 mmol) and amide (19.9 mg, 0.1 mmol, 1.0 equiv) in DMF (2 mL). After stirring at rt for 16 h, the mixture was subject to HPLC purification to afford compound JJ243: 34.9 mg, 97% yield. ¹H NMR (600 MHz, Acetone-d₆) δ 7.81-7.75 (m, 3H), 7.73 (d, J=1.8 Hz, 1H), 7.39 (d, J=8.3 Hz, 2H), 7.33 (d, J=8.3 Hz, 1H), 7.13 (d, J=7.8 Hz, 1H), 6.79 (d, J=7.8 Hz, 1H), 6.69 (dd, J=624.9, 7.5 Hz, 0H), 4.39 (s, 2H). ESI m/z=719.3 [2M+H]⁺.

### Example 231

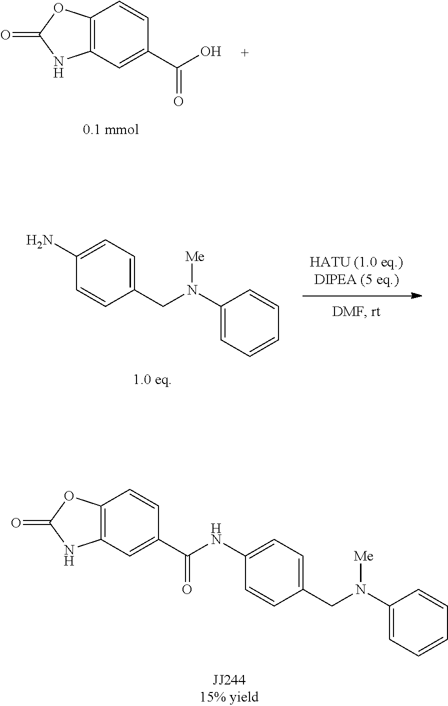 [SMILES: O=C(O)c1ccc2oc(=O)[nH]c2c1 ; CN(Cc1ccc(N)cc1)c1ccccc1 ; CN(Cc1ccc(NC(=O)c2ccc3oc(=O)[nH]c3c2)cc1)c1ccccc1]

HATU (1.0 equiv) and DIPEA (5.0 equiv) were added to a solution of acid (20.3 mg, 0.1 mmol) and amide (22.5 mg, 0.1 mmol, 1.0 equiv) in DMF (2 mL). After stirring at rt for 16 h, the mixture was subject to HPLC purification to afford compound JJ244: 6.1 mg, 15% yield. ¹H NMR (600 MHz, Acetone-d₆) δ 7.82-7.70 (m, 4H), 7.32 (d, J=8.4 Hz, 1H), 7.29-7.22 (m, 4H), 7.04 (d, J=8.1 Hz, 2H), 6.87 (t, J=7.3 Hz, 1H), 4.64 (s, 2H), 3.14 (s, 3H). ESI m/z=374.2 [M+H]⁺.

### Example 232

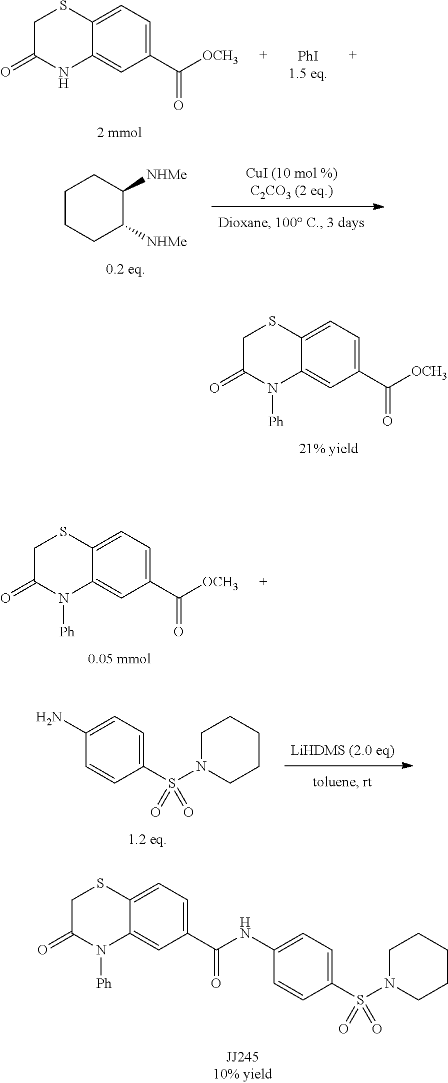

A suspension of 1,4-benzothiazin-3-one (2 mmol), iodobenzene (3 mmol), copper (I) iodide (0.1 mmol), trans-N,N-dimethylcyclohexane-1,2-diamine (0.4 mmol), and cesium carbonate (4 mmol) in dry 1,4-dioxane (4 mL) in a vial. The vial was then capped tightly. The mixture was heated at 100° C. (oil bath temperature) for 3 days. After cooling to rt, filtration was carried out. The combined filtrates were concentrated on rotavap and the residue was subjected to silica gel column chromatograph purification (20-30% ethyl acetate in hexane), furnishing desired products as a solid: 135.1 mg, 21% yield.

An oven-dried vial equipped with a stir bar was charged with an ester substrate (1.0 equiv), amine (1.2 equiv) placed under a positive pressure of nitrogen, and subjected to three evacuation/backfilling cycles. Toluene (0.25 M) and LiHMDS (1.0 M in THF, 2.0 equiv) were sequentially added with vigorous stirring at room temperature, and the reaction mixture was stirred at room temperature. Then the reaction mixture was quenched with aqueous NH₄Cl solution (1.0 M, 1 mL), diluted with EtOAc (10 mL), the organic layer was washed with water (1×10 mL), brine (1×10 mL), dried and concentrated. Purification via preparative HPLC (MeOH/H₂O) afforded the title product JJ245: 2.7 mg, 10% yield. ¹H NMR (600 MHz, Acetone-d₆) δ 9.86 (s, 1H), 7.96 (d, J=8.9 Hz, 2H), 7.76-7.65 (m, 3H), 7.60 (d, J=8.0 Hz, 1H), 7.55 (t, J=7.8 Hz, 2H), 7.46 (t, J=7.4 Hz, 1H), 7.29 (d, J=7.8 Hz, 2H), 7.16 (d, J=1.8 Hz, 1H), 3.72 (s, 2H), 2.92 (d, J=5.7 Hz, 4H), 1.63-1.56 (m, 4H), 1.47-1.36 (m, 2H). ESI m/z=508.2 [M+H]⁺.

TABLE 2
Additional compounds that can be made according to the above methods.

| Examples | Structure | Name |
|---|---|---|
| 233 | 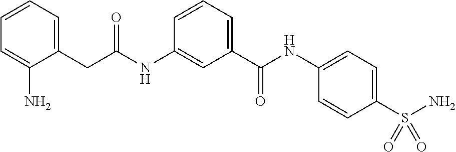 [SMILES: Nc1ccccc1CC(=O)Nc1cccc(C(=O)Nc2ccc(S(N)(=O)=O)cc2)c1] | 3-(2-(2-aminophenyl)acetamido)-N- (4-sulfamoylphenyl)benzamide |
| 234 | 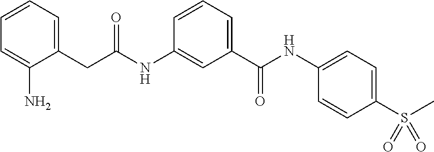 [SMILES: CS(=O)(=O)c1ccc(NC(=O)c2cccc(NC(=O)Cc3ccccc3N)c2)cc1] | 3-(2-(2-aminophenyl)acetamido)-N- (4- (methylsulfonyl)phenyl)benzamide |
| 235 | 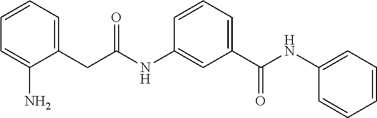 [SMILES: Nc1ccccc1CC(=O)Nc1cccc(C(=O)Nc2ccccc2)c1] | 3-(2-(2-aminophenyl)acetamido)-N- phenylbenzamide |
| 236 | 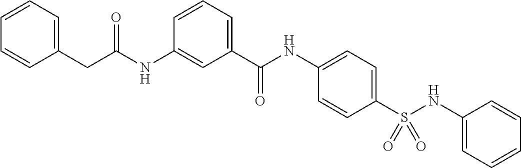 [SMILES: O=C(Cc1ccccc1)Nc1cccc(C(=O)Nc2ccc(S(=O)(=O)Nc3ccccc3)cc2)c1] | 3-(2-phenylacetamido)-N-(4-(N- phenylsulfamoyl)phenyl)benzamide |
| 237 | 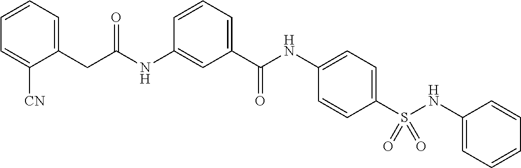 [SMILES: N#Cc1ccccc1CC(=O)Nc1cccc(C(=O)Nc2ccc(S(=O)(=O)Nc3ccccc3)cc2)c1] | 3-(2-(2-cyanophenyl)acetamido)-N- (4-(N- phenylsulfamoyl)phenyl)benzamide |
| 238 | 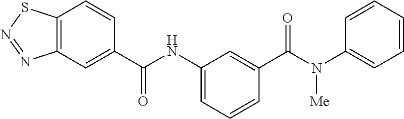 [SMILES: CN(C(=O)c1cccc(NC(=O)c2ccc3snnc3c2)c1)c1ccccc1] | N-(3- (methyl(phenyl)carbamoyl)phenyl) benzo[d][1,2,3]thiadiazole-5- carboxamide |
| 239 | 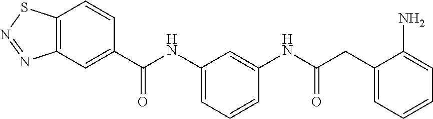 [SMILES: Nc1ccccc1CC(=O)Nc1cccc(NC(=O)c2ccc3snnc3c2)c1] | N-(3-(2-(2- aminophenyl)acetamido)phenyl)benzo [d][1,2,3]thiadiazole-5-carboxamide |
| 240 | 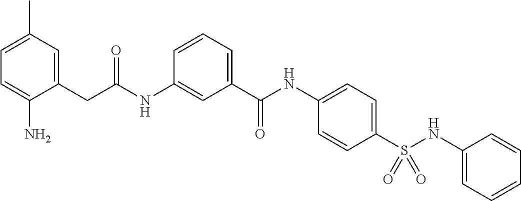 [SMILES: Cc1ccc(N)c(CC(=O)Nc2cccc(C(=O)Nc3ccc(S(=O)(=O)Nc4ccccc4)cc3)c2)c1] | 3-(2-(2-amino-5- methylphenyl)acetamido)-N-(4-(N- phenylsulfamoyl)phenyl)benzamide |
| 241 | 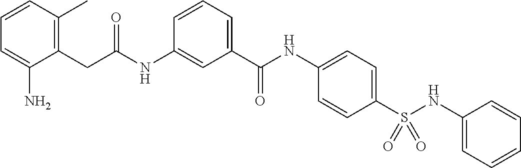 [SMILES: Cc1cccc(N)c1CC(=O)Nc1cccc(C(=O)Nc2ccc(S(=O)(=O)Nc3ccccc3)cc2)c1] | 3-(2-(2-amino-6- methylphenyl)acetamido)-N-(4-(N- phenylsulfamoyl)phenyl)benzamide |
| 242 | 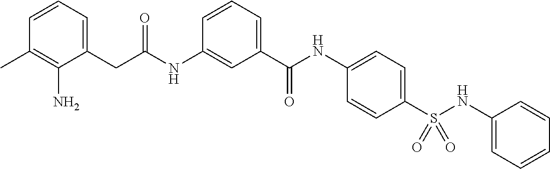 [SMILES: Cc1cccc(CC(=O)Nc2cccc(C(=O)Nc3ccc(S(=O)(=O)Nc4ccccc4)cc3)c2)c1N] | 3-(2-(2-amino-3- methylphenyl)acetamido)-N-(4-(N- phenylsulfamoyl)phenyl)benzamide |
| 243 | 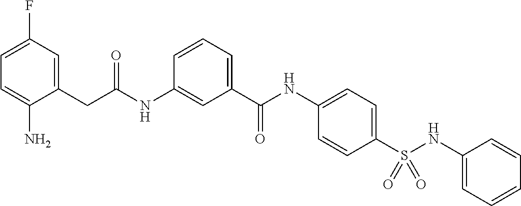 [SMILES: Nc1ccc(F)cc1CC(=O)Nc1cccc(C(=O)Nc2ccc(S(=O)(=O)Nc3ccccc3)cc2)c1] | 3-(2-(2-amino-5- fluorophenyl)acetamido)-N-(4-(N- phenylsulfamoyl)phenyl)benzamide |
| 244 | 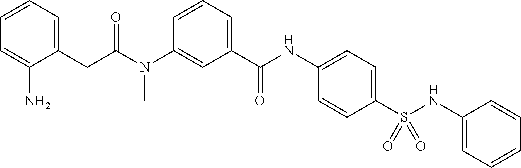 [SMILES: CN(C(=O)Cc1ccccc1N)c1cccc(C(=O)Nc2ccc(S(=O)(=O)Nc3ccccc3)cc2)c1] | 3-(2-(2-aminophenyl)-N- methylacetamido)-N-(4-(N- phenylsulfamoyl)phenyl)benzamide |
| 245 | 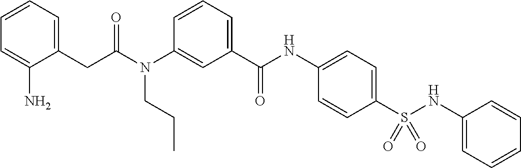 [SMILES: CCCN(C(=O)Cc1ccccc1N)c1cccc(C(=O)Nc2ccc(S(=O)(=O)Nc3ccccc3)cc2)c1] | 3-(2-(2-aminophenyl)-N- propylacetamido)-N-(4-(N- phenylsulfamoyl)phenyl)benzamide |
| 246 | 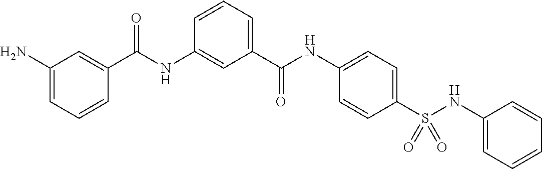 [SMILES: Nc1cccc(C(=O)Nc2cccc(C(=O)Nc3ccc(S(=O)(=O)Nc4ccccc4)cc3)c2)c1] | 3-amino-N-(3-((4-(N- phenylsulfamoyl)phenyl)carbamoyl) phenyl)benzamide |
| 247 | 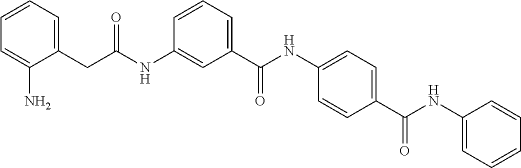 [SMILES: Nc1ccccc1CC(=O)Nc1cccc(C(=O)Nc2ccc(C(=O)Nc3ccccc3)cc2)c1] | 3-(2-(2-aminophenyl)acetamido)-N- (4- (phenylcarbamoyl)phenyl)benzamide |
| 248 | 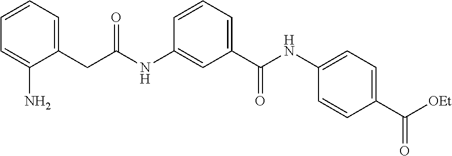 [SMILES: CCOC(=O)c1ccc(NC(=O)c2cccc(NC(=O)Cc3ccccc3N)c2)cc1] | ethyl 4-(3-(2-(2- aminophenyl)acetamido)benzamido) benzoate |
| 249 | 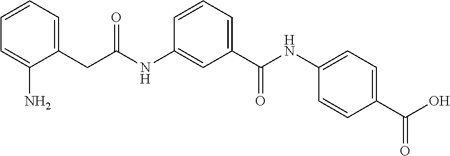 [SMILES: Nc1ccccc1CC(=O)Nc1cccc(C(=O)Nc2ccc(C(=O)O)cc2)c1] | 4-(3-(2-(2- aminophenyl)acetamido)benzamido) benzoic acid |
| 250 | 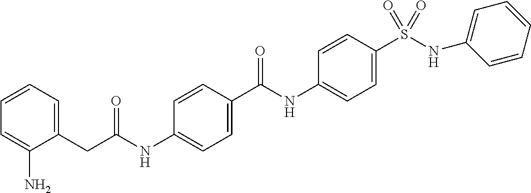 [SMILES: Nc1ccccc1CC(=O)Nc1ccc(C(=O)Nc2ccc(S(=O)(=O)Nc3ccccc3)cc2)cc1] | 4-(2-(2-aminophenyl)acetamido)-N- (4-(N- phenylsulfamoyl)phenyl)benzamide |
| 251 | 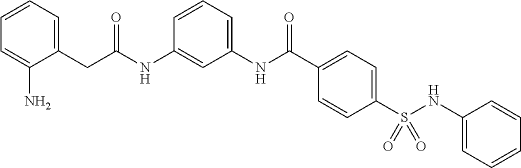 [SMILES: Nc1ccccc1CC(=O)Nc1cccc(NC(=O)c2ccc(S(=O)(=O)Nc3ccccc3)cc2)c1] | N-(3-(2-(2- aminophenyl)acetamido)phenyl)-4- (N-phenylsulfamoyl)benzamide |
| 252 | 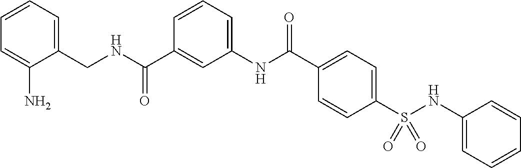 [SMILES: Nc1ccccc1CNC(=O)c1cccc(NC(=O)c2ccc(S(=O)(=O)Nc3ccccc3)cc2)c1] | N-(2-aminobenzyl)-3-(4-(N- phenylsulfamoyl)benzamido) benzamide |
| 253 | 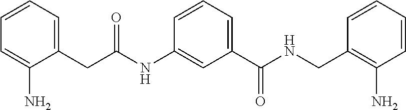 [SMILES: Nc1ccccc1CNC(=O)c1cccc(NC(=O)Cc2ccccc2N)c1] | N-(2-aminobenzyl)-3-(2-(2- aminophenyl)acetamido)benzamide |
| 254 | 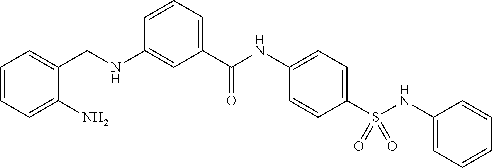 [SMILES: Nc1ccccc1CNc1cccc(C(=O)Nc2ccc(S(=O)(=O)Nc3ccccc3)cc2)c1] | 3-((2-aminobenzyl)amino)-N-(4-(N- phenylsulfamoyl)phenyl)benzamide |
| 255 | 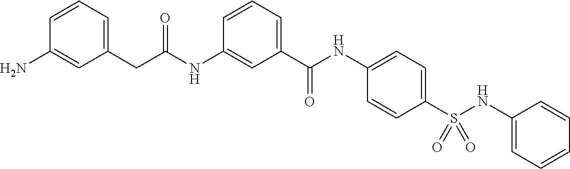 [SMILES: Nc1cccc(CC(=O)Nc2cccc(C(=O)Nc3ccc(S(=O)(=O)Nc4ccccc4)cc3)c2)c1] | 3-(2-(3-aminophenyl)acetamido)-N- (4-(N- phenylsulfamoyl)phenyl)benzamide |
| 256 | 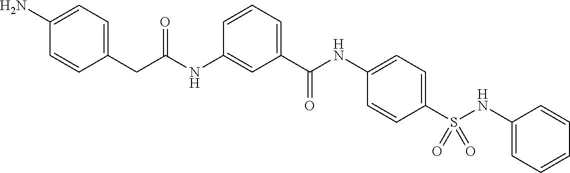 [SMILES: Nc1ccc(CC(=O)Nc2cccc(C(=O)Nc3ccc(S(=O)(=O)Nc4ccccc4)cc3)c2)cc1] | 3-(2-(4-aminophenyl)acetamido)-N- (4-(N- phenylsulfamoyl)phenyl)benzamide |
| 257 | 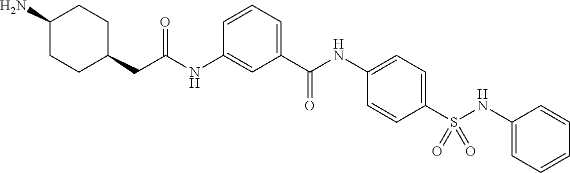 [SMILES: N[C@H]1CC[C@@H](CC(=O)Nc2cccc(C(=O)Nc3ccc(S(=O)(=O)Nc4ccccc4)cc3)c2)CC1] | 3-(2-((1S,4S)-4- aminocyclohexyl)acetamido)-N-(4- (N- phenylsulfamoyl)phenyl)benzamide |
| 258 | 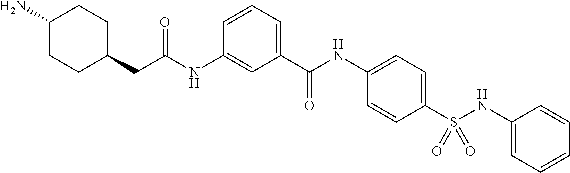 [SMILES: N[C@H]1CC[C@H](CC(=O)Nc2cccc(C(=O)Nc3ccc(S(=O)(=O)Nc4ccccc4)cc3)c2)CC1] | 3-(2-((1R,4R)-4- aminocyclohexyl)acetamido)-N-(4- (N- phenylsulfamoyl)phenyl)benzamide |
| 259 | 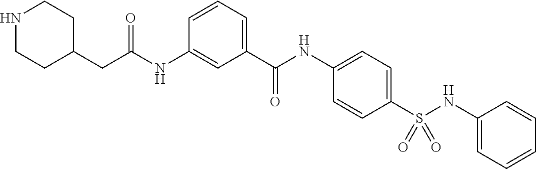 [SMILES: O=C(CC1CCNCC1)Nc1cccc(C(=O)Nc2ccc(S(=O)(=O)Nc3ccccc3)cc2)c1] | tert-butyl 4-(2-oxo-2-((3-((4-(N- phenylsulfamoyl)phenyl)carbamoyl) phenyl)amino)ethyl)piperidine-1- carboxylate |
| 260 | 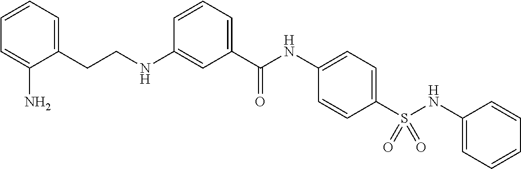 [SMILES: Nc1ccccc1CCNc1cccc(C(=O)Nc2ccc(S(=O)(=O)Nc3ccccc3)cc2)c1] | 3-((2-aminophenethyl)amino)-N-(4- (N- phenylsulfamoyl)phenyl)benzamide |
| 261 | 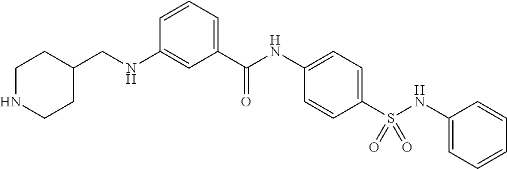 [SMILES: O=C(Nc1ccc(S(=O)(=O)Nc2ccccc2)cc1)c1cccc(NCC2CCNCC2)c1] | N-(4-(N-phenylsulfamoyl)phenyl)-3- ((piperidin-4- ylmethyl)amino)benzamide |
| 262 | 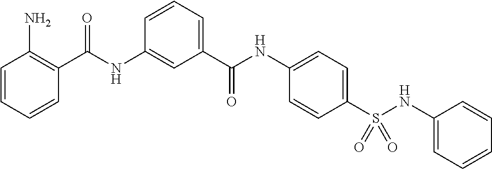 [SMILES: Nc1ccccc1C(=O)Nc1cccc(C(=O)Nc2ccc(S(=O)(=O)Nc3ccccc3)cc2)c1] | 2-amino-N-(3-((4-(N- phenylsulfamoyl)phenyl)carbamoyl) phenyl)benzamide |
| 263 | 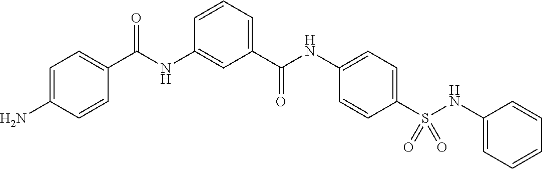 [SMILES: Nc1ccc(C(=O)Nc2cccc(C(=O)Nc3ccc(S(=O)(=O)Nc4ccccc4)cc3)c2)cc1] | 3-(4-aminobenzamido)-N-(4-(N- phenylsulfamoyl)phenyl)benzamide |

### Biology

### Cell Viability Assay

Cell viability was determined by a fluorometric resazurin reduction method (CellTiter-Blue; Promega, Madison, Wis.) following the manufacturer's instructions. 100,000 cells in 100 μl of RPMI 1640 medium were plated in 96 well flat bottom Falcon Polystyrene Microplates (Corning, Corning, N.Y., USA) and treated with compounds in the concentration range of 0-40 μM (8 replicates per condition). Cells were incubated for 72 hours. After incubation, 20 μl CellTiter-Blue Cell was added to each well and incubated for another 2 hours. Plates were put into a fluorescence plate reader that records fluorescence at 560/590 nm to get optical density (OD) values. The number of viable cells in each treated well was calculated, based on the linear least squares regression of the standard curve (OD vs. cell concentration). The viability of cells treated with compounds was normalized to the viability of cells treated with 0.2% Dimethyl sulfoxide (DMSO). Cell counts were confirmed with Trypan Blue Exclusion Assay on the Countess automated cell counter (Invitrogen) according to the manufacturer's specifications. The average and mean standard deviation were calculated from three separate experiments. IC₅₀ values were calculated using the GraphPad Prism (version 4.00) based on a sigmoidal dose-response equation. The data is shown in Table 3 below (JeKo-1 Viability at 20 μM (%) and JeKo-1 IC₅₀ (μM)).

### Surface Plasmon Resonance (SPR) Assay

Recombinant (His)₆-SOX11-DBD protein was immobilized at a flow rate of 10 μl/min for 180 seconds to the right channel of a gold-coated SCR NiHC200M sensor chip (XanTec bioanalytics) placed in a Reichert 2SPR instrument (Reichert Technologies). The left channel, without immobilized protein, was used as a reference. For binding kinetic affinity measurements, compounds were subject to a 2-fold dilution series (range 5-40 μM) in assay buffer (25 mM Tris pH 7.4, 150 mM NaCl, 5 mM MgCl2, 0.005% Tween-20, 2% DMSO) and injected at a flow rate of 25 μl/min at 25° C. Compounds flowed sequentially from the left to the right channel over the prepared sensor chip surface. 120 seconds association phase with each compound concentration was followed by 300 seconds dissociation phase with the assay buffer alone. The kₐ, k_{d}, and KD values were determined from the sensograms for at least 3 concentrations of each compound by curve fitting the data using Reichert's TraceDrawer software. The mean KD value for each compound was calculated from three separate experiments. The data is shown in Table 3 below (KD (M)).

TABLE 3

| | | Jeko-1 | Jeko-1 |
|---|---|---|---|
| | | Viability at | (SOX11+) |
| Structure | KD (M) | 20 μM (%) | IC50 (μM) |
| 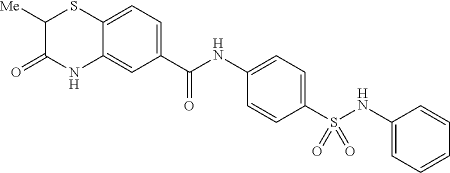 [SMILES: CC1Sc2ccc(C(=O)Nc3ccc(S(=O)(=O)Nc4ccccc4)cc3)cc2NC1=O] | 3.80E−05 | 0.83 | 12.5 |
| 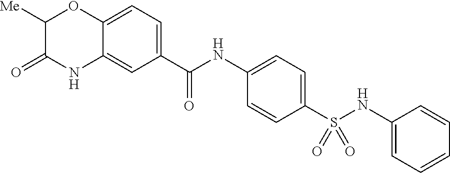 [SMILES: CC1Oc2ccc(C(=O)Nc3ccc(S(=O)(=O)Nc4ccccc4)cc3)cc2NC1=O] | 1.53E−05 | 9.09 | 12.0 |
| 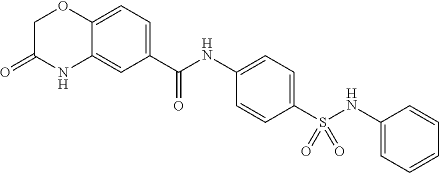 [SMILES: O=C1COc2ccc(C(=O)Nc3ccc(S(=O)(=O)Nc4ccccc4)cc3)cc2N1] | 1.79E−05 | 61.11 | >20 |
| 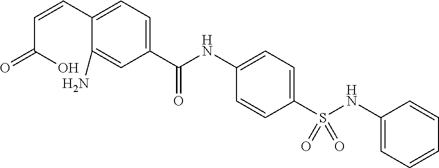 [SMILES: Nc1cc(C(=O)Nc2ccc(S(=O)(=O)Nc3ccccc3)cc2)ccc1/C=C\C(=O)O] | 2.22E−06 | 12.41 | 9.0 |
| 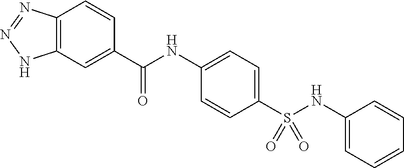 [SMILES: O=C(Nc1ccc(S(=O)(=O)Nc2ccccc2)cc1)c1ccc2nn[nH]c2c1] | ND | 5.75 | 12.0 |
| 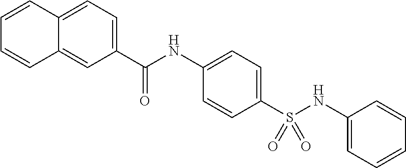 [SMILES: O=C(Nc1ccc(S(=O)(=O)Nc2ccccc2)cc1)c1ccc2ccccc2c1] | 3.42E−06 | 96.34 | >20 |
| 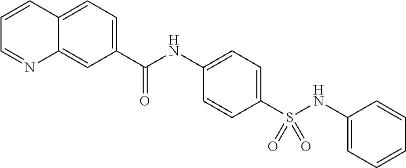 [SMILES: O=C(Nc1ccc(S(=O)(=O)Nc2ccccc2)cc1)c1ccc2cccnc2c1] | 2.01E−06 | 94.20 | >20 |
| 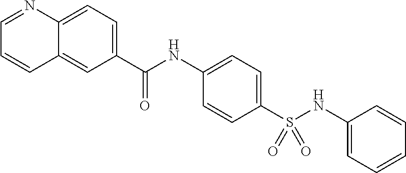 [SMILES: O=C(Nc1ccc(S(=O)(=O)Nc2ccccc2)cc1)c1ccc2ncccc2c1] | 7.00E−05 | 26.01 | <20 |
| 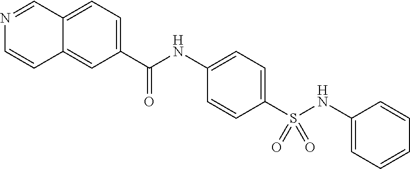 [SMILES: O=C(Nc1ccc(S(=O)(=O)Nc2ccccc2)cc1)c1ccc2cnccc2c1] | 1.34E−04 | 6.45 | 15.0 |
| 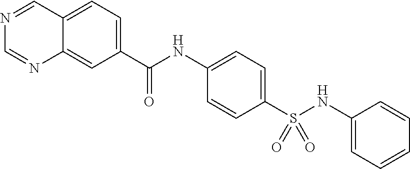 [SMILES: O=C(Nc1ccc(S(=O)(=O)Nc2ccccc2)cc1)c1ccc2cncnc2c1] | 2.26E−04 | 99.96 | >20 |
| 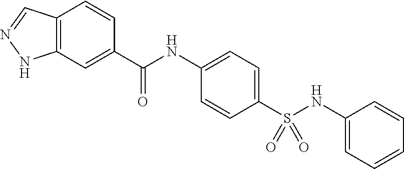 [SMILES: O=C(Nc1ccc(S(=O)(=O)Nc2ccccc2)cc1)c1ccc2cn[nH]c2c1] | 4.84E−05 | 17.21 | 10.0 |
| 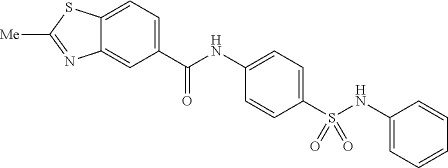 [SMILES: Cc1nc2cc(C(=O)Nc3ccc(S(=O)(=O)Nc4ccccc4)cc3)ccc2s1] | 2.12E−05 | 16.64 | 12.0 |
| 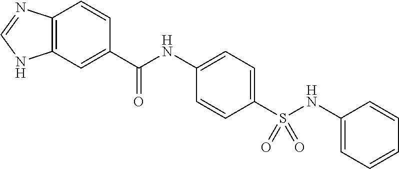 [SMILES: O=C(Nc1ccc(S(=O)(=O)Nc2ccccc2)cc1)c1ccc2nc[nH]c2c1] | 2.30E−04 | 93.60 | >20 |
| 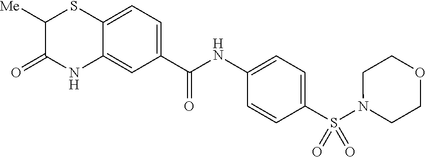 [SMILES: CC1Sc2ccc(C(=O)Nc3ccc(S(=O)(=O)N4CCOCC4)cc3)cc2NC1=O] | 1.37E−04 | 18.94 | 11.0 |
| 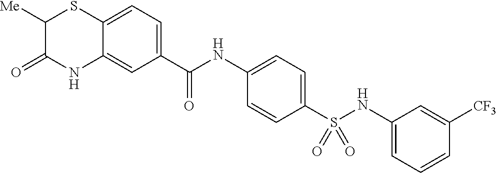 [SMILES: CC1Sc2ccc(C(=O)Nc3ccc(S(=O)(=O)Nc4cccc(C(F)(F)F)c4)cc3)cc2NC1=O] | 5.78E−06 | 9.74 | 21.0 |
| 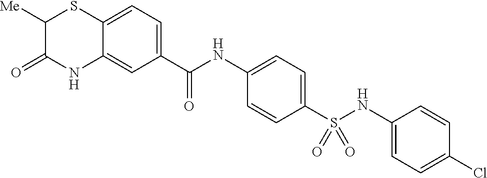 [SMILES: CC1Sc2ccc(C(=O)Nc3ccc(S(=O)(=O)Nc4ccc(Cl)cc4)cc3)cc2NC1=O] | 8.61E−07 | 9.99 | 15.0 |
| 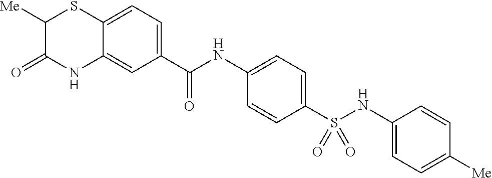 [SMILES: Cc1ccc(NS(=O)(=O)c2ccc(NC(=O)c3ccc4c(c3)NC(=O)C(C)S4)cc2)cc1] | 1.24E−05 | 0.51 | 10.0 |
| 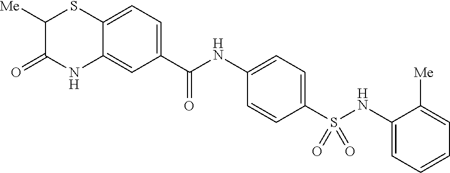 [SMILES: Cc1ccccc1NS(=O)(=O)c1ccc(NC(=O)c2ccc3c(c2)NC(=O)C(C)S3)cc1] | 1.41E−05 | 9.87 | 10.0 |
| 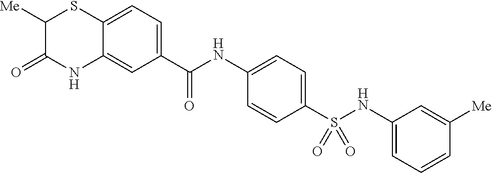 [SMILES: Cc1cccc(NS(=O)(=O)c2ccc(NC(=O)c3ccc4c(c3)NC(=O)C(C)S4)cc2)c1] | 1.16E−06 | 0.58 | 11.0 |
| 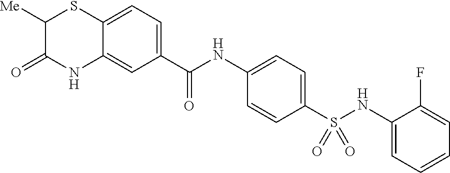 [SMILES: CC1Sc2ccc(C(=O)Nc3ccc(S(=O)(=O)Nc4ccccc4F)cc3)cc2NC1=O] | ND | 21.17 | 15.0 |
| 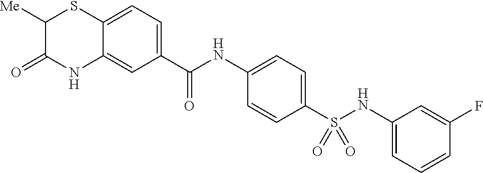 [SMILES: CC1Sc2ccc(C(=O)Nc3ccc(S(=O)(=O)Nc4cccc(F)c4)cc3)cc2NC1=O] | 1.26E−06 | 28.17 | <20 |
| 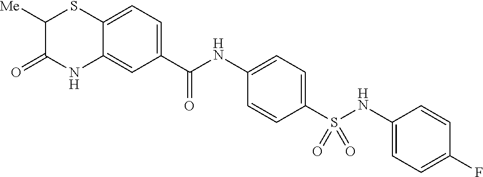 [SMILES: CC1Sc2ccc(C(=O)Nc3ccc(S(=O)(=O)Nc4ccc(F)cc4)cc3)cc2NC1=O] | 1.59E−05 | 80.08 | >20 |
| 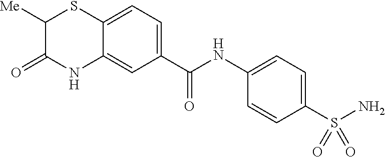 [SMILES: CC1Sc2ccc(C(=O)Nc3ccc(S(N)(=O)=O)cc3)cc2NC1=O] | 8.33E−05 | 100.76 | >20 |
| 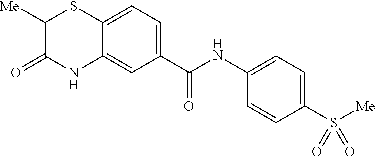 [SMILES: CC1Sc2ccc(C(=O)Nc3ccc(S(C)(=O)=O)cc3)cc2NC1=O] | 7.13E−06 | 30.65 | 15.0 |
| 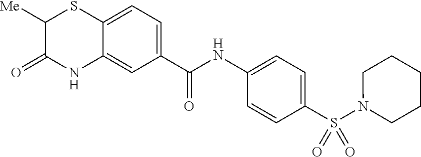 [SMILES: CC1Sc2ccc(C(=O)Nc3ccc(S(=O)(=O)N4CCCCC4)cc3)cc2NC1=O] | 3.63E−06 | 7.38 | 8.0 |
| 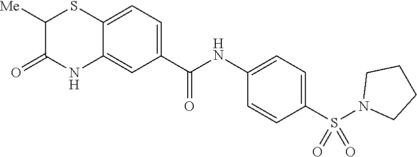 [SMILES: CC1Sc2ccc(C(=O)Nc3ccc(S(=O)(=O)N4CCCC4)cc3)cc2NC1=O] | 1.38E−04 | 2.97 | 8.5 |
| 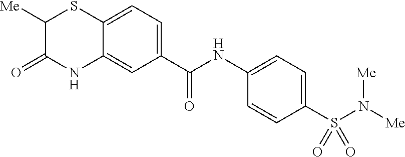 [SMILES: CC1Sc2ccc(C(=O)Nc3ccc(S(=O)(=O)N(C)C)cc3)cc2NC1=O] | 1.54E−05 | 19.60 | 15.0 |
| 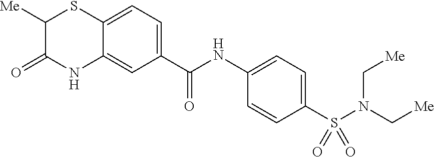 [SMILES: CCN(CC)S(=O)(=O)c1ccc(NC(=O)c2ccc3c(c2)NC(=O)C(C)S3)cc1] | 3.09E−05 | 0.60 | 7.5 |
| 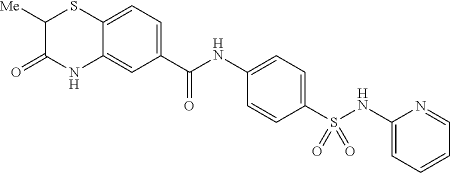 [SMILES: CC1Sc2ccc(C(=O)Nc3ccc(S(=O)(=O)Nc4ccccn4)cc3)cc2NC1=O] | 1.30E−04 | 96.38 | >20 |
| 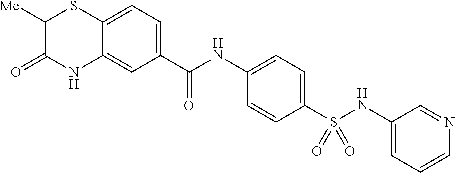 [SMILES: CC1Sc2ccc(C(=O)Nc3ccc(S(=O)(=O)Nc4cccnc4)cc3)cc2NC1=O] | 7.23E−06 | 92.03 | >20 |
| 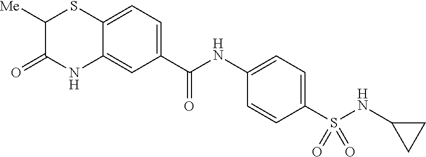 [SMILES: CC1Sc2ccc(C(=O)Nc3ccc(S(=O)(=O)NC4CC4)cc3)cc2NC1=O] | 1.89E−05 | 14.94 | 7.0 |
| 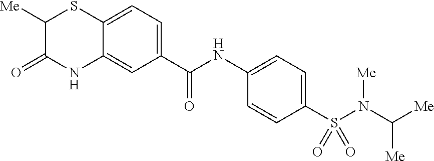 [SMILES: CC1Sc2ccc(C(=O)Nc3ccc(S(=O)(=O)N(C)C(C)C)cc3)cc2NC1=O] | 3.89E−05 | 0.92 | 8.0 |
| 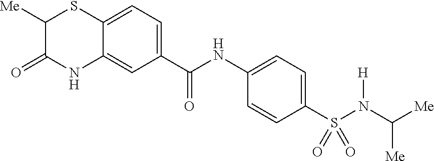 [SMILES: CC(C)NS(=O)(=O)c1ccc(NC(=O)c2ccc3c(c2)NC(=O)C(C)S3)cc1] | 9.79E−06 | 20.27 | 8.0 |
| 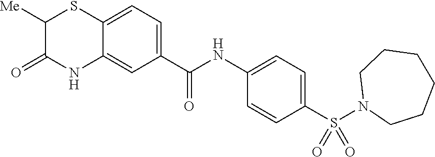 [SMILES: CC1Sc2ccc(C(=O)Nc3ccc(S(=O)(=O)N4CCCCCC4)cc3)cc2NC1=O] | 2.63E−06 | 0.46 | 7.5 |
| 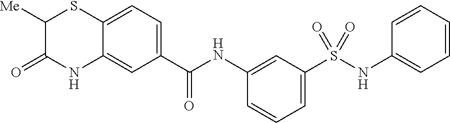 [SMILES: CC1Sc2ccc(C(=O)Nc3cccc(S(=O)(=O)Nc4ccccc4)c3)cc2NC1=O] | 1.86E−05 | 2.81 | 10.0 |
| 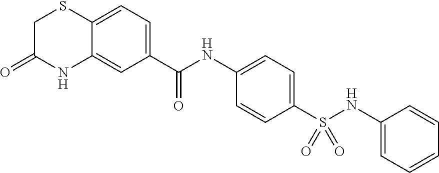 [SMILES: O=C1CSc2ccc(C(=O)Nc3ccc(S(=O)(=O)Nc4ccccc4)cc3)cc2N1] | 1.92E−04 | 2.73 | 7.5 |
| 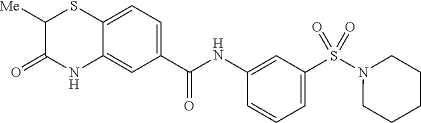 [SMILES: CC1Sc2ccc(C(=O)Nc3cccc(S(=O)(=O)N4CCCCC4)c3)cc2NC1=O] | 4.15E−05 | 0.60 | 10.0 |
| 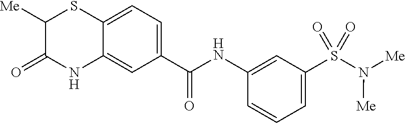 [SMILES: CC1Sc2ccc(C(=O)Nc3cccc(S(=O)(=O)N(C)C)c3)cc2NC1=O] | 6.95E−05 | 1.43 | 8.5 |
| 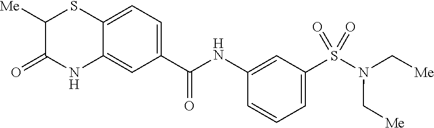 [SMILES: CCN(CC)S(=O)(=O)c1cccc(NC(=O)c2ccc3c(c2)NC(=O)C(C)S3)c1] | 3.12E−05 | 0.44 | 8.0 |
| 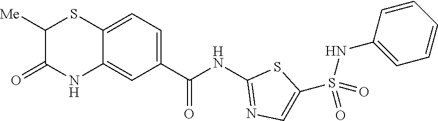 [SMILES: CC1Sc2ccc(C(=O)Nc3ncc(S(=O)(=O)Nc4ccccc4)s3)cc2NC1=O] | 3.12E−06 | 87.98 | >20 |
| 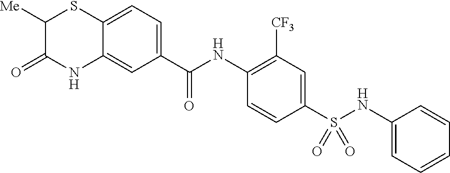 [SMILES: CC1Sc2ccc(C(=O)Nc3ccc(S(=O)(=O)Nc4ccccc4)cc3C(F)(F)F)cc2NC1=O] | 3.09E−06 | 1.38 | 5.0 |
| 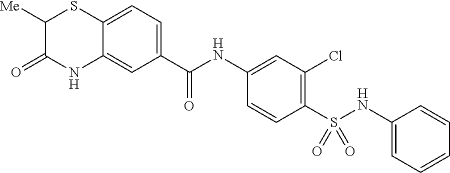 [SMILES: CC1Sc2ccc(C(=O)Nc3ccc(S(=O)(=O)Nc4ccccc4)c(Cl)c3)cc2NC1=O] | 4.36E−06 | 1.80 | 5.0 |
| 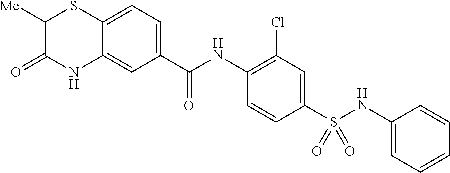 [SMILES: CC1Sc2ccc(C(=O)Nc3ccc(S(=O)(=O)Nc4ccccc4)cc3Cl)cc2NC1=O] | 3.10E−06 | 36.24 | <20 |
| 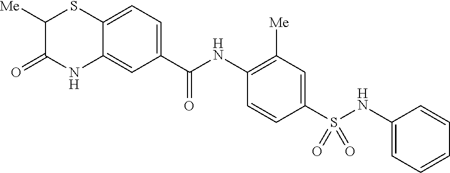 [SMILES: Cc1cc(S(=O)(=O)Nc2ccccc2)ccc1NC(=O)c1ccc2c(c1)NC(=O)C(C)S2] | ND | 51.08 | 20.0 |
| 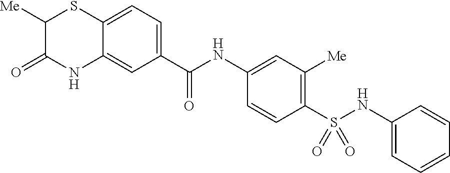 [SMILES: Cc1cc(NC(=O)c2ccc3c(c2)NC(=O)C(C)S3)ccc1S(=O)(=O)Nc1ccccc1] | 2.50E−06 | 1.22 | 10.0 |
| 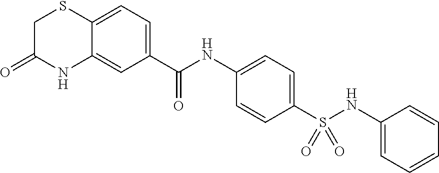 [SMILES: O=C1CSc2ccc(C(=O)Nc3ccc(S(=O)(=O)Nc4ccccc4)cc3)cc2N1] | 3.48E−05 | 79.21 | >20 |
| 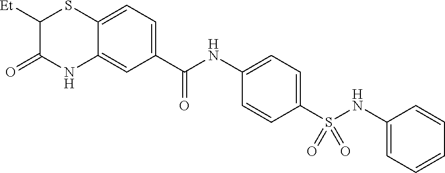 [SMILES: CCC1Sc2ccc(C(=O)Nc3ccc(S(=O)(=O)Nc4ccccc4)cc3)cc2NC1=O] | 1.43E−06 | 36.69 | 12.0 |
| 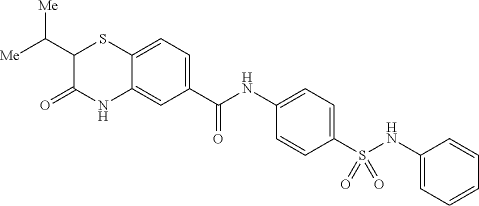 [SMILES: CC(C)C1Sc2ccc(C(=O)Nc3ccc(S(=O)(=O)Nc4ccccc4)cc3)cc2NC1=O] | 1.18E−06 | 1.01 | 2.5 |
| 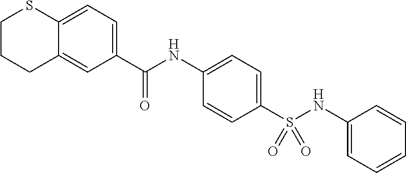 [SMILES: O=C(Nc1ccc(S(=O)(=O)Nc2ccccc2)cc1)c1ccc2c(c1)CCCS2] | 1.62E−06 | 1.95 | 12.0 |
| 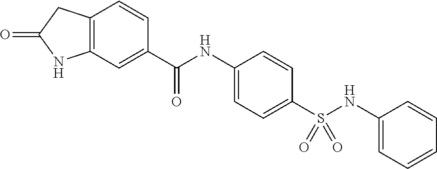 [SMILES: O=C1Cc2ccc(C(=O)Nc3ccc(S(=O)(=O)Nc4ccccc4)cc3)cc2N1] | 2.09E−05 | 81.50 | >20 |
| 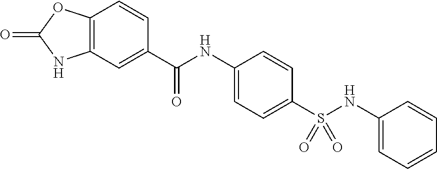 [SMILES: O=C(Nc1ccc(S(=O)(=O)Nc2ccccc2)cc1)c1ccc2oc(=O)[nH]c2c1] | 8.68E−06 | 51.81 | 20.0 |
| 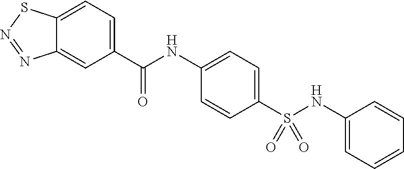 [SMILES: O=C(Nc1ccc(S(=O)(=O)Nc2ccccc2)cc1)c1ccc2snnc2c1] | 5.65E−06 | 34.34 | 5.0−10.0 |
| 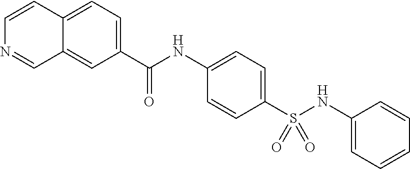 [SMILES: O=C(Nc1ccc(S(=O)(=O)Nc2ccccc2)cc1)c1ccc2ccncc2c1] | 3.79E−05 | 24.83 | 5.0 |
| 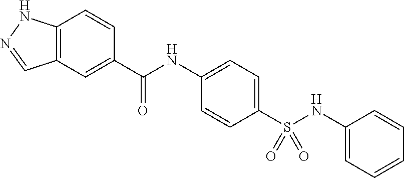 [SMILES: O=C(Nc1ccc(S(=O)(=O)Nc2ccccc2)cc1)c1ccc2[nH]ncc2c1] | 7.85E−05 | 47.61 | 20.0 |
| 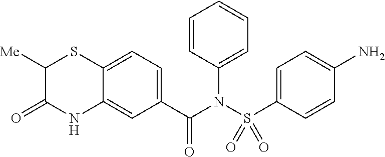 [SMILES: CC1Sc2ccc(C(=O)N(c3ccccc3)S(=O)(=O)c3ccc(N)cc3)cc2NC1=O] | 7.75E−06 | 91.25 | >20 |
| 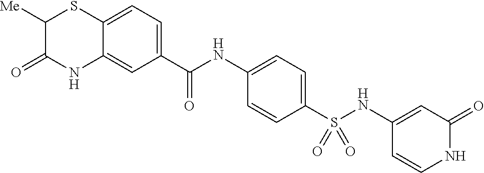 [SMILES: CC1Sc2ccc(C(=O)Nc3ccc(S(=O)(=O)Nc4cc[nH]c(=O)c4)cc3)cc2NC1=O] | 2.48E−05 | 34.75 | 18.0 |
| 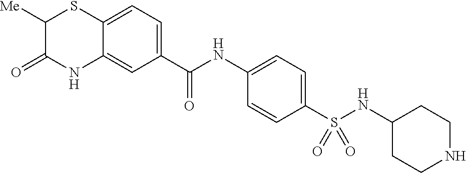 [SMILES: CC1Sc2ccc(C(=O)Nc3ccc(S(=O)(=O)NC4CCNCC4)cc3)cc2NC1=O] | 1.00E−04 | 80.72 | >20 |
| 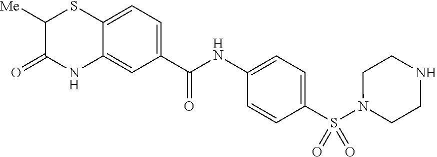 [SMILES: CC1Sc2ccc(C(=O)Nc3ccc(S(=O)(=O)N4CCNCC4)cc3)cc2NC1=O] | 1.75E−05 | 92.44 | >20 |
| 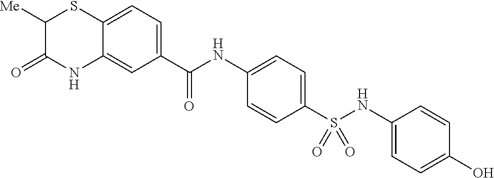 [SMILES: CC1Sc2ccc(C(=O)Nc3ccc(S(=O)(=O)Nc4ccc(O)cc4)cc3)cc2NC1=O] | 3.20E−05 | 13.72 | <20 |
| 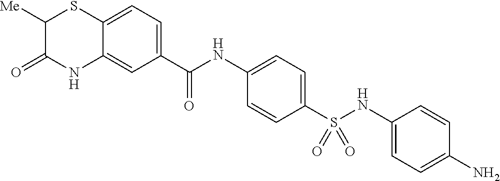 [SMILES: CC1Sc2ccc(C(=O)Nc3ccc(S(=O)(=O)Nc4ccc(N)cc4)cc3)cc2NC1=O] | 1.96E−05 | 87.98 | >20 |
| 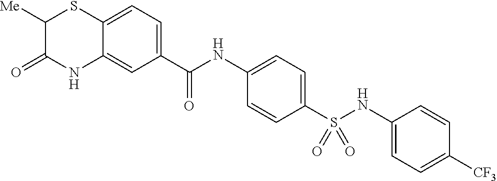 [SMILES: CC1Sc2ccc(C(=O)Nc3ccc(S(=O)(=O)Nc4ccc(C(F)(F)F)cc4)cc3)cc2NC1=O] | 1.57E−06 | 63.62 | >20 |
| 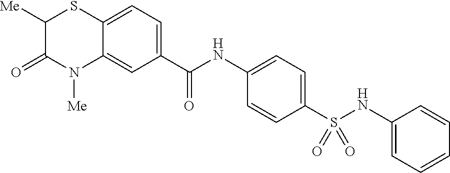 [SMILES: CC1Sc2ccc(C(=O)Nc3ccc(S(=O)(=O)Nc4ccccc4)cc3)cc2N(C)C1=O] | 1.95E−06 | 1.23 | 8.0 |
| 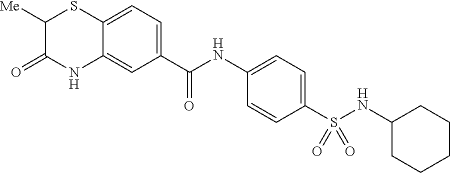 [SMILES: CC1Sc2ccc(C(=O)Nc3ccc(S(=O)(=O)NC4CCCCC4)cc3)cc2NC1=O] | 1.90E−05 | 30.40 | 8.0 |
| 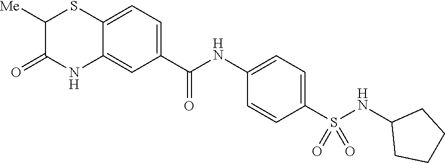 [SMILES: CC1Sc2ccc(C(=O)Nc3ccc(S(=O)(=O)NC4CCCC4)cc3)cc2NC1=O] | 1.34E−05 | 36.31 | 15.0 |
| 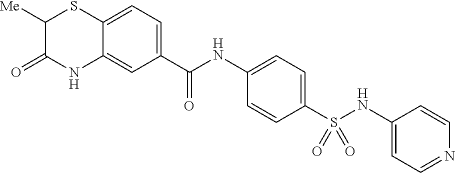 [SMILES: CC1Sc2ccc(C(=O)Nc3ccc(S(=O)(=O)Nc4ccncc4)cc3)cc2NC1=O] | 3.09E−05 | 102.57 | >20 |
| 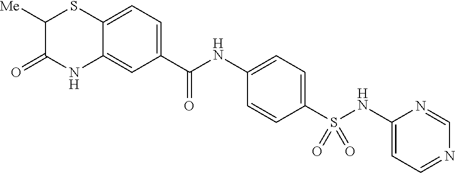 [SMILES: CC1Sc2ccc(C(=O)Nc3ccc(S(=O)(=O)Nc4ccncn4)cc3)cc2NC1=O] | 5.51E−06 | 105.51 | >20 |
| 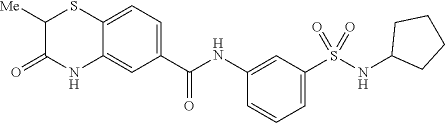 [SMILES: CC1Sc2ccc(C(=O)Nc3cccc(S(=O)(=O)NC4CCCC4)c3)cc2NC1=O] | 1.54E−05 | 38.54 | 16.0 |
| 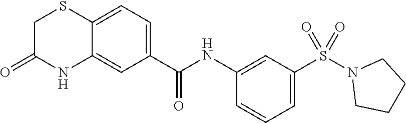 [SMILES: O=C1CSc2ccc(C(=O)Nc3cccc(S(=O)(=O)N4CCCC4)c3)cc2N1] | 3.86E−05 | 45.37 | 20.0 |
| 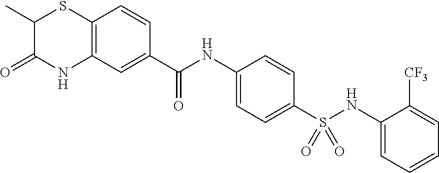 [SMILES: CC1Sc2ccc(C(=O)Nc3ccc(S(=O)(=O)Nc4ccccc4C(F)(F)F)cc3)cc2NC1=O] | 1.10E−06 | 8.61 | 10.0 |
| 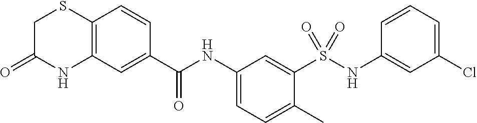 [SMILES: Cc1ccc(NC(=O)c2ccc3c(c2)NC(=O)CS3)cc1S(=O)(=O)Nc1cccc(Cl)c1] | 3.37E−06 | 26.27 | 10.0 |
| 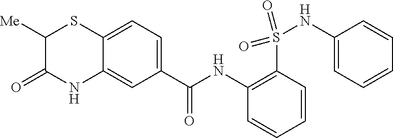 [SMILES: CC1Sc2ccc(C(=O)Nc3ccccc3S(=O)(=O)Nc3ccccc3)cc2NC1=O] | 2.92E−05 | 14.24 | <20 |
| 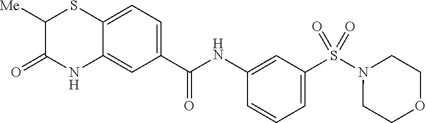 [SMILES: CC1Sc2ccc(C(=O)Nc3cccc(S(=O)(=O)N4CCOCC4)c3)cc2NC1=O] | 8.50E−05 | 30.78 | 8.0 |
| 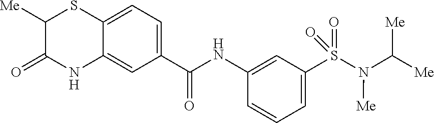 [SMILES: CC1Sc2ccc(C(=O)Nc3cccc(S(=O)(=O)N(C)C(C)C)c3)cc2NC1=O] | 6.59E−05 | 9.64 | 6.0 |
| 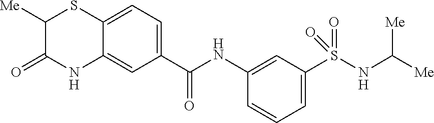 [SMILES: CC(C)NS(=O)(=O)c1cccc(NC(=O)c2ccc3c(c2)NC(=O)C(C)S3)c1] | 9.09E−05 | 17.58 | 8.0 |
| 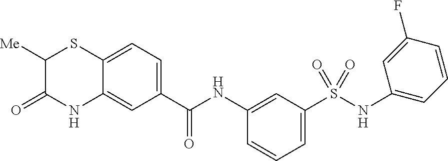 [SMILES: CC1Sc2ccc(C(=O)Nc3cccc(S(=O)(=O)Nc4cccc(F)c4)c3)cc2NC1=O] | 6.29E−07 | 13.06 | 10.0 |
| 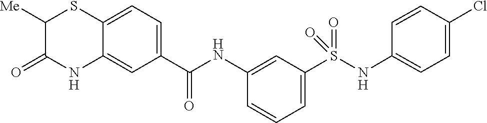 [SMILES: CC1Sc2ccc(C(=O)Nc3cccc(S(=O)(=O)Nc4ccc(Cl)cc4)c3)cc2NC1=O] | 4.24E−06 | 10.37 | 11.5 |
| 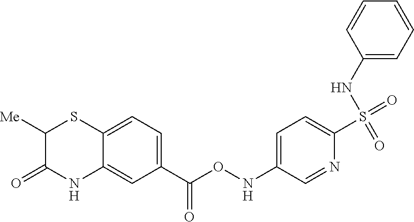 [SMILES: CC1Sc2ccc(C(=O)ONc3ccc(S(=O)(=O)Nc4ccccc4)nc3)cc2NC1=O] | 5.07E−06 | 89.99 | >20 |
| 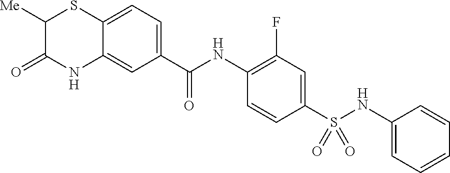 [SMILES: CC1Sc2ccc(C(=O)Nc3ccc(S(=O)(=O)Nc4ccccc4)cc3F)cc2NC1=O] | 3.10E−05 | 89.97 | >20 |
| 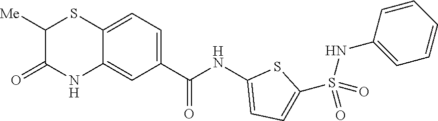 [SMILES: CC1Sc2ccc(C(=O)Nc3ccc(S(=O)(=O)Nc4ccccc4)s3)cc2NC1=O] | 6.63E−06 | 57.44 | >20 |
| 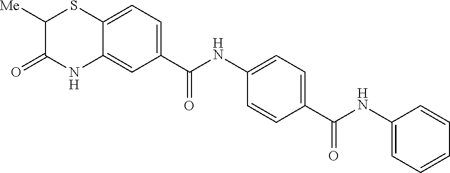 [SMILES: CC1Sc2ccc(C(=O)Nc3ccc(C(=O)Nc4ccccc4)cc3)cc2NC1=O] | 1.37E−05 | 97.48 | >20 |
| 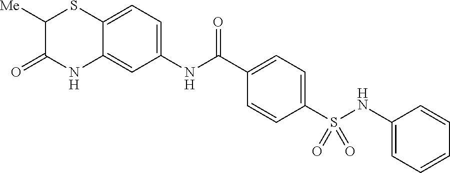 [SMILES: CC1Sc2ccc(NC(=O)c3ccc(S(=O)(=O)Nc4ccccc4)cc3)cc2NC1=O] | 3.32E−05 | 33.54 | 9.0 |
| 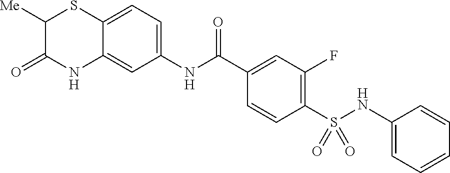 [SMILES: CC1Sc2ccc(NC(=O)c3ccc(S(=O)(=O)Nc4ccccc4)c(F)c3)cc2NC1=O] | 1.61E−05 | 75.82 | >20 |
| 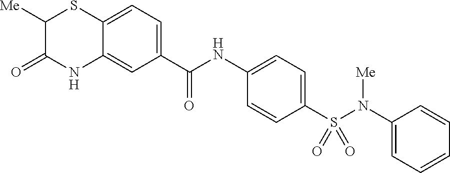 [SMILES: CC1Sc2ccc(C(=O)Nc3ccc(S(=O)(=O)N(C)c4ccccc4)cc3)cc2NC1=O] | 1.47E−06 | 18.00 | 9.0 |
| 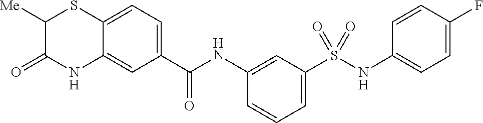 [SMILES: CC1Sc2ccc(C(=O)Nc3cccc(S(=O)(=O)Nc4ccc(F)cc4)c3)cc2NC1=O] | 8.48E−06 | 55.61 | >20 |
| 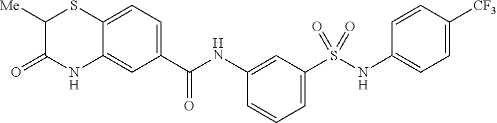 [SMILES: CC1Sc2ccc(C(=O)Nc3cccc(S(=O)(=O)Nc4ccc(C(F)(F)F)cc4)c3)cc2NC1=O] | 2.12E−06 | 22.22 | <20 |
| 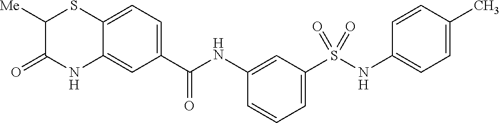 [SMILES: Cc1ccc(NS(=O)(=O)c2cccc(NC(=O)c3ccc4c(c3)NC(=O)C(C)S4)c2)cc1] | 6.08E−06 | 2.16 | 7.0 |
| 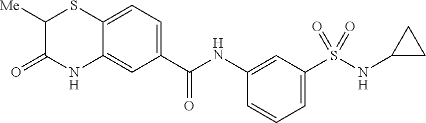 [SMILES: CC1Sc2ccc(C(=O)Nc3cccc(S(=O)(=O)NC4CC4)c3)cc2NC1=O] | 9.69E−05 | 11.92 | 4.0 |
| 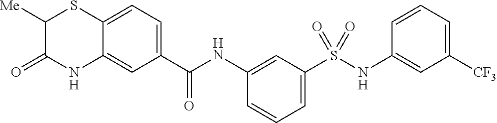 [SMILES: CC1Sc2ccc(C(=O)Nc3cccc(S(=O)(=O)Nc4cccc(C(F)(F)F)c4)c3)cc2NC1=O] | ND | 16.81 | 10.0 |
| 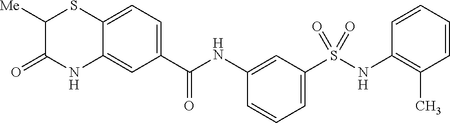 [SMILES: Cc1ccccc1NS(=O)(=O)c1cccc(NC(=O)c2ccc3c(c2)NC(=O)C(C)S3)c1] | 7.42E−06 | 6.11 | 6.0 |
| 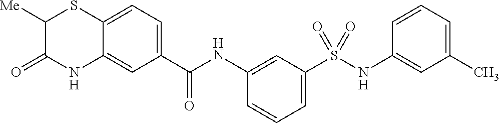 [SMILES: Cc1cccc(NS(=O)(=O)c2cccc(NC(=O)c3ccc4c(c3)NC(=O)C(C)S4)c2)c1] | 2.47E−06 | 3.73 | 10.0 |
| 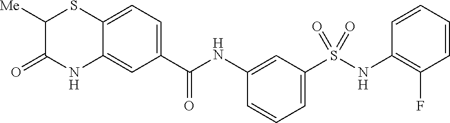 [SMILES: CC1Sc2ccc(C(=O)Nc3cccc(S(=O)(=O)Nc4ccccc4F)c3)cc2NC1=O] | 2.87E−05 | 22.67 | 13.0 |
| 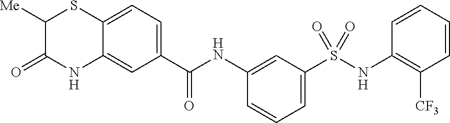 [SMILES: CC1Sc2ccc(C(=O)Nc3cccc(S(=O)(=O)Nc4ccccc4C(F)(F)F)c3)cc2NC1=O] | 9.17E−05 | 73.57 | >20 |
| 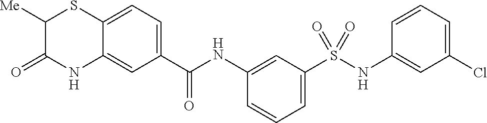 [SMILES: CC1Sc2ccc(C(=O)Nc3cccc(S(=O)(=O)Nc4cccc(Cl)c4)c3)cc2NC1=O] | 3.15E−06 | 28.81 | <20 |
| 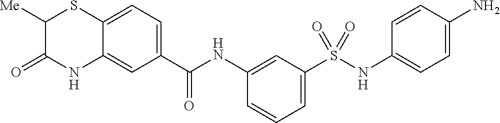 [SMILES: CC1Sc2ccc(C(=O)Nc3cccc(S(=O)(=O)Nc4ccc(N)cc4)c3)cc2NC1=O] | 5.97E−05 | 59.66 | >20 |
| 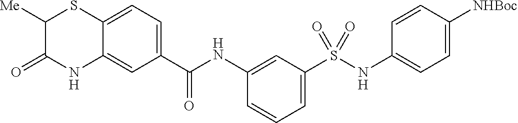 [SMILES: CC1Sc2ccc(C(=O)Nc3cccc(S(=O)(=O)Nc4ccc(NC(=O)OC(C)(C)C)cc4)c3)cc2NC1=O] | 5.07E−06 | 67.38 | >20 |
| 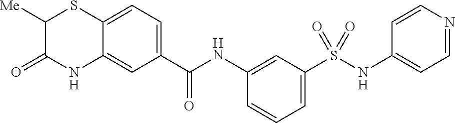 [SMILES: CC1Sc2ccc(C(=O)Nc3cccc(S(=O)(=O)Nc4ccncc4)c3)cc2NC1=O] | 2.12E−05 | 95.78 | >20 |
| 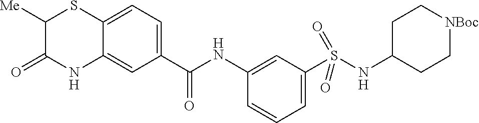 [SMILES: CC1Sc2ccc(C(=O)Nc3cccc(S(=O)(=O)NC4CCN(C(=O)OC(C)(C)C)CC4)c3)cc2NC1=O] | 1.42E−04 | 12.98 | 15.0 |
| 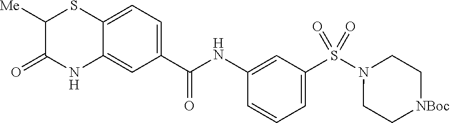 [SMILES: CC1Sc2ccc(C(=O)Nc3cccc(S(=O)(=O)N4CCN(C(=O)OC(C)(C)C)CC4)c3)cc2NC1=O] | 1.76E−06 | 36.40 | 18.0 |
| 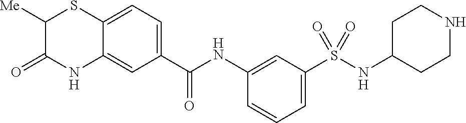 [SMILES: CC1Sc2ccc(C(=O)Nc3cccc(S(=O)(=O)NC4CCNCC4)c3)cc2NC1=O] | 5.46E−05 | 96.59 | >20 |
| 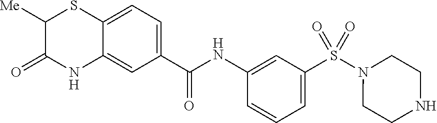 [SMILES: CC1Sc2ccc(C(=O)Nc3cccc(S(=O)(=O)N4CCNCC4)c3)cc2NC1=O] | 1.31E−04 | 113.54 | >20 |
| 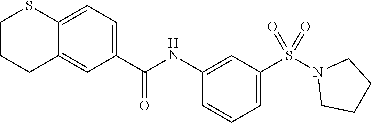 [SMILES: O=C(Nc1cccc(S(=O)(=O)N2CCCC2)c1)c1ccc2c(c1)CCCS2] | 1.98E−06 | 27.14 | |
| 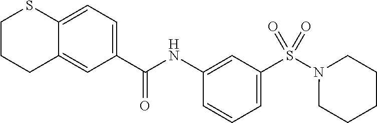 [SMILES: O=C(Nc1cccc(S(=O)(=O)N2CCCCC2)c1)c1ccc2c(c1)CCCS2] | 3.01E−06 | 54.22 | |
| 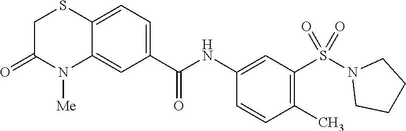 [SMILES: Cc1ccc(NC(=O)c2ccc3c(c2)N(C)C(=O)CS3)cc1S(=O)(=O)N1CCCC1] | 3.59E−05 | 90.92 | |
| 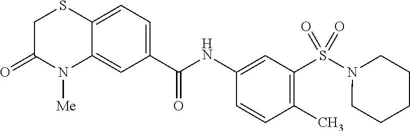 [SMILES: Cc1ccc(NC(=O)c2ccc3c(c2)N(C)C(=O)CS3)cc1S(=O)(=O)N1CCCCC1] | 8.89E−06 | 3.42 | |
| 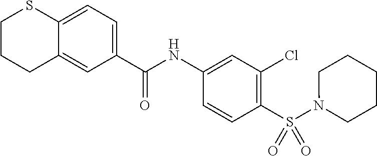 [SMILES: O=C(Nc1ccc(S(=O)(=O)N2CCCCC2)c(Cl)c1)c1ccc2c(c1)CCCS2] | 1.76E−05 | 35.10 | |
| 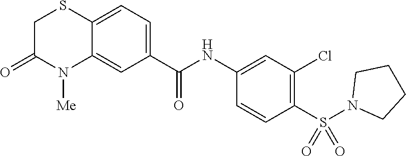 [SMILES: CN1C(=O)CSc2ccc(C(=O)Nc3ccc(S(=O)(=O)N4CCCC4)c(Cl)c3)cc21] | 2.74E−05 | 16.02 | |
| 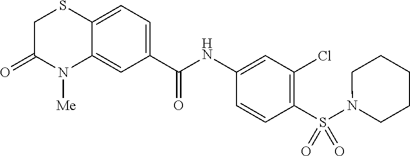 [SMILES: CN1C(=O)CSc2ccc(C(=O)Nc3ccc(S(=O)(=O)N4CCCCC4)c(Cl)c3)cc21] | 2.75E−06 | 4.13 | |
| 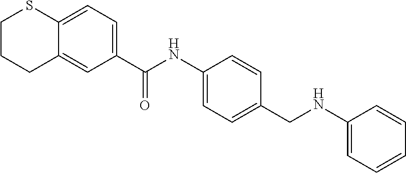 [SMILES: O=C(Nc1ccc(CNc2ccccc2)cc1)c1ccc2c(c1)CCCS2] | 1.35E−05 | 51.88 | |
| 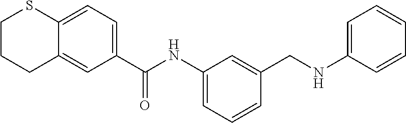 [SMILES: O=C(Nc1cccc(CNc2ccccc2)c1)c1ccc2c(c1)CCCS2] | 7.34E−06 | 85.31 | |
| 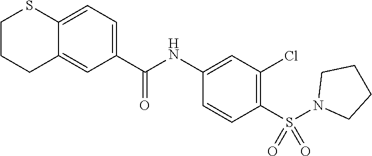 [SMILES: O=C(Nc1ccc(S(=O)(=O)N2CCCC2)c(Cl)c1)c1ccc2c(c1)CCCS2] | 3.86E−06 | 1.26 | |
| 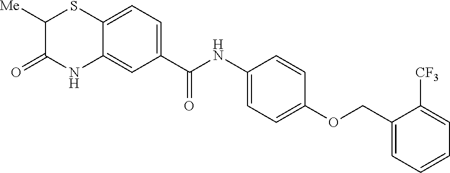 [SMILES: CC1Sc2ccc(C(=O)Nc3ccc(OCc4ccccc4C(F)(F)F)cc3)cc2NC1=O] | ND | 106.06 | |
| 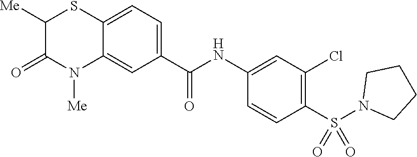 [SMILES: CC1Sc2ccc(C(=O)Nc3ccc(S(=O)(=O)N4CCCC4)c(Cl)c3)cc2N(C)C1=O] | 2.04E−05 | 8.44 | |
| 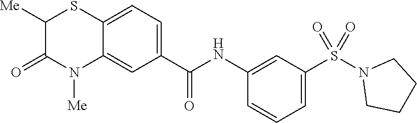 [SMILES: CC1Sc2ccc(C(=O)Nc3cccc(S(=O)(=O)N4CCCC4)c3)cc2N(C)C1=O] | 3.46E−04 | 13.86 | |
| 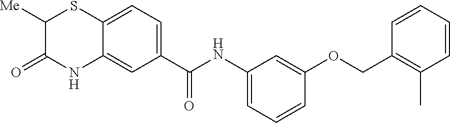 [SMILES: Cc1ccccc1COc1cccc(NC(=O)c2ccc3c(c2)NC(=O)C(C)S3)c1] | 5.41E−06 | 110.41 | |
| 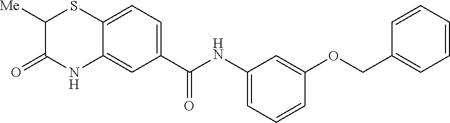 [SMILES: CC1Sc2ccc(C(=O)Nc3cccc(OCc4ccccc4)c3)cc2NC1=O] | 1.37E−05 | 106.68 | |
| 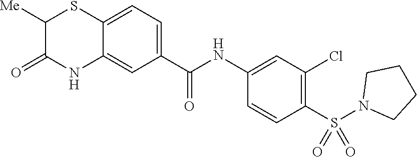 [SMILES: CC1Sc2ccc(C(=O)Nc3ccc(S(=O)(=O)N4CCCC4)c(Cl)c3)cc2NC1=O] | 1.38E−05 | 8.93 | |
| 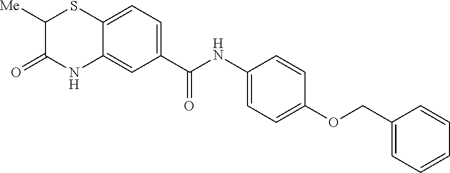 [SMILES: CC1Sc2ccc(C(=O)Nc3ccc(OCc4ccccc4)cc3)cc2NC1=O] | ND | 103.93 | |
| 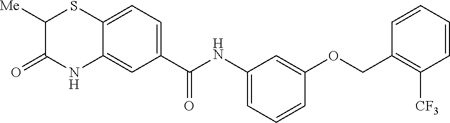 [SMILES: CC1Sc2ccc(C(=O)Nc3cccc(OCc4ccccc4C(F)(F)F)c3)cc2NC1=O] | ND | 87.11 | |
| 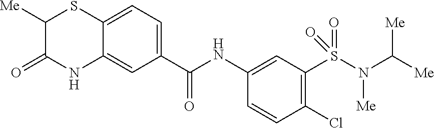 [SMILES: CC1Sc2ccc(C(=O)Nc3ccc(Cl)c(S(=O)(=O)N(C)C(C)C)c3)cc2NC1=O] | 1.34E−05 | 11.98 | |
| 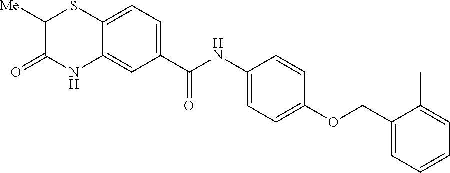 [SMILES: Cc1ccccc1COc1ccc(NC(=O)c2ccc3c(c2)NC(=O)C(C)S3)cc1] | 7.91E−06 | 99.24 | |
| 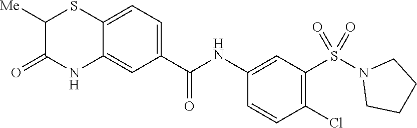 [SMILES: CC1Sc2ccc(C(=O)Nc3ccc(Cl)c(S(=O)(=O)N4CCCC4)c3)cc2NC1=O] | 2.39E−05 | 1.39 | |
| 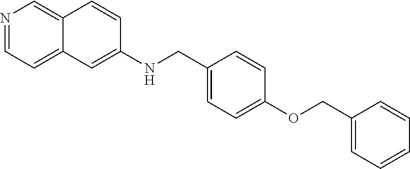 [SMILES: c1ccc(COc2ccc(CNc3ccc4cnccc4c3)cc2)cc1] | 3.37E−05 | 1.20 | |
| 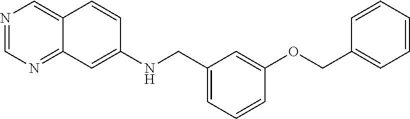 [SMILES: c1ccc(COc2cccc(CNc3ccc4cncnc4c3)c2)cc1] | 2.42E−05 | 1.40 | |
| 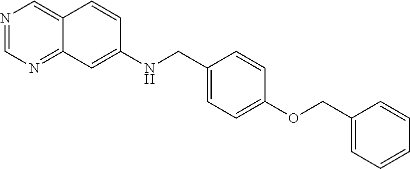 [SMILES: c1ccc(COc2ccc(CNc3ccc4cncnc4c3)cc2)cc1] | 5.20E−06 | 1.09 | |
| 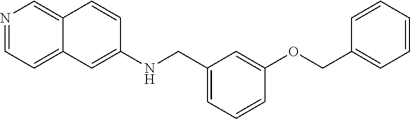 [SMILES: c1ccc(COc2cccc(CNc3ccc4cnccc4c3)c2)cc1] | 4.78E−05 | 1.12 | |
| 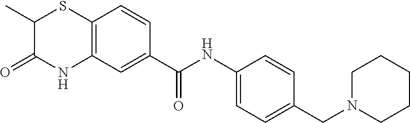 [SMILES: CC1Sc2ccc(C(=O)Nc3ccc(CN4CCCCC4)cc3)cc2NC1=O] | 3.43E−06 | 107.96 | |
| 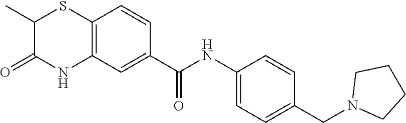 [SMILES: CC1Sc2ccc(C(=O)Nc3ccc(CN4CCCC4)cc3)cc2NC1=O] | 7.15E−05 | 104.38 | |
| 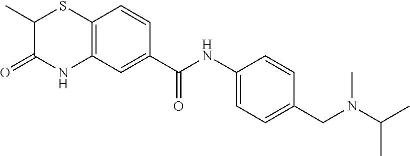 [SMILES: CC1Sc2ccc(C(=O)Nc3ccc(CN(C)C(C)C)cc3)cc2NC1=O] | 1.17E−06 | 104.64 | |
| 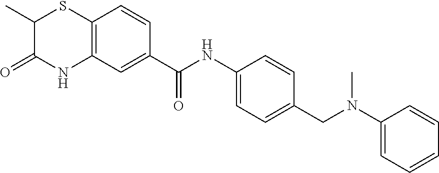 [SMILES: CC1Sc2ccc(C(=O)Nc3ccc(CN(C)c4ccccc4)cc3)cc2NC1=O] | 3.62E−06 | 84.94 | |
| 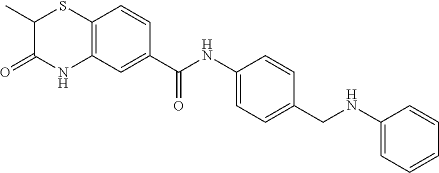 [SMILES: CC1Sc2ccc(C(=O)Nc3ccc(CNc4ccccc4)cc3)cc2NC1=O] | 2.58E−07 | 107.73 | |
| 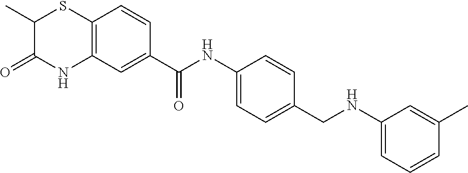 | 8.16E−06 | 78.97 | |
| 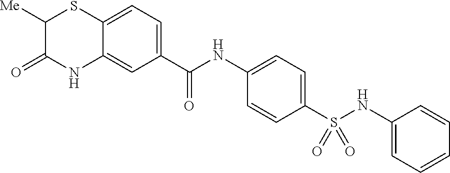 [SMILES: CC1Sc2ccc(C(=O)Nc3ccc(S(=O)(=O)Nc4ccccc4)cc3)cc2NC1=O] | ND | 6.36 | 12.5 |
| 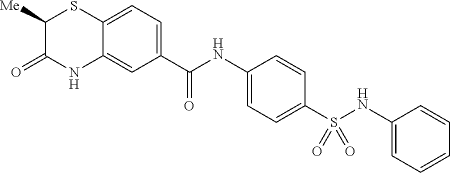 [SMILES: C[C@H]1Sc2ccc(C(=O)Nc3ccc(S(=O)(=O)Nc4ccccc4)cc3)cc2NC1=O] | ND | ND | 12.5 |
| 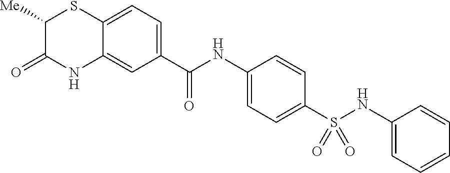 [SMILES: C[C@@H]1Sc2ccc(C(=O)Nc3ccc(S(=O)(=O)Nc4ccccc4)cc3)cc2NC1=O] | 3.19E−05 | 12.81 | 12.5 |
| 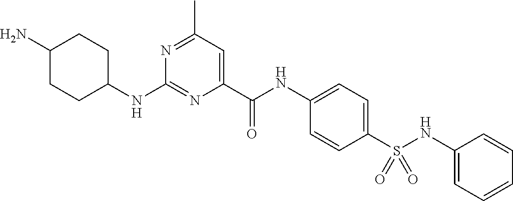 [SMILES: Cc1cc(C(=O)Nc2ccc(S(=O)(=O)Nc3ccccc3)cc2)nc(NC2CCC(N)CC2)n1] | 9.46E−05 | | |
| 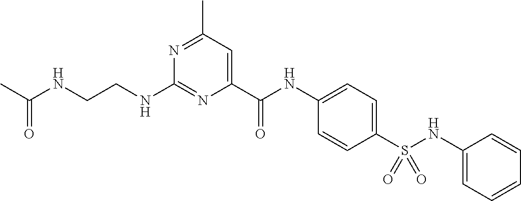 [SMILES: CC(=O)NCCNc1nc(C)cc(C(=O)Nc2ccc(S(=O)(=O)Nc3ccccc3)cc2)n1] | 8.96E−05 | | |
| 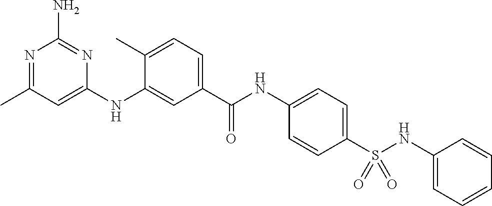 [SMILES: Cc1cc(Nc2cc(C(=O)Nc3ccc(S(=O)(=O)Nc4ccccc4)cc3)ccc2C)nc(N)n1] | 8.07E−05 | | |
| 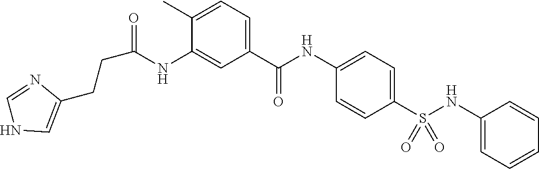 [SMILES: Cc1ccc(C(=O)Nc2ccc(S(=O)(=O)Nc3ccccc3)cc2)cc1NC(=O)CCc1c[nH]cn1] | 7.21E−05 | | |
| 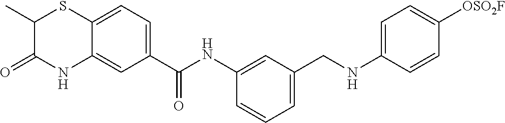 | 3.53E−06 | 4.81 | 10.0 |
| 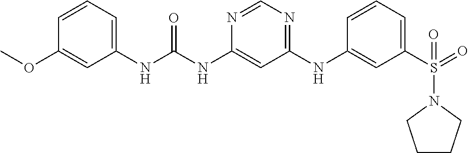 [SMILES: COc1cccc(NC(=O)Nc2cc(Nc3cccc(S(=O)(=O)N4CCCC4)c3)ncn2)c1] | 1.65E−05 | | |
| 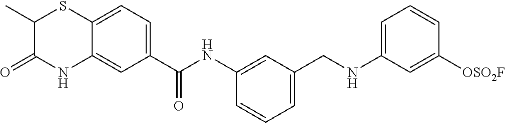 | 1.12E−05 | | |
| 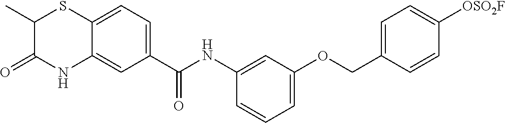 | 1.91E−05 | | |
| 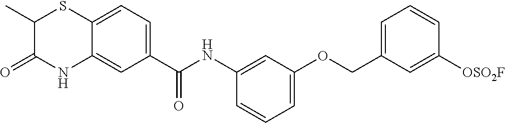 | 1.69E−05 | | |
| 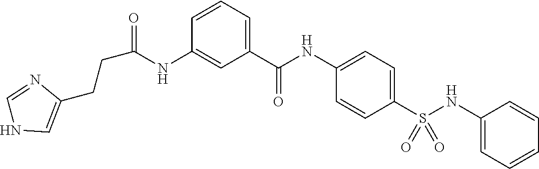 [SMILES: O=C(CCc1c[nH]cn1)Nc1cccc(C(=O)Nc2ccc(S(=O)(=O)Nc3ccccc3)cc2)c1] | 6.46E−06 | 90.68 | >20.0 |
| 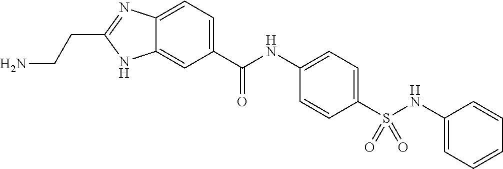 [SMILES: NCCc1nc2ccc(C(=O)Nc3ccc(S(=O)(=O)Nc4ccccc4)cc3)cc2[nH]1] | 7.93E−05 | | |
| 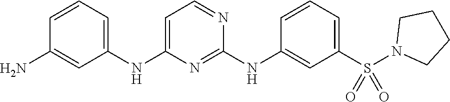 [SMILES: Nc1cccc(Nc2ccnc(Nc3cccc(S(=O)(=O)N4CCCC4)c3)n2)c1] | 1.18E−04 | | |
| 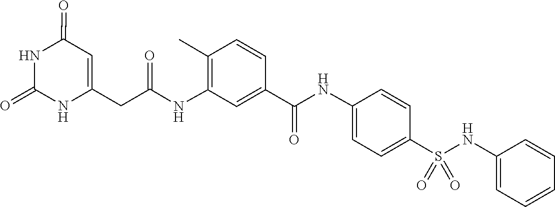 [SMILES: Cc1ccc(C(=O)Nc2ccc(S(=O)(=O)Nc3ccccc3)cc2)cc1NC(=O)Cc1cc(=O)[nH]c(=O)[nH]1] | 6.19E−05 | | |
| 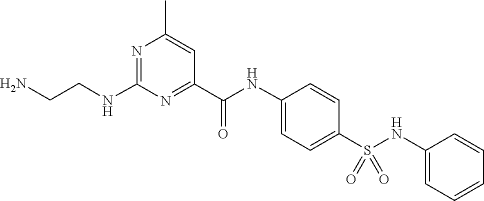 [SMILES: Cc1cc(C(=O)Nc2ccc(S(=O)(=O)Nc3ccccc3)cc2)nc(NCCN)n1] | 2.21E−05 | | |
| 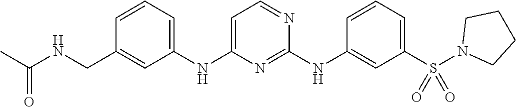 [SMILES: CC(=O)NCc1cccc(Nc2ccnc(Nc3cccc(S(=O)(=O)N4CCCC4)c3)n2)c1] | 3.80E−05 | | |
| 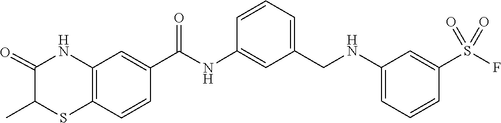 [SMILES: CC1Sc2ccc(C(=O)Nc3cccc(CNc4cccc(S(=O)(=O)F)c4)c3)cc2NC1=O] | 1.90E−06 | 44.31 | 19.0 |
| 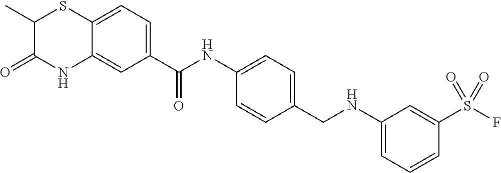 [SMILES: CC1Sc2ccc(C(=O)Nc3ccc(CNc4cccc(S(=O)(=O)F)c4)cc3)cc2NC1=O] | 1.65E−06 | 19.74 | 16.0 |
| 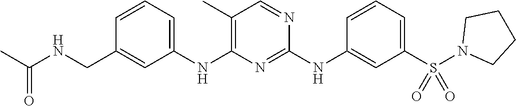 [SMILES: CC(=O)NCc1cccc(Nc2nc(Nc3cccc(S(=O)(=O)N4CCCC4)c3)ncc2C)c1] | 1.63E−04 | | |
| 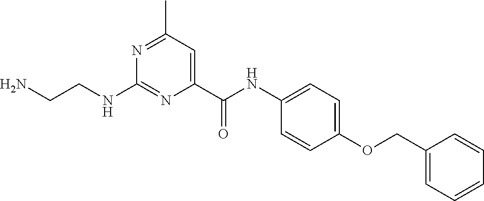 [SMILES: Cc1cc(C(=O)Nc2ccc(OCc3ccccc3)cc2)nc(NCCN)n1] | 1.22E−04 | | |
| 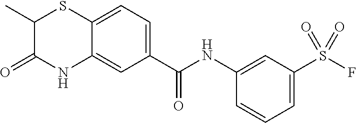 [SMILES: CC1Sc2ccc(C(=O)Nc3cccc(S(=O)(=O)F)c3)cc2NC1=O] | 3.55E−05 | | |
| 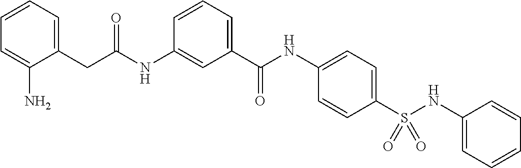 [SMILES: Nc1ccccc1CC(=O)Nc1cccc(C(=O)Nc2ccc(S(=O)(=O)Nc3ccccc3)cc2)c1] | 8.05E−07 | 9.92 | 11.0 |
| 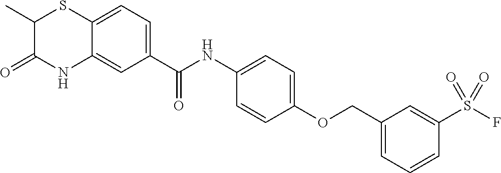 [SMILES: CC1Sc2ccc(C(=O)Nc3ccc(OCc4cccc(S(=O)(=O)F)c4)cc3)cc2NC1=O] | 7.17E−06 | | |
| 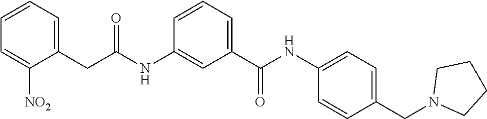 [SMILES: O=C(Cc1ccccc1[N+](=O)[O-])Nc1cccc(C(=O)Nc2ccc(CN3CCCC3)cc2)c1] | 2.47E−04 | | |
| 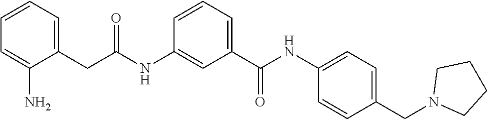 [SMILES: Nc1ccccc1CC(=O)Nc1cccc(C(=O)Nc2ccc(CN3CCCC3)cc2)c1] | 1.54E−05 | | |
| 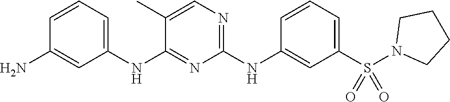 [SMILES: Cc1cnc(Nc2cccc(S(=O)(=O)N3CCCC3)c2)nc1Nc1cccc(N)c1] | 1.05E−04 | | |
| 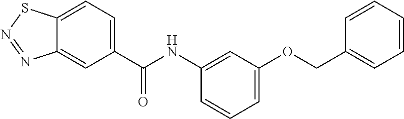 [SMILES: O=C(Nc1cccc(OCc2ccccc2)c1)c1ccc2snnc2c1] | 9.61E−06 | 98.09 | >20.0 |
| 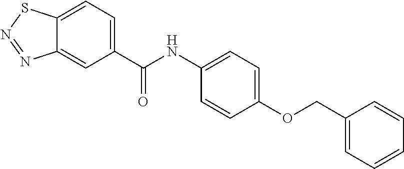 [SMILES: O=C(Nc1ccc(OCc2ccccc2)cc1)c1ccc2snnc2c1] | 1.65E−06 | 93.61 | >20.0 |
| 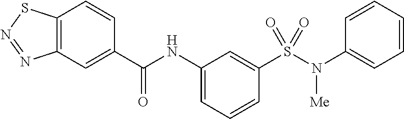 [SMILES: CN(c1ccccc1)S(=O)(=O)c1cccc(NC(=O)c2ccc3snnc3c2)c1] | 8.33E−06 | 10.57 | 10.0 |
| 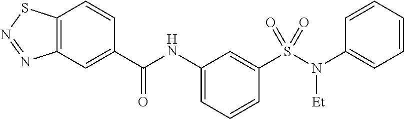 [SMILES: CCN(c1ccccc1)S(=O)(=O)c1cccc(NC(=O)c2ccc3snnc3c2)c1] | 2.65E−06 | 4.86 | 8.5 |
| 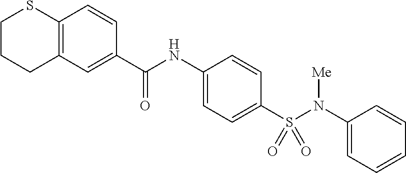 [SMILES: CN(c1ccccc1)S(=O)(=O)c1ccc(NC(=O)c2ccc3c(c2)CCCS3)cc1] | 2.17E−05 | | |
| 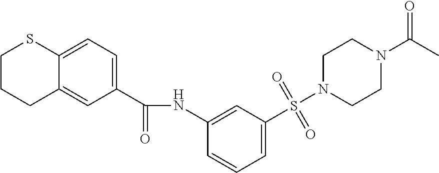 [SMILES: CC(=O)N1CCN(S(=O)(=O)c2cccc(NC(=O)c3ccc4c(c3)CCCS4)c2)CC1] | 1.54E−05 | | |
| 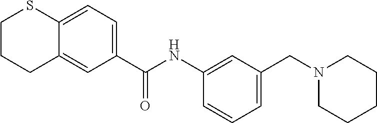 [SMILES: O=C(Nc1cccc(CN2CCCCC2)c1)c1ccc2c(c1)CCCS2] | 2.40E−05 | | |
| 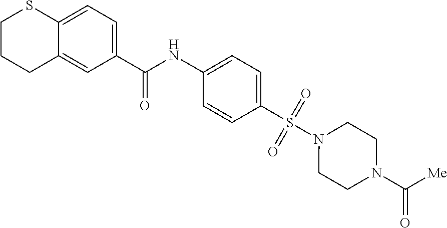 [SMILES: CC(=O)N1CCN(S(=O)(=O)c2ccc(NC(=O)c3ccc4c(c3)CCCS4)cc2)CC1] | 2.41E−05 | | |
| 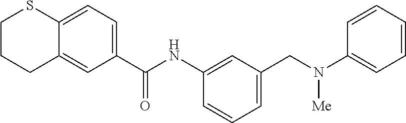 [SMILES: CN(Cc1cccc(NC(=O)c2ccc3c(c2)CCCS3)c1)c1ccccc1] | 3.75E−05 | | |
| 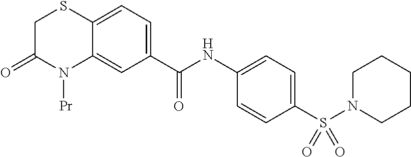 | 5.87E−06 | 39.94 | 17.0 |
| 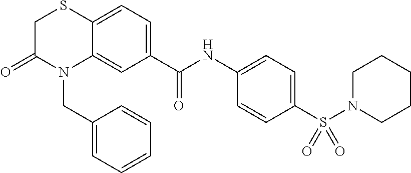 [SMILES: O=C(Nc1ccc(S(=O)(=O)N2CCCCC2)cc1)c1ccc2c(c1)N(Cc1ccccc1)C(=O)CS2] | 1.57E−05 | | |
| 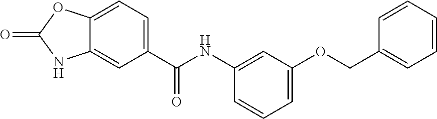 [SMILES: O=C(Nc1cccc(OCc2ccccc2)c1)c1ccc2oc(=O)[nH]c2c1] | 1.24E−05 | | |
| 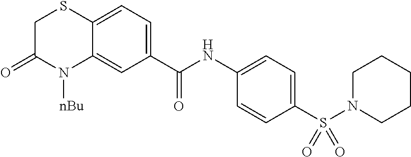 | 2.52E−05 | | |
| 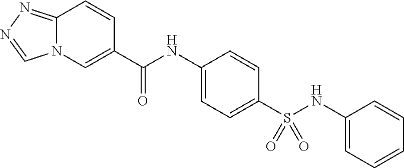 [SMILES: O=C(Nc1ccc(S(=O)(=O)Nc2ccccc2)cc1)c1ccc2nncn2c1] | 3.67E−04 | | |
| 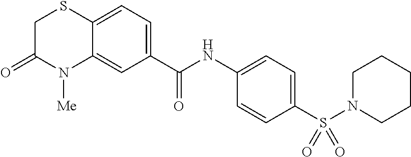 [SMILES: CN1C(=O)CSc2ccc(C(=O)Nc3ccc(S(=O)(=O)N4CCCCC4)cc3)cc21] | 2.79E−05 | | |
| 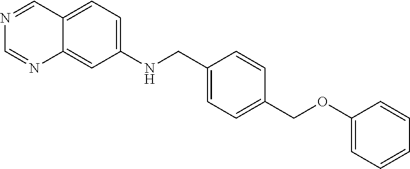 [SMILES: c1ccc(OCc2ccc(CNc3ccc4cncnc4c3)cc2)cc1] | 1.20E−05 | | |
| 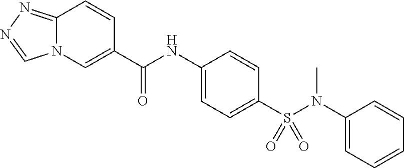 [SMILES: CN(c1ccccc1)S(=O)(=O)c1ccc(NC(=O)c2ccc3nncn3c2)cc1] | 4.07E−06 | 125.90 | >20.0 |
| 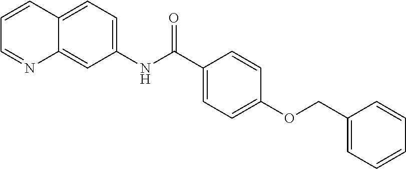 [SMILES: O=C(Nc1ccc2cccnc2c1)c1ccc(OCc2ccccc2)cc1] | 3.58E−06 | 22.75 | 8.0 |
| 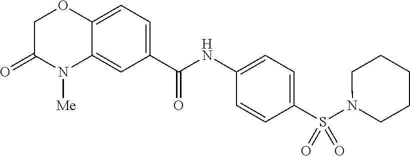 [SMILES: CN1C(=O)COc2ccc(C(=O)Nc3ccc(S(=O)(=O)N4CCCCC4)cc3)cc21] | 5.35E−06 | 90.59 | >20.0 |
| 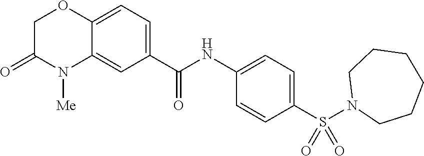 [SMILES: CN1C(=O)COc2ccc(C(=O)Nc3ccc(S(=O)(=O)N4CCCCCC4)cc3)cc21] | 4.94E−06 | | |
| 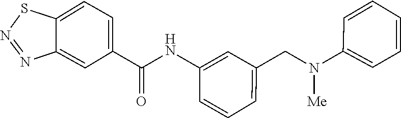 [SMILES: CN(Cc1cccc(NC(=O)c2ccc3snnc3c2)c1)c1ccccc1] | 3.88E−05 | | |
| 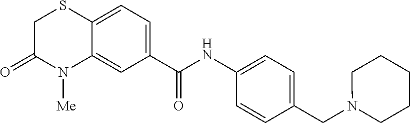 [SMILES: CN1C(=O)CSc2ccc(C(=O)Nc3ccc(CN4CCCCC4)cc3)cc21] | 1.29E−04 | | |
| 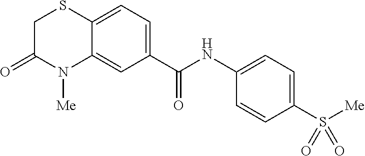 [SMILES: CN1C(=O)CSc2ccc(C(=O)Nc3ccc(S(C)(=O)=O)cc3)cc21] | 8.21E−05 | | |
| 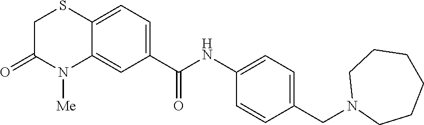 [SMILES: CN1C(=O)CSc2ccc(C(=O)Nc3ccc(CN4CCCCCC4)cc3)cc21] | 6.93E−06 | 102.08 | >20.0 |
| 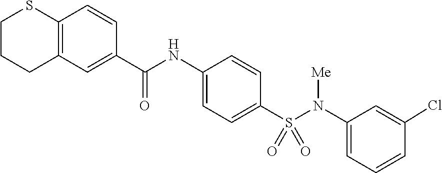 [SMILES: CN(c1cccc(Cl)c1)S(=O)(=O)c1ccc(NC(=O)c2ccc3c(c2)CCCS3)cc1] | 1.42E−05 | | |
| 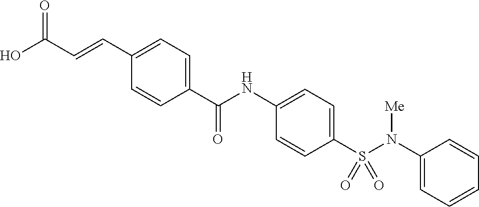 [SMILES: CN(c1ccccc1)S(=O)(=O)c1ccc(NC(=O)c2ccc(/C=C/C(=O)O)cc2)cc1] | 1.06E−04 | | |
| 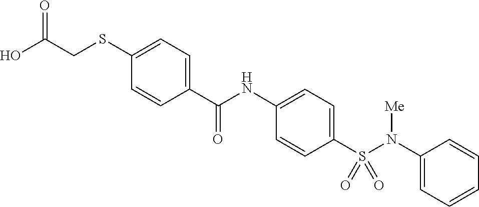 [SMILES: CN(c1ccccc1)S(=O)(=O)c1ccc(NC(=O)c2ccc(SCC(=O)O)cc2)cc1] | 1.10E−05 | | |
| 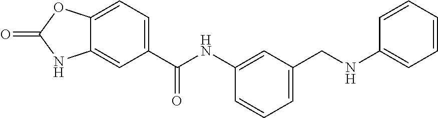 [SMILES: O=C(Nc1cccc(CNc2ccccc2)c1)c1ccc2oc(=O)[nH]c2c1] | 1.82E−05 | | |
| 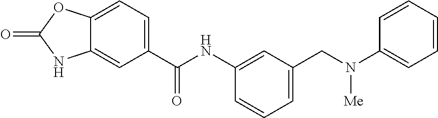 [SMILES: CN(Cc1cccc(NC(=O)c2ccc3oc(=O)[nH]c3c2)c1)c1ccccc1] | 1.36E−05 | | |
| 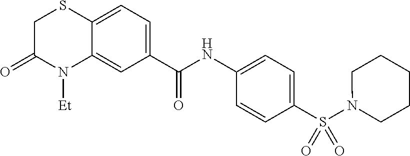 [SMILES: CCN1C(=O)CSc2ccc(C(=O)Nc3ccc(S(=O)(=O)N4CCCCC4)cc3)cc21] | 7.52E−06 | | |
| 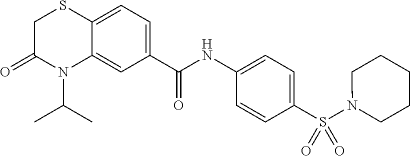 [SMILES: CC(C)N1C(=O)CSc2ccc(C(=O)Nc3ccc(S(=O)(=O)N4CCCCC4)cc3)cc21] | 6.37E−06 | | |
| 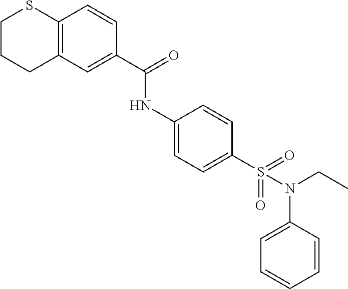 [SMILES: CCN(c1ccccc1)S(=O)(=O)c1ccc(NC(=O)c2ccc3c(c2)CCCS3)cc1] | 1.30E−04 | | |
| 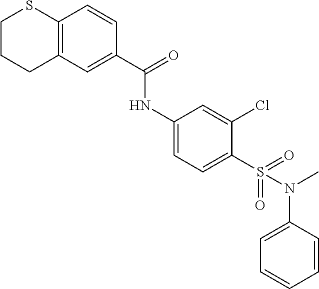 [SMILES: CN(c1ccccc1)S(=O)(=O)c1ccc(NC(=O)c2ccc3c(c2)CCCS3)cc1Cl] | 1.02E−05 | | |
| 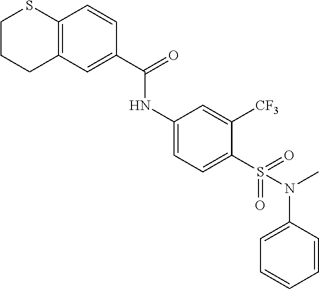 [SMILES: CN(c1ccccc1)S(=O)(=O)c1ccc(NC(=O)c2ccc3c(c2)CCCS3)cc1C(F)(F)F] | 5.62E−06 | | |
| 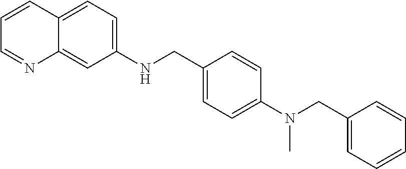 [SMILES: CN(Cc1ccccc1)c1ccc(CNc2ccc3cccnc3c2)cc1] | 9.27E−06 | | |
| 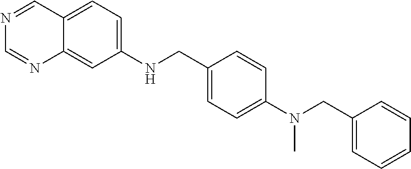 [SMILES: CN(Cc1ccccc1)c1ccc(CNc2ccc3cncnc3c2)cc1] | 2.07E−05 | | |
| 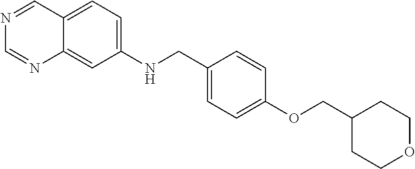 [SMILES: c1ncc2ccc(NCc3ccc(OCC4CCOCC4)cc3)cc2n1] | 2.44E−05 | | |
| 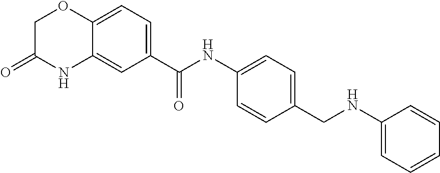 [SMILES: O=C1COc2ccc(C(=O)Nc3ccc(CNc4ccccc4)cc3)cc2N1] | 5.28E−06 | | |
| 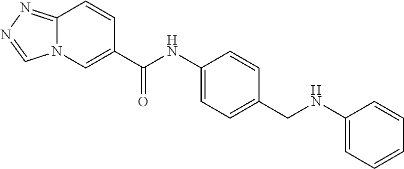 [SMILES: O=C(Nc1ccc(CNc2ccccc2)cc1)c1ccc2nncn2c1] | 4.61E−05 | | |
| 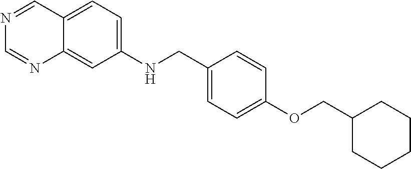 [SMILES: c1ncc2ccc(NCc3ccc(OCC4CCCCC4)cc3)cc2n1] | 8.28E−06 | | |
| 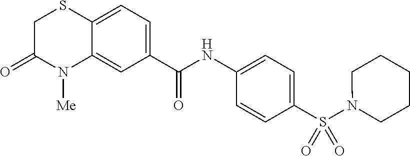 [SMILES: CN1C(=O)CSc2ccc(C(=O)Nc3ccc(S(=O)(=O)N4CCCCC4)cc3)cc21] | 3.11E−06 | | |
| 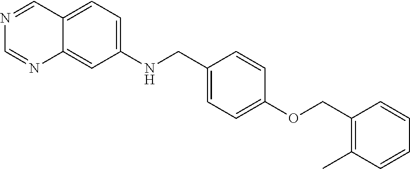 [SMILES: Cc1ccccc1COc1ccc(CNc2ccc3cncnc3c2)cc1] | 1.11E−05 | | |
| 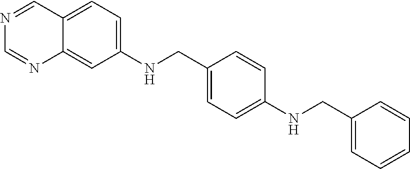 [SMILES: c1ccc(CNc2ccc(CNc3ccc4cncnc4c3)cc2)cc1] | 3.06E−05 | | |
| 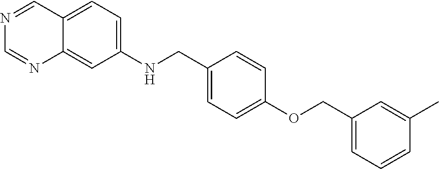 [SMILES: Cc1cccc(COc2ccc(CNc3ccc4cncnc4c3)cc2)c1] | 7.63E−06 | | |
| 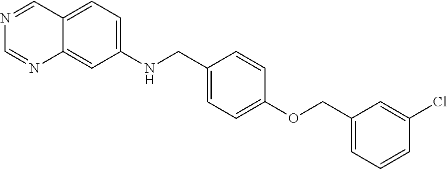 [SMILES: Clc1cccc(COc2ccc(CNc3ccc4cncnc4c3)cc2)c1] | 3.28E−06 | | |
| 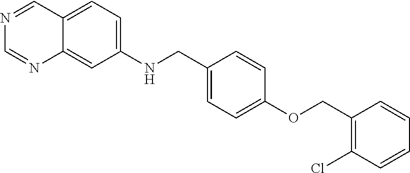 [SMILES: Clc1ccccc1COc1ccc(CNc2ccc3cncnc3c2)cc1] | 7.88E−06 | | |
| 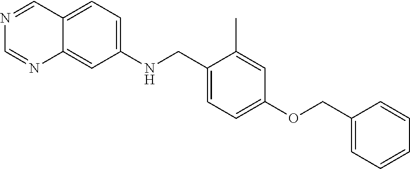 [SMILES: Cc1cc(OCc2ccccc2)ccc1CNc1ccc2cncnc2c1] | 9.24E−06 | | |
| 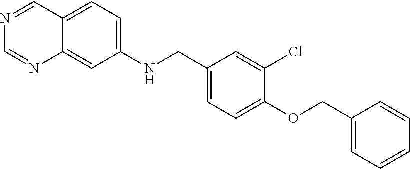 [SMILES: Clc1cc(CNc2ccc3cncnc3c2)ccc1OCc1ccccc1] | 4.01E−06 | | |
| 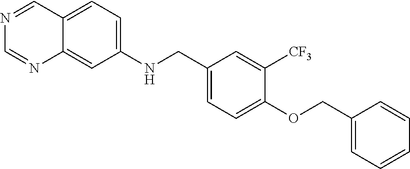 [SMILES: FC(F)(F)c1cc(CNc2ccc3cncnc3c2)ccc1OCc1ccccc1] | 4.89E−06 | | |
| 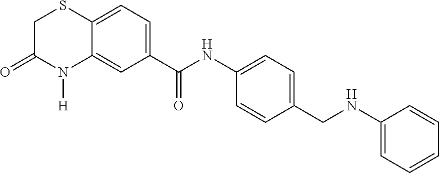 [SMILES: O=C1CSc2ccc(C(=O)Nc3ccc(CNc4ccccc4)cc3)cc2N1] | 1.74E−06 | | |
| 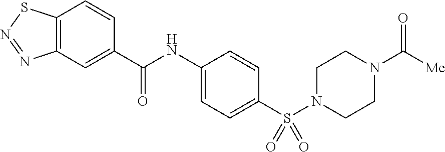 [SMILES: CC(=O)N1CCN(S(=O)(=O)c2ccc(NC(=O)c3ccc4snnc4c3)cc2)CC1] | 1.69E−05 | | |
| 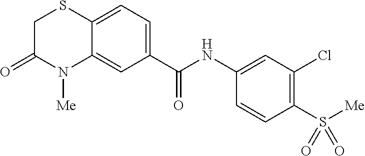 [SMILES: CN1C(=O)CSc2ccc(C(=O)Nc3ccc(S(C)(=O)=O)c(Cl)c3)cc21] | 1.41E−04 | | |
| 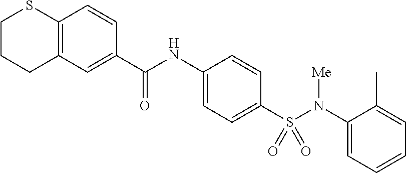 [SMILES: Cc1ccccc1N(C)S(=O)(=O)c1ccc(NC(=O)c2ccc3c(c2)CCCS3)cc1] | 1.03E−05 | | |
| 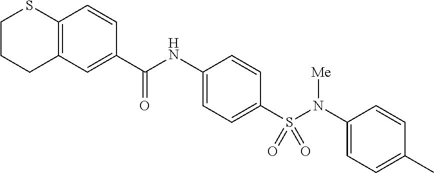 [SMILES: Cc1ccc(N(C)S(=O)(=O)c2ccc(NC(=O)c3ccc4c(c3)CCCS4)cc2)cc1] | 2.73E−06 | | |
| 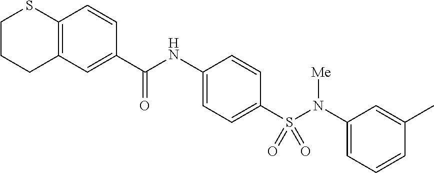 [SMILES: Cc1cccc(N(C)S(=O)(=O)c2ccc(NC(=O)c3ccc4c(c3)CCCS4)cc2)c1] | 4.55E−06 | | |
| 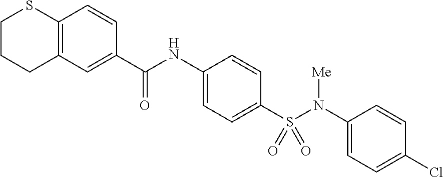 [SMILES: CN(c1ccc(Cl)cc1)S(=O)(=O)c1ccc(NC(=O)c2ccc3c(c2)CCCS3)cc1] | 1.08E−05 | | |
| 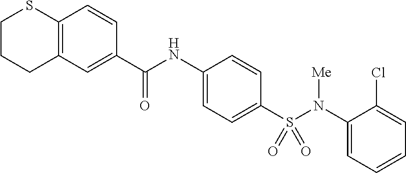 [SMILES: CN(c1ccccc1Cl)S(=O)(=O)c1ccc(NC(=O)c2ccc3c(c2)CCCS3)cc1] | 2.39E−06 | | |
| 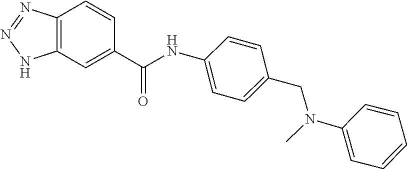 [SMILES: CN(Cc1ccc(NC(=O)c2ccc3nn[nH]c3c2)cc1)c1ccccc1] | 2.90E−06 | | |
| 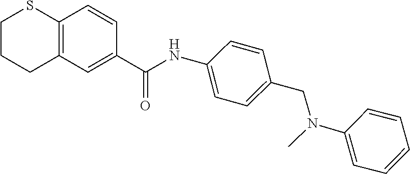 [SMILES: CN(Cc1ccc(NC(=O)c2ccc3c(c2)CCCS3)cc1)c1ccccc1] | 1.15E−05 | | |
| 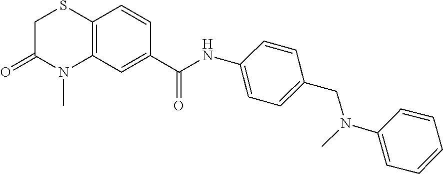 [SMILES: CN(Cc1ccc(NC(=O)c2ccc3c(c2)N(C)C(=O)CS3)cc1)c1ccccc1] | 8.10E−06 | | |
| 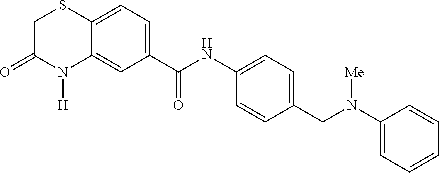 [SMILES: CN(Cc1ccc(NC(=O)c2ccc3c(c2)NC(=O)CS3)cc1)c1ccccc1] | 2.48E−05 | | |
| 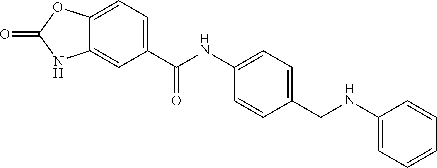 [SMILES: O=C(Nc1ccc(CNc2ccccc2)cc1)c1ccc2oc(=O)[nH]c2c1] | 7.91E−06 | | |
| 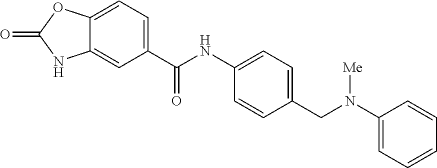 [SMILES: CN(Cc1ccc(NC(=O)c2ccc3oc(=O)[nH]c3c2)cc1)c1ccccc1] | 1.56E−05 | | |
| 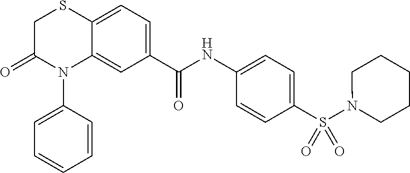 [SMILES: O=C(Nc1ccc(S(=O)(=O)N2CCCCC2)cc1)c1ccc2c(c1)N(c1ccccc1)C(=O)CS2] | 6.67E−06 | | |
| 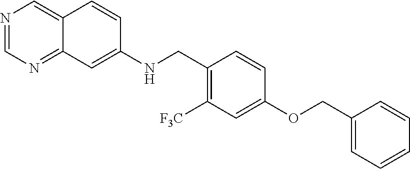 [SMILES: FC(F)(F)c1cc(OCc2ccccc2)ccc1CNc1ccc2cncnc2c1] | 5.28E−06 | | |

While typical embodiments have been set forth for the purpose of illustration, the foregoing descriptions and examples should not be deemed to be a limitation on the scope of the invention. Accordingly, various modifications, adaptations, and alternatives may occur to one skilled in the art without departing from the spirit and scope of the present invention. While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. A compound of formula I: 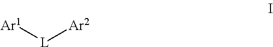
wherein:
i) Ar¹ is 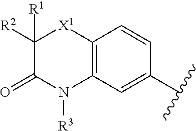
wherein:
X¹ is S or O;
R¹ and R² are independently selected from optionally substituted (C₁-C₆)alkyl and H; and
R³ is H or (C₁-C₁₀)hydrocarbyl;
L is —CONH—, —NHCO—, or —NHCH₂—; and
Ar² is a mono- or di-substituted monocyclic aryl or heteroaryl, wherein the substituents are selected from —SO₂—R¹⁰, —OSO₂—R¹⁰, perfluoro(C₁-C₃)alkyl, halo, (C₁-C₃)alkyl, —C(═O)R¹⁰, —OCH₂R¹¹, —OR¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰;
wherein R¹⁰ is selected from arylamino, perfluoro(C₁-C₃)alkyl-substituted arylamino, halo-substituted arylamino, (C₁-C₃)alkyl-substituted arylamino, amino, (C₁-C₃)alkyl, heterocyclyl, (C₁-C₆)dialkylamino, pyridylamino, (C₁-C₆)alkylamino, (C₁-C₆)cycloalkylamino, arylamino, oxo-substituted heteroarylamino, heterocyclylamino, hydroxy-substituted arylamino, amino-substituted arylamino, pyridin-2(1H)-one-amino, (C₁-C₆)dihydrocarbylamino, fluoro, (C₁-C₃)alkylarylamino, acetyl-substituted heterocyclyl, and (C₁-C₃)alkylhaloarylamino; and
wherein R¹¹ is selected from optionally substituted aryl, unsubstituted benzyl, perfluoro(C₁-C₃)alkyl-substituted benzyl, halo-substituted benzyl, (C₁-C₃)alkyl-substituted benzyl, (C₁-C₃)alkyl, heterocyclyl, (C₁-C₆)dialkyl, pyridyl, (C₁-C₆)alkyl, (C₁-C₆)cycloalkyl, benzyl, oxo-substituted heteroarylbenzyl, heterocyclyl, hydroxy-substituted benzyl, amino-substituted benzyl, pyridin-2(1H)-one, (C₁-C₆)dihydrocarbyl, (C₁-C₃)alkylbenzyl, acetyl-substituted heterocyclyl, and (C₁-C₃)alkylhalobenzyl; or
ii) Ar¹ is 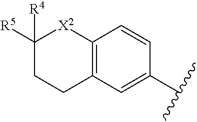
wherein:
X² is S or O; and
R⁴ and R⁵ are independently selected from H and (C₁-C₆)alkyl;
L is —CONH— or —NHCO—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰;
wherein R¹⁰ is selected from arylamino, perfluoro(C₁-C₃)alkyl-substituted arylamino, halo-substituted arylamino, (C₁-C₃)alkyl-substituted arylamino, amino, (C₁-C₃)alkyl, heterocyclyl, (C₁-C₆)dialkylamino, pyridylamino, (C₁-C₆)alkylamino, (C₁-C₆)cycloalkylamino, arylamino, oxo-substituted heteroarylamino, heterocyclylamino, hydroxy-substituted arylamino, amino-substituted arylamino, pyridin-2(1H)-one-amino, (C₁-C₆)dihydrocarbylamino, fluoro, (C₁-C₃)alkylarylamino, acetyl-substituted heterocyclyl, and (C₁-C₃)alkylhaloarylamino; and
wherein R¹¹ is selected from benzyl, perfluoro(C₁-C₃)alkyl-substituted benzyl, halo-substituted benzyl, (C₁-C₃)alkyl-substituted benzyl, (C₁-C₃)alkyl, heterocyclyl, (C₁-C₆)dialkyl, pyridyl, (C₁-C₆)alkyl, (C₁-C₆)cycloalkyl, benzyl, oxo-substituted heteroarylbenzyl, heterocyclyl, hydroxy-substituted benzyl, amino-substituted benzyl, pyridin-2(1H)-one, (C₁-C₆)dihydrocarbyl, (C₁-C₃)alkylbenzyl, acetyl-substituted heterocyclyl, and (C₁-C₃)alkylhalobenzyl; or
iii) Ar¹ is 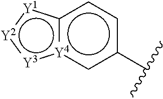
wherein:
all backbone atoms of the 6,5-bicyclic structure are sp²-hybridized;
Y¹ is selected from S, CH, N, NH, and O;
Y² is selected from N, NH, C—R⁶, and C═O; wherein R⁶ is H, (C₁-C₃)alkyl, or amino(C₁-C₃)alkyl;
Y³ is selected from N, NH, CH, and C—CH₃; and
Y⁴ is C or N;
L is —CONH— or —NHCO—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰, or
iv) Ar¹ is 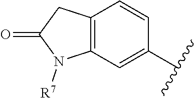
wherein R⁷ is H or optionally substituted (C₁-C₃)alkyl;
L is —CONH— or —NHCO—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰, or
v) Ar¹ is 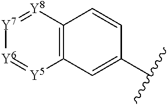
wherein Y⁵, Y⁶, Y⁷, and Y⁸ are independently chosen from C and N;
L is —CONH—, —NHCO—, or —NHCH₂—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰; or
vi) Ar¹ is 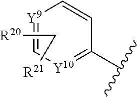
wherein Y⁹ and Y¹⁰ are independently chosen from C and N;
R²⁰ and R²¹ are independently chosen from hydrogen, (C₁-C₃)alkyl, amino, aryl-substituted heterocyclic amino, heteroaryl-substituted heterocyclic amino, unsubstituted heterocyclic amino, —CH═CHCOOH, 4-aminocyclohexylamino, acetylethylenediamino, amino- and/or (C₁-C₃)alkyl-substituted heterocyclic amino, —NHC(═O)(CH₂)ₙ-heterocyclyl wherein n is either 1 or 2, ethylenediamino, (C₁-C₃)alkoxy, and acetylmethylamino;
L is —CONH—, 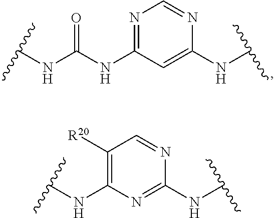
wherein R²⁰ is H or methyl; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰.

2. A compound of formula II: 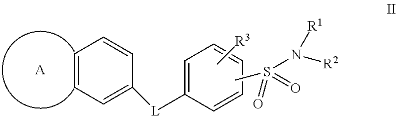
wherein:
R¹ is selected from hydrogen and optionally substituted C₁-C₄ alkyl;
R² is selected from C₁-C₄ alkyl; C₃-C₆ cycloalkyl; tert-butyl piperidine-1-carboxylate; pyridin-2(1H)-one or phenyl optionally substituted with C₁-C₄ alkyl, C₁-C₄ haloalkyl, —OH, or halogen; or
taken together with the nitrogen to which they are attached, R¹ and R² form a five- to seven-membered, non-aromatic heterocyclic ring optionally substituted with tert-butyl carboxylate, wherein said heterocyclic ring contains no additional —NH— group.
R³ is selected from hydrogen, halogen, C₁-C₄ alkyl, or C₁-C₄ haloalkyl;
L is selected from 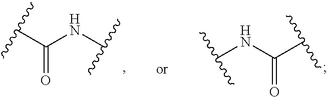 [SMILES: *NC(*)=O ; *NC(*)=O]
Ring A is selected from 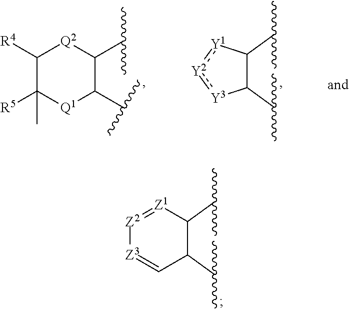
wherein:
Q¹ is selected from NH, NCH₃, or CH₂;
Q² is selected from S or O;
R⁴ is selected from hydrogen and C₁-C₄ alkyl;
R⁵ and R⁶ are each independently hydrogen; or
R⁵ and R⁶ taken together form ═O; [symbol]
represents a single bond or a double bond;
Y¹ is selected from S, CH, NR^{Y1}, or O;
Y² is selected from NR^{Y1}, CR^{Y2}, or C═O;
Y³ is selected from NR^{Y1} or CR^{Y2};
wherein at least one of Y¹, Y², and Y³ is NR^{Y1};
R^{Y1} is either hydrogen or a lone pair on the nitrogen atom to which it is attached;
R^{Y2} is selected from hydrogen or CH₃;
Z¹, Z², and Z³ are each independently selected from CH and N; wherein one of Z¹, Z², and Z³ is N and the remaining two of Z¹, Z², and Z³ are CH;

3. A compound according to claim 1, wherein L is

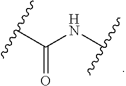 [SMILES: *NC(*)=O]

4. A compound selected from the group consisting of:

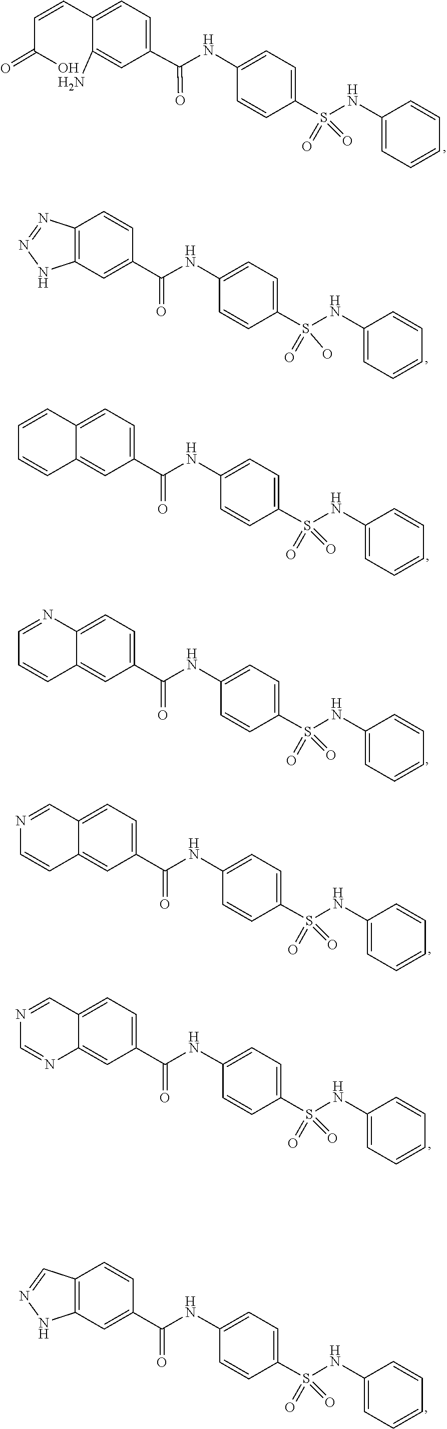

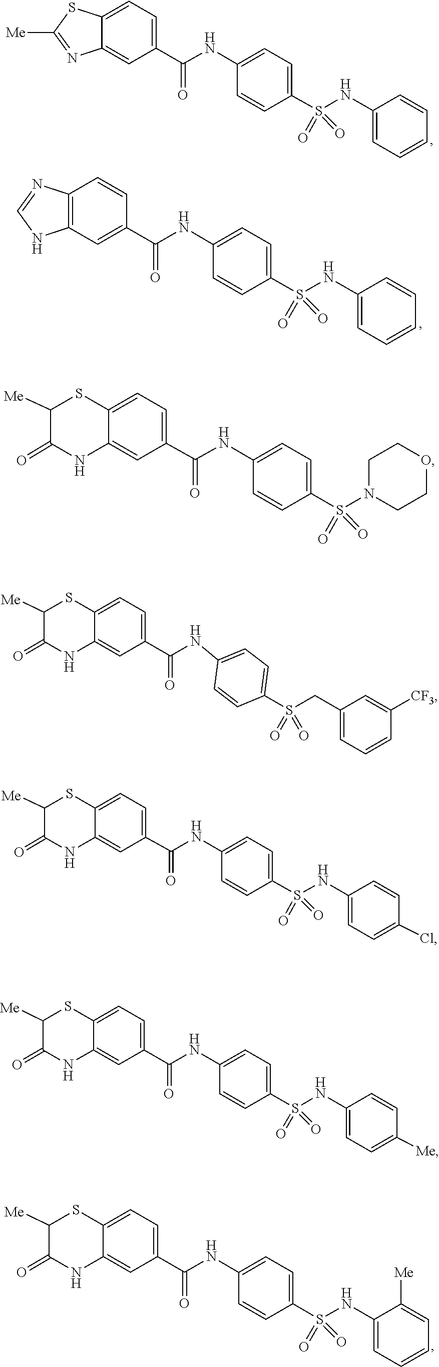

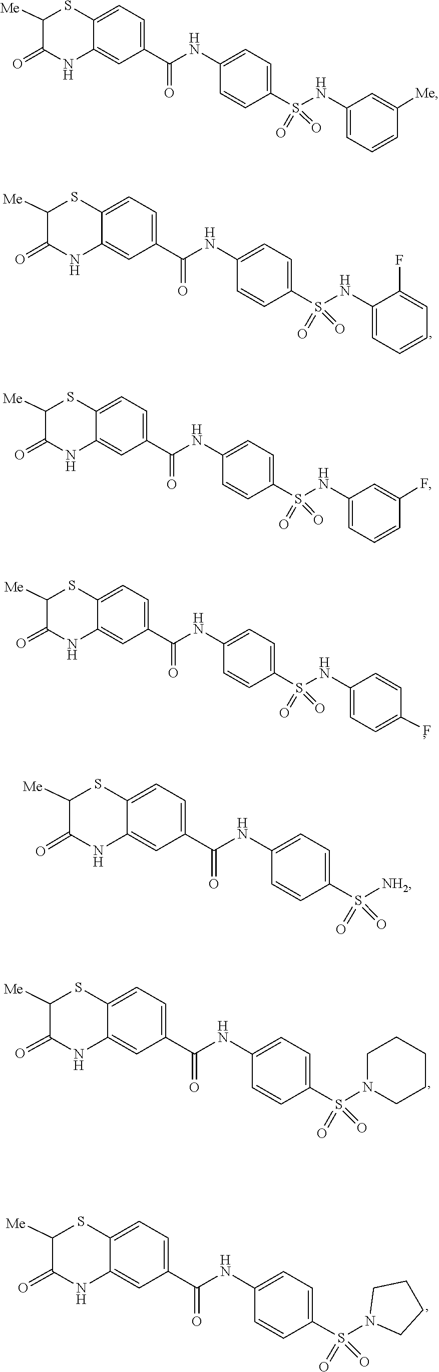

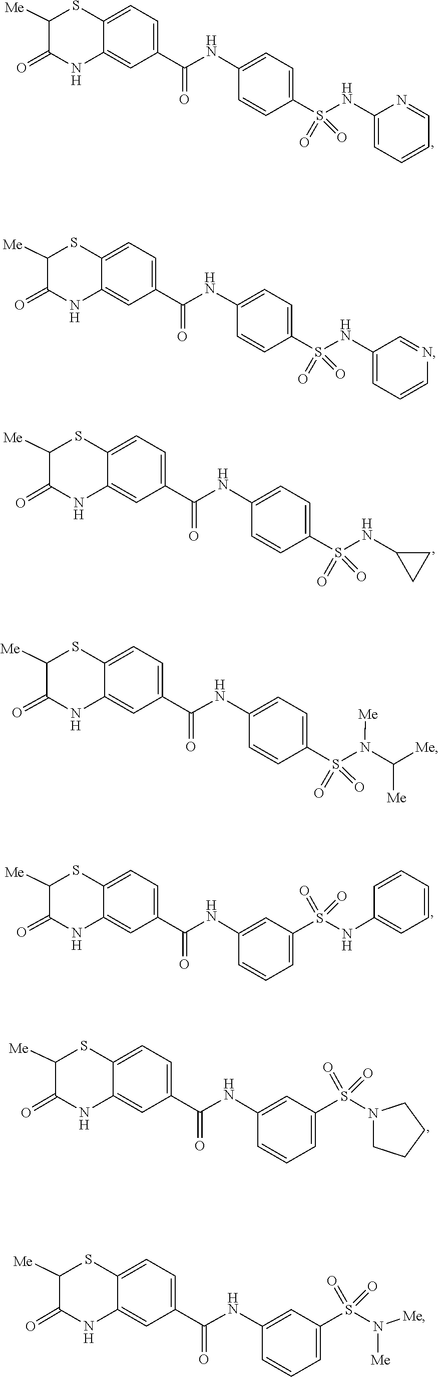

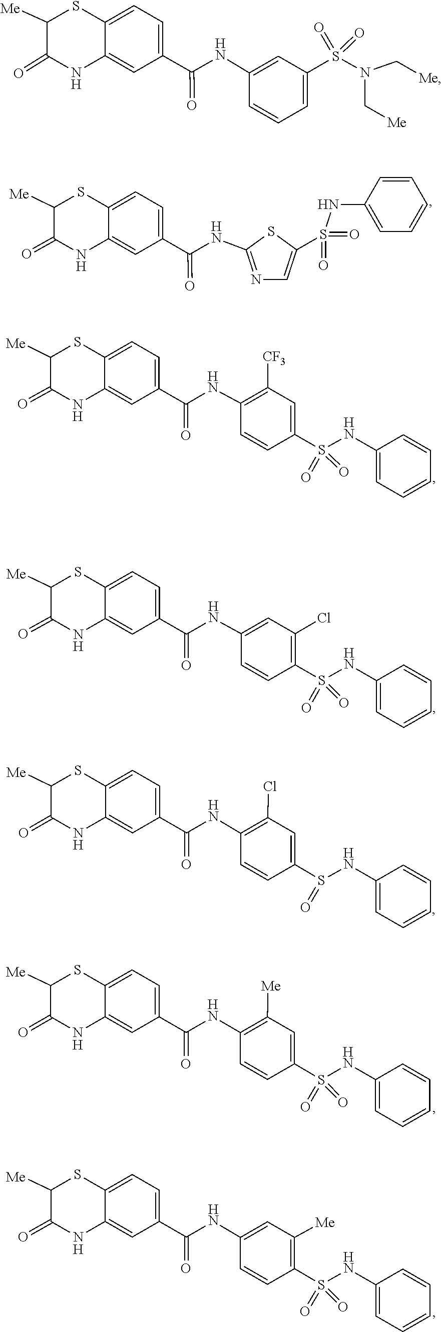

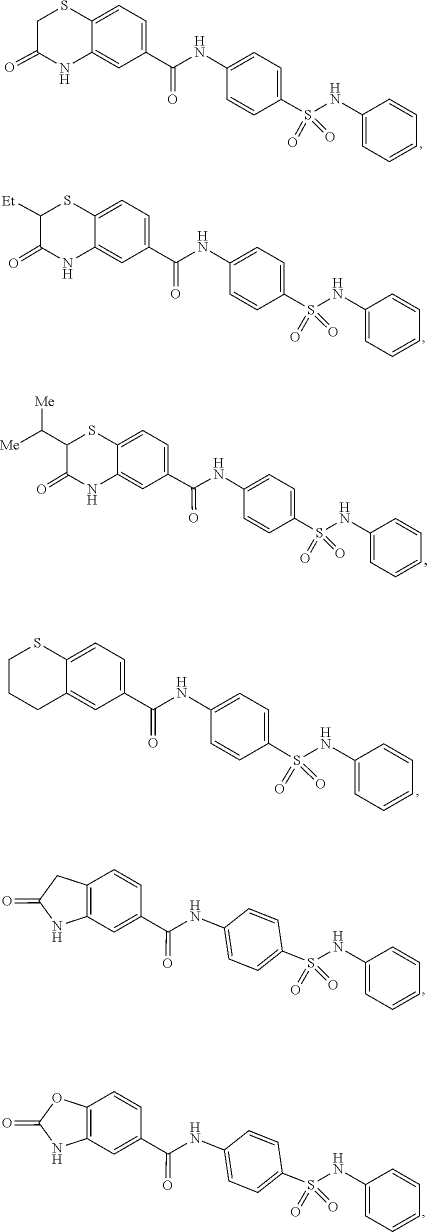 [SMILES: O=C1CSc2ccc(C(=O)Nc3ccc(S(=O)(=O)Nc4ccccc4)cc3)cc2N1 ; CCC1Sc2ccc(C(=O)Nc3ccc(S(=O)(=O)Nc4ccccc4)cc3)cc2NC1=O ; CC(C)C1Sc2ccc(C(=O)Nc3ccc(S(=O)(=O)Nc4ccccc4)cc3)cc2NC1=O ; O=C(Nc1ccc(S(=O)(=O)Nc2ccccc2)cc1)c1ccc2c(c1)CCCS2 ; O=C1Cc2ccc(C(=O)Nc3ccc(S(=O)(=O)Nc4ccccc4)cc3)cc2N1 ; O=C(Nc1ccc(S(=O)(=O)Nc2ccccc2)cc1)c1ccc2oc(=O)[nH]c2c1]

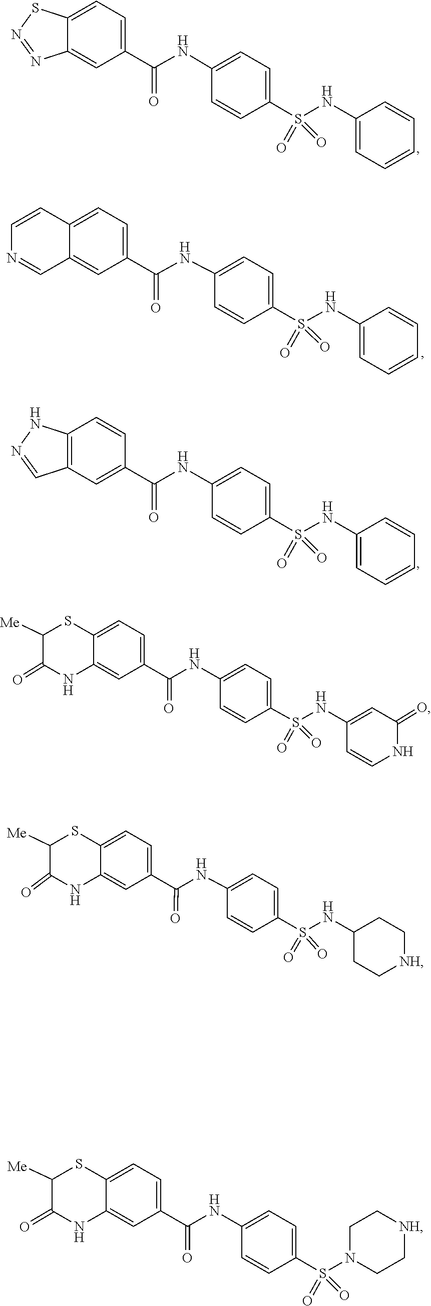 [SMILES: O=C(Nc1ccc(S(=O)(=O)Nc2ccccc2)cc1)c1ccc2snnc2c1 ; O=C(Nc1ccc(S(=O)(=O)Nc2ccccc2)cc1)c1ccc2ccncc2c1 ; O=C(Nc1ccc(S(=O)(=O)Nc2ccccc2)cc1)c1ccc2[nH]ncc2c1 ; CC1Sc2ccc(C(=O)Nc3ccc(S(=O)(=O)Nc4cc[nH]c(=O)c4)cc3)cc2NC1=O ; CC1Sc2ccc(C(=O)Nc3ccc(S(=O)(=O)NC4CCNCC4)cc3)cc2NC1=O ; CC1Sc2ccc(C(=O)Nc3ccc(S(=O)(=O)N4CCNCC4)cc3)cc2NC1=O]

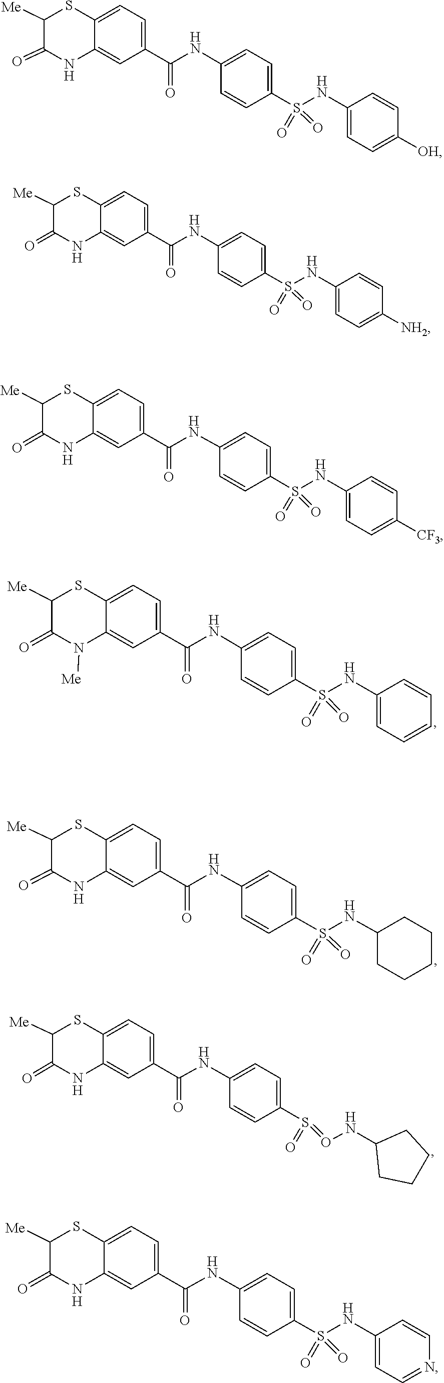

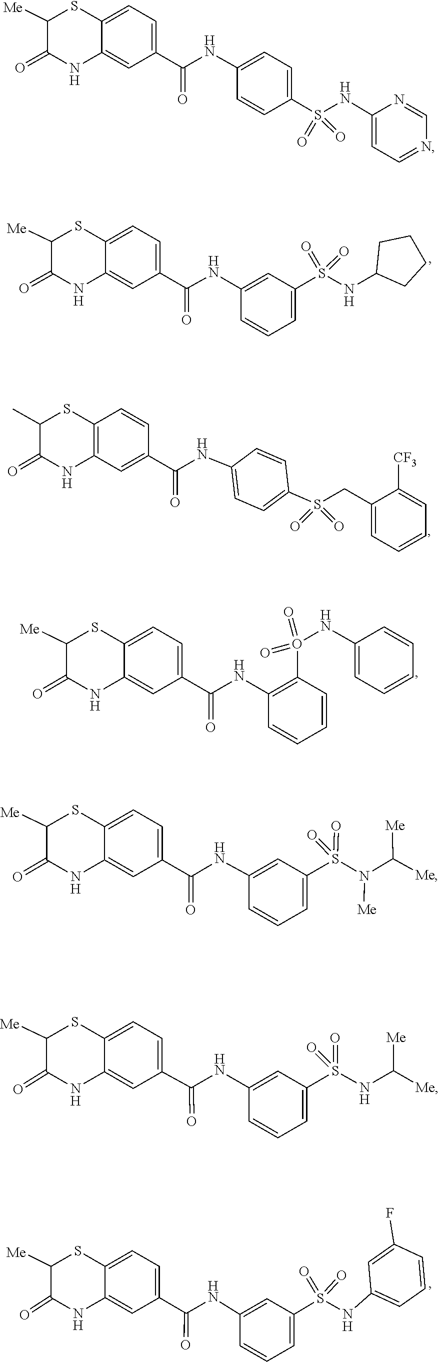

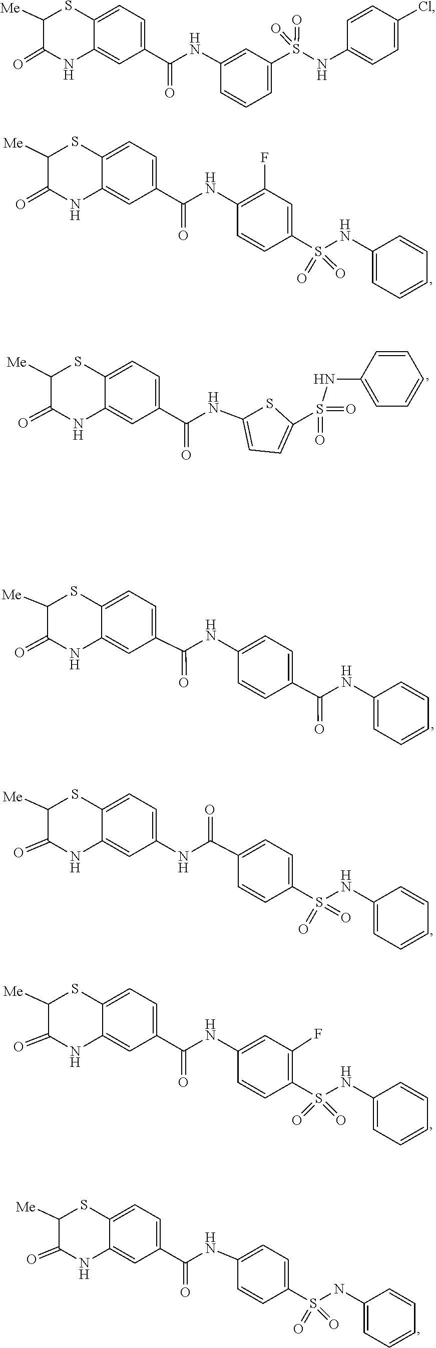

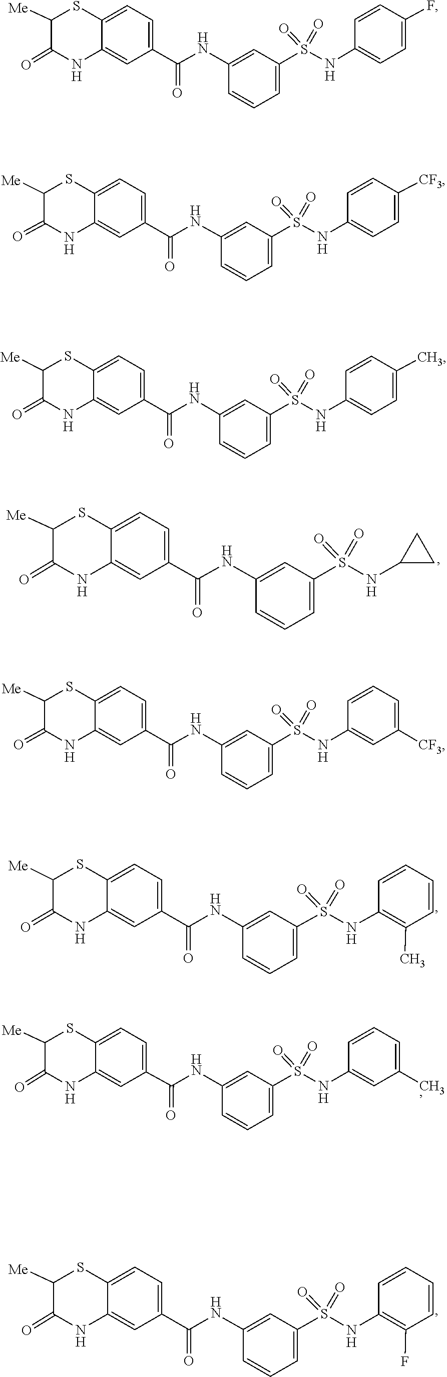

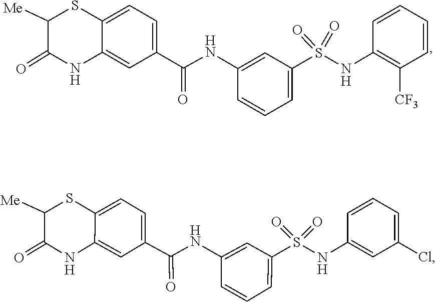 [SMILES: CC1Sc2ccc(C(=O)Nc3cccc(S(=O)(=O)Nc4ccccc4C(F)(F)F)c3)cc2NC1=O ; CC1Sc2ccc(C(=O)Nc3cccc(S(=O)(=O)Nc4cccc(Cl)c4)c3)cc2NC1=O]

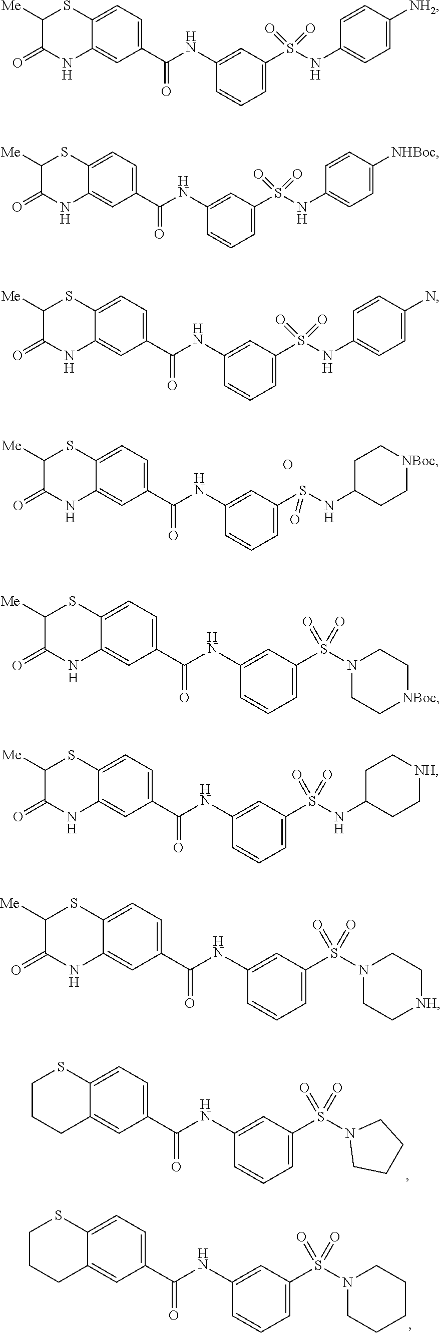

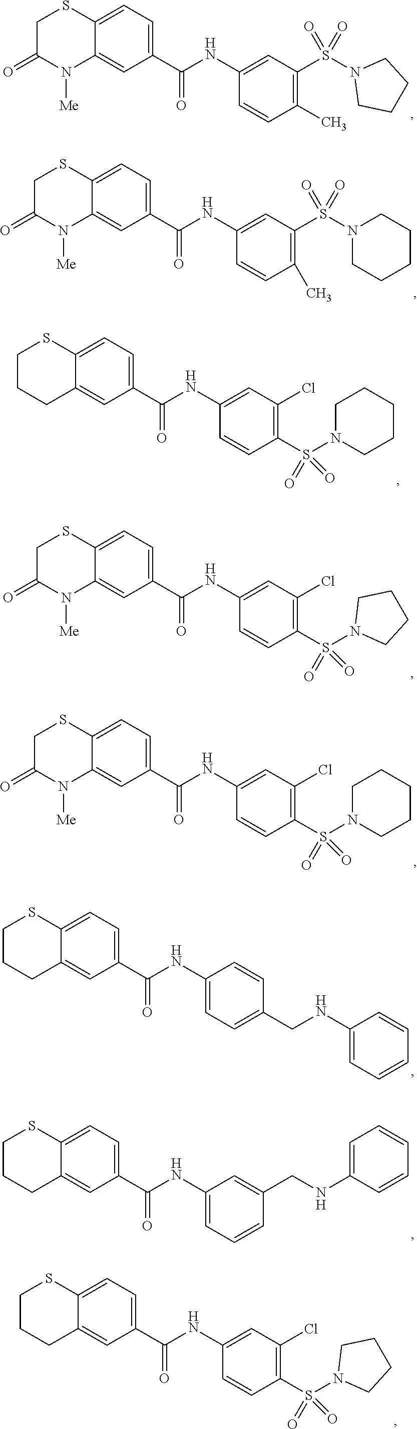

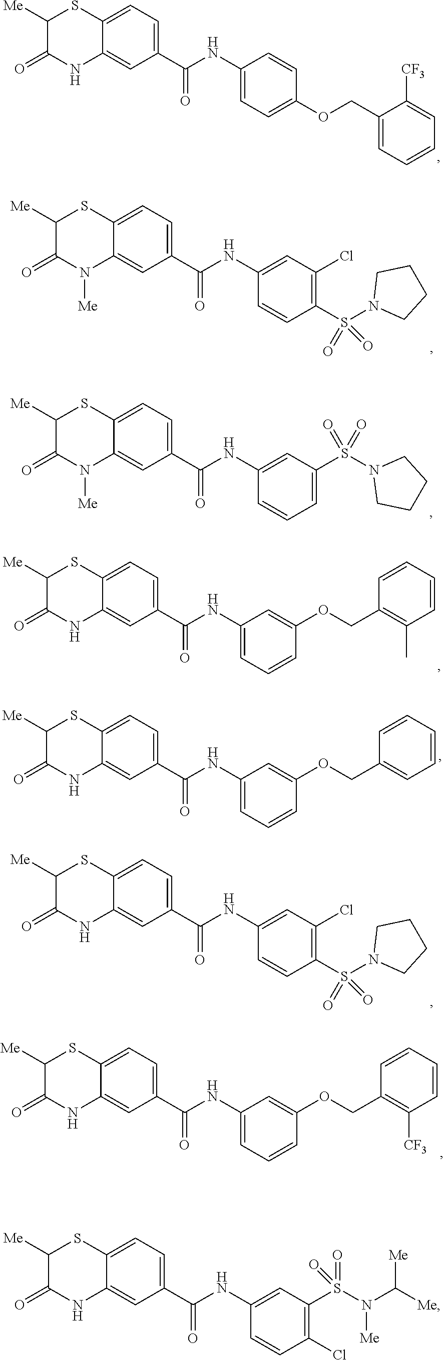

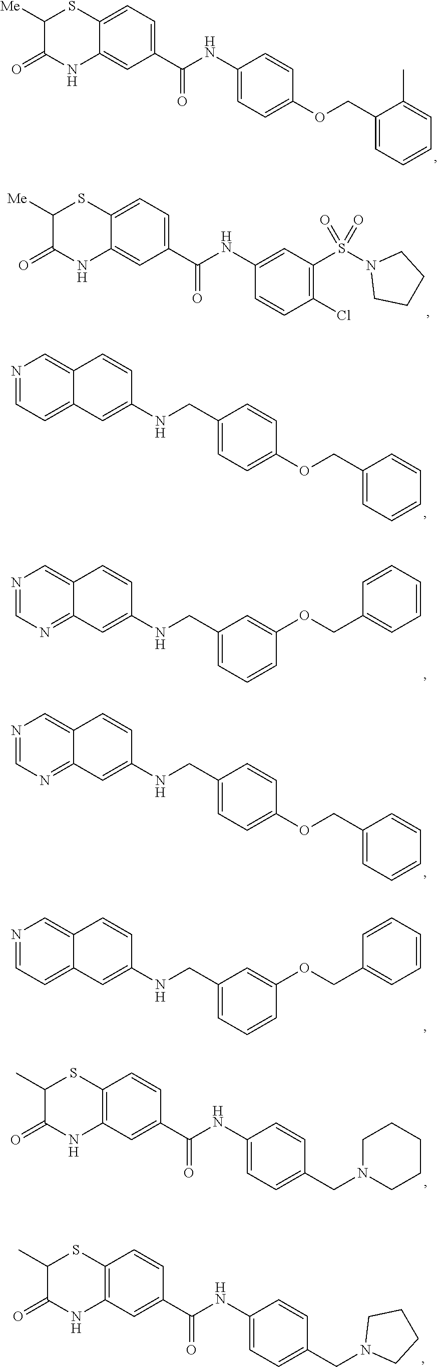

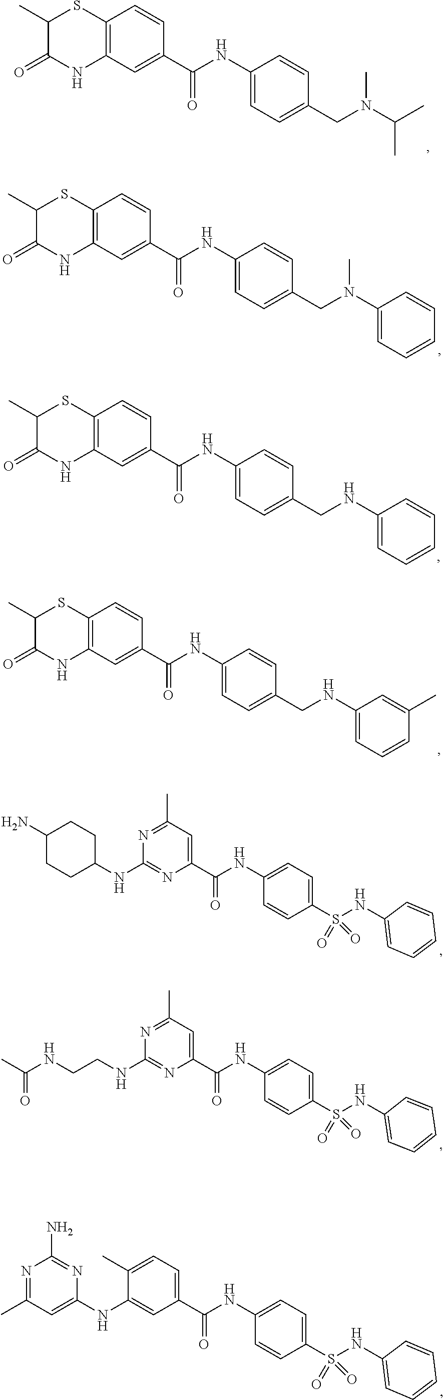

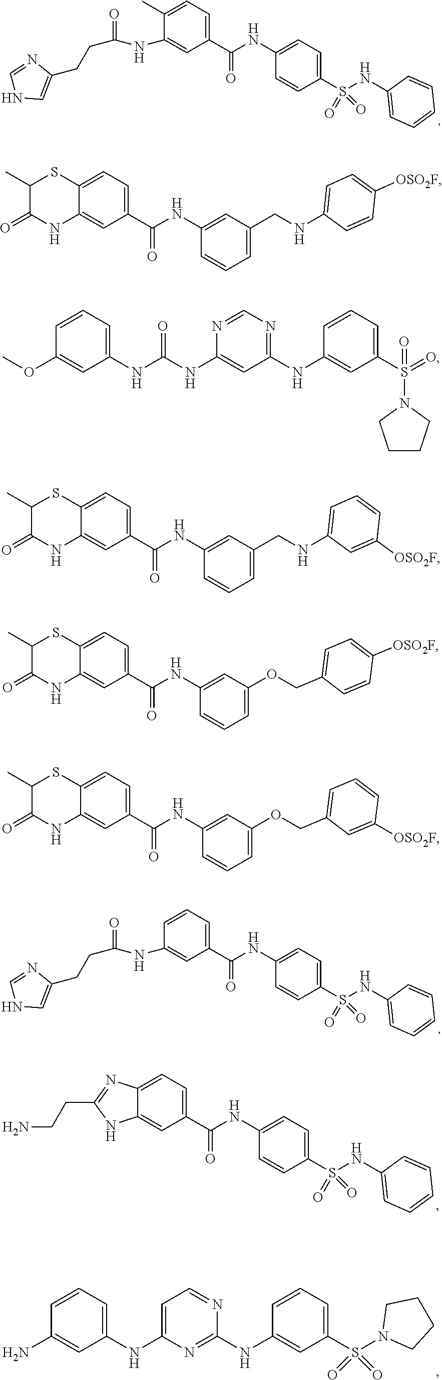

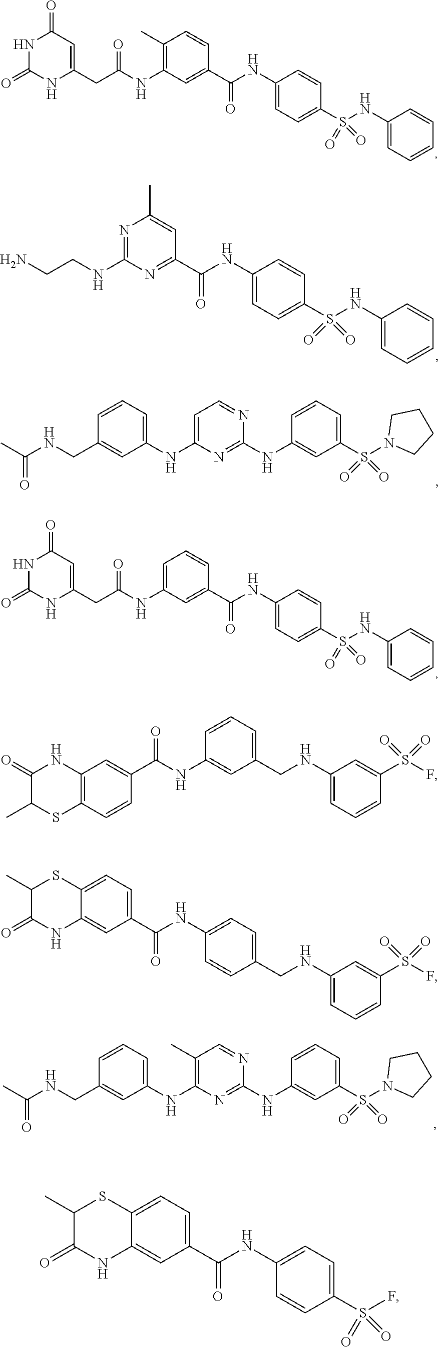

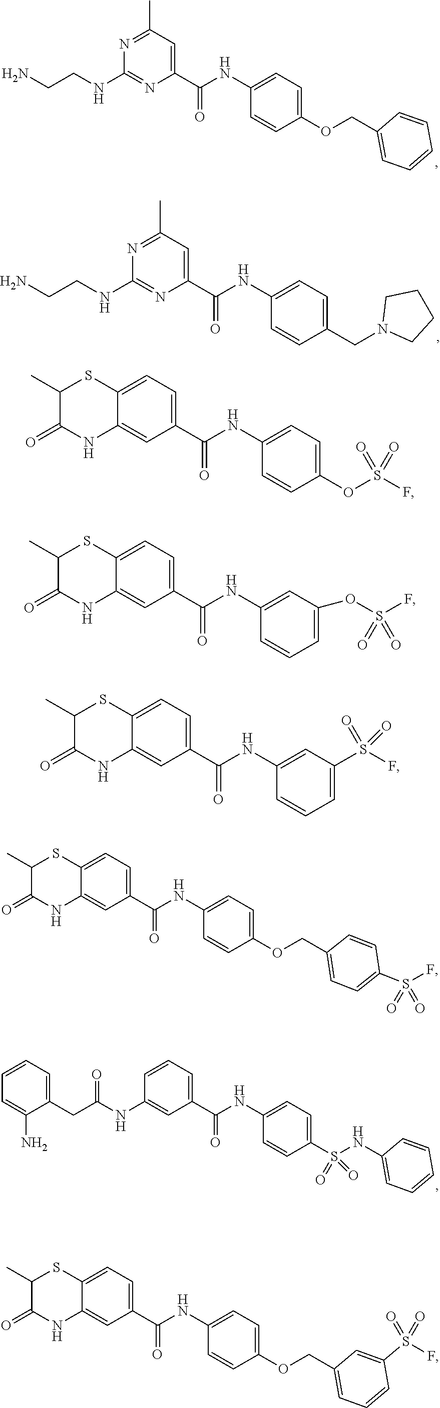

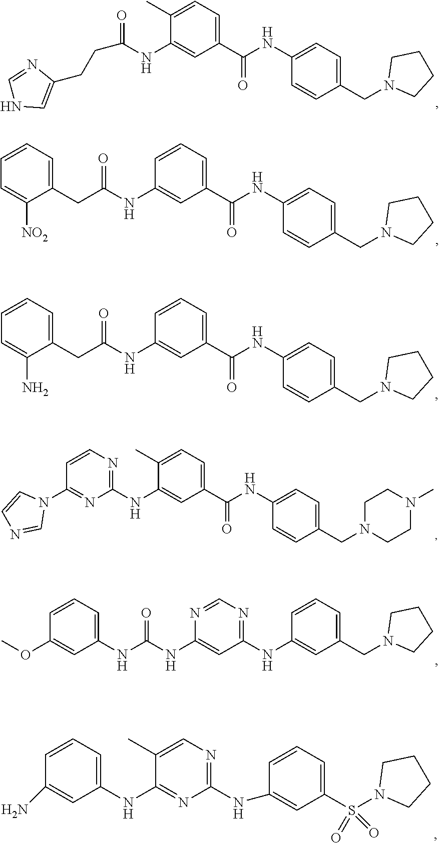

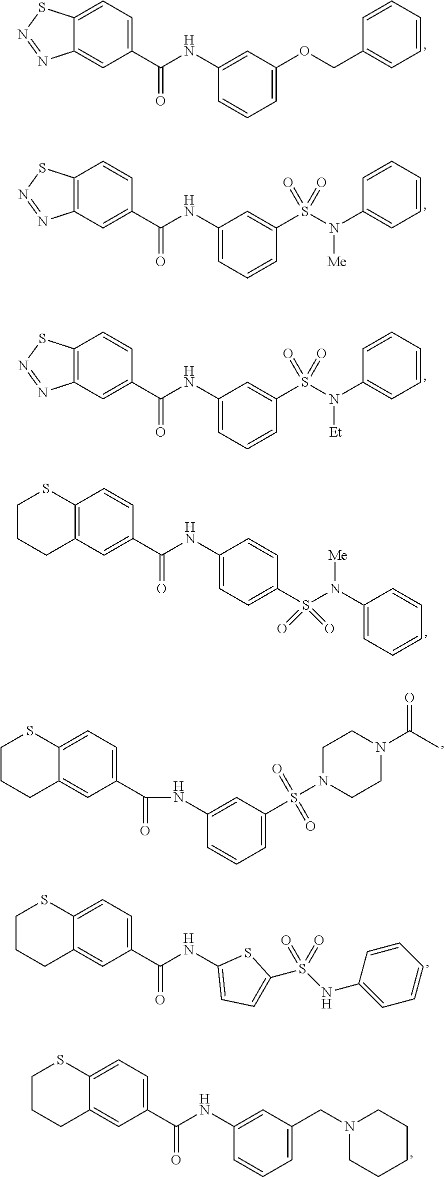

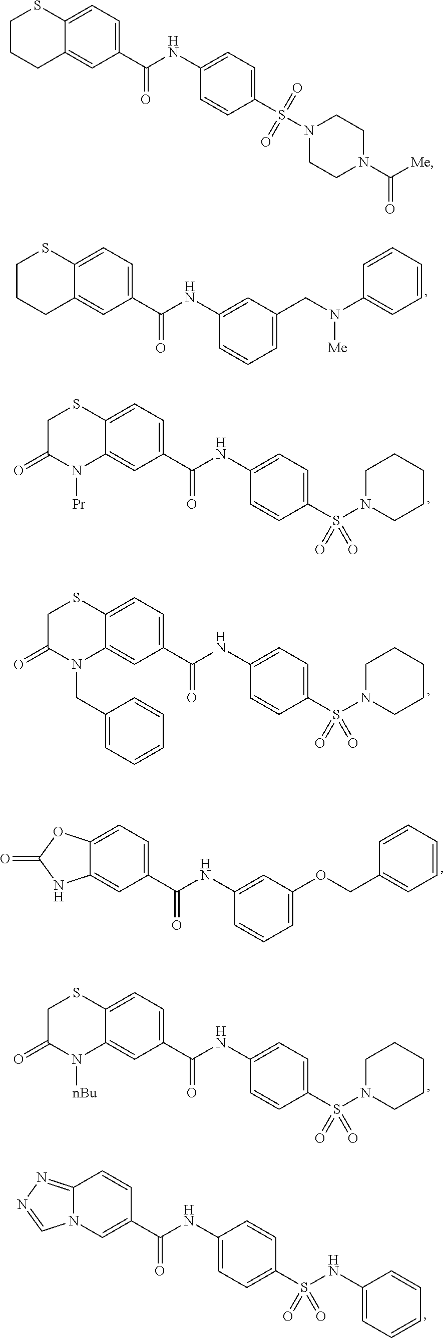

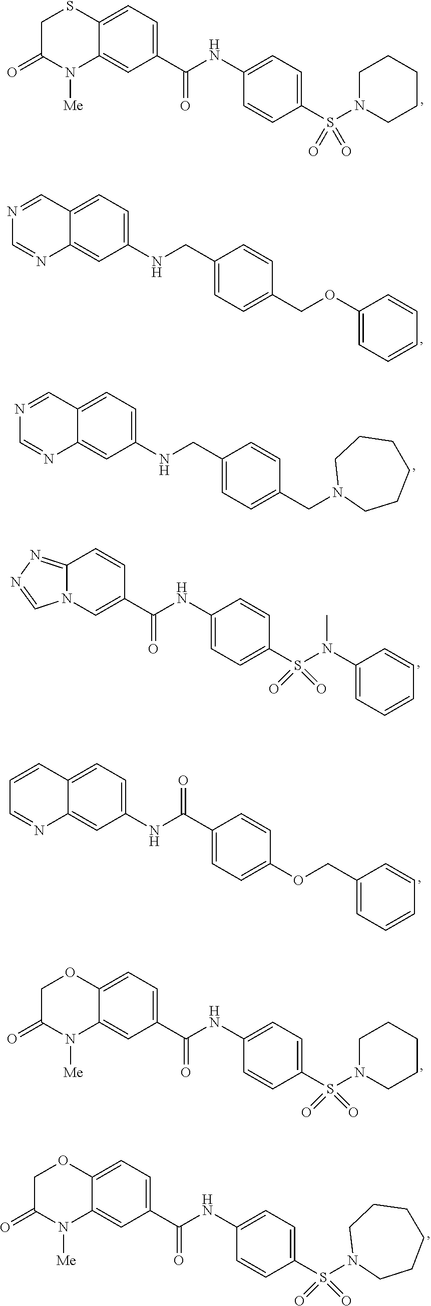

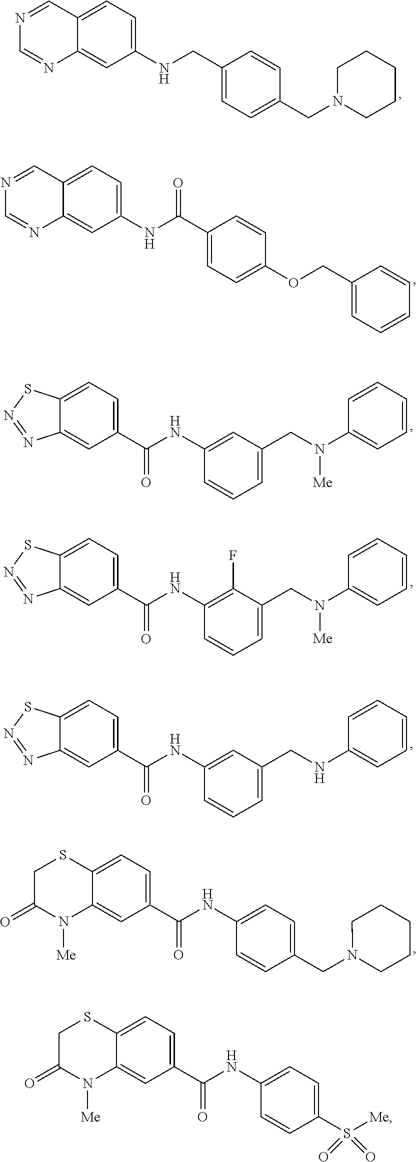

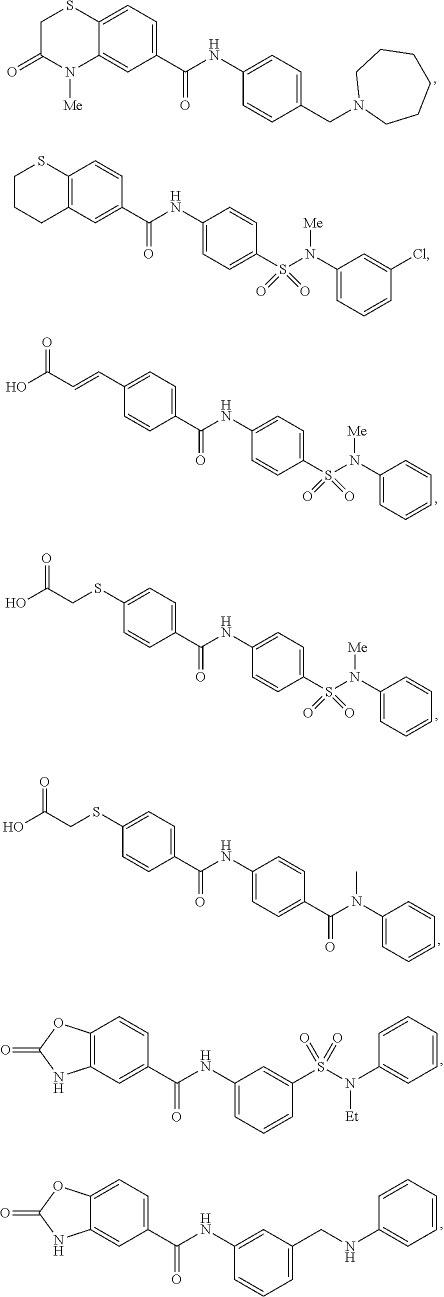

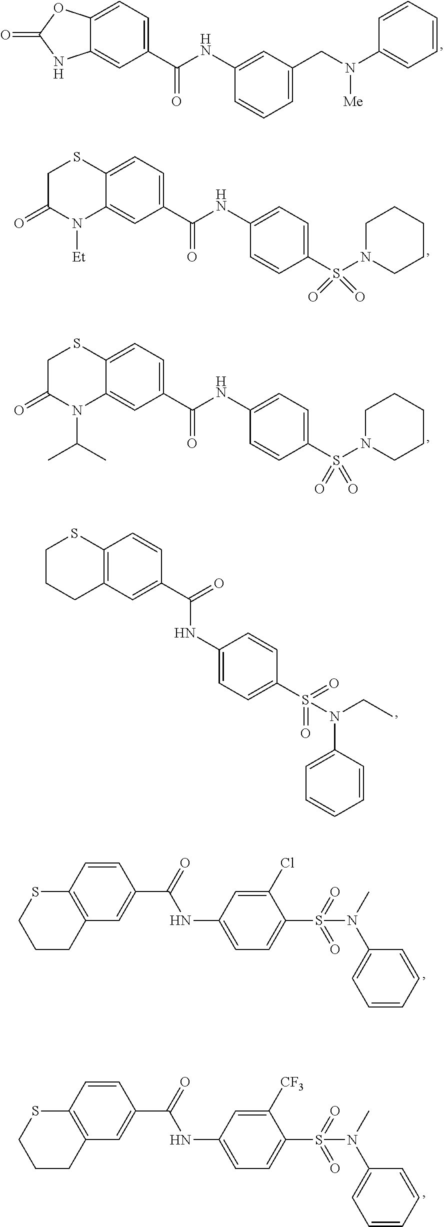 [SMILES: CN(Cc1cccc(NC(=O)c2ccc3oc(=O)[nH]c3c2)c1)c1ccccc1 ; CCN1C(=O)CSc2ccc(C(=O)Nc3ccc(S(=O)(=O)N4CCCCC4)cc3)cc21 ; CC(C)N1C(=O)CSc2ccc(C(=O)Nc3ccc(S(=O)(=O)N4CCCCC4)cc3)cc21 ; CCN(c1ccccc1)S(=O)(=O)c1ccc(NC(=O)c2ccc3c(c2)CCCS3)cc1 ; CN(c1ccccc1)S(=O)(=O)c1ccc(NC(=O)c2ccc3c(c2)CCCS3)cc1Cl ; CN(c1ccccc1)S(=O)(=O)c1ccc(NC(=O)c2ccc3c(c2)CCCS3)cc1C(F)(F)F]

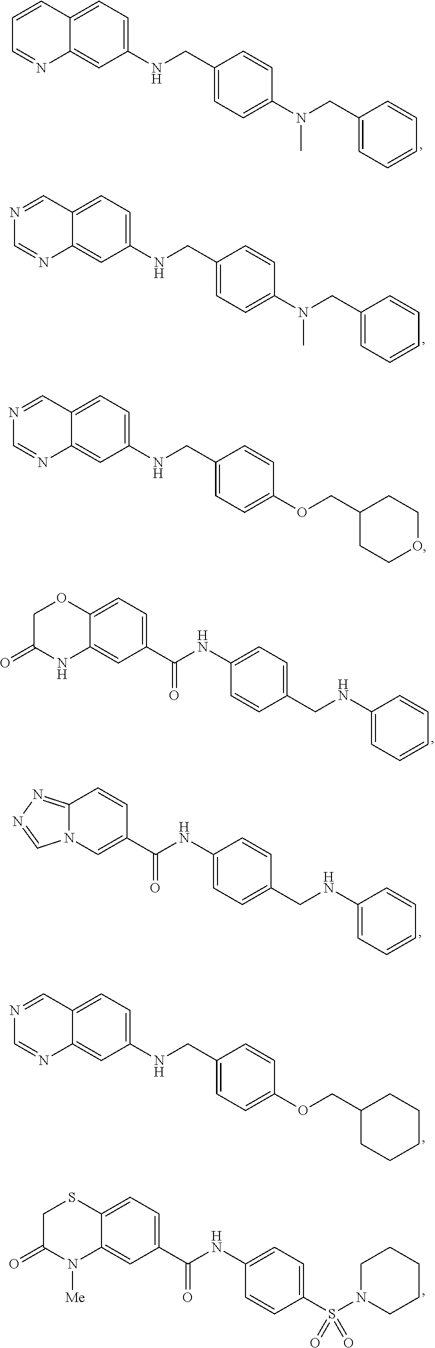

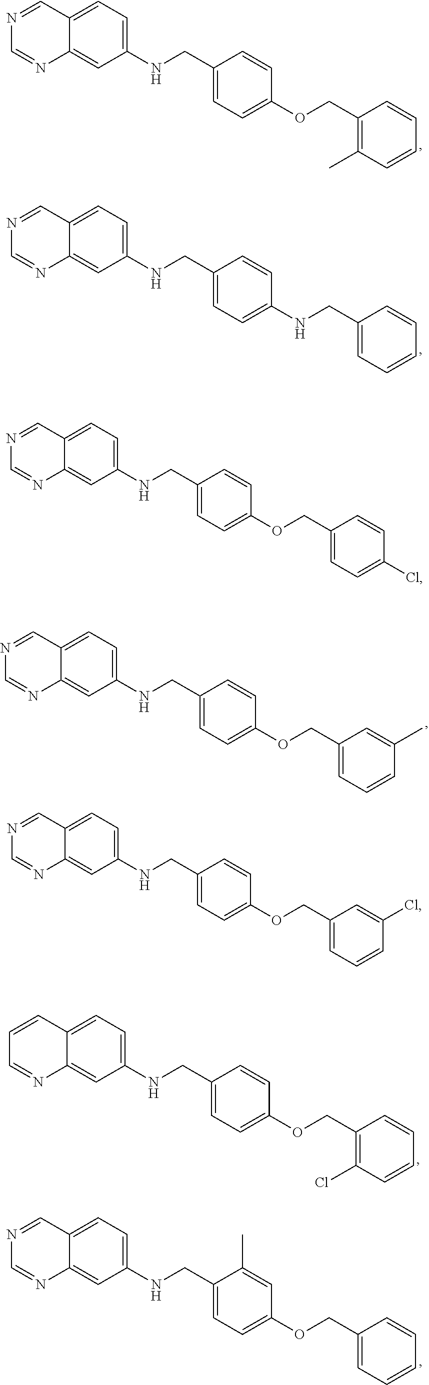

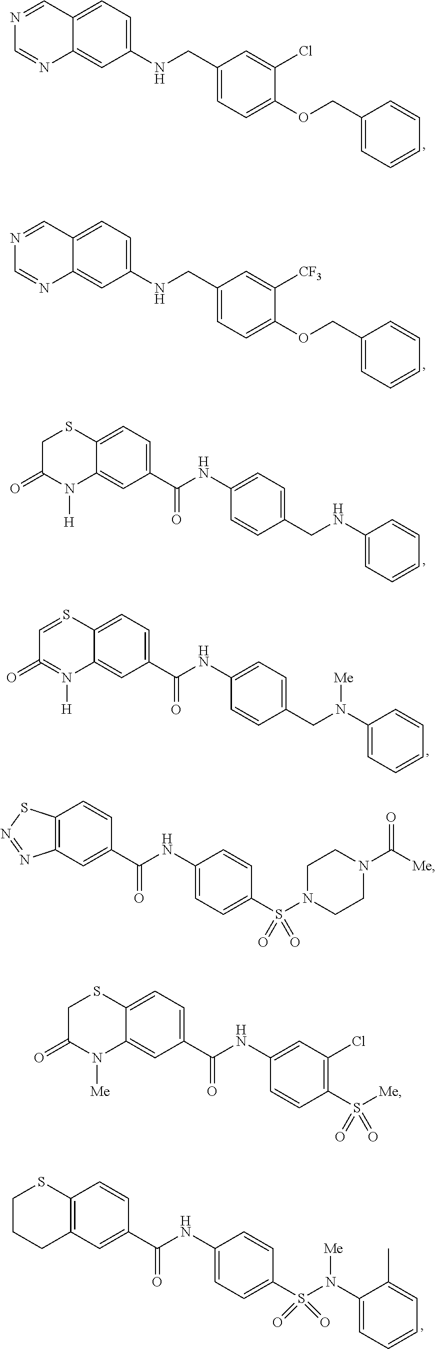

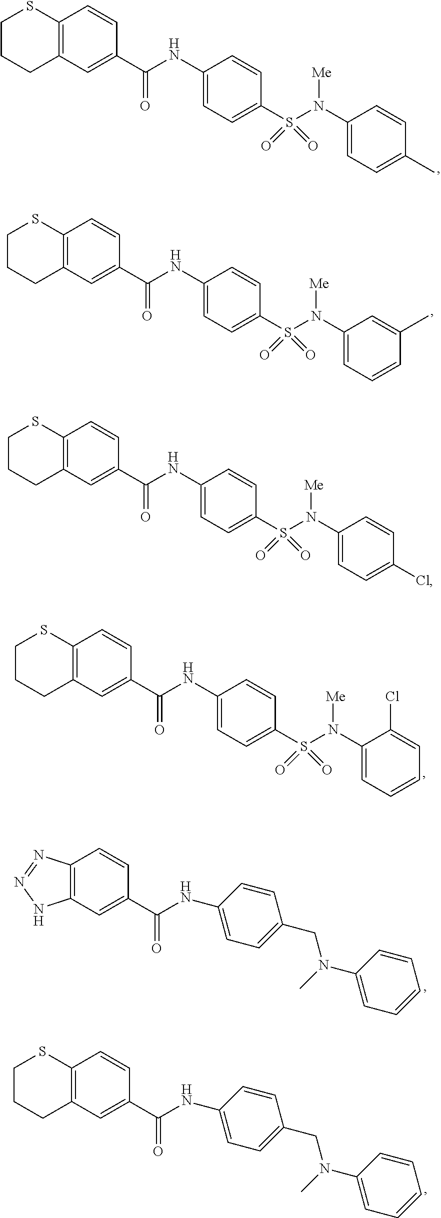 [SMILES: Cc1ccc(N(C)S(=O)(=O)c2ccc(NC(=O)c3ccc4c(c3)CCCS4)cc2)cc1 ; Cc1cccc(N(C)S(=O)(=O)c2ccc(NC(=O)c3ccc4c(c3)CCCS4)cc2)c1 ; CN(c1ccc(Cl)cc1)S(=O)(=O)c1ccc(NC(=O)c2ccc3c(c2)CCCS3)cc1 ; CN(c1ccccc1Cl)S(=O)(=O)c1ccc(NC(=O)c2ccc3c(c2)CCCS3)cc1 ; CN(Cc1ccc(NC(=O)c2ccc3nn[nH]c3c2)cc1)c1ccccc1 ; CN(Cc1ccc(NC(=O)c2ccc3c(c2)CCCS3)cc1)c1ccccc1]

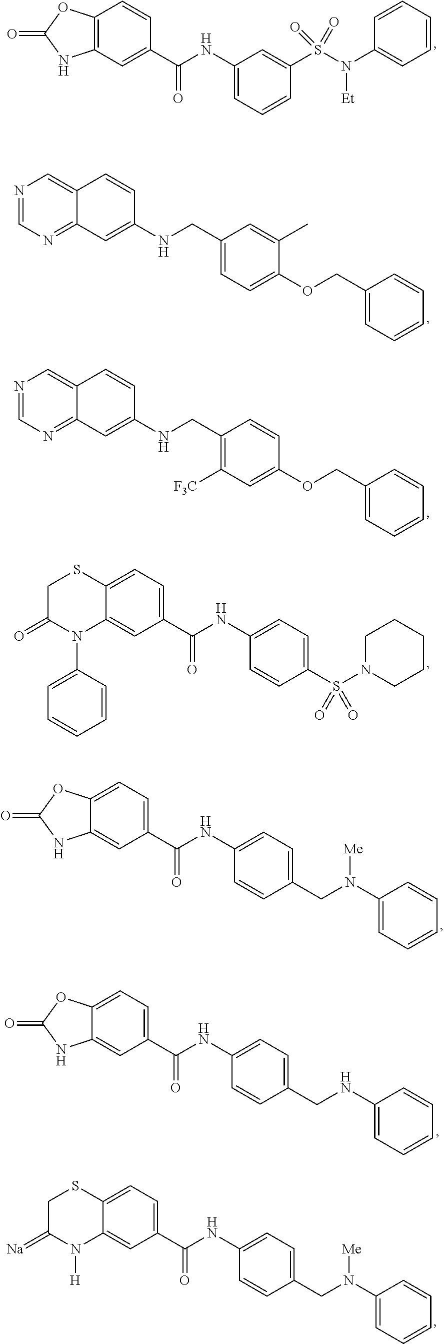

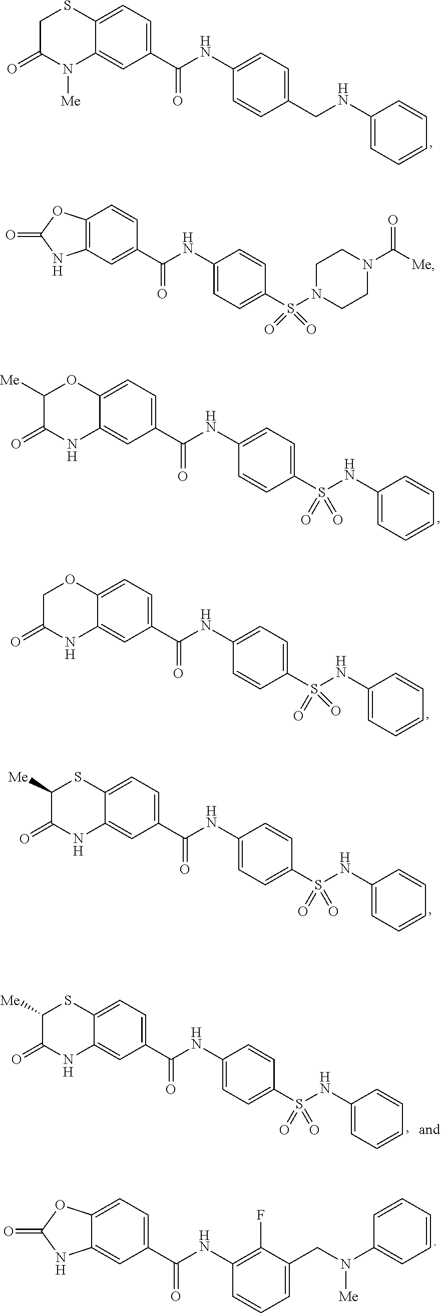

5. A pharmaceutical composition comprising a pharmaceutically acceptable carrier and a compound according to claim 1.

6. A method for treating cancer in a patient comprising administering to the patient a therapeutically effective amount of a compound according to claim 1.

7. The method according to claim 6, wherein said cancer is selected from mantle cell lymphoma, basal-cell like breast cancer, and neuroblastoma.

8. A method for treating a disease or disorder in a patient where the disease or disorder involves the inhibition of SOX-11, comprising administering to the patient a therapeutically effective amount of a compound according to claim 1.

9. A method for inhibiting SOX-11 expression, said method comprising bringing a compound according to claim 1 into contact with a SOX-11 receptor.

10. A compound according to claim 1, wherein
i) Ar¹ is 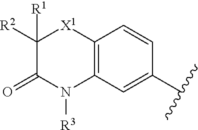
wherein:
X¹ is S or O;
R¹ and R² are independently selected from optionally substituted (C₁-C₆)alkyl and H; and
R³ is H or (C₁-C₁₀)hydrocarbyl;
L is —CONH—, —NHCO—, or —NHCH₂—; and
Ar² is a mono- or di-substituted monocyclic aryl or heteroaryl, wherein the substituents are selected from —SO₂—R¹⁰, —OSO₂—R¹⁰, perfluoro(C₁-C₃)alkyl, halo, (C₁-C₃)alkyl, —C(═O)R¹⁰, —OCH₂R¹¹, —OR¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰;
wherein R¹⁰ is selected from arylamino, perfluoro(C₁-C₃)alkyl-substituted arylamino, halo-substituted arylamino, (C₁-C₃)alkyl-substituted arylamino, amino, (C₁-C₃)alkyl, heterocyclyl, (C₁-C₆)dialkylamino, pyridylamino, (C₁-C₆)alkylamino, (C₁-C₆)cycloalkylamino, arylamino, oxo-substituted heteroarylamino, heterocyclylamino, hydroxy-substituted arylamino, amino-substituted arylamino, pyridin-2(1H)-one-amino, (C₁-C₆)dihydrocarbylamino, fluoro, (C₁-C₃)alkylarylamino, acetyl-substituted heterocyclyl, and (C₁-C₃)alkylhaloarylamino; and
wherein R¹¹ is selected from optionally substituted aryl, unsubstituted benzyl, perfluoro(C₁-C₃)alkyl-substituted benzyl, halo-substituted benzyl, (C₁-C₃)alkyl-substituted benzyl, (C₁-C₃)alkyl, heterocyclyl, (C₁-C₆)dialkyl, pyridyl, (C₁-C₆)alkyl, (C₁-C₆)cycloalkyl, benzyl, oxo-substituted heteroarylbenzyl, heterocyclyl, hydroxy-substituted benzyl, amino-substituted benzyl, pyridin-2(1H)-one, (C₁-C₆)dihydrocarbyl, (C₁-C₃)alkylbenzyl, acetyl-substituted heterocyclyl, and (C₁-C₃)alkylhalobenzyl.

11. A compound according to claim 1, wherein
ii) Ar¹ is 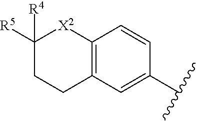
wherein:
X² is S or O; and
R⁴ and R⁵ are independently selected from H and (C₁-C₆)alkyl;
L is —CONH— or —NHCO—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰;
wherein R¹⁰ is selected from arylamino, perfluoro(C₁-C₃)alkyl-substituted arylamino, halo-substituted arylamino, (C₁-C₃)alkyl-substituted arylamino, amino, (C₁-C₃)alkyl, heterocyclyl, (C₁-C₆)dialkylamino, pyridylamino, (C₁-C₆)alkylamino, (C₁-C₆)cycloalkylamino, arylamino, oxo-substituted heteroarylamino, heterocyclylamino, hydroxy-substituted arylamino, amino-substituted arylamino, pyridin-2(1H)-one-amino, (C₁-C₆)dihydrocarbylamino, fluoro, (C₁-C₃)alkylarylamino, acetyl-substituted heterocyclyl, and (C₁-C₃)alkylhaloarylamino; and
wherein R¹¹ is selected from benzyl, perfluoro(C₁-C₃)alkyl-substituted benzyl, halo-substituted benzyl, (C₁-C₃)alkyl-substituted benzyl, (C₁-C₃)alkyl, heterocyclyl, (C₁-C₆)dialkyl, pyridyl, (C₁-C₆)alkyl, (C₁-C₆)cycloalkyl, benzyl, oxo-substituted heteroarylbenzyl, heterocyclyl, hydroxy-substituted benzyl, amino-substituted benzyl, pyridin-2(1H)-one, (C₁-C₆)dihydrocarbyl, (C₁-C₃)alkylbenzyl, acetyl-substituted heterocyclyl, and (C₁-C₃)alkylhalobenzyl.

12. A compound according to claim 1, wherein
iii) Ar¹ is 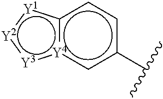
wherein:
all backbone atoms of the 6,5-bicyclic structure are sp²-hybridized;
Y¹ is selected from S, CH, N, NH, and O;
Y² is selected from N, NH, C—R⁶, and C═O; wherein R⁶ is H, (C₁-C₃)alkyl, or amino(C₁-C₃)alkyl;
Y³ is selected from N, NH, CH, and C—CH₃; and
Y⁴ is C or N;
L is —CONH— or —NHCO—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰.

13. A compound according to claim 1, wherein
iv) Ar¹ is 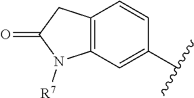
wherein R⁷ is H or optionally substituted (C₁-C₃)alkyl;
L is —CONH— or —NHCO—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰, or

14. A compound according to claim 1, wherein
v) Ar¹ is 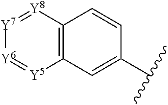
wherein Y⁵, Y⁶, Y⁷, and Y⁸ are independently chosen from C and N;
L is —CONH—, —NHCO—, or —NHCH₂—; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰.

15. A compound according to claim 1, wherein
vi) Ar¹ is 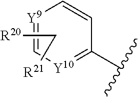
wherein Y⁹ and Y¹⁰ are independently chosen from C and N;
R²⁰ and R²¹ are independently chosen from hydrogen, (C₁-C₃)alkyl, amino, aryl-substituted heterocyclic amino, heteroaryl-substituted heterocyclic amino, unsubstituted heterocyclic amino, —CH═CHCOOH, 4-aminocyclohexylamino, acetylethylenediamino, amino- and/or (C₁-C₃)alkyl-substituted heterocyclic amino, —NHC(═O)(CH₂)ₙ-heterocyclyl wherein n is either 1 or 2, ethylenediamino, (C₁-C₃)alkoxy, and acetylmethylamino;
L is —CONH—, 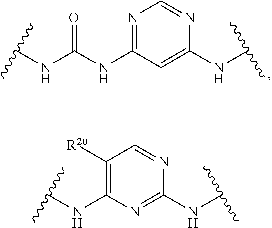
wherein R²⁰ is H or methyl; and
Ar² is a mono- or di-substituted monocyclic aryl or heteroaryl, wherein the substituents are selected from —SO₂—R¹⁰, perfluoro(C₁-C₃)alkyl, halo, (C₁-C₃)alkyl, —C(═O)R¹⁰, —OCH₂R¹¹, arylamino(C₁-C₃)alkyl, amino(C₁-C₃)alkyl, (C₁-C₆)alkylamino(C₁-C₃)alkyl, (C₁-C₆)dialkylamino(C₁-C₃)alkyl, (C₁-C₁₀)dihydrocarbylamino(C₁-C₃)alkyl, and —CH₂R¹⁰.
